(19) United States
(12) Patent Application Publication
(10) Pub. No.: US 2026/0167719 A1
(43) Pub. Date: Jun. 18, 2026

(54) NECTIN-4 ANTIBODIES

(71) Applicant: Merck Sharp & Dohme LLC, Rahway, NJ (US)

(72) Inventors: Andrew B. Waight, Belmont, CA (US); Mehabaw Getahun Derebe, Burlingame, CA (US); Daniel Malashock, Santa Clara, CA (US); Karin Vroom, San Mateo, CA (US); Sandra Isabel Vanegas, Fremont, CA (US); David Alan Campbell, San Diego, CA (US); Thomas Robert DiRaimondo, San Diego, CA (US)

(73) Assignee: Merck Sharp & Dohme LLC, Rahway, NJ (US)

(21) Appl. No.: 19/419,547

(22) Filed: Dec. 15, 2025

Related U.S. Application Data
(60) Provisional application No. 63/734,998, filed on Dec. 17, 2024.

(51) Int. Cl. C07K 16/28; A61P 35/00

(52) U.S. Cl. CPC C07K 16/2803; A61P 35/00; C07K 2317/14; C07K 2317/24; C07K 2317/565; C07K 2317/73

(57) ABSTRACT

Disclosed herein are Nectin-4 antibodies. In some embodiments, the Nectin-4 antibodies can be used in treatments such as, for example, cancer treatments.

### CROSS-REFERENCE TO RELATED APPLICATIONS

This application claims the benefit of U.S. Provisional Patent Application Ser. No. 63/734,998, filed Dec. 17, 2024, the entire contents of which are incorporated by reference herein.

### REFERENCE TO SEQUENCE LISTING SUBMITTED ELECTRONICALLY

The contents of the electronic sequence listing (26127-WO-PCT_SL.xml; Size: 220,949 bytes, created on Jan. 24, 2025) are herein incorporated by reference in their entirety.

### FIELD

This disclosure relates generally to Nectin-4 antibodies and methods of using the antibodies.

### BACKGROUND

Nectin-4 protein is cell surface protein that functions in cell-cell adhesion. Nectin-4 has high expression in embryonic tissue, but its expression level is low in healthy adult tissue. However, several studies have shown that Nectin-4 is overexpressed in several malignant tumors and several cancer types such as breast cancer, lung cancer, colorectal cancer, pancreatic cancer, and ovarian cancer. Studies have also shown that Nectin-4 has various roles in the progression of tumors including metastasis, DNA repair, and angiogenesis. Nectin-4 is expressed on a number of different cancer cells. As a result, Nectin-4 can function as a target for the development of therapies that treat cancer.

### SUMMARY

The present disclosure provide antibodies that bind to Nectin-4.

In an aspect, an antibody or antigen binding fragment thereof that binds to Nectin-4 is provided, comprising:
(a) a heavy chain variable region comprising: a CDRH1 comprising the amino acid sequence of SEQ ID NO: 15; a CDRH2 comprising the amino acid sequence of SEQ ID NO: 16; and a CDRH3 comprising the amino acid sequence of SEQ ID NO: 17; and a light chain variable comprising: a CDR1-L1 comprising the amino acid sequence of SEQ ID NO: 20; a CDR1-L2 comprising the amino acid sequence of SEQ ID NO: 21; and a CDR1-L3 comprising the amino acid sequence of SEQ ID NO: 22;
(b) a heavy chain variable region comprising: a CDRH1 comprising the amino acid sequence of SEQ ID NO: 52; a CDR1-H2 comprising the amino acid sequence of SEQ ID NO: 53; and a CDRH3 comprising the amino acid sequence of SEQ ID NO: 54; and a light chain variable comprising: a CDRL1 comprising the amino acid sequence of SEQ ID NO: 28; a CDRL2 comprising the amino acid sequence of SEQ ID NO: 29; and a CDRL3 comprising the amino acid sequence of SEQ ID NO: 30;
(c) a heavy chain variable region comprising: a CDRH1 comprising the amino acid sequence of SEQ ID NO: 59; a CDR1-H2 comprising the amino acid sequence of SEQ ID NO: 60; and a CDRH3 comprising the amino acid sequence of SEQ ID NO: 61; and a light chain variable comprising: a CDRL1 comprising the amino acid sequence of SEQ ID NO: 35; a CDRL2 comprising the amino acid sequence of SEQ ID NO: 36; and a CDRL3 comprising the amino acid sequence of SEQ ID NO: 37; or
(d) a heavy chain variable region comprising: a CDRH1 comprising the amino acid sequence of SEQ ID NO: 66; a CDR1-H2 comprising the amino acid sequence of SEQ ID NO: 67; and a CDRH3 comprising the amino acid sequence of SEQ ID NO: 68; and a light chain variable comprising: a CDRL1 comprising the amino acid sequence of SEQ ID NO: 42; a CDRL2 comprising the amino acid sequence of SEQ ID NO: 29; and a CDRL3 comprising the amino acid sequence of SEQ ID NO: 30.

In some embodiments, the light chain variable region has at least 90% amino acid sequence identity to a V_{L} of any one of SEQ ID NOs: 43, 44, 45, 46, and 47, and the heavy chain variable region has at least 90% amino acid sequence identity to a V_{H} domain of any one of SEQ ID NOs: 69, 70, 71, and 72, wherein the antibody or antibody fragment comprising the light chain variable region and the heavy chain variable region binds Nectin-4.

In some embodiments, light chain variable region has at least 90% amino acid sequence identity to a V_{L} domain of any one of SEQ ID NOs: 24, 25, 26, and 27, and the heavy chain variable region has at least 90% amino acid sequence identity to a V_{H} domain of any one of SEQ ID NOs: 48, 49, 50, and 51, wherein the antibody or antibody fragment comprising the light chain variable region and the heavy chain variable region binds Nectin-4.

In some embodiments, the light chain variable region has at least 90% amino acid sequence identity to a V_{L} domain of any one of SEQ ID NOs: 31, 32, 33, and 34, and the heavy chain variable region has at least 90% amino acid sequence identity to a V_{H} domain of any one of SEQ ID NOs: 55, 56, 57, and 58, wherein the antibody or antibody fragment comprising the light chain variable region and the heavy chain variable region binds Nectin-4.

In some embodiments, the light chain variable region has at least 90% amino acid sequence identity to a V_{L} domain of any one of SEQ ID NOs: 38, 39, 40, and 41, and the heavy chain variable region has at least 90% amino acid sequence identity to a V_{H} domain of any one of SEQ ID NOs: 62, 63, 64, and 65, wherein the antibody or antibody fragment comprising the light chain variable region and the heavy chain variable region binds Nectin-4.

In an aspect, an antibody or antigen binding fragment thereof that binds to Nectin-4 is provided, comprising:
(a) a V_{L} domain comprising the amino acid sequence set forth in any one of SEQ ID NOs: 43, 44, 45, 46, and 47 and a V_{H} domain comprising the amino acid sequence set forth in any one of SEQ ID NOs: 69, 70, 71, and 72;
(b) a V_{L} domain comprising the amino acid sequence set forth in any one of SEQ ID NOs: 24, 25, 26, and 27 and a V_{H} domain comprising the amino acid sequence set forth in any one of SEQ ID NOs: 48, 49, 50, and 51;
(c) a V_{L} domain comprising the amino acid sequence set forth in any one of SEQ ID NOs: 31, 32, 33, and 34 and a V_{H} domain comprising the amino acid sequence set forth in any one of SEQ ID NOs: 55, 56, 57, and 58; or
(d) a V_{L} domain comprising the amino acid sequence set forth in any one of SEQ ID NOs: 38, 39, 40, and 41 and a V_{H} domain comprising the amino acid sequence set forth in any one of SEQ ID NOs: 62, 63, 64, and 65.

In an aspect, an antibody that binds to Nectin-4 is provided, comprising:
(a) a light chain having at least 90% sequence identity to any one of SEQ ID NOs: 139, 140, 141, 142, and 143, and a heavy chain having at least 90% sequence identity to any one of SEQ ID NOs: 156, 157, 158, and 159;
(b) a light chain having at least 90% sequence identity to any one of SEQ ID NOs: 127, 128, 129, and 130 and a heavy chain having at least 90% sequence identity to any one of SEQ ID NOs: 144, 145, 146, and 147;
(c) a light chain having at least 90% sequence identity to any one of SEQ ID NOs: 131, 132, 133, and 134 and a heavy chain having at least 90% sequence identity to any one of SEQ ID NOs: 148, 149, 150, and 151; or
(d) a light chain having at least 90% sequence identity to any one of SEQ ID NOs: 135, 136, 137, and 138 and a heavy chain having at least 90% sequence identity to any one of SEQ ID NOs: 152, 153, 154, and 155.

In an aspect, an antibody that binds to Nectin-4 is provided, consisting of:
(a) two light chains having at least 90% sequence identity to any one of SEQ ID NOs: 139, 140, 141, 142, and 143, and two heavy chains having at least 90% sequence identity to any one of SEQ ID NOs: 156, 157, 158, and 159;
(b) two light chains having at least 90% sequence identity to any one of SEQ ID NOs: 127, 128, 129, and 130 and two heavy chains having at least 90% sequence identity to any one of SEQ ID NOs: 144, 145, 146, and 147;
(c) two light chains having at least 90% sequence identity to any one of SEQ ID NOs: 131, 132, 133, and 134 and two heavy chains having at least 90% sequence identity to any one of SEQ ID NOs: 148, 149, 150, and 151; or
(d) two light chains having at least 90% sequence identity to any one of SEQ ID NOs: 135, 136, 137, and 138 and two heavy chains having at least 90% sequence identity to any one of SEQ ID NOs: 152, 153, 154, and 155.

In an aspect, an antibody is provided consisting of:
(a) two light chains consisting of the amino acid sequence set forth in any one of SEQ ID NOs: 139, 140, 141, 142, and 143 and two heavy chains consisting of the amino acid sequence set forth in any one of SEQ ID NOs: 156, 157, 158, and 159;
(b) two light chains consisting of the amino acid sequence set forth in any one of SEQ ID NOs: 127, 128, 129, and 130 and two heavy chains consisting of the amino acid sequence set forth in any one of SEQ ID NOs: 144, 145, 146, and 147;
(c) two light chains consisting of the amino acid sequence set forth in any one of SEQ ID NOs: 131, 132, 133, and 134 and two heavy chains consisting of the amino acid sequence set forth in of any one of SEQ ID NOs: 148, 149, 150, and 151; or
(d) two light chains consisting of the amino acid sequence set forth in any one of SEQ ID NOs: 135, 136, 137, and 138 and two heavy chains consisting of the amino acid sequence set forth in any one of SEQ ID NOs: 152, 153, 154, and 155.

In an aspect, a polypeptide is provided comprising the V_{L} or the V_{H} domains of any one of the antibodies or antigen binding fragments described herein.

In an aspect, an isolated nucleic acid is provided, wherein the isolated nucleic acid encodes the V_{L} domains or the V_{H} domains, or the V_{L} and the V_{H} domains of any one of the antibodies or antigen binding fragments described herein.

In an aspect, an expression vector is provided, wherein the expression vector comprises any one of the polynucleotides described herein.

In an aspect, a host cell is provided, wherein the host cell comprises any of the isolated nucleic acids or any of the expression vectors described herein.

In an aspect, a composition is provided wherein the composition comprises any of the antibodies or antigen binding fragments or any of the polynucleotides described herein, and a pharmaceutically acceptable carrier.

In an aspect, a method of treating cancer in a subject in need thereof is provided, comprising administering to the subject an effective amount any of the antibodies or antigen binding fragments described herein.

In an aspect, a method of producing any of the antibodies or antigen binding fragments described herein is provided, the method comprising:
(a) culturing a host cell in culture medium, wherein the host cell comprises one or more vectors comprising a polynucleotide encoding any one of the V_{L} domains described herein and any one of the V_{H} domains described herein, under conditions that allow expression of the V_{L} domain and V_{H} domain, and
(b) optionally, recovering the antibody or antigen binding fragment from the host cell or the culture medium. In some embodiments, the host cell comprises a single vector comprising a polynucleotide encoding any one of the V_{L} domains and any one of the V_{H} domains described herein.

The summary of the technology described above is non-limiting and other features and advantages of the technology will be apparent from the following detailed description, and from the claims.

### BRIEF DESCRIPTION OF THE DRAWINGS

FIG. 1 provides fluorescence-activated cell sorting (FACS) data showing purified rat antibodies that target Nectin-4 (See Example 2).

FIGS. 2A and 2B provide binding data of purified rat antibodies to Nectin-4 (See Example 2).

FIGS. 3A-3D, 4A-4D, and 5A-5D provide data showing in vitro cytotoxicity on T cells, when the cells are treated with the Nectin-4 TRACTr molecule (See Example 9).

FIGS. 6A and 6B provide data comparing in vitro cytotoxicity between (i) T cells and (ii) Nectin-4 knockout T cells, after treatment with the Nectin-4 TRACTr molecule (See Example 9).

FIGS. 7A-7D provide data showing in vitro cytotoxicity on peripheral blood mononuclear cells (PBMCs), when the cells are treated with the Nectin-4 TRACTr molecule (See Example 9).

FIGS. 8A-8D and 9A-9D provide data showing cytokine concentration from TDCC supernatants after treatment with the TRACTr molecule (See Example 10).

FIGS. 10A-10D, 11A-11C, and 12A-12C provide data showing in vitro cytotoxicity on PBMCs, when the cells are treated with the Nectin-4 TRACTr molecule (See Example 9).

### DETAILED DESCRIPTION

The present disclosure is directed to Nectin-4 antibodies. The present disclosure is also directed to methods of making and using the Nectin-4 antibodies.

### Definitions

Listed below are definitions of various terms used herein. These definitions apply to the terms as they are used throughout this specification and claims, unless otherwise limited in specific instances, either individually or as part of a larger group.

Unless defined otherwise, all technical and scientific terms used herein have the same meaning as commonly understood by one of ordinary skill in the art. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “about” in quantitative terms refers to plus or minus 10% of the value it modifies (rounded up to the nearest whole number if the value is not sub-dividable, such as a number of molecules or nucleotides).

All ranges disclosed herein are inclusive of the recited endpoint and independently combinable (for example, the range of “from 50 mg to 500 mg” is inclusive of the endpoints, 50 mg and 500 mg, and all the intermediate values). The endpoints of the ranges and any values disclosed herein are not limited to the precise range or value; they are sufficiently imprecise to include values approximating these ranges and/or values.

As used herein, the term “comprising” may include the embodiments “consisting of” and “consisting essentially of.” The terms “comprise(s),” “include(s),” “having,” “has,” “may,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated components, which allows the presence of only the named components or compounds, along with any acceptable carriers or fluids, and excludes other components or compounds.

“Activity” of a molecule may describe or refer to the binding of the molecule to a ligand or to a receptor, to catalytic activity; to the ability to stimulate gene expression or cell signaling, differentiation, or maturation; to antigenic activity, to the modulation of activities of other molecules, and the like. “Activity” of a molecule may also refer to activity in modulating or maintaining cell-to-cell interactions, e.g., adhesion, or activity in maintaining a structure of a cell, e.g., cell membranes or cytoskeleton. “Activity” can also mean specific activity, e.g., [catalytic activity]/[mg protein], or [immunological activity]/[mg protein], concentration in a biological compartment, or the like. “Activity” may refer to modulation of components of the innate or the adaptive immune systems.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including KinExA and Biacore. Specific illustrative and exemplary embodiments for measuring binding affinity are described in the following.

“Administration” or “treatment,” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. “Administration” or “treatment” also can be performed in vitro and ex vivo. The term “subject” includes any organism, preferably an animal, more preferably a mammal (e.g., human, rat, mouse, dog, cat, rabbit). In an embodiment, the term “subject” refers to a human.

As used herein, the term “antibody,” “immunoglobulin,” or “Ig,” refers to any form of antibody that exhibits the desired biological activity. Thus, it is used in the broadest sense and specifically covers, but is not limited to, monoclonal antibodies (including full length monoclonal antibodies), polyclonal antibodies, multispecific antibodies (e.g., bispecific antibodies), humanized, fully human antibodies, and chimeric antibodies.

As used herein, unless otherwise indicated, “antigen binding fragment,” “antigen binding domain,” or “antigen binding region” refers to the portion of antibodies, i.e., antibody fragments that retain the ability to bind specifically to the antigen bound by the full-length antibody, e.g., fragments that retain one or more CDR regions. Examples of antibody binding fragments include, but are not limited to, Fab, Fab′, F(ab′)₂, and Fv fragments.

A “Fab fragment” is comprised of one light chain and the C_{H}1 and variable regions of one heavy chain. The heavy chain of a Fab molecule cannot form a disulfide bond with another heavy chain molecule. An “Fab fragment” can be the product of papain cleavage of an antibody.

An “Fc” region contains two heavy chain fragments comprising the C_{H}1 and C_{H}2 domains of an antibody. The two heavy chain fragments are held together by two or more disulfide bonds and by hydrophobic interactions of the C_{H}3 domains.

A “Fab′ fragment” contains one light chain and a portion or fragment of one heavy chain that contains the V_{H} domain and the C_{H}1 domain and also the region between the C_{H}1 and C_{H}2 domains, such that an interchain disulfide bond can be formed between the two heavy chains of two Fab′ fragments to form a F(ab′)₂ molecule.

A “F(ab′)₂ fragment” contains two light chains and two heavy chains containing a portion of the constant region between the C_{H}1 and C_{H}2 domains, such that an interchain disulfide bond is formed between the two heavy chains. A F(ab′)₂ fragment thus is composed of two Fab′ fragments that are held together by a disulfide bond between the two heavy chains. An “F(ab′)₂ fragment” can be the product of pepsin cleavage of an antibody.

An “Fv fragment” or “Fv region” comprises the variable regions from both the heavy and light chains, but lacks the constant regions.

“Isolated” antibodies or antigen-binding fragments thereof are at least partially free of other biological molecules from the cells or cell cultures in which they are produced. Such biological molecules include nucleic acids, proteins, lipids, carbohydrates, or other material such as cellular debris and growth medium. An isolated antibody or antigen-binding fragment may further be at least partially free of expression system components such as biological molecules from a host cell or of the growth medium thereof. Generally, the term “isolated” is not intended to refer to a complete absence of such biological molecules or to an absence of water, buffers, or salts or to components of a pharmaceutical formulation that includes the antibodies or fragments.

The term “monoclonal antibody,” as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains that are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256:495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352:624-628 and Marks et al. (1991) J. Mol. Biol. 222:581-597, for example. See also Presta (2005) J. Allergy Clin. Immunol. 116:731.

As used herein, a “chimeric antibody” is an antibody having the variable domain from a first antibody and the constant domain from a second antibody, where the first and second antibodies are from different species. (U.S. Pat. No. 4,816,567; and Morrison et al., (1984) Proc. Natl. Acad. Sci. USA 81:6851-6855). Typically the variable domains are obtained from an antibody from an experimental animal (the “parental antibody”), such as a rodent, and the constant domain sequences are obtained from human antibodies, so that the resulting chimeric antibody will be less likely to elicit an adverse immune response in a human subject than the parental (e.g., rodent) antibody.

As used herein, the term “humanized antibody” refers to forms of antibodies that contain sequences from both human and non-human (e.g., murine, rat) antibodies. In general, the humanized antibody will comprise substantially all of at least one, and typically two, variable domains, in which all or substantially all of the hypervariable loops correspond to those of a non-human immunoglobulin, and all or substantially all of the framework (FR) regions are those of a human immunoglobulin sequence. The humanized antibody may optionally comprise at least a portion of a human immunoglobulin constant region (Fc).

The term “fully human antibody” or “human antibody” refers to an antibody that comprises human immunoglobulin protein sequences only. A fully human antibody may contain murine carbohydrate chains if produced in a mouse, in a mouse cell, or in a hybridoma derived from a mouse cell. Similarly, “mouse antibody” refers to an antibody that comprises mouse immunoglobulin sequences only. Alternatively, a fully human antibody may contain rat carbohydrate chains if produced in a rat, in a rat cell, or in a hybridoma derived from a rat cell. Similarly, “rat antibody” refers to an antibody that comprises rat immunoglobulin sequences only.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation.

Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989).

“Variable regions” or “V region” or “V chain” as used herein means the segment of IgG chains which is variable in sequence between different antibodies. A “variable region” of an antibody refers to the variable region of the antibody light chain or the variable region of the antibody heavy chain, either alone or in combination. The variable region of the heavy chain may be referred to as “V_{H}.” The variable region of the light chain may be referred to as “V_{L}.” Typically, the variable regions of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3, and FR4. The assignment of amino acids to each domain is, generally, in accordance with the definitions of Sequences of Proteins of Immunological Interest, Kabat, et al.; National Institutes of Health, Bethesda, Md.; 5th ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

A “CDR” refers to one of three hypervariable regions (H1, H2, or H3) within the non-framework region of the antibody V_{H} β-sheet framework, or one of three hypervariable regions (L1, L2, or L3) within the non-framework region of the antibody V_{L} β-sheet framework. Accordingly, CDRs are variable region sequences interspersed within the framework region sequences. CDR regions are well known to those skilled in the art and have been defined by, for example, Kabat as the regions of most hypervariability within the antibody variable domains. CDR region sequences also have been defined structurally by Chothia as those residues that are not part of the conserved b-sheet framework, and thus are able to adapt to different conformation. Both terminologies are well recognized in the art. CDR region sequences have also been defined by AbM, Contact, and IMGT. The positions of CDRs within a canonical antibody variable region have been determined by comparison of numerous structures (Al-Lazikani et al., 1997, J. Mol. Biol. 273:927-48; Morea et al., 2000, Methods 20:267-79). Because the number of residues within a hypervariable region varies in different antibodies, additional residues relative to the canonical positions are conventionally numbered with a, b, c and so forth next to the residue number in the canonical variable region numbering scheme (Al-Lazikani et al., supra). Such nomenclature is similarly well known to those skilled in the art. Correspondence between the numbering system, including, for example, the Kabat numbering and the IMGT unique numbering system, is well known to one skilled in the art and shown below in Table 1. In some embodiments, the CDRs are as defined by the Kabat numbering system. In other embodiments, the CDRs are as defined by the IMGT numbering system. In yet other embodiments, the CDRs are as defined by the AbM numbering system. In still other embodiments, the CDRs are as defined by the Chothia numbering system. In yet other embodiments, the CDRs are as defined by the Contact numbering system.

TABLE 1
Correspondence between the CDR Numbering Systems

| | Kabat + | | | | | |
|---|---|---|---|---|---|---|
| | Chothia | IMGT | Kabat | AbM | Chothia | Contact |
| V_{H} CDR1 | 26-35 | 27-38 | 31-35 | 26-35 | 26-32 | 30-35 |
| V_{H} CDR2 | 50-65 | 56-65 | 50-65 | 50-58 | 52-56 | 47-58 |
| V_{H} CDR3 | 95-102 | 105-117 | 95-102 | 95-102 | 95-102 | 93-101 |
| V_{L} CDR1 | 24-34 | 27-38 | 24-34 | 24-34 | 24-34 | 30-36 |
| V_{L} CDR2 | 50-56 | 56-65 | 50-56 | 50-56 | 50-56 | 46-55 |
| V_{L} CDR3 | 89-97 | 105-117 | 89-97 | 89-97 | 89-97 | 89-96 |

“Conservatively modified variants” or “conservative substitution” refers to substitutions of amino acids in a protein with other amino acids having similar characteristics (e.g., charge, side-chain size, hydrophobicity/hydrophilicity, backbone conformation and rigidity, etc.), such that the changes can frequently be made without altering the biological activity of the protein. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 2.

TABLE 2
Exemplary Conservative Amino Acid Substitutions

| Original | Conservative |
|---|---|
| residue | substitution |
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

Epitope and antigen: The term “epitope”, as used herein, is defined in the context of a molecular interaction between an “antigen binding molecule”, such as an antibody (Ab), and its corresponding “antigen” (Ag). Generally, “epitope” refers to the area or region on an Ag to which an Ab specifically binds, i.e., the area or region in physical contact with the Ab. Physical contact may be defined through distance criteria (e.g., a distance cut-off of 4 Å) for atoms in the Ab and Ag molecules.

The epitope for a given antibody (Ab)/antigen (Ag) pair can be defined and characterized at different levels of detail using a variety of experimental and computational epitope mapping methods. The experimental methods include mutagenesis, X-ray crystallography, Nuclear Magnetic Resonance (NMR) spectroscopy and Hydrogen deuterium exchange Mass Spectrometry (HX-MS), methods that are known in the art. As each method relies on a unique principle, the description of an epitope is intimately linked to the method by which it has been determined. Thus, depending on the epitope mapping method employed, the epitope for a given Ab/Ag pair will be described differently.

The epitope for a given antibody (Ab)/antigen (Ag) pair may be described by routine methods. For example, the overall location of an epitope may be determined by assessing the ability of an antibody to bind to different fragments or variants of the antigen. The specific amino acids within the antigen that make contact with an antibody (epitope) may also be determined using routine methods. For example, the Ab and Ag molecules may be combined and the Ab/Ag complex may be crystallized. The crystal structure of the complex may be determined and used to identify specific sites of interaction between the Ab and Ag.

“Treat” or “treatment” means to administer a therapeutic agent, such as a composition containing any of the antibodies or antigen binding fragments of the present invention, internally or externally to a subject or patient having one or more disease symptoms, or being suspected of having a disease, for which the agent has therapeutic activity. Typically, the agent is administered in an amount effective to alleviate one or more disease symptoms in the treated subject or population, whether by inducing the regression of or inhibiting, delaying or slowing the progression of such symptom(s) by any clinically measurable degree. The amount of a therapeutic agent that is effective to alleviate any particular disease symptom may vary according to factors such as the disease state, age, and weight of the patient, and the ability of the drug to elicit a desired response in the subject. Whether a disease symptom has been alleviated can be assessed by any clinical measurement typically used by physicians or other skilled healthcare providers to assess the severity or progression status of that symptom. The term further includes a postponement of development of the symptoms associated with a disorder and/or a reduction in the severity of the symptoms of such disorder. The terms further include ameliorating existing uncontrolled or unwanted symptoms, preventing additional symptoms, and ameliorating or preventing the underlying causes of such symptoms. Thus, the terms denote that a beneficial result has been conferred on a vertebrate subject with a disorder, disease or symptom, or with the potential to develop such a disorder, disease or symptom.

The terms “% identical”, “% identity” or similar terms are intended to refer, in particular to the percentage of nucleotides or amino acids which are identical in an optimal alignment between the sequences to be compared and introducing gaps, if necessary, to achieve maximum percent sequence identity. Said percentage is purely statistical, and the differences between the two sequences may be but are not necessarily randomly distributed over the entire length of the sequences to be compared. Comparisons of two sequences are usually carried out by comparing said sequences, after optimal alignment, with respect to a segment or “window of comparison”, in order to identify local regions of corresponding sequences. Two sequences can be optimally aligned for comparison even when the sequences are of different lengths, which includes optimally aligning sequences in which one sequence is truncated on either or both of its 5′ and 3′ ends relative to the sequence to which it is being aligned. The optimal alignment for a comparison may be carried out manually or with the aid of the local homology algorithm by Smith and Waterman, 1981, Ads App. Math. 2, 482, with the aid of the local homology algorithm by Needleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, and TFASTA in Wisconsin Genetics Software Package, Genetics Computer Group, 575 Science Drive, Madison, Wis).

As used herein, a “patient” (alternatively referred to herein as a “subject”) refers to a mammal capable of suffering from a disease or disorder and/or one or more symptoms that are associated with the disease or disorder. In embodiments, the patient is a human. A patient can be treated prophylactically or therapeutically. Those “in need of treatment” include those diagnosed with or suspected of having a disease or disorder and/or one or more symptoms that are associated with a disease or disorder, and those who were previously suffering from a disease or disorder, or one or more symptoms thereof, and any person in which prevention of recurrence, lessening in the number or severity of symptoms, or a delay of the progression, onset or reduction in the likelihood of progression or onset of a disease, disorder or the symptoms thereof is desired.

### Physical and Functional Properties of the Exemplary Anti-Nectin-4 Antibodies

In one aspect, the invention provides isolated anti-Nectin-4 antibodies and antigen binding fragments thereof. In one embodiment, the invention provides fully humanized anti-Nectin-4 antibodies and antigen binding fragments thereof.

In some embodiments, the invention provides antibodies or antigen binding fragments thereof that specifically bind to Nectin-4. In some embodiments, an antibody is provided that specifically binds to Nectin-4, or an antibody that specifically binds to a polypeptide comprising the amino acid sequence of Nectin-4.

In some embodiments, the anti-Nectin-4 antibodies or antigen binding fragments thereof are derived from any species. In some embodiments, the anti-Nectin-4 antibodies and anti-Nectin-4 antibody fragments are derived from a rat. In some embodiments, the anti-Nectin-4 antibodies and anti-Nectin-4 antibody fragments are derived from a mouse. In some embodiments, anti-Nectin-4 antibodies and anti-Nectin-4 antibody fragments are humanized using any technique known in the art that is used to humanize antibodies.

In some embodiments, disclosed herein are polypeptides comprising V_{L} domains comprising the amino acid sequence set forth in of any one of SEQ ID NOs: 24, 25, 26, 27, 31, 32, 33, 34, 38, 39, 40, 41, 43, 44, 45, 46, and 47, and polypeptides comprising the V_{H} domains comprising the amino acid sequence set forth in of any one of SEQ ID NOs: 48, 49, 50, 51, 55, 56, 57, 58, 62, 63, 64, 65, 69, 70, 71, and 72.

In some embodiments, disclosed herein are polypeptides comprising a light chain comprising the amino acid sequence set forth in of any one of SEQ ID NOs: 127, 128, 129, 130, 131, 132, 133, 134, 135, 136, 137, 138, 139, 140, 141, 142, and 143 or a heavy chain comprising the amino acid sequence set forth in of any one of SEQ ID NOs: 144, 145, 146, 147, 148, 149, 150, 151, 152, 153, 154, 155, 156, 157, 158, and 159.

In some embodiments, disclosed herein are antibodies or antigen binding fragments thereof that bind to Nectin-4 and have a V_{L} comprising an amino acid sequence having at least 90%, at least 91%, at least 92%, at least 93%, at least 94%, at least 95%, at least 96%, at least 97%, at least 98%, or at least 99% sequence identity with the amino acid sequence of any one of SEQ ID NOs: 24, 25, 26, 27, 31, 32, 33, 34, 38, 39, 40, 41, 43, 44, 45, 46, and 47, and a V_{H} comprising an amino acid sequence having at least 90%, at least 91%, at least 92%, at least 93%, at least 94%, at least 95%, at least 96%, at least 97%, at least 98%, or at least 99% sequence identity with the amino acid sequence of any one of SEQ ID NOs: 48, 49, 50, 51, 55, 56, 57, 58, 62, 63, 64, 65, 69, 70, 71, and 72.

In some embodiments, disclosed herein are antibodies or antigen binding fragments comprising a V_{L} domain having up to 0, 1, 2, 3, 4, 5, 6, 7, 8, 9, or 10 or more conservative or non-conservative amino acid substitutions relative to the amino acid sequence set forth in any one of SEQ ID NOs: 24, 25, 26, 27, 31, 32, 33, 34, 38, 39, 40, 41, 43, 44, 45, 46, and 47, wherein the antibody or antigen-binding fragment comprising the V_{L} domain binds Nectin-4. In some embodiments, disclosed herein are antibodies or antigen binding fragments comprising a V_{H} domain having up to 0, 1, 2, 3, 4, 5, 6, 7, 8, 9, or 10 or more conservative or non-conservative amino acid substitutions relative to the amino acid sequence set forth in any one of SEQ ID NOs: 48, 49, 50, 51, 55, 56, 57, 58, 62, 63, 64, 65, 69, 70, 71, and 72, wherein the antibody or antigen-binding fragment comprising the V_{H} domain binds Nectin-4. In some embodiments, any of the sequence variation occurs in the framework region of the antibodies or antigen binding fragments thereof. In some embodiments, the amino acid substitutions are conservative amino acid substitutions.

In some embodiments, disclosed herein are anti-Nectin-4 antibodies that comprise a heavy chain that is at least 90% identical to the amino acid sequence set forth in any one of SEQ ID NOs: 144, 145, 146, 147, 148, 149, 150, 151, 152, 153, 154, 155, 156, 157, 158, or 159, for example, about 90% identical, about 91% identical, about 92% identical, about 93% identical, about 94% identical, about 95% identical, about 96% identical, about 97% identical, about 98% identical, or about 99% identical to any one of SEQ ID NOs: 144, 145, 146, 147, 148, 149, 150, 151, 152, 153, 154, 155, 156, 157, 158, or 159. In some embodiments, the anti-Nectin-4 antibody comprises a heavy chain that comprises an amino acid sequence as set forth in any one of SEQ ID NOs: 144, 145, 146, 147, 148, 149, 150, 151, 152, 153, 154, 155, 156, 157, 158, or 159.

In some embodiments, disclosed herein are anti-Nectin-4 antibodies that comprise a light chain that is at least 90% identical to any one of SEQ ID NOs: 127, 128, 129, 130, 131, 132, 133, 134, 135, 136, 137, 138, 139, 140, 141, 142, and 143, for example, about 90% identical, about 91% identical, about 92% identical, about 93% identical, about 94% identical, about 95% identical, about 96% identical, about 97% identical, about 98% identical, or about 99% identical to any one of SEQ ID NOs: 127, 128, 129, 130, 131, 132, 133, 134, 135, 136, 137, 138, 139, 140, 141, 142, and 143. In some embodiments, the Nectin-4 antibodies comprise a light chain that comprises any one of SEQ ID NOs: 127, 128, 129, 130, 131, 132, 133, 134, 135, 136, 137, 138, 139, 140, 141, 142, and 143.

In some embodiments, disclosed herein are antibodies or antigen binding fragments thereof that comprise an anti-Nectin-4 V_{H} region comprising a CDRH1 comprising SEQ ID NO: 15, a CDRH2 comprising SEQ ID NO: 16, and a CDRH3 comprising SEQ ID NO: 17. In some embodiments CDRH1 comprises a sequence that has one or two amino acid substitutions relative to SEQ ID NO: 15. In some embodiments, CDRH2 comprises a sequence that has one or two amino acid substitutions relative to SEQ ID NO: 16. In some embodiments, CDRH3 comprises a sequence that has one or two amino acid substitutions relative to SEQ ID NO: 17. In some embodiments, the one or two amino acid substitutions in CDRH1, CDRH2, and/or CDRH3 are conservative amino acid substitutions.

In some embodiments, disclosed herein are antibodies or antigen binding fragments thereof that comprise an anti-Nectin-4 V_{H} region comprising CDRH1 comprising SEQ ID NO: 52, CDRH2 comprising SEQ ID NO: 53, CDRH3 comprising SEQ ID NO: 54. In some embodiments, CDRH1 comprises a sequence that has one or two amino acid substitutions relative to SEQ ID NO: 52. In some embodiments, CDRH2 comprises a sequence that has one or two amino acid substitutions relative to SEQ ID NO: 53. In some embodiments, CDRH3 comprises a sequence that has one or two amino acid substitutions relative to SEQ ID NO: 54. In some embodiments, the one or two amino acid substitutions in CDRH1, CDRH2, and/or CDRH3 are conservative amino acid substitutions.

In some embodiments, disclosed herein are antibodies or antigen binding fragments thereof that comprises an anti-Nectin-4 V_{H} region comprising CDRH1 comprising SEQ ID NO: 59, CDRH2 comprising SEQ ID NO: 60, CDRH3 comprising SEQ ID NO: 61. In some embodiments, CDRH1 comprises a sequence that has one or two amino acid substitutions relative to SEQ ID NO: 59. In some embodiments, CDRH2 comprises a sequence that has one or two amino acid substitutions relative to SEQ ID NO: 60. In some embodiments, CDRH3 comprises a sequence that has one or two amino acid substitutions relative to SEQ ID NO: 61. In some embodiments, the one or two amino acid substitutions in CDRH1, CDRH2, and/or CDRH3 are conservative amino acid substitutions.

In some embodiments, disclosed herein are antibodies or antigen binding fragments thereof that comprises an anti-Nectin-4 V_{H} region comprising CDRH1 comprising SEQ ID NO: 66, CDRH2 comprising SEQ ID NO: 67, CDRH3 comprising SEQ ID NO: 68. In some embodiments, CDRH1 comprises a sequence that has one or two amino acid substitutions relative to SEQ ID NO: 66. In some embodiments, CDRH2 comprises a sequence that has one or two amino acid substitutions relative to SEQ ID NO: 67. In some embodiments, CDRH3 comprises a sequence that has one or two amino acid substitutions relative to SEQ ID NO: 68. In some embodiments, the one or two amino acid substitutions in CDRH1, CDRH2, and/or CDRH3 are conservative amino acid substitutions.

In some embodiments, disclosed herein are antibodies or antigen binding fragments thereof that comprises an anti-Nectin-4 V_{L} region comprising CDRL1 comprising SEQ ID NO: 20, CDRL2 comprising SEQ ID NO: 21, CDRL3 comprising SEQ ID NO: 22. In some embodiments, CDRL1 comprises a sequence that has one or two amino acid substitutions relative to SEQ ID NO: 20. In some embodiments, CDRL2 comprises a sequence that has one or two amino acid substitutions relative to SEQ ID NO: 21. In some embodiments, CDRL3 comprises a sequence that has one or two amino acid substitutions relative to SEQ ID NO: 22. In some embodiments, the one or two amino acid substitutions in CDRH1, CDRH2, and/or CDRH3 are conservative amino acid substitutions.

In some embodiments, disclosed herein are antibodies or antigen binding fragments thereof that comprises an anti-Nectin-4 V_{L} region comprising CDRL1 comprising SEQ ID NO: 28, CDRL2 comprising SEQ ID NO: 29, CDRL3 comprising SEQ ID NO: 30. In some embodiments, CDRL1 comprises a sequence that has one or two amino acid substitutions relative to SEQ ID NO: 28. In some embodiments, CDRL2 comprises a sequence that has one or two amino acid substitutions relative to SEQ ID NO: 29. In some embodiments, CDRL3 comprises a sequence that has one or two amino acid substitutions relative to SEQ ID NO: 30. In some embodiments, the one or two amino acid substitutions in CDRL1, CDRL2, and/or CDRL3 are conservative amino acid substitutions.

In some embodiments, disclosed herein are antibodies or antigen binding fragments thereof that comprises an anti-Nectin-4 V_{L} region comprising CDRL1 comprising SEQ ID NO: 35, CDRL2 comprising SEQ ID NO: 36, CDRL3 comprising SEQ ID NO: 37. In some embodiments, CDRL1 comprises a sequence that has one or two amino acid substitutions relative to SEQ ID NO: 35. In some embodiments, CDRL2 comprises a sequence that has one or two amino acid substitutions relative to SEQ ID NO: 36. In some embodiments, CDRL3 comprises a sequence that has one or two amino acid substitutions relative to SEQ ID NO: 37. In some embodiments, the one or two amino acid substitutions in CDRH1, CDRH2, and/or CDRH3 are conservative amino acid substitutions.

In some embodiments, disclosed herein are antibodies or antigen binding fragments thereof that comprises an anti-Nectin-4 V_{L} region comprising CDRL1 comprising SEQ ID NO: 42, CDRL2 comprising SEQ ID NO: 29, CDRL3 comprising SEQ ID NO: 30. In some embodiments, CDRL1 comprises a sequence that has one or two amino acid substitutions relative to SEQ ID NO: 42. In some embodiments, CDRL2 comprises a sequence that has one or two amino acid substitutions relative to SEQ ID NO: 29. In some embodiments, CDRL3 comprises a sequence that has one or two amino acid substitutions relative to SEQ ID NO: 30. In some embodiments, the one or two amino acid substitutions in CDRL1, CDRL2, and/or CDRL3 are conservative amino acid substitutions.

### Binding Affinity

By way of example, and not limitation, any of the Nectin-4 antibodies and antibody binding fragments disclosed herein may bind human Nectin-4 with a K_{D} value of at least 1×10⁻⁹ M. In some embodiments, the K_{D} value is 2×10⁻⁹ M. In some embodiments, the K_{D} value is 3×10⁻⁹ M. In some embodiments, the K_{D} value is 4×10⁻⁹ M. In some embodiments, the K_{D} value is 5×10⁻⁹ M. In some embodiments, the K_{D} value is 6×10⁻⁹ M. In some embodiments, the K_{D} value is 7×10⁻⁹ M. In some embodiments, the K_{D} value is 8×10⁻⁹ M. In some embodiments, the K_{D} value is 9×10⁻⁹ M. In some embodiments, the K_{D} value is 10×10⁻⁹ M.

### Nucleic Acids

The present disclosure further comprises nucleic acids encoding one or both of the immunoglobulin chains of the anti-Nectin-4 antibodies and antigen binding fragments thereof disclosed herein.

In another embodiment, the invention provides an isolated nucleic acid or nucleic acids, for example DNA, encoding one or both of the polypeptide chains of the isolated antibodies or antigen binding fragments described herein. In one embodiment, the isolated nucleic acid encodes an antibody or antigen binding fragment thereof comprising at least one antibody light chain variable (V_{L}) domain and at least one antibody heavy chain variable (V_{H}) domain, wherein the V_{L} domain comprises an amino acid sequence selected from any one of SEQ ID NOs: 24, 25, 26, 27, 31, 32, 33, 34, 38, 39, 40, 41, 43, 44, 45, 46, and 47, and the V_{H} domain comprises an amino acid sequence selected from any one of SEQ ID NOs: 48, 49, 50, 51, 55, 56, 57, 58, 62, 63, 64, 65, 69, 70, 71, and 72. In some embodiments, the isolated nucleic acid encodes both a V_{L} and a V_{H} on a single nucleic acid molecule, and in other embodiments the V_{L} and V_{H} are encoded by separate nucleic acid molecules. In some embodiments, the nucleic acids further encode a signal sequence.

In some embodiments, the isolated nucleic acid encodes a light chain having an amino acid sequence selected from any one of SEQ ID NOs: 127, 128, 129, 130, 131, 132, 133, 134, 135, 136, 137, 138, 139, 140, 141, 142, and 143. In some embodiments, the isolated nucleic acid encodes a heavy chain having an amino acid sequence selected from any one of SEQ ID NOs: 144, 145, 146, 147, 148, 149, 150, 151, 152, 153, 154, 155, 156, 157, 158, and 159. In some embodiments, the isolated nucleic acid encodes both a light chain and a heavy chain on a single nucleic acid molecule, and in other embodiments the light and heavy chains are encoded by separate nucleic acid molecules. In some embodiments, the nucleic acids further encode a signal sequence.

In some embodiments, the polynucleotides encode a light chain having an amino acid sequence that is at least 90% identical to any one of SEQ ID NOs: 160, 161, 162, 163, 164, 165, 166, 167, 168, 169, 170, 171, 172, 173, 174, 175, and 176, for example, about 90% identical, about 91% identical, about 92% identical, about 93% identical, about 94% identical, about 95% identical, about 96% identical, about 97% identical, about 98% identical, or about 99% identical to any one of SEQ ID NOs: 160, 161, 162, 163, 164, 165, 166, 167, 168, 169, 170, 171, 172, 173, 174, 175, and 176.

In some embodiments, the polynucleotides encode a heavy chain having an amino acid sequence that is at least 90% identical to any one of SEQ ID NOs: 177, 178, 179, 180, 181, 182, 183, 184, 185, 186, 187, 188, 189, 190, 191, and 192 for example, about 90% identical, about 91% identical, about 92% identical, about 93% identical, about 94% identical, about 95% identical, about 96% identical, about 97% identical, about 98% identical, or about 99% identical to any one of SEQ ID NOs: 177, 178, 179, 180, 181, 182, 183, 184, 185, 186, 187, 188, 189, 190, 191, and 192.

In some embodiments, the isolated nucleic acid encodes an antibody or antigen binding fragment comprising an antibody light chain variable (V_{L}) domain comprising a CDRL1 comprising SEQ ID NO: 20, a CDRL2 comprising SEQ ID NO: 21, and a CDRL3 comprising SEQ ID NO: 22. In some embodiments, the isolated nucleic acid encodes a V_{L} domain comprising a CDRL1 that has one or two amino acid substitutions relative to SEQ ID NO: 20. In some embodiments, the isolated nucleic acid encodes a V_{L} domain comprising a CDRL2 that has one or two amino acid substitutions relative to SEQ ID NO: 21. In some embodiments, the isolated nucleic acid encodes a V_{L} domain comprising a CDRL3 that has one or two amino acid substitutions relative to SEQ ID NO: 22. In some embodiments, the one or two amino acid substitutions in CDRL1, CDRL2, and/or CDRL3 are conservative amino acid substitutions.

In some embodiments, the isolated nucleic acid encodes an antibody or antigen binding fragment thereof comprising an antibody light chain variable (V_{L}) domain comprising a CDRL1 comprising SEQ ID NO: 28, a CDRL2 comprising SEQ ID NO: 29, and a CDRL3 comprising SEQ ID NO: 30. In some embodiments, the isolated nucleic acid encodes a V_{L} domain comprising a CDRL1 that has one or two amino acid substitutions relative to SEQ ID NO: 28. In some embodiments, the isolated nucleic acid encodes a V_{L} domain comprising a CDRL2 that has one or two amino acid substitutions relative to SEQ ID NO: 29. In some embodiments, the isolated nucleic acid encodes a V_{L} domain comprising a CDRL3 that has one or two amino acid substitutions relative to SEQ ID NO: 30. In some embodiments, the one or two amino acid substitutions in CDRL1, CDRL2, and/or CDRL3 are conservative amino acid substitutions.

In some embodiments, the isolated nucleic acid encodes an antibody or antigen binding fragment thereof comprising an antibody light chain variable (V_{L}) domain comprising the CDRL1 comprising SEQ ID NO: 35, CDRL2 comprising SEQ ID NO: 36, and CDRL3 comprising SEQ ID NO: 37. In some embodiments, the isolated nucleic acid encodes a V_{L} domain comprising a CDRL1 that has one or two amino acid substitutions relative to SEQ ID NO: 35. In some embodiments, the isolated nucleic acid encodes a V_{L} domain comprising a CDRL2 that has one or two amino acid substitutions relative to SEQ ID NO: 36. In some embodiments, the isolated nucleic acid encodes a V_{L} domain comprising a CDRL3 that has one or two amino acid substitutions relative to SEQ ID NO: 37. In some embodiments, the one or two amino acid substitutions in CDRL1, CDRL2, and/or CDRL3 are conservative amino acid substitutions.

In some embodiments, the isolated nucleic acid encodes an antibody or antigen binding fragment thereof comprising an antibody light chain variable (V_{L}) domain comprising a CDRL1 comprising SEQ ID NO: 42, aCDRL2 comprising SEQ ID NO: 29, and aCDRL3 comprising SEQ ID NO: 30. In some embodiments, the isolated nucleic acid encodes a V_{L} domain comprising a CDRL1 that has one or two amino acid substitutions relative to SEQ ID NO: 42. In some embodiments, the isolated nucleic acid encodes a V_{L} domain comprising a CDRL2 that has one or two amino acid substitutions relative to SEQ ID NO: 29. In some embodiments, the isolated nucleic acid encodes a V_{L} domain comprising a CDRL3 that has one or two amino acid substitutions relative to SEQ ID NO: 30. In some embodiments, the one or two amino acid substitutions in CDRL1, CDRL2, and/or CDRL3 are conservative amino acid substitutions.

In some embodiments, the isolated nucleic acid encodes an antibody or antigen binding fragment thereof comprising an antibody heavy chain variable (V_{H}) domain comprising a CDRH1 comprising SEQ ID NO: 15, a CDRH2 comprising SEQ ID NO: 16, and a CDRH3 comprising SEQ ID NO: 17. In some embodiments, the isolated nucleic acid encodes a V_{H} domain comprising a CDRH1 that has one or two amino acid substitutions relative to SEQ ID NO: 15. In some embodiments, the isolated nucleic acid encodes a V_{H} domain comprising a CDRH2 that has one or two amino acid substitutions relative to SEQ ID NO: 16. In some embodiments, the isolated nucleic acid encodes a V_{H} domain comprising a CDRH3 that has one or two amino acid substitutions relative to SEQ ID NO: 17. In some embodiments, the one or two amino acid substitutions in CDRL1, CDRL2, and/or CDRL3 are conservative amino acid substitutions.

In some embodiments, the isolated nucleic acid encodes an antibody or antigen binding fragment thereof comprising an antibody heavy chain variable (V_{H}) domain comprising a CDRH1 comprising SEQ ID NO: 52, a CDRH2 comprising SEQ ID NO: 53, and a CDRH3 comprising SEQ ID NO: 54. In some embodiments, the isolated nucleic acid encodes a V_{H} domain comprising a CDRH1 that has one or two amino acid substitutions relative to SEQ ID NO: 52. In some embodiments, the isolated nucleic acid encodes a V_{H} domain comprising a CDRH2 that has one or two amino acid substitutions relative to SEQ ID NO: 53. In some embodiments, the isolated nucleic acid encodes a V_{H} domain comprising a CDRH3 that has one or two amino acid substitutions relative to SEQ ID NO: 54. In some embodiments, the one or two amino acid substitutions in CDRH1, CDRH2, and/or CDRH3 are conservative amino acid substitutions.

In some embodiments, the isolated nucleic acid encodes an antibody or antigen binding fragment thereof comprising an antibody heavy chain variable (V_{H}) domain comprising a CDRH1 comprising SEQ ID NO: 59, a CDRH2 comprising SEQ ID NO: 60, and a CDRH3 comprising SEQ ID NO: 61. In some embodiments, the isolated nucleic acid encodes a V_{H} domain comprising a CDRH1 that has one or two amino acid substitutions relative to SEQ ID NO: 59. In some embodiments, the isolated nucleic acid encodes a V_{H} domain comprising a CDRH2 that has one or two amino acid substitutions relative to SEQ ID NO: 60. In some embodiments, the isolated nucleic acid encodes a V_{H} domain comprising a CDRH3 that has one or two amino acid substitutions relative to SEQ ID NO: 61. In some embodiments, the one or two amino acid substitutions in CDRH1, CDRH2, and/or CDRH3 are conservative amino acid substitutions.

In some embodiments, the isolated nucleic acid encodes an antibody or antigen binding fragment thereof comprising an antibody heavy chain variable (V_{H}) domain comprising a CDRH1 comprising SEQ ID NO: 66, a CDRH2 comprising SEQ ID NO: 67, and a CDRH3 comprising SEQ ID NO: 68. In some embodiments, the isolated nucleic acid encodes a V_{H} domain comprising a CDRH1 that has one or two amino acid substitutions relative to SEQ ID NO: 66. In some embodiments, the isolated nucleic acid encodes a V_{H} domain comprising a CDRH2 that has one or two amino acid substitutions relative to SEQ ID NO: 67. In some embodiments, the isolated nucleic acid encodes a V_{H} domain comprising a CDRH3 that has one or two amino acid substitutions relative to SEQ ID NO: 68. In some embodiments, the one or two amino acid substitutions in CDRH1, CDRH2, and/or CDRH3 are conservative amino acid substitutions.

### Methods of Making Antibodies and Antigen Binding Fragments Thereof

The anti-Nectin-4 antibodies disclosed herein may be produced using any method known in the art. For example, the anti-target antibodies disclosed herein may be produced recombinantly. In some embodiments, nucleic acids encoding the antibody molecules of the invention (e.g., V_{H} or V_{L} domains described herein) may be inserted into a vector and expressed in a recombinant host cell. There are several methods by which to produce recombinant antibodies which are known in the art.

Mammalian cell lines available as hosts for expression of the antibodies or fragments disclosed herein are well known in the art and include many immortalized cell lines available from the American Type Culture Collection (ATCC). These include, inter alia, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, 3T3 cells, HEK-293 cells and a number of other cell lines. Cell lines of particular preference are selected through determining which cell lines have high expression levels. Other cell lines that may be used are insect cell lines, such as Sf9 cells, amphibian cells, bacterial cells, plant cells and fungal cells. When recombinant expression vectors encoding the heavy chain or antigen-binding portion or fragment thereof, the light chain and/or antigen-binding fragment thereof are introduced into host cells, the antibodies are produced by culturing the host cells for a period of time sufficient to allow for expression of the antibody in the host cells or, more preferably, secretion of the antibody into the culture medium in which the host cells are grown.

Antibodies can be recovered from the culture medium using standard protein purification methods. Further, expression of antibodies of the invention (or other moieties therefrom) from production cell lines can be enhanced using a number of known techniques. For example, the glutamine synthetase gene expression system (the GS system) is a common approach for enhancing expression under certain conditions.

In general, glycoproteins produced in a particular cell line or transgenic animal will have a glycosylation pattern that is characteristic for glycoproteins produced in the cell line or transgenic animal. Therefore, the particular glycosylation pattern of an antibody will depend on the particular cell line or transgenic animal used to produce the antibody. However, all antibodies encoded by the nucleic acid molecules provided herein, or comprising the amino acid sequences provided herein, are included in the embodiments disclosed herein, independent of the glycosylation pattern the antibodies may have. Similarly, in particular embodiments, antibodies with a glycosylation pattern comprising only non-fucosylated N-glycans may be advantageous, because these antibodies have been shown to typically exhibit more potent efficacy than their fucosylated counterparts both in vitro and in vivo (See for example, Shinkawa et al., J. Biol. Chem. 278:3466-3473 (2003); U.S. Pat. Nos. 6,946,292 and 7,214,775). These antibodies with non-fucosylated N-glycans are not likely to be immunogenic because their carbohydrate structures are a normal component of the population that exists in human serum IgG.

The present disclosure further includes antibody fragments of the anti-target antibodies disclosed herein. The antibody fragments include F(ab)₂ fragments, which may be produced by enzymatic cleavage of an IgG by, for example, pepsin. Fab fragments may be produced by, for example, reduction of F(ab)₂ with dithiothreitol or mercaptoethylamine. A Fab fragment is a V_{L}-C_{L} chain appended to a V_{H}-C_{H1} chain by a disulfide bridge. A F(ab)₂ fragment is two Fab fragments which, in turn, are appended by two disulfide bridges. The Fab portion of an F(ab)₂ molecule includes a portion of the Fe region between which disulfide bridges are located. An Fv fragment is a V_{L} or V_{H} region.

Immunoglobulins may be assigned to different classes depending on the amino acid sequences of the constant domain of their heavy chains. There are at least five major classes of immunoglobulins: IgA, IgD, IgE, IgG and IgM, and several of these may be further divided into subclasses (isotypes), e.g., IgG-1, IgG-2, IgG-3 and IgG-4; IgA-1 and IgA-2. The disclosure comprises antibodies and antigen binding fragments of any of these classes or subclasses of antibodies.

In some embodiments, the antibodies or antigen binding fragments disclosed herein comprise a heavy chain constant region, e.g., a human constant region, such as γ1, γ2, 3, or γ4 human heavy chain constant region or a variant thereof. In some embodiments, the antibodies or antigen binding fragments comprise a light chain constant region, e.g., a human light chain constant region, such as lambda or kappa human light chain region or variant thereof. By way of example, and not limitation the human heavy chain constant region can be γ1 and the human light chain constant region can be kappa. In an alternative embodiment, the Fc region of the antibody is γ4 with a Ser228Pro mutation (Schuurman, J et al., Mol. Immunol. 38:1-8, 2001).

In some embodiments, different constant domains may be appended to humanized V_{L} and V_{H} regions derived from the CDRs provided herein. For example, if a particular intended use of an antibody (or fragment) of the present invention were to call for altered effector functions, a heavy chain constant domain other than human IgG1 may be used, or hybrid IgG1/IgG4 may be utilized.

### Therapeutic Uses of Anti-Nectin-4 Antibodies

Further provided are methods for treating subjects, including human subjects, in need of treatment with the anti-Nectin-4 antibodies or antigen binding fragments thereof disclosed herein. In some embodiments, the anti-Nectin-4 antibodies or antigen binding fragments thereof are used to treat cancer. In some embodiments, the cancer includes any one or more of head and neck cancer, skin cancer, breast cancer, lung cancer, urothelial, colorectal cancer, cervical, pancreatic cancer, bladder cancer, and ovarian cancer. In some embodiments, the head and neck cancer is head and neck squamous cell carcinoma (HNSCC). In some embodiments, the anti-Nectin-3 antibodies or antigen binding fragments thereof are used to treat carcinoma or sarcoma. In some embodiments, the anti-Nectin-4 antibodies or antigen-binding fragments thereof disclosed herein may be used alone, or in combination with other agents, for treating or preventing any disease or condition in a subject in need of such treatment or prevention.

In some embodiments, the invention comprises a method of treating cancer in a patient in need thereof comprising administering to the patient an effective amount of any of the antibodies or antigen binding fragments, or pharmaceutical composition disclosed herein. In some embodiments, the cancer is head and neck cancer. In some embodiments, the cancer is any of breast cancer, bladder cancer, lung cancer, urothelial cancer, colorectal cancer, cervical cancer, pancreatic cancer, head and neck squamous cell carcinoma (HNSCC), or ovarian cancer. In some embodiments, the cancer is any of bladder cancer, HNSCC, cervical cancer, breast cancer, or pancreatic cancer. In some embodiments, the cancer is bladder cancer. In some embodiments, the cancer is skin cancer (e.g., melanoma). In some embodiments, the cancer is breast cancer (e.g., triple negative breast cancer) In some embodiments, the cancer is lung cancer (e.g., non-small cell lung cancer). In some embodiments, the cancer is urothelial cancer. In some embodiments, the cancer is colorectal cancer. In some embodiments, the cancer is cervical cancer. In some embodiments, the cancer is pancreatic cancer. In some embodiments, the cancer is bladder cancer. In some embodiments, the cancer is ovarian cancer. In some embodiments, the cancer is head and neck squamous cell carcinoma (HNSCC).

In some embodiments, any of the antibodies or antigen binding fragments described herein can be used in treating cancer. In some embodiments, the cancer is any of breast cancer, bladder cancer, lung cancer, urothelial cancer, colorectal cancer, cervical cancer, pancreatic cancer, head and neck squamous cell carcinoma (HNSCC), or ovarian cancer. In some embodiments, the cancer is any of bladder cancer, HNSCC, cervical cancer, breast cancer, or pancreatic cancer. In some embodiments, the cancer is bladder cancer. In some embodiments, the cancer is skin cancer (e.g., melanoma). In some embodiments, the cancer is breast cancer (e.g., triple negative breast cancer) In some embodiments, the cancer is lung cancer (e.g., non-small cell lung cancer). In some embodiments, the cancer is urothelial cancer. In some embodiments, the cancer is colorectal cancer. In some embodiments, the cancer is cervical cancer. In some embodiments, the cancer is pancreatic cancer. In some embodiments, the cancer is bladder cancer. In some embodiments, the cancer is ovarian cancer. In some embodiments, the cancer is head and neck squamous cell carcinoma (HNSCC).

### Pharmaceutical Compositions and Administration

To prepare pharmaceutical or sterile compositions, the anti-Nectin-4 antibodies or antigen binding fragment thereof are admixed with a pharmaceutically acceptable carrier or excipient. See, e.g., Remington's Pharmaceutical Sciences and U.S. Pharmacopeia: National Formulary, Mack Publishing Company, Easton, PA (1984).

Formulations of therapeutic and diagnostic agents may be prepared by mixing with acceptable carriers, excipients, or stabilizers in the form of, e.g., lyophilized powders, slurries, aqueous solutions or suspensions (see, e.g., Hardman, et al. (2001) Goodman and Gilman's The Pharmacological Basis of Therapeutics, McGraw-Hill, New York, NY; Gennaro (2000) Remington: The Science and Practice of Pharmacy, Lippincott, Williams, and Wilkins, New York, NY; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, NY).

Toxicity and therapeutic efficacy of the antibody compositions, administered alone or in combination with another agent, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index (LD₅₀/ED₅₀). In particular aspects, antibodies exhibiting high therapeutic indices are desirable. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration.

In a further embodiment, a composition comprising an antibody or antibody fragment disclosed herein is administered to a subject in accordance with the Physicians' Desk Reference 2003 (Thomson Healthcare; 57th edition (Nov. 1, 2002)).

The mode of administration can vary. Suitable routes of administration include oral, rectal, transmucosal, intestinal, parenteral; intramuscular, subcutaneous, intradermal, intramedullary, intrathecal, direct intraventricular, intravenous, intraperitoneal, intranasal, intraocular, inhalation, insufflation, topical, cutaneous, transdermal, or intra-arterial).

### EXAMPLES

The following examples are meant to be illustrative and should not be construed as further limiting of the embodiments described herein.

### Example 1: Synthesis and Characterization of Mouse Antibodies that Target Nectin-4

### Mouse Antibody Discovery Against Nectin-4

BALB/cJ and SJL/J mice were immunized with Nectin4-Fc immunogen using a standard 28-Day RIMMS protocol and 50 μg doses of the immunogen administered subcutaneously. The mice with the highest titer from each strain after RIMMs protocol were boosted with 25 μg immunogen. 3 to 5 days later mice splenocytes and lymphocytes were harvested and fused with NS1 myeloma cells using standard PEG fusion procedures. Fusion cell products were plated into 96 well plates and expanded in hybridoma medium. 10 to 11 days after plating supernatants were screened for their ability to bind plate coated human and cynomolgus monkey Nectin-4 antigen in a standard ELISA format using an anti-mouse secondary detection antibody. Positive wells were selected and hybridoma pools expanded. 3 to 5 days later a confirmatory ELISA was performed prior to subcloning the hybridoma pools. Each parental hybridoma pool of interest was subcloned into 96 well plates by the standard limiting dilution method. Subcloning plates were allowed to grow for 10 to 11 days prior to screening supernatants for Nectin-4 binding by ELISA. Clones were selected and further expanded. 10 mL supernatants of selected clones were purified using disposable proteinG drip columns using a standard bind, wash, elute method. Eluted antibodies were brought to neutral pH and measured for protein concentration prior to binding affinity measurements against human and cyno Nectin-4. Purified hybridoma antibodies were also characterized for their ability to bind particular domains within human Nectin-4. Clonal hybridomas cell lines of interest were then sequenced and cryopreserved. Hybridoma antibody sequences were determined using the standard 5′-RACE method. The light chain variable sequences and the light chain CDR sequences of the hybridoma antibody sequences are shown in Table 3. The heavy chain variable sequences and the heavy chain CDR sequences are shown in Table 4.

TABLE 3
Light Chain Variable and Light Chain CDR Sequences (Mouse Hybridoma
Clones)

| Mouse | Light chain variable | | | |
|---|---|---|---|---|
| Clone | sequences | CDRL1 | CDRL2 | CDRL3 |
| 14E4.H3 | DIVMTQSHKFMSTSVGGRVT | KASQDVSIAVA | WASTRQT | QQYSSYPFT |
| | ITCKASQDVSIAVAWYQQKP | (SEQ ID NO: 28) | (SEQ ID NO: 29) | (SEQ ID NO: 30) |
| | GQSPKLLIYWASTRQTGVPD | | | |
| | RFAGSGSGTDFTLTISNVQSE | | | |
| | DLADYFCQQYSSYPFTFGSG | | | |
| | TKLEIK | | | |
| | (SEQ ID NO: 115) | | | |
| 14F6.B1 | DIVMTQSQKFMSTTVGDRVS | KASQNVVTAVA | SASHRFT | QQYSSYPYT |
| | ITCKASQNVVTAVAWYQQK | (SEQ ID NO: 35) | (SEQ ID NO: 36) | (SEQ ID NO: 37) |
| | PGQSPKLLIYSASHRFTGVPD | | | |
| | RFTGSGSGTDFTLIISNMQSE | | | |
| | DLADYFCQQYSSYPYTFGG | | | |
| | GTKLEIK | | | |
| | (SEQ ID NO: 117) | | | |
| 16H8.B1 | DIVMTQSHKFMSTSVGGRV | KASQDVSTTVA | WASTRQT | QQYSSYPFT |
| | TITCKASQDVSTTVAWYQQ | (SEQ ID NO: 42) | (SEQ ID NO: 29) | (SEQ ID NO: 30) |
| | KPGQSPKLLIYWASTRQTG | | | |
| | VPDRFTGSGSGTDFTLTISN | | | |
| | VQSEDLADYFCQQYSSYPF | | | |
| | TFGSGTKLEIK | | | |
| | (SEQ ID NO: 119) | | | |

TABLE 4
Heavy Chain Variable and Heavy Chain CDR Sequences (Mouse Hybridoma
Clones)

| Mouse | Heavy chain variable | | | |
|---|---|---|---|---|
| Clone | sequence | CDRH1 | CDRH2 | CDRH3 |
| 14E4.H3 | EVQLQESGGDLVQPGGSLKLSC | GFTFSDYY | YISNGGGNTYY | ASPEARYYGN |
| | AASGFTFSDYYMFWIRQTPQKR | (SEQ ID NO: 120) | PDTVKG | YPFPY |
| | LEWVAYISNGGGNTYYPDTVK | | (SEQ ID NO: 53) | (SEQ ID NO: |
| | GRFTISRDNAKNTLYLQMSRLK | | | 121) |
| | SEDTAMYYCASPEARYYGNYP | | | |
| | FPYWGQGTLVTVSA (SEQ ID | | | |
| | NO: 114) | | | |
| 14F6.B1 | QVQLKQSGPGLVQPSQSLSITCT | GFSLTTYG | VIWSGGSTDYN | ARTSHWYFDV |
| | VSGFSLTTYGVHWVRQSPGKG | (SEQ ID NO: 122) | AAFIS | (SEQ ID NO: |
| | LEWLGVIWSGGSTDYNAAFISR | | (SEQ ID NO: 60) | 123) |
| | LSISKDNSKSQVFFEMYSLQAD | | | |
| | DTAIYYCARTSHWYFDVWGTG | | | |
| | TTVTVSS (SEQ ID NO: 116) | | | |
| 16H8.B1 | EVQLQESGGGLVQPGGSLKLSC | GFTFSDYY | YISNGGGNTYY | ASPEARYYGN |
| | AASGFTFSDYYMYWIRQTPQK | (SEQ ID NO: 120) | SDTVKG | FPFPY |
| | RLEWVAYISNGGGNTYYSDTV | | (SEQ ID NO: 67) | (SEQ ID NO: |
| | KGRFTISRDNAKNTLYLQMSRL | | | 124) |
| | KSEDTAMYYCASPEARYYGNF | | | |
| | PFPYWGQGTLVTVSA (SEQ ID | | | |
| | NO: 118) | | | |

### Hybridoma Antibody Epitope Mapping

Purified hybridoma antibodies were screened for their ability to bind full length Nectin-4 antigen and truncated versions thereof fused to human Fc, including Nectin-4 V-C1 domains, and Nectin-4 C1-C2 domains in a standard ELISA format. Full length and truncated Nectin-4 antigens were plate coated, washed, blocked, and washed prior to addition of titrated purified clonal hybridoma antibodies. After a short incubation with purified hybridoma antibodies plates were washed again and bound antibody was detected using a horse radish peroxidase conjugated anti-mouse secondary antibody. Results are shown in Table 5.

TABLE 5
Antibody Epitope Mapping (Mouse Hybridoma Clones)
Domain mapping (ELISA EC50 mM)

| Hybridoma | Full-length | Nectin-4 VC1 | Nectin-4 C1-C2 |
|---|---|---|---|
| Clone | Nectin-4 | domain | domain |
| 14.E4H3 | 0.02 | 0.03 | 0.03 |
| 14.F6B1 | 0.03 | 129.10 | 0.03 |
| 16H8.B1 | 0.03 | 0.03 | 0.03 |

### Hybridoma Antibody Binding Specificity

Purified hybridoma antibodies were screened for their binding specificity by comparing binding potency to closely related family members of Nectin-4 (Nectin-1, Nectin-2, and Nectin-3) by ELISA. Full length Nectin antigens fused to a human Fc were plate coated, washed, blocked, and washed prior to addition of titrated purified clonal hybridoma antibodies (Full length EGFR fused to a human Fc was used as a control). After a short incubation with purified hybridoma antibodies plates were washed again and bound antibody was detected using a horse radish peroxidase conjugated anti-mouse secondary antibody. Results of these ELISA experiments are shown in Table 6.

TABLE 6
Antibody Binding Specificity (Mouse Hybridoma Clones)

| | Selectivity of Binding to Nectin-1, Nectin-2, | | | |
|---|---|---|---|---|
| | and Nectin-3 (ELISA EC50 mM | | | |
| Mouse Clone | EGFR Fc | Nectin-1 Fc | Nectin-2 Fc | Nectin-3 Fc |
| 14E4.E12 | 2067 | 1629 | 1004 | 2294 |
| 14F6.B1 | 556.4 | 584.8 | 319.5 | 39.34 |
| 16H8.B1 | 8459 | 3561 | 3103 | 4597 |

Kinetic binding of polypeptide molecules to human and cynomolgus monkey Nectin-4 was evaluated by bio-layer interferometry using an Octet instrument. Briefly, biosensors were loaded with antigen and baselined in buffer. Polypeptide molecules were titrated in solution at 50 nM, 25 nM, 12.5 nM, and 6.25 nM then associated onto the antigen loaded sensors. After a short association period, sensors were transferred into buffer and the dissociation of bound polypeptide molecules was measured. Association and dissociation signals were recorded in real time and analyzed using a 1:1 binding model within the instrument software. Analysis using a 1:1 binding model enabled the calculation of the on and off rate constants as well as affinity, KD. Results of the Octet binding assays are shown in Tables 7 and 8.

TABLE 7
Octet Binding Assays - Human Nectin-4
(Mouse Hybridoma Clones)

| | Human Nectin-4 (Octet) | | | |
|---|---|---|---|---|
| Mouse Clone | KD (M) | kon (1/Ms) | koff (½) | t½ min |
| 14E4.E12 | 1.22E−09 | 3.97E+05 | 4.82E−04 | 23.97 |
| 14F6.B1 | 3.34E−09 | 1.15E+05 | 3.85E−04 | 30.01 |
| 16H8.B1 | 3.07E−09 | 2.67E+05 | 8.19E−04 | 14.11 |

TABLE 8
Octet Binding Assays - Cyano Nectin-4
(Mouse Hybridoma Clones)

| | Cyano Nectin-4 (Octet) | | | |
|---|---|---|---|---|
| Mouse Clone | KD (M) | kon (1/Ms) | koff (½) | t½ min |
| 14E4.E12 | 8.56E−10 | 4.14E+05 | 3.54E−04 | 32.63 |
| 14F6.B1 | 2.13E−09 | 1.55E+05 | 3.30E−04 | 35.01 |
| 16H8.B1 | 2.68E−09 | 2.96E+05 | 7.95E−04 | 14.53 |

### Example 2: Synthesis and Characterization of Rat Antibodies that Target Nectin-4

### Immunization and Hybridoma Screening

Six-week-old Lewis rats were co-immunized with a 1:1 mixed of human and mouse His tagged Nectin-4 recombinant protein (R&D systems human Nectin-4 catalog #2659-N4 and mouse Nectin-4 catalog #3116-N4.) The rats were injected weekly, for a total of seven injections, using a total of 25 μg of protein across three injection sites: subcutaneous, intraperitoneal, and footpad. Positive immunization titers were confirmed using protein ELISA with the human and mouse Nectin-4 recombinant protein. Splenocytes and lymph nodes were harvested from the rats and cells were isolated using gentle homogenization with a glass mortal/pestle. Red blood cells were then lysed using ACK lysing buffer for 5 minutes. Harvested cells were counted and fused with the myeloma partner cell line, SP2/0-Ag14, using a Nepa Gene Bulldog electroporator, 2-pulse setting. The fused hybridoma cells were then plated in 96-well tissue culture plates using limited dilution. After two weeks of growth, the hybridoma supernatants were screened for binding to human/mouse Nectin-4 by protein ELISA and by Biacore, to human Nectin-1 recombinant protein by protein ELISA, and to human Nectin-4 BaF3 cells by flow cytometry.

### Candidate Selection

Candidates that showed strong binding to human and mouse Nectin-4 protein and human Nectin-4 BAF3 cells and no binding to human Nectin-1 protein at the hybridoma supernatant stage were selected for hybridoma subcloning. Antibodies from the selected hybridomas were either produced recombinantly from small scale hybridoma cultures or as rat human chimeric antibodies using sequences derived from barcoded Next Generation Sequencing. The purified antibodies were then titrated and tested for binding to human/mouse Nectin-4 by protein ELISA and to T47D endogenous cells using flow cytometry. Additionally, the purified antibodies were characterized for affinity using Biacore and epitope binning using the Octet. The hybridoma candidates were narrowed down to six clones (SV011.3H9.1G1; SV011.55D10.1C1; SVO11.15H3.1A1; SV011.54H11.1A4; SV011.52E1.1B2; and SV011.54E9.1F1) with strong protein and cell binding properties to human Nectin-4 protein and cells that bound to diverse epitopes on the Nectin-4 IgV, IgC1, and IgC2 domains. The light variable sequences and light chain CDR sequences of the clones are shown in Table 9. The heavy chain variable sequences and the heavy chain CDR sequences of the clones are shown in Table 10.

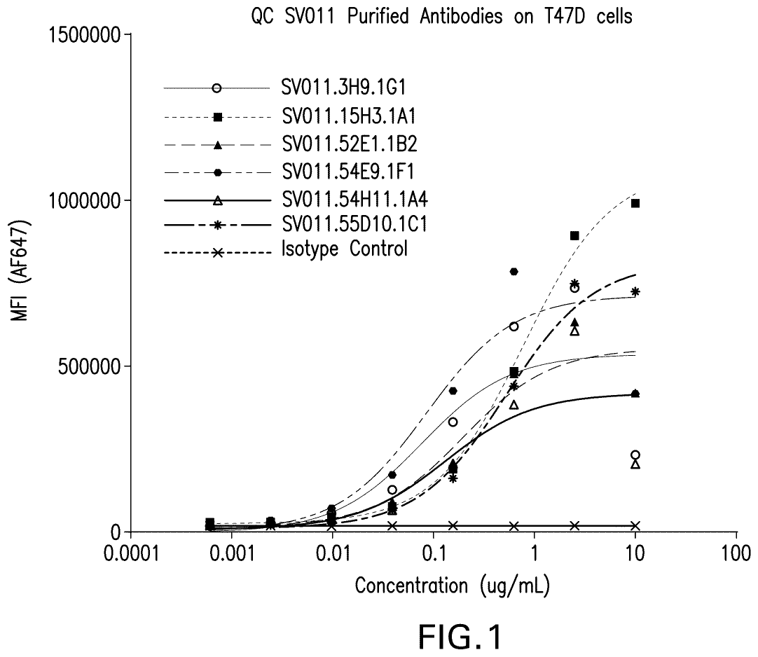

FIG. 1 shows FACS binding data of the purified antibodies from each of the six clones (SV011.3H9.1G1; SV011.55D10.1C1; SVO11.15H3.1A1; SV011.54H11.1A4; SV011.52E1.1B2; and SV011.54E9.1F1) to T47D cells.

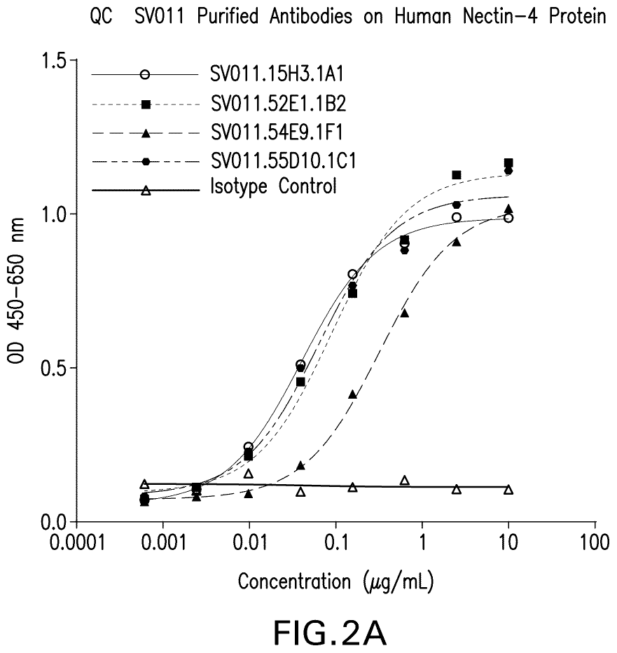

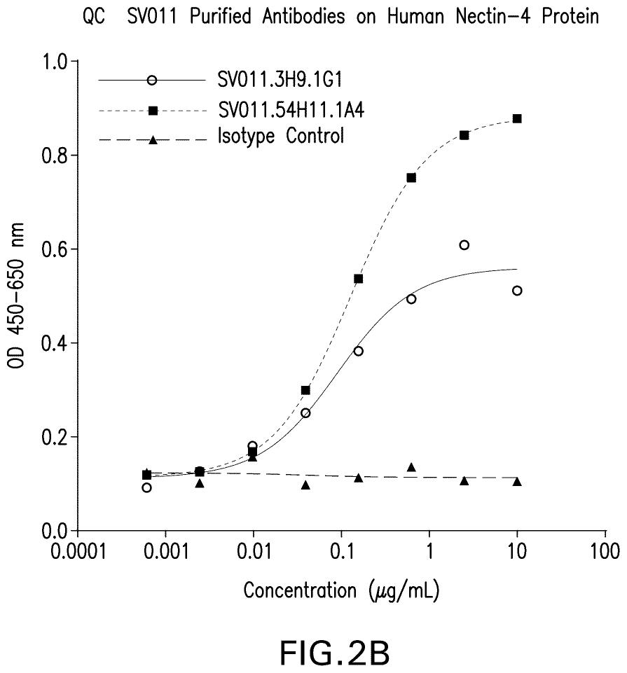

FIGS. 2A and 2B show ELISA binding to of the purified antibodies of the six clones (SV011.3H9.1G1; SV011.55D10.1C1; SVO11.15H3.1A1; SV011.54H11.1A4; SV011.52E1.1B2; and SV011.54E9.1F1) to the human Nectin-4 protein. A summary of the EC50 values is shown in Table 11.

TABLE 9
Light Chain Variable and Light Chain CDR Sequences (Rat Hybridoma Clones)

| Rat Clone | Light chain variable sequence | CDRL1 | CDRL2 | CDRL3 |
|---|---|---|---|---|
| 3H9.1G1 | DIQMTQSPASLSVYLGETVSIECLATEDIFSY | LATEDIFSY | GANRLK | LQGAKFPL |
| | LAWYQQKPGKSPQLLIYGANRLKDGVPSRF | LA (SEQ ID | D (SEQ ID | T (SEQ ID |
| | SGSGTGTQYSLRISGMQPEDEGDYYCLQGA | NO: 20) | NO: 21) | NO: 22) |
| | KFPLTFGSGTKLEIK (SEQ ID NO: 73) | | | |
| 55D10.1C1 | DIQMTQSPASLSASLGETVSIECLASEDIYSY | LASEDIYSY | AANRLQ | LQGSQFP |
| | LAWFQQKSGKSPQLLIYAANRLQDGVPSRF | LA (SEQ ID | D (SEQ ID | WT (SEQ |
| | SGSGSGTQYSLRISGMQPEDEGDYFCLQGS | NO: 85) | NO: 86) | ID NO: 87) |
| | QFPWTFGGGTKLELK (SEQ ID NO: 74) | | | |
| 15H3.1A1 | DIQMTQSPASLSASLGETVSIECLASEDIHNK | LASEDIHN | YGSNLQ | LQDSKNP |
| | LAWYQQKPGKSPQLLIYYGSNLQDGVPSRF | KLA (SEQ | D (SEQ ID | WT (SEQ |
| | SGSGSGTQYYLKINSLESEDVATYFCLQDSK | ID NO: 88) | NO: 89) | ID NO: 90) |
| | NPWTFGGGTKLEMK (SEQ ID NO: 75) | | | |
| 54H11.1A4 | DIVLTQSPALDVSLGQRATISCSASQSVSISR | SASQSVSIS | RTSILTS | QQSRESPF |
| | YNLIHWYQQKPGQQPKLLIWRTSILTSGIPA | RYNLIH | (SEQ ID | T (SEQ ID |
| | RFSGRGSGTDFTLTINPVQADDIATYYCQQS | (SEQ ID NO: | NO: 92) | NO: 93) |
| | RESPFTFGAGTRLELK (SEQ ID NO: 76) | 91) | | |
| 52E1.1B2 | NIVLTQSPATLSVTPGESVSLSCRASQSISTGI | RASQSISTG | FASQSIS | QQRDSSLF |
| | HWYQQKSNESPRLLIKFASQSISGIPSRFSGS | IH (SEQ ID | (SEQ ID | T (SEQ ID |
| | GSGTDFTLSINRVESEDFSIYYCQQRDSSLFT | NO: 94) | NO: 95) | NO: 96) |
| | FGAGTKLELK (SEQ ID NO: 77) | | | |
| 54E9.1F1 | TYELIQPPSTSVTLGNTVSLTCVGDDLPRRY | VGDDLPRR | EDSKRPS | HSTYSDD |
| | AYWYQQKPDQSIVRVIYEDSKRPSGISDRFS | YAY (SEQ | (SEQ ID | KVRV (SEQ |
| | GSSSGTTATLTIRDAQAEDEADYYCHSTYS | ID NO: 97) | NO: 98) | ID NO: 99) |
| | DDKVRVFGGGTKLTVL (SEQ ID NO: 78) | | | |

TABLE 10
Heavy Chain Variable and Heavy Chain CDR Sequences (Rat Hybridoma
Clones)

| Rat Clone | Heavy chain variable sequence | CDRH1 | CDRH2 | CDRH3 |
|---|---|---|---|---|
| 3H9.1G1 | EVQLVESGGGLVQPGRSMRLSCAASGFSFS | NYYMA | SISTGGG | QTAYYVM |
| | NYYMAWVRQAPRKGLDWVASISTGGGNIY | (SEQ ID NO: | NIYYRD | DA (SEQ ID |
| | YRDSVKGRFTISRDNAKSTLYLQMDSLRSE | 15) | SVKG | NO: 17) |
| | DTATYYCARQTAYYVMDAWGQGASVTVS | | (SEQ ID | |
| | S (SEQ ID NO: 79) | | NO: 16) | |
| 55D10.1C1 | EVQLVESGGGLVQPGRSLKLSCAASGFTFS | NYDMA | SISPRGG | IHHGYWYF |
| | NYDMAWVRQVPTKGLEWVASISPRGGRIY | (SEQ ID NO: | RIYYRD | DF (SEQ ID |
| | YRDSVKGRFTVSRDNAKSCLYLQMESLRSD | 100) | SVKG | NO: 102) |
| | DTATYYCARIHHGYWYFDFWGPGTMVTVS | | (SEQ ID | |
| | S (SEQ ID NO: 80) | | NO: 101) | |
| 15H3.1A1 | EVKLLESGGGLVQPGGSMRLSCAASGFTFT | DFYMI | FIRNKA | PLYYGYTP |
| | DFYMIWIRQPAGKAPEWLGFIRNKANGYTT | (SEQ ID NO: | NGYTTD | RY (SEQ ID |
| | DYNPSVKGRFTISRDNTQNMLYLQMITLRA | 103) | YNPSVK | NO: 105) |
| | EDTATYYCARPLYYGYTPRYWGQGVMVT | | G (SEQ | |
| | VSS (SEQ ID NO: 81) | | ID NO: | |
| | | | 104) | |
| 54H11.1A4 | EVQLVESGGGLVQPGRPLKLSCAASGFSFS | HYDMA | AISPSGG | QGPSYGYY |
| | HYDMAWVRQAPTKGLEWVAAISPSGGSTY | (SEQ ID NO: | STYYRD | FDY (SEQ |
| | YRDSVKGRFTVSRDKAKNSLYLQMDSLRSE | 106) | SVKG | ID NO: 108) |
| | DTATYYCARQGPSYGYYFDYWGQGVMVT | | (SEQ ID | |
| | VSS (SEQ ID NO: 82) | | NO: 107) | |
| 52E1.1B2 | EVQLVESGGGLVQPGRSLKVSCAASGFTFS | NYDMA | SINPGGI | RQPYFDY |
| | NYDMAWVRQAPTKGLEWVASINPGGISTY | (SEQ ID NO: | STYYRD | (SEQ ID NO: |
| | YRDSVKGRFTVSRDNEKSTLYLQMDSLRSE | 100) | SVKG | 110) |
| | DTATYYCARRQPYFDYWGQGVMVTVSS | | (SEQ ID | |
| | (SEQ ID NO: 83) | | NO: 109) | |
| 54E9.1F1 | QVTLKESGTGILQPSQALSLTCSISGFSLNTT | VGDDLPRR | EDSKRP | HSTYSDDK |
| | GICVSWIRQPLGQGLEWLADICWDDGKGY | YAY (SEQ | S (SEQ | VRV (SEQ |
| | NPSLKNRLSISKDTSNNQAFLKITRVDTTDT | ID NO: 111) | ID NO: | ID NO: 113) |
| | ATYYCARNYGGNPFDYWGQGVMVTVSS | | 112) | |
| | (SEQ ID NO: 84) | | | |

TABLE 11
EC50 values for purified Nectin-4 antibodies binding to T47D
cells and human Nectin-4 protein (Rat Hybridoma Clones)

| | T47D cells | Human-Nectin4 |
|---|---|---|
| | EC50 | EC50 |
| Clone | (ug/mL) | (ug/mL) |
| SV011.3H9.1G1 | 0.077 | 0.090 |
| SV011.15H3.1A1 | 0.772 | 0.040 |
| SV011.52E1.1B2 | 0.195 | 0.090 |
| SV011.54E9.1F1 | 0.085 | 0.319 |
| SV011.54H11.1A4 | 0.131 | 0.127 |
| SV011.55D10.1C1 | 0.524 | 0.063 |
| Isotype Control | NB | NB |

### Hybridoma Supernatant or Purified Antibody Protein ELISA Assay

96-well half area plates were coated with either 25 μL/well with recombinant protein (1 μg/mL in PBS buffer) and incubated overnight at 4° C. The next day, plates were washed 3 times with PBST (PBS+0.05% Tween 20) and blocked with 25 μL/well of blocking buffer (PBS with 5% FBS) for 30 minutes at room temperature. Either Hybridoma supernatant or titrated purified antibody was then transferred at 25 μl/well to the 96-well plates and incubated for 60 minutes at room temperature. The plates were then washed 3 times with PBST. Then 25 μl/well of anti-rat or anti-human IgG HRP conjugate (diluted in blocking buffer) was added to the plates and incubated for 60 minutes at room temperature. Finally, the plates were washed 5 times with PBST and developed by adding TMB reagent (Thermo cat #34029) to the plates for 2-3 minutes. The reactions were stopped with 0.16M sulfuric acid and the absorbance read at 450 nm and 650 nm using a spectrophometer.

### Hybridoma Supernatant or Purified Antibody Cell Binding Assay

Either recombinant BaF3 cells expressing human Nectin-4 or T47D endogenous cells were cultured for flow staining. The harvested cells were stained with either 50 μL of titrated purified antibodies or hybridoma supernatant for 30 minutes and then spun down and washed 1× with flow buffer (5% fetal bovine serum in PBS.) Next, the cells were stained with a fluorescently labeled secondary antibody (specific to the Fc domain of the hybridoma supernatant or purified recombinant antibody) for 30 minutes and spun down and washed 2× with flow buffer. Cells resuspended in 50 μL flow buffer were then analyzed using an Intellicyt.

### Next Generation Sequencing of Lysed Hybridomas

Hybridoma candidates of interest were lysed in 85 μL of Qiagen TCL buffer with 1% B-mercaptoethanol and RNA was isolated using Qiagen Turbocapture tubes (Qiagen, Catalog #72251). cDNA was generated using SuperScript IV reverse transcriptase (Thermo, Catalog #18090050) in the presence of a template switching oligo (TSO) for 5′RACE. A first round PCR reaction was then performed using GoTaq polymerase (Promega, Catalog #M7422), a forward R1 primer, and a reverse equimolar mixture of primers specific for the constant regions of the rat heavy and kappa/lambda light chains. A second round of PCR, also using GoTaq polymerase, further amplified the PCR products and introduced Illumina MiSeq NGS adaptors and indices for high-throughput, multiplexed NGS sequencing. Mixed PCR2 pools of heavy, kappa, and lambda chain samples were bead purified at a ratio of 0.8× using Agencourt AMPure XP beads (Beckman Coulter, Catalog #A63881). Individual, indexed PCR products were then pooled, and gel purified using a Qiagen gel purification kit (Qiagen, Catalog #28704). The NGS libraries were sequenced with an Illumina MiSeq and antibody variable regions were determined using a bioinformatic analysis pipeline. Sequences that showed unique variable regions (framework and CDR regions) were recombinantly expressed for further testing.

### Affinity Characterization of Hybridoma Supernatants and Purified Protein

Six-week-old Lewis rats were co-immunized with a 1:1 mixture of human and mouse His-tagged Nectin-4 recombinant proteins. The rats were injected weekly, for a total of 7 injections, using a total of 25 μg of protein across three injection sites: subcutaneous, intraperitoneal, and footpad. Positive immunization titers were confirmed using ELISA assay with the human and mouse Nectin-4 recombinant proteins. Splenocytes and lymph nodes were harvested from the rats and cells were isolated using gentle homogenization with a glass mortal/pestle. Red blood cells were then lysed using ACK lysing buffer for 5 minutes. Harvested cells were counted and fused with the myeloma partner cell line, SP2/0-Ag14, using a Nepa Gene Bulldog electroporator, 2-pulse setting. The fused hybridoma cells were then plated in 96-well tissue culture plates using limited dilution. After 2 weeks of culture, the hybridoma supernatants were screened for binding to human or mouse Nectin-4 by ELISA and by SPR, to human Nectin-1 recombinant protein by ELISA, and to human Nectin-4-expressing BaF3 cells by flow cytometry.

### Screening and Selection of Anti-Nectin-4 Hybridomas

Hybridoma supernatants that showed strong binding to human and mouse Nectin-4 proteins and to human Nectin-4-expressing BaF3 cells, and no binding to human Nectin-1 protein were selected for subcloning. Antibodies from the selected hybridomas were produced either recombinantly from small scale hybridoma cultures or as rat human chimeric antibodies using sequences derived from barcoded Next Generation Sequencing. The purified antibodies were then titrated and tested for binding to human or mouse Nectin-4 by ELISA and to T47D endogenous cells using flow cytometry. Additionally, the purified antibodies were characterized for affinity by SPR and epitope binning using the Octet.

### Hybridoma Supernatant or Purified Antibody Binding by ELISA

Microtiter 96-well assay plates were coated with either 25 μL/well with recombinant Nectin-4 protein (1 μg/mL in PBS buffer) and incubated overnight at 4° C. The next day, plates were washed 3 times with PBST (PBS+0.05% Tween® 20) and blocked with 25 μL/well of blocking buffer PBSF for 30 minutes at room temperature. Either hybridoma supernatant or titrated purified antibody was then transferred at 25 μL/well to the 96-well plates and incubated for 60 minutes at ambient temperature. The plates were then washed 3 times with PBST. Then 25 μL/well of anti-rat or anti-human IgG HRP conjugate (diluted in blocking buffer) was added to the plates and incubated for 60 minutes at ambient temperature. Plates were washed 5 times with PBST and developed by adding TMB reagent to the plates for 2 to 3 minutes. The reactions were stopped with 0.16 M sulfuric acid and the absorbance read at 450 nm and 650 nm using a spectrophotometer.

### Hybridoma Supernatant or Purified Antibody Cell Binding Assay by Flow Cytometry

Recombinant BaF3 cells expressing human Nectin-4 or T47D endogenous cells were cultured for flow staining. The harvested cells were stained with either 50 μL of titrated purified antibodies or hybridoma supernatant for 30 minutes and then washed once with PBSF flow buffer. Cells were stained with a fluorescently labeled secondary antibody (specific to the Fc domain of the hybridoma supernatant or purified recombinant antibody) for 30 minutes, spun down, and washed twice with PBSF. Cells were resuspended in 50 μL PBSF and then analyzed using an Intellicyt instrument (Essen Bioscience Inc (Sartorius), Michigan, USA).

### Example 3: Recombinant Antibody Production

The rat hybridoma antibodies clones SV011.3H9.1G1, SV011.15H3.1A1, SV011.52E1.1B2, SV011.43E9.1F1, SV011.54H11.1A4, and SV011.55D10.1C1, and mouse hybridoma clones, 14E4. E12, 14F6. B1, and 16H8. B1 were reformatted into Fabs and bispecific T cell engagers. The mouse Vh and Vl sequences of the hybridoma antibodies were placed into a standard Fab format with human light (kappa or lambda) and human heavy constant (CH1) domains to form a chimeric Fab. The chimeric Fab sequences were further reformatted into bispecific T cell engagers by appending an anti-CD3 scFv to the N-terminal light or N-terminal heavy chain of the chimer Fabs. DNA encoding the chimeric Fabs and T cell engagers were cloned into the pcDNA3.4 vector used to transiently express the proteins in ExpiCHO cells. Fabs and T cell engagers were then screened for binding to Nectin-4 and when relevant CD3 antigen binding. TCEs were screened for their ability to redirect T cell mediated target cell lysis in tumor cell co-culture assays. Fabs and T cell engagers were selected based on potency and activity then humanized. Humanized Fabs and TCEs were produced as described for the chimeric Fabs and TCEs. During humanization any potential detrimental PTMs within or near the CDRs of the binding domains were mutated out.

### Example 4: Humanization of Antibodies

Humanization of 3 mouse sequences (14E4. H3, 14F6. B1, 16H8. B) and one Rat sequence (SV011.3H1.1G1) Nectin-4 binding regions were performed using the BioPhi Sapiens software package. Structural models were built of the input sequence using the AbodyBuilder2 software package, and suggested highest probability mutations within 5 Angstroms of were iteratively compared to mutations of observed but lower probability using the total score feature of the Rosetta modelling software. The two lowest total score set of mutations were compared to the highest log likelihood from the Sapiens software, as well as the Best Single Mutations output from the Biovia Discovery Antibody Humanization workflow. The humanized light chain variable sequences and light chain CDR sequences are shown in Table 12. The humanized heavy chain variable sequences heavy chain CDR sequences are shown in Table 13. The full-length light sequences and heavy sequences of the humanized antibodies are shown in Tables 14 and 15, respectively. The nucleotide sequences that encode the full-length light sequences and heavy sequences of the humanized antibodies are shown in Tables 16, and 17, respectively.

TABLE 12
Light Chain Variable Sequences and Light Chain CDR Sequences of
Humanized Antibodies

| Sequence | Light chain variable | | | |
|---|---|---|---|---|
| Name | sequence | CDRL1 | CDRL2 | CDRL3 |
| DAB012120 | EIQMTQSHKTFSVSTGQRVTITCKA | KASQDVSIAVA | WASTRQT | QQYSSYPFT |
| | SQDVSIAVAWYQQKPGKAPKLLIY | (SEQ ID NO: 28) | (SEQ ID | (SEQ ID NO: |
| | WASTRQTGVPSRFSGSGSGTDFTL | | NO: 29) | 30) |
| | TISCVQSEDFAVYYCQQYSSYPFTF | | | |
| | GQGTKLEIK (SEQ ID NO: 24) | | | |
| DAB012121 | DIQMTQSPSFLSASVGDRVTITCKA | KASQDVSIAVA | WASTRQT | QQYSSYPFT |
| | SQDVSIAVAWYQQKPGKAPKLLIY | (SEQ ID NO: 28) | (SEQ ID | (SEQ ID NO: |
| | WASTRQTGVPSRFSGSGSGTDFTL | | NO: 29) | 30) |
| | TISSLQPEDFATYFCQQYSSYPFTF | | | |
| | GQGTKLEIK (SEQ ID NO: 25) | | | |
| DAB012122 | DIQMTQSPSFLSASVGERVTITCKA | KASQDVSIAVA | WASTRQT | QQYSSYPFT |
| | SQDVSIAVAWYQQKPGKAPKLLIY | (SEQ ID NO: 28) | (SEQ ID | (SEQ ID NO: |
| | WASTRQTGVPSRFSGSGSGTDFTL | | NO: 29) | 30) |
| | TISSLQPEDIATYFCQQYSSYPFTFG | | | |
| | QGTKLEIK (SEQ ID NO: 26) | | | |
| DAB012123 | DIQMTQSPSFLSASVGDRVTITCKA | KASQDVSIAVA | WASTRQT | QQYSSYPFT |
| | SQDVSIAVAWYQQKPGKAPKLLIY | (SEQ ID NO: 28) | (SEQ ID | (SEQ ID NO: |
| | WASTRQTGVPSRFSGSGSGTDFTL | | NO: 29) | 30) |
| | TISSLQPEDSATYFCQQYSSYPFTF | | | |
| | GQGTKVDIK (SEQ ID NO: 27) | | | |
| DAB012124 | EIQMTQSQSTLSVSLGDRVTITCKA | KASQNVVTAVA | SASHRFT | QQYSSYPYT |
| | SQNVVTAVAWYQQKPGQAPKLLI | (SEQ ID NO: 35) | (SEQ ID | (SEQ ID NO: |
| | YSASHRFTGVPSRFSGSGSGTDFTL | | NO: 36) | 37) |
| | IISCMQSEDFAVYYCQQYSSYPYTF | | | |
| | GQGTKLEIK (SEQ ID NO: 31) | | | |
| DAB012125 | DIQMTQSPSFLSASVGDRVTITCKA | KASQNVVTAVA | SASHRFT | QQYSSYPYT |
| | SQNVVTAVAWYQQKPGKAPKLLI | (SEQ ID NO: 35) | (SEQ ID | (SEQ ID NO: |
| | YSASHRFTGVPSRFSGSGSGTDFTL | | NO: 36) | 37) |
| | TISNLQSEDLATYFCQQYSSYPYTF | | | |
| | GQGTKLEIK (SEQ ID NO: 32) | | | |
| DAB012126 | DIQMTQSPSFLSASVGDRVTITCKA | KASQNVVTAVA | SASHRFT | QQYSSYPYT |
| | SQNVVTAVAWYQQKPGKAPKLLI | (SEQ ID NO: 35) | (SEQ ID | (SEQ ID NO: |
| | YSASHRFTGVPSRFSGSGSGTDFTL | | NO: 36) | 37) |
| | TISNLQPEDSATYFCQQYSSYPYTF | | | |
| | GQGTKLEIK (SEQ ID NO: 33) | | | |
| DAB012127 | DIQMTQSPSFLSASVGDRVTITCKA | KASQNVVTAVA | SASHRFT | QQYSSYPYT |
| | SQNVVTAVAWYQQKPGKAPKLLI | (SEQ ID NO: 35) | (SEQ ID | (SEQ ID NO: |
| | YSASHRFTGVPSRFSGSGSGTDFTL | | NO: 36) | 37) |
| | TISNLQPDDAATYFCQQYSSYPYTF | | | |
| | GQGTKLEIK (SEQ ID NO: 34) | | | |
| DAB012128 | AIQMTQSHKSFSVSTGQRVTITCK | KASQDVSTTVA | WASTRQT | QQYSSYPFT |
| | ASQDVSTTVAWYQQKPGQAPKLL | (SEQ ID NO: 42) | (SEQ ID | (SEQ ID NO: |
| | IYWASTRQTGVPSRFTGSGSGTDF | | NO: 29) | 30) |
| | TLTISCVQSEDFAVYYCQQYSSYPF | | | |
| | TFGQGTKLEIK (SEQ ID NO: 38) | | | |
| DAB012129 | DIQMTQSPSFLSASVGDRVTITCKA | KASQDVSTTVA | WASTRQT | QQYSSYPFT |
| | SQDVSTTVAWYQQKPGKAPKLLI | (SEQ ID NO: 42) | (SEQ ID | (SEQ ID NO: |
| | YWASTRQTGVPSRFSGSGSGTDFT | | NO: 29) | 30) |
| | LTISNLQPEDFATYFCQQYSSYPFT | | | |
| | FGQGTKLEIK (SEQ ID NO: 39) | | | |
| DAB012130 | DIQMTQSPSFLSASVGERVTITCKA | KASQDVSTTVA | WASTRQT | QQYSSYPFT |
| | SQDVSTTVAWYQQKPGKAPKLLI | (SEQ ID NO: 42) | (SEQ ID | (SEQ ID NO: |
| | YWASTRQTGVPSRFSGSGSGTDFT | | NO: 29) | 30) |
| | LTINSLOPEDVATYFCQQYSSYPFT | | | |
| | FGQGTKLEIK (SEQ ID NO: 40) | | | |
| DAB012131 | DIQMTQSPSFLSASVGERVTITCKA | KASQDVSTTVA | WASTRQT | QQYSSYPFT |
| | SQDVSTTVAWYQQKPGKAPKLLI | (SEQ ID NO: 42) | (SEQ ID | (SEQ ID NO: |
| | YWASTRQTGVPSRFSGSGSGTDFT | | NO: 29) | 30) |
| | LTISNLQPEDVATYFCQQYSSYPFT | | | |
| | FGQGTKLEIK (SEQ ID NO: 41) | | | |
| DAB012132 | DIQMTQSPSSVSLTVGQRVTITCLA | LATEDIFSYLA | GANRLKD | LQGAKFPLT |
| | TEDIFSYLAWYQQKPGKAPRLLIY | (SEQ ID NO: 20) | (SEQ ID | (SEQ ID NO: |
| | GANRLKDGVPSRFSGSGSGTQYTL | | NO: 21) | 22) |
| | RISSMQPEDFGVYYCLQGAKFPLT | | | |
| | FGPGTKLEIK (SEQ ID NO: 43) | | | |
| DAB012133 | DIQMTQSPSSLSASIGDTVTITCLAT | LATEDIFSYLA | GANRLKD | LQGAKFPLT |
| | EDIFSYLAWYQQRPGKAPKLLIYG | (SEQ ID NO: 20) | (SEQ ID | (SEQ ID NO: |
| | ANRLKDGVPSRFSGSGSGTDYTLTI | | NO: 21) | 22) |
| | SGLQPEDFVTYYCLQGAKFPLTFG | | | |
| | QGTKLDMK (SEQ ID NO: 44) | | | |
| DAB012134 | DIQMTQSPSSLSASVGDTVTITCLA | LATEDIFSYLA | GANRLKD | LQGAKFPLT |
| | TEDIFSYLAWYQQKPGKAPKLLIY | (SEQ ID NO: 20) | (SEQ ID | (SEQ ID NO: |
| | GANRLKDGVPSRFSGSGSGTDYTL | | NO: 21) | 22) |
| | TISGLQPEDFGTYYCLQGAKFPLTF | | | |
| | GQGTKLEIK (SEQ ID NO: 45) | | | |
| DAB012135 | DIQMTQSPSSLSASVGDTVTITCLA | LATEDIFSYLA | GANRLKD | LOGAKFPLT |
| | TEDIFSYLAWYQQKPGKAPKLLIY | (SEQ ID NO: 20) | (SEQ ID | (SEQ ID NO: |
| | GANRLKDGVPSRFSGGGSGTEYSL | | NO: 21) | 22) |
| | TISGLQPEDFGTYYCLQGAKFPLTF | | | |
| | GQGTKLDMK (SEQ ID NO: 46) | | | |
| DAB012135 | DIQMTQSPSSLSASVGDTVTITCLA | LATEDIFSYLA | GANRLKD | LQGAKFPLT |
| (modified) | TEDIFSYLAWYQQKPGKAPKLLIY | (SEQ ID NO: 20) | (SEQ ID | (SEQ ID NO: |
| | GANRLKDTVPSRFSGGGSGTEYSL | | NO: 21) | 22) |
| | TISGLQPEDFGTYYCLQGAKFPLTF | | | |
| | GQGTKLDMKR (SEQ ID NO: 47) | | | |

TABLE 13
Heavy Chain Variable Sequences and Heavy Chain CDR Sequences of
Humanized Antibodies

| Sequence | | | | |
|---|---|---|---|---|
| Name | Heavy chain variable sequence | CDRH1 | CDRH2 | CDRH3 |
| DAB012120 | QVQLLESGGELVQPGGSLRLSCAA | DYYMF | YISNGGGNTYY | PEARYYGN |
| | SGFTFSDYYMFWIRQTPQKRLEWV | (SEQ ID NO: | PDTVKG (SEQ | YPFPY (SEQ |
| | AYISNGGGNTYYPDTVKGRFTISR | 52) | ID NO: 53) | ID NO: 54) |
| | DNAKNQLYLQMRSLTPEDTAIYYC | | | |
| | ASPEARYYGNYPFPYWGQGTLVT | | | |
| | VSS (SEQ ID NO: 48) | | | |
| DAB012121 | QVQLVESGGGLVKPGGSLRLSCAA | DYYMF | YISNGGGNTYY | PEARYYGN |
| | SGFTFSDYYMFWIRQAPGKGLEW | (SEQ ID NO: | PDTVKG (SEQ | YPFPY |
| | VAYISNGGGNTYYPDTVKGRFTIS | 52) | ID NO: 53) | (SEQ ID NO: |
| | RDNAKNTLYLQMNSLRAEDTAVY | | | 54) |
| | YCASPEARYYGNYPFPYWGQGTL | | | |
| | VTVSS (SEQ ID NO: 49) | | | |
| DAB012122 | QVQLVESGGGVVKPGGSLRLSCA | DYYMF | YISNGGGNTYY | PEARYYGN |
| | ASGFTFSDYYMFWIRQAPGKGLE | (SEQ ID NO: | PDTVKG (SEQ | YPFPY |
| | WVAYISNGGGNTYYPDTVKGRFTI | 52) | ID NO: 53) | (SEQ ID NO: |
| | SRDNAKNTLYLQMNSLRAEDTAV | | | 54) |
| | YYCASPEARYYGNYPFPYWGQGT | | | |
| | LVTVSS (SEQ ID NO: 50) | | | |
| DAB012123 | QVQLVESGGGVVQPGGSLRLSCA | DYYMF | YISNGGGNTYY | PEARYYGN |
| | ASGFTFSDYYMFWIRQAPGKGLE | (SEQ ID NO: | PDTVKG (SEQ | YPFPY |
| | WVAYISNGGGNTYYPDTVKGRFTI | 52) | ID NO: 53) | (SEQ ID NO: |
| | SRDNAKNTLYLQMNSLRAEDTAV | | | 54) |
| | YYCASPEARYYGNYPFPYWGQGT | | | |
| | LVTVSS (SEQ ID NO: 51) | | | |
| DAB012124 | QVQLLQSGPGLVKPSATLSLTCTV | TYGVH (SEQ | VIWSGGSTDYN | TSHWYFDV |
| | SGFSLTTYGVHWIRQPPGKGLEW | ID NO: 59) | AAFIS (SEQ ID | (SEQ ID NO: |
| | MGVIWSGGSTDYNAAFISRLTISK | | NO: 60) | 61) |
| | DNSKNQVFLQLYSLRAEDTAIYYC | | | |
| | ARTSHWYFDVWGRGTLVTVSS | | | |
| | (SEQ ID NO: 55) | | | |
| DAB012125 | QVQLQESGPGLVKPSQTLSLTCTV | TYGVH (SEQ | VIWSGGSTDYN | TSHWYFDV |
| | SGFSLTTYGVHWVRQSPGKGLEW | ID NO: 59) | AAFIS (SEQ ID | (SEQ ID NO: |
| | LGVIWSGGSTDYNAAFISRLSISKD | | NO: 60) | 61) |
| | NSKSQVFLEMTSLTAADTAIYYCA | | | |
| | RTSHWYFDVWGRGTLVTVSS | | | |
| | (SEQ ID NO: 56) | | | |
| DAB012126 | QVQLQESGPGLVKPSQTLSLTCTV | TYGVH (SEQ | VIWSGGSTDYN | TSHWYFDV |
| | SGFSLTTYGVHWVRQAPEKGLEW | ID NO: 59) | AAFIS (SEQ ID | (SEQ ID NO: |
| | LGVIWSGGSTDYNAAFISRLSISKD | | NO: 60) | 61) |
| | NSKSQVFLEMTSLTADDTAIYYCA | | | |
| | RTSHWYFDVWGRGTLVTVSS | | | |
| | (SEQ ID NO: 57) | | | |
| DAB012127 | QVQLQESGPGLVKPSQTLSLTCTV | TYGVH (SEQ | VIWSGGSTDYN | TSHWYFDV |
| | SGFSLTTYGVHWVRQTPEKGLEW | ID NO: 59) | AAFIS (SEQ ID | (SEQ ID NO: |
| | LGVIWSGGSTDYNAAFISRLSISKD | | NO: 60) | 61) |
| | NSKSQVFLEMTSLTADDTAIYYCA | | | |
| | RTSHWYFDVWGRGTLVTVSS | | | |
| | (SEQ ID NO: 58) | | | |
| DAB012128 | QVQLLESGGGLVQPGGSLRLSCAA | DYYMY (SEQ | YISNGGGNTYY | PEARYYGN |
| | SGFTFSDYYMYWIRQTPQKRLEW | ID NO: 66) | SDTVKG (SEQ | FPFPY (SEQ |
| | VAYISNGGGNTYYSDTVKGRFTIS | | ID NO: 67) | ID NO: 68) |
| | RDNAKNQLYLQMRSLRPEDTAIY | | | |
| | YCASPEARYYGNFPFPYWGQGTL | | | |
| | VTVSS (SEQ ID NO: 62) | | | |
| DAB012129 | QVQLVESGGGLVKPGGSLRLSCAA | DYYMY (SEQ | YISNGGGNTYY | PEARYYGN |
| | SGFTFSDYYMYWIRQAPGKGLEW | ID NO: 66) | SDTVKG (SEQ | FPFPY (SEQ |
| | VAYISNGGGNTYYSDTVKGRFTIS | | ID NO: 67) | ID NO: 68) |
| | RDNAKNTLYLQMNSLRAEDTAVY | | | |
| | YCASPEARYYGNFPFPYWGQGTL | | | |
| | VTVSS (SEQ ID NO: 63) | | | |
| DAB012130 | QVQLVESGGGLVKPGGSLRLSCAA | DYYMY (SEQ | YISNGGGNTYY | PEARYYGN |
| | SGFTFSDYYMYWIRQAPGKGLEW | ID NO: 66) | SDTVKG (SEQ | FPFPY (SEQ |
| | VAYISNGGGNTYYSDTVKGRFTIS | | ID NO: 67) | ID NO: 68) |
| | RDNAKNTLYLQMNSLRAEDTAVY | | | |
| | YCASPEARYYGNFPFPYWGQGTL | | | |
| | VTVSS (SEQ ID NO: 64) | | | |
| DAB012131 | QVQLVESGGGLVKPGGSLRLSCAA | DYYMY (SEQ | YISNGGGNTYY | PEARYYGN |
| | SGFTFSDYYMYWIRQAPGKGLEW | ID NO: 66) | SDTVKG (SEQ | FPFPY (SEQ |
| | VAYISNGGGNTYYSDTVKGRFTIS | | ID NO: 67) | ID NO: 68) |
| | RDNAKNTLYLQMNSLRAEDTAVY | | | |
| | YCASPEARYYGNFPFPYWGQGTL | | | |
| | VTVSS (SEQ ID NO: 65) | | | |
| DAB012132 | QVQLVESGGGVVQPGRSLRLSCA | NYYMA (SEQ | SISTGGGNIYYR | QTAYYVM |
| | ASGFSFSNYYMAWVRQAPGKGLE | ID NO: 15) | DSVKG (SEQ ID | DA (SEQ ID |
| | WVASISTGGGNIYYRDSVKGRFTIS | | NO: 16) | NO: 17) |
| | RDNSKSTLYLQMRSLRSEDTAIYY | | | |
| | CARQTAYYVMDAWGQGTMVTVS | | | |
| | S (SEQ ID NO: 69) | | | |
| DAB012133 | EVQLVESGGGLVQPGGSLRLSCAA | NYYMA (SEQ | SISTGGGNIYYR | QTAYYVM |
| | SGFSFSNYYMAWVRQAPGKGLEW | ID NO: 15) | DSVKG (SEQ ID | DA (SEQ ID |
| | VASISTGGGNIYYRDSVKGRFTISR | | NO: 16) | NO: 17) |
| | DNAKNSLYLQMNSLRAEDTAVYY | | | |
| | CARQTAYYVMDAWGQGTTVTVS | | | |
| | S (SEQ ID NO: 70) | | | |
| DAB012134 | EVQLVESGGGLVQPGGSLRLSCAA | NYYMA (SEQ | SISTGGGNIYYR | QTAYYVM |
| | SGFSFSNYYMAWVRQAPGKGLEW | ID NO: 15) | DSVKG (SEQ ID | DA (SEQ ID |
| | VASISTGGGNIYYRDSVKGRFTISR | | NO: 16) | NO: 17) |
| | DNAKNSLYLQMNSLRAEDTAVYY | | | |
| | CARQTAYYVMDAWGQGTTVTVS | | | |
| | S (SEQ ID NO: 71) | | | |
| DAB012135 | EVQLVESGGGLVQPGGSLRLSCAA | NYYMA (SEQ | SISTGGGNIYYR | QTAYYVM |
| | SGFSFSNYYMAWVRQAPGKGLEW | ID NO: 15) | DSVKG (SEQ ID | DA (SEQ ID |
| | VASISTGGGNIYYRDSVKGRFTISR | | NO: 16) | NO: 17) |
| | DNAKNSLYLQMNSLRAEDTAVYY | | | |
| | CARQTAYYVMDAWGQGTTVTVS | | | |
| | S (SEQ ID NO: 72) | | | |

TABLE 14
Full Length Light Chain Sequences of Humanized Antibodies

| Sequence | |
|---|---|
| Name | Light chain sequence |
| DAB012120 | EIQMTQSHKTFSVSTGQRVTITCKASQDVSIAVAWYQQKPGKAPKLLIYWASTRQTGVP |
| | SRFSGSGSGTDFTLTISCVQSEDFAVYYCQQYSSYPFTFGQGTKLEIKRTVAAPSVFIFPPS |
| | DEQLKSGTASVVCLLNNFYPREAKVQWKVDNALQSGNSQESVTEQDSKDSTYSLSSTL |
| | TLSKADYEKHKVYACEVTHQGLSSPVTKSFNRGEC (SEQ ID NO: 127) |
| DAB012121 | DIQMTQSPSFLSASVGDRVTITCKASQDVSIAVAWYQQKPGKAPKLLIYWASTRQTGVP |
| | SRFSGSGSGTDFTLTISSLQPEDFATYFCQQYSSYPFTFGQGTKLEIKRTVAAPSVFIFPPS |
| | DEQLKSGTASVVCLLNNFYPREAKVQWKVDNALQSGNSQESVTEQDSKDSTYSLSSTL |
| | TLSKADYEKHKVYACEVTHQGLSSPVTKSFNRGEC (SEQ ID NO: 128) |
| DAB012122 | DIQMTQSPSFLSASVGERVTITCKASQDVSIAVAWYQQKPGKAPKLLIYWASTRQTGVP |
| | SRFSGSGSGTDFTLTISSLQPEDIATYFCQQYSSYPFTFGQGTKLEIKRTVAAPSVFIFPPSD |
| | EQLKSGTASVVCLLNNFYPREAKVQWKVDNALQSGNSQESVTEQDSKDSTYSLSSTLTL |
| | SKADYEKHKVYACEVTHQGLSSPVTKSFNRGEC (SEQ ID NO: 129) |
| DAB012123 | DIQMTQSPSFLSASVGDRVTITCKASQDVSIAVAWYQQKPGKAPKLLIYWASTRQTGVP |
| | SRFSGSGSGTDFTLTISSLQPEDSATYFCQQYSSYPFTFGQGTKVDIKRTVAAPSVFIFPPS |
| | DEQLKSGTASVVCLLNNFYPREAKVQWKVDNALQSGNSQESVTEQDSKDSTYSLSSTL |
| | TLSKADYEKHKVYACEVTHQGLSSPVTKSFNRGEC (SEQ ID NO: 130) |
| DAB012124 | EIQMTQSQSTLSVSLGDRVTITCKASQNVVTAVAWYQQKPGQAPKLLIYSASHRFTGVP |
| | SRFSGSGSGTDFTLIISCMQSEDFAVYYCQQYSSYPYTFGQGTKLEIKRTVAAPSVFIFPPS |
| | DEQLKSGTASVVCLLNNFYPREAKVQWKVDNALQSGNSQESVTEQDSKDSTYSLSSTL |
| | TLSKADYEKHKVYACEVTHQGLSSPVTKSFNRGEC (SEQ ID NO: 131) |
| DAB012125 | DIQMTQSPSFLSASVGDRVTITCKASQNVVTAVAWYQQKPGKAPKLLIYSASHRFTGVP |
| | SRFSGSGSGTDFTLTISNLQSEDLATYFCQQYSSYPYTFGQGTKLEIKRTVAAPSVFIFPPS |
| | DEQLKSGTASVVCLLNNFYPREAKVQWKVDNALQSGNSQESVTEQDSKDSTYSLSSTL |
| | TLSKADYEKHKVYACEVTHQGLSSPVTKSFNRGEC (SEQ ID NO: 132) |
| DAB012126 | DIQMTQSPSFLSASVGDRVTITCKASQNVVTAVAWYQQKPGKAPKLLIYSASHRFTGVP |
| | SRFSGSGSGTDFTLTISNLQPEDSATYFCQQYSSYPYTFGQGTKLEIKRTVAAPSVFIFPPS |
| | DEQLKSGTASVVCLLNNFYPREAKVQWKVDNALQSGNSQESVTEQDSKDSTYSLSSTL |
| | TLSKADYEKHKVYACEVTHQGLSSPVTKSFNRGEC (SEQ ID NO: 133) |
| DAB012127 | DIQMTQSPSFLSASVGDRVTITCKASQNVVTAVAWYQQKPGKAPKLLIYSASHRFTGVP |
| | SRFSGSGSGTDFTLTISNLQPDDAATYFCQQYSSYPYTFGQGTKLEIKRTVAAPSVFIFPPS |
| | DEQLKSGTASVVCLLNNFYPREAKVQWKVDNALQSGNSQESVTEQDSKDSTYSLSSTL |
| | TLSKADYEKHKVYACEVTHQGLSSPVTKSFNRGEC (SEQ ID NO: 134) |
| DAB012128 | AIQMTQSHKSFSVSTGQRVTITCKASQDVSTTVAWYQQKPGQAPKLLIYWASTRQTGVP |
| | SRFTGSGSGTDFTLTISCVQSEDFAVYYCQQYSSYPFTFGQGTKLEIKRTVAAPSVFIFPPS |
| | DEQLKSGTASVVCLLNNFYPREAKVQWKVDNALQSGNSQESVTEQDSKDSTYSLSSTL |
| | TLSKADYEKHKVYACEVTHQGLSSPVTKSFNRGEC (SEQ ID NO: 135) |
| DAB012129 | DIQMTQSPSFLSASVGDRVTITCKASQDVSTTVAWYQQKPGKAPKLLIYWASTRQTGVP |
| | SRFSGSGSGTDFTLTISNLQPEDFATYFCQQYSSYPFTFGQGTKLEIKRTVAAPSVFIFPPS |
| | DEQLKSGTASVVCLLNNFYPREAKVQWKVDNALQSGNSQESVTEQDSKDSTYSLSSTL |
| | TLSKADYEKHKVYACEVTHQGLSSPVTKSFNRGEC (SEQ ID NO: 136) |
| DAB012130 | DIQMTQSPSFLSASVGERVTITCKASQDVSTTVAWYQQKPGKAPKLLIYWASTRQTGVP |
| | SRFSGSGSGTDFTLTINSLQPEDVATYFCQQYSSYPFTFGQGTKLEIKRTVAAPSVFIFPPS |
| | DEQLKSGTASVVCLLNNFYPREAKVQWKVDNALQSGNSQESVTEQDSKDSTYSLSSTL |
| | TLSKADYEKHKVYACEVTHQGLSSPVTKSFNRGEC (SEQ ID NO: 137) |
| DAB012131 | DIQMTQSPSFLSASVGERVTITCKASQDVSTTVAWYQQKPGKAPKLLIYWASTRQTGVP |
| | SRFSGSGSGTDFTLTISNLQPEDVATYFCQQYSSYPFTFGQGTKLEIKRTVAAPSVFIFPPS |
| | DEQLKSGTASVVCLLNNFYPREAKVQWKVDNALQSGNSQESVTEQDSKDSTYSLSSTL |
| | TLSKADYEKHKVYACEVTHQGLSSPVTKSFNRGEC (SEQ ID NO: 138) |
| DAB012132 | DIQMTQSPSSVSLTVGQRVTITCLATEDIFSYLAWYQQKPGKAPRLLIYGANRLKDGVPS |
| | RFSGSGSGTQYTLRISSMQPEDFGVYYCLQGAKFPLTFGPGTKLEIKRTVAAPSVFIFPPS |
| | DEQLKSGTASVVCLLNNFYPREAKVQWKVDNALQSGNSQESVTEQDSKDSTYSLSSTL |
| | TLSKADYEKHKVYACEVTHQGLSSPVTKSFNRGEC (SEQ ID NO: 139) |
| DAB012133 | DIQMTQSPSSLSASIGDTVTITCLATEDIFSYLAWYQQRPGKAPKLLIYGANRLKDGVPSR |
| | FSGSGSGTDYTLTISGLQPEDFVTYYCLQGAKFPLTFGQGTKLDMKRTVAAPSVFIFPPS |
| | DEQLKSGTASVVCLLNNFYPREAKVQWKVDNALQSGNSQESVTEQDSKDSTYSLSSTL |
| | TLSKADYEKHKVYACEVTHQGLSSPVTKSFNRGEC (SEQ ID NO: 140) |
| DAB012134 | DIQMTQSPSSLSASVGDTVTITCLATEDIFSYLAWYQQKPGKAPKLLIYGANRLKDGVPS |
| | RFSGSGSGTDYTLTISGLQPEDFGTYYCLQGAKFPLTFGQGTKLEIKRTVAAPSVFIFPPS |
| | DEQLKSGTASVVCLLNNFYPREAKVQWKVDNALQSGNSQESVTEQDSKDSTYSLSSTL |
| | TLSKADYEKHKVYACEVTHQGLSSPVTKSFNRGEC (SEQ ID NO: 141) |
| DAB012135 | DIQMTQSPSSLSASVGDTVTITCLATEDIFSYLAWYQQKPGKAPKLLIYGANRLKDGVPS |
| | RFSGGGSGTEYSLTISGLQPEDFGTYYCLQGAKFPLTFGQGTKLDMKRTVAAPSVFIFPP |
| | SDEQLKSGTASVVCLLNNFYPREAKVQWKVDNALQSGNSQESVTEQDSKDSTYSLSST |
| | LTLSKADYEKHKVYACEVTHQGLSSPVTKSFNRGEC (SEQ ID NO: 142) |
| DAB012135 | DIQMTQSPSSLSASVGDTVTITCLATEDIFSYLAWYQQKPGKAPKLLIYGANRLKDTVPS |
| (modified) | RFSGGGSGTEYSLTISGLQPEDFGTYYCLQGAKFPLTFGQGTKLDMKRTVAAPSVFIFPP |
| | SDEQLKSGTASVVCLLNNFYPREAKVQWKVDNALQSGNSQESVTEQDSKDSTYSLSST |
| | LTLSKADYEKHKVYACEVTHQGLSSPVTKSFNRGEC (SEQ ID NO: 143) |

TABLE 15
Full Length Heavy Chain Sequences of Humanized Antibodies

| Sequence | |
|---|---|
| Name | Heavy chain sequence |
| DAB012120 | QVQLLESGGELVQPGGSLRLSCAASGFTFSDYYMFWIRQTPQKRLEWVAYISNGGGNT |
| | YYPDTVKGRFTISRDNAKNQLYLQMRSLTPEDTAIYYCASPEARYYGNYPFPYWGQGT |
| | LVTVSSASTKGPSVFPLAPSSKSTSGGTAALGCLVKDYFPEPVTVSWNSGALTSGVHTFP |
| | AVLQSSGLYSLSSVVTVPSSSLGTQTYICNVNHKPSNTKVDKKVEPKSCDKTHTCPPCPA |
| | PELLGGPSVFLFPPKPKDTLMISRTPEVTCVVVDVSHEDPEVKFNWYVDGVEVHNAKTK |
| | PREEQYNSTYRVVSVLTVLHQDWLNGKEYKCKVSNKALPAPIEKTISKAKGQPREPQV |
| | YTLPPSRDELTKNQVSLTCLVKGFYPSDIAVEWESNGQPENNYKTTPPVLDSDGSFFLYS |
| | KLTVDKSRWQQGNVFSCSVMHEALHNHYTQKSLSLSPGK (SEQ ID NO: 144) |
| DAB012121 | QVQLVESGGGLVKPGGSLRLSCAASGFTFSDYYMFWIRQAPGKGLEWVAYISNGGGNT |
| | YYPDTVKGRFTISRDNAKNTLYLQMNSLRAEDTAVYYCASPEARYYGNYPFPYWGQG |
| | TLVTVSSASTKGPSVFPLAPSSKSTSGGTAALGCLVKDYFPEPVTVSWNSGALTSGVHTF |
| | PAVLQSSGLYSLSSVVTVPSSSLGTQTYICNVNHKPSNTKVDKKVEPKSCDKTHTCPPCP |
| | APELLGGPSVFLFPPKPKDTLMISRTPEVTCVVVDVSHEDPEVKFNWYVDGVEVHNAKT |
| | KPREEQYNSTYRVVSVLTVLHQDWLNGKEYKCKVSNKALPAPIEKTISKAKGQPREPQ |
| | VYTLPPSRDELTKNQVSLTCLVKGFYPSDIAVEWESNGQPENNYKTTPPVLDSDGSFFLY |
| | SKLTVDKSRWQQGNVFSCSVMHEALHNHYTQKSLSLSPGK (SEQ ID NO: 145) |
| DAB012122 | QVQLVESGGGVVKPGGSLRLSCAASGFTFSDYYMFWIRQAPGKGLEWVAYISNGGGNT |
| | YYPDTVKGRFTISRDNAKNTLYLQMNSLRAEDTAVYYCASPEARYYGNYPFPYWGQG |
| | TLVTVSSASTKGPSVFPLAPSSKSTSGGTAALGCLVKDYFPEPVTVSWNSGALTSGVHTF |
| | PAVLQSSGLYSLSSVVTVPSSSLGTQTYICNVNHKPSNTKVDKKVEPKSCDKTHTCPPCP |
| | APELLGGPSVFLFPPKPKDTLMISRTPEVTCVVVDVSHEDPEVKFNWYVDGVEVHNAKT |
| | KPREEQYNSTYRVVSVLTVLHQDWLNGKEYKCKVSNKALPAPIEKTISKAKGQPREPQ |
| | VYTLPPSRDELTKNQVSLTCLVKGFYPSDIAVEWESNGQPENNYKTTPPVLDSDGSFFLY |
| | SKLTVDKSRWQQGNVFSCSVMHEALHNHYTQKSLSLSPGK (SEQ ID NO: 146) |
| DAB012123 | QVQLVESGGGVVQPGGSLRLSCAASGFTFSDYYMFWIRQAPGKGLEWVAYISNGGGNT |
| | YYPDTVKGRFTISRDNAKNTLYLQMNSLRAEDTAVYYCASPEARYYGNYPFPYWGQG |
| | TLVTVSSASTKGPSVFPLAPSSKSTSGGTAALGCLVKDYFPEPVTVSWNSGALTSGVHTF |
| | PAVLQSSGLYSLSSVVTVPSSSLGTQTYICNVNHKPSNTKVDKKVEPKSCDKTHTCPPCP |
| | APELLGGPSVFLFPPKPKDTLMISRTPEVTCVVVDVSHEDPEVKFNWYVDGVEVHNAKT |
| | KPREEQYNSTYRVVSVLTVLHQDWLNGKEYKCKVSNKALPAPIEKTISKAKGQPREPQ |
| | VYTLPPSRDELTKNQVSLTCLVKGFYPSDIAVEWESNGQPENNYKTTPPVLDSDGSFFLY |
| | SKLTVDKSRWQQGNVFSCSVMHEALHNHYTQKSLSLSPGK (SEQ ID NO: 147) |
| DAB012124 | QVQLLQSGPGLVKPSATLSLTCTVSGFSLTTYGVHWIRQPPGKGLEWMGVIWSGGSTD |
| | YNAAFISRLTISKDNSKNQVFLQLYSLRAEDTAIYYCARTSHWYFDVWGRGTLVTVSSA |
| | STKGPSVFPLAPSSKSTSGGTAALGCLVKDYFPEPVTVSWNSGALTSGVHTFPAVLQSSG |
| | LYSLSSVVTVPSSSLGTQTYICNVNHKPSNTKVDKKVEPKSCDKTHTCPPCPAPELLGGP |
| | SVFLFPPKPKDTLMISRTPEVTCVVVDVSHEDPEVKFNWYVDGVEVHNAKTKPREEQY |
| | NSTYRVVSVLTVLHQDWLNGKEYKCKVSNKALPAPIEKTISKAKGQPREPQVYTLPPSR |
| | DELTKNQVSLTCLVKGFYPSDIAVEWESNGQPENNYKTTPPVLDSDGSFFLYSKLTVDK |
| | SRWQQGNVFSCSVMHEALHNHYTQKSLSLSPGK (SEQ ID NO: 148) |
| DAB012125 | QVQLQESGPGLVKPSQTLSLTCTVSGFSLTTYGVHWVRQSPGKGLEWLGVIWSGGSTD |
| | YNAAFISRLSISKDNSKSQVFLEMTSLTAADTAIYYCARTSHWYFDVWGRGTLVTVSSA |
| | STKGPSVFPLAPSSKSTSGGTAALGCLVKDYFPEPVTVSWNSGALTSGVHTFPAVLQSSG |
| | LYSLSSVVTVPSSSLGTQTYICNVNHKPSNTKVDKKVEPKSCDKTHTCPPCPAPELLGGP |
| | SVFLFPPKPKDTLMISRTPEVTCVVVDVSHEDPEVKFNWYVDGVEVHNAKTKPREEQY |
| | NSTYRVVSVLTVLHQDWLNGKEYKCKVSNKALPAPIEKTISKAKGQPREPQVYTLPPSR |
| | DELTKNQVSLTCLVKGFYPSDIAVEWESNGQPENNYKTTPPVLDSDGSFFLYSKLTVDK |
| | SRWQQGNVFSCSVMHEALHNHYTQKSLSLSPGK (SEQ ID NO: 149) |
| DAB012126 | QVQLQESGPGLVKPSQTLSLTCTVSGFSLTTYGVHWVRQAPEKGLEWLGVIWSGGSTD |
| | YNAAFISRLSISKDNSKSQVFLEMTSLTADDTAIYYCARTSHWYFDVWGRGTLVTVSSA |
| | STKGPSVFPLAPSSKSTSGGTAALGCLVKDYFPEPVTVSWNSGALTSGVHTFPAVLQSSG |
| | LYSLSSVVTVPSSSLGTQTYICNVNHKPSNTKVDKKVEPKSCDKTHTCPPCPAPELLGGP |
| | SVFLFPPKPKDTLMISRTPEVTCVVVDVSHEDPEVKFNWYVDGVEVHNAKTKPREEQY |
| | NSTYRVVSVLTVLHQDWLNGKEYKCKVSNKALPAPIEKTISKAKGQPREPQVYTLPPSR |
| | DELTKNQVSLTCLVKGFYPSDIAVEWESNGQPENNYKTTPPVLDSDGSFFLYSKLTVDK |
| | SRWQQGNVFSCSVMHEALHNHYTQKSLSLSPGK (SEQ ID NO: 150) |
| DAB012127 | QVQLQESGPGLVKPSQTLSLTCTVSGFSLTTYGVHWVRQTPEKGLEWLGVIWSGGSTD |
| | YNAAFISRLSISKDNSKSQVFLEMTSLTADDTAIYYCARTSHWYFDVWGRGTLVTVSSA |
| | STKGPSVFPLAPSSKSTSGGTAALGCLVKDYFPEPVTVSWNSGALTSGVHTFPAVLQSSG |
| | LYSLSSVVTVPSSSLGTQTYICNVNHKPSNTKVDKKVEPKSCDKTHTCPPCPAPELLGGP |
| | SVFLFPPKPKDTLMISRTPEVTCVVVDVSHEDPEVKFNWYVDGVEVHNAKTKPREEQY |
| | NSTYRVVSVLTVLHQDWLNGKEYKCKVSNKALPAPIEKTISKAKGQPREPQVYTLPPSR |
| | DELTKNQVSLTCLVKGFYPSDIAVEWESNGQPENNYKTTPPVLDSDGSFFLYSKLTVDK |
| | SRWQQGNVFSCSVMHEALHNHYTQKSLSLSPGK (SEQ ID NO: 151) |
| DAB012128 | QVQLLESGGGLVQPGGSLRLSCAASGFTFSDYYMYWIRQTPQKRLEWVAYISNGGGNT |
| | YYSDTVKGRFTISRDNAKNQLYLQMRSLRPEDTAIYYCASPEARYYGNFPFPYWGQGTL |
| | VTVSSASTKGPSVFPLAPSSKSTSGGTAALGCLVKDYFPEPVTVSWNSGALTSGVHTFPA |
| | VLQSSGLYSLSSVVTVPSSSLGTQTYICNVNHKPSNTKVDKKVEPKSCDKTHTCPPCPAP |
| | ELLGGPSVFLFPPKPKDTLMISRTPEVTCVVVDVSHEDPEVKFNWYVDGVEVHNAKTKP |
| | REEQYNSTYRVVSVLTVLHQDWLNGKEYKCKVSNKALPAPIEKTISKAKGQPREPQVY |
| | TLPPSRDELTKNQVSLTCLVKGFYPSDIAVEWESNGQPENNYKTTPPVLDSDGSFFLYSK |
| | LTVDKSRWQQGNVFSCSVMHEALHNHYTQKSLSLSPGK (SEQ ID NO: 152) |
| DAB012129 | QVQLVESGGGLVKPGGSLRLSCAASGFTFSDYYMYWIRQAPGKGLEWVAYISNGGGNT |
| | YYSDTVKGRFTISRDNAKNTLYLQMNSLRAEDTAVYYCASPEARYYGNFPFPYWGQGT |
| | LVTVSSASTKGPSVFPLAPSSKSTSGGTAALGCLVKDYFPEPVTVSWNSGALTSGVHTFP |
| | AVLQSSGLYSLSSVVTVPSSSLGTQTYICNVNHKPSNTKVDKKVEPKSCDKTHTCPPCPA |
| | PELLGGPSVFLFPPKPKDTLMISRTPEVTCVVVDVSHEDPEVKFNWYVDGVEVHNAKTK |
| | PREEQYNSTYRVVSVLTVLHQDWLNGKEYKCKVSNKALPAPIEKTISKAKGQPREPQV |
| | YTLPPSRDELTKNQVSLTCLVKGFYPSDIAVEWESNGQPENNYKTTPPVLDSDGSFFLYS |
| | KLTVDKSRWQQGNVFSCSVMHEALHNHYTQKSLSLSPGK (SEQ ID NO: 153) |
| DAB012130 | QVQLVESGGGLVKPGGSLRLSCAASGFTFSDYYMYWIRQAPGKGLEWVAYISNGGGNT |
| | YYSDTVKGRFTISRDNAKNTLYLQMNSLRAEDTAVYYCASPEARYYGNFPFPYWGQGT |
| | LVTVSSASTKGPSVFPLAPSSKSTSGGTAALGCLVKDYFPEPVTVSWNSGALTSGVHTFP |
| | AVLQSSGLYSLSSVVTVPSSSLGTQTYICNVNHKPSNTKVDKKVEPKSCDKTHTCPPCPA |
| | PELLGGPSVFLFPPKPKDTLMISRTPEVTCVVVDVSHEDPEVKFNWYVDGVEVHNAKTK |
| | PREEQYNSTYRVVSVLTVLHQDWLNGKEYKCKVSNKALPAPIEKTISKAKGQPREPQV |
| | YTLPPSRDELTKNQVSLTCLVKGFYPSDIAVEWESNGQPENNYKTTPPVLDSDGSFFLYS |
| | KLTVDKSRWQQGNVFSCSVMHEALHNHYTQKSLSLSPGK (SEQ ID NO: 154) |
| DAB012131 | QVQLVESGGGLVKPGGSLRLSCAASGFTFSDYYMYWIRQAPGKGLEWVAYISNGGGNT |
| | YYSDTVKGRFTISRDNAKNTLYLQMNSLRAEDTAVYYCASPEARYYGNFPFPYWGQGT |
| | LVTVSSASTKGPSVFPLAPSSKSTSGGTAALGCLVKDYFPEPVTVSWNSGALTSGVHTFP |
| | AVLQSSGLYSLSSVVTVPSSSLGTQTYICNVNHKPSNTKVDKKVEPKSCDKTHTCPPCPA |
| | PELLGGPSVFLFPPKPKDTLMISRTPEVTCVVVDVSHEDPEVKFNWYVDGVEVHNAKTK |
| | PREEQYNSTYRVVSVLTVLHQDWLNGKEYKCKVSNKALPAPIEKTISKAKGQPREPQV |
| | YTLPPSRDELTKNQVSLTCLVKGFYPSDIAVEWESNGQPENNYKTTPPVLDSDGSFFLYS |
| | KLTVDKSRWQQGNVFSCSVMHEALHNHYTQKSLSLSPGK (SEQ ID NO: 155) |
| DAB012132 | QVQLVESGGGVVQPGRSLRLSCAASGFSFSNYYMAWVRQAPGKGLEWVASISTGGGNI |
| | YYRDSVKGRFTISRDNSKSTLYLQMRSLRSEDTAIYYCARQTAYYVMDAWGQGTMVT |
| | VSSASTKGPSVFPLAPSSKSTSGGTAALGCLVKDYFPEPVTVSWNSGALTSGVHTFPAVL |
| | QSSGLYSLSSVVTVPSSSLGTQTYICNVNHKPSNTKVDKKVEPKSCDKTHTCPPCPAPEL |
| | LGGPSVFLFPPKPKDTLMISRTPEVTCVVVDVSHEDPEVKFNWYVDGVEVHNAKTKPRE |
| | EQYNSTYRVVSVLTVLHQDWLNGKEYKCKVSNKALPAPIEKTISKAKGQPREPQVYTLP |
| | PSRDELTKNQVSLTCLVKGFYPSDIAVEWESNGQPENNYKTTPPVLDSDGSFFLYSKLTV |
| | DKSRWQQGNVFSCSVMHEALHNHYTQKSLSLSPGK (SEQ ID NO: 156) |
| DAB012133 | EVQLVESGGGLVQPGGSLRLSCAASGFSFSNYYMAWVRQAPGKGLEWVASISTGGGNI |
| | YYRDSVKGRFTISRDNAKNSLYLQMNSLRAEDTAVYYCARQTAYYVMDAWGQGTTV |
| | TVSSASTKGPSVFPLAPSSKSTSGGTAALGCLVKDYFPEPVTVSWNSGALTSGVHTFPAV |
| | LQSSGLYSLSSVVTVPSSSLGTQTYICNVNHKPSNTKVDKKVEPKSCDKTHTCPPCPAPE |
| | LLGGPSVFLFPPKPKDTLMISRTPEVTCVVVDVSHEDPEVKFNWYVDGVEVHNAKTKPR |
| | EEQYNSTYRVVSVLTVLHQDWLNGKEYKCKVSNKALPAPIEKTISKAKGQPREPQVYT |
| | LPPSRDELTKNQVSLTCLVKGFYPSDIAVEWESNGQPENNYKTTPPVLDSDGSFFLYSKL |
| | TVDKSRWQQGNVFSCSVMHEALHNHYTQKSLSLSPGK (SEQ ID NO: 157) |
| DAB012134 | EVQLVESGGGLVQPGGSLRLSCAASGFSFSNYYMAWVRQAPGKGLEWVASISTGGGNI |
| | YYRDSVKGRFTISRDNAKNSLYLQMNSLRAEDTAVYYCARQTAYYVMDAWGQGTTV |
| | TVSSASTKGPSVFPLAPSSKSTSGGTAALGCLVKDYFPEPVTVSWNSGALTSGVHTFPAV |
| | LQSSGLYSLSSVVTVPSSSLGTQTYICNVNHKPSNTKVDKKVEPKSCDKTHTCPPCPAPE |
| | LLGGPSVFLFPPKPKDTLMISRTPEVTCVVVDVSHEDPEVKFNWYVDGVEVHNAKTKPR |
| | EEQYNSTYRVVSVLTVLHQDWLNGKEYKCKVSNKALPAPIEKTISKAKGQPREPQVYT |
| | LPPSRDELTKNQVSLTCLVKGFYPSDIAVEWESNGQPENNYKTTPPVLDSDGSFFLYSKL |
| | TVDKSRWQQGNVFSCSVMHEALHNHYTQKSLSLSPGK (SEQ ID NO: 158) |
| DAB012135 | EVQLVESGGGLVQPGGSLRLSCAASGFSFSNYYMAWVRQAPGKGLEWVASISTGGGNI |
| | YYRDSVKGRFTISRDNAKNSLYLQMNSLRAEDTAVYYCARQTAYYVMDAWGQGTTV |
| | TVSSASTKGPSVFPLAPSSKSTSGGTAALGCLVKDYFPEPVTVSWNSGALTSGVHTFPAV |
| | LQSSGLYSLSSVVTVPSSSLGTQTYICNVNHKPSNTKVDKKVEPKSCDKTHTCPPCPAPE |
| | LLGGPSVFLFPPKPKDTLMISRTPEVTCVVVDVSHEDPEVKFNWYVDGVEVHNAKTKPR |
| | EEQYNSTYRVVSVLTVLHQDWLNGKEYKCKVSNKALPAPIEKTISKAKGQPREPQVYT |
| | LPPSRDELTKNQVSLTCLVKGFYPSDIAVEWESNGQPENNYKTTPPVLDSDGSFFLYSKL |
| | TVDKSRWQQGNVFSCSVMHEALHNHYTQKSLSLSPGK (SEQ ID NO: 159) |

TABLE 16
Full Length Light Chain Nucleotide Sequences of Humanized Antibodies

| Sequence | |
|---|---|
| Name | Light chain sequence |
| DAB012120 | GAGATCCAGATGACCCAGAGCCACAAGACCTTCAGCGTGAGCACCGGCCAGAGGGT |
| | GACCATCACCTGCAAGGCCAGCCAGGACGTGAGCATCGCCGTGGCCTGGTACCAGC |
| | AGAAGCCCGGCAAGGCCCCCAAGCTGCTGATCTACTGGGCCAGCACCAGGCAGACC |
| | GGCGTGCCCAGCAGGTTCAGCGGCAGCGGCAGCGGCACCGACTTCACCCTGACCAT |
| | CAGCTGCGTGCAGAGCGAGGACTTCGCCGTGTACTACTGCCAGCAGTACAGCAGCT |
| | ACCCCTTCACCTTCGGCCAGGGCACCAAGCTGGAGATCAAGCGAACGGTGGCTGCC |
| | CCCTCCGTGTTCATCTTCCCCCCCAGCGATGAGCAGCTGAAGAGCGGCACAGCCAGC |
| | GTGGTGTGCCTGCTGAACAACTTCTACCCCAGGGAGGCCAAGGTGCAGTGGAAGGT |
| | GGACAACGCCCTGCAGAGCGGCAACAGCCAGGAGTCCGTGACCGAGCAGGACAGC |
| | AAGGACAGCACCTACAGCCTGAGCAGCACACTGACCCTGAGCAAGGCCGACTACGA |
| | GAAGCACAAGGTGTACGCCTGCGAGGTGACCCATCAGGGCCTGAGCAGCCCCGTGA |
| | CCAAGAGCTTCAACAGGGGCGAGTGC (SEQ ID NO: 160) |
| DAB012121 | GACATCCAGATGACCCAGAGCCCCAGCTTCCTGAGCGCCAGCGTGGGCGACAGGGT |
| | GACCATCACCTGCAAGGCCAGCCAGGACGTGAGCATCGCCGTGGCCTGGTACCAGC |
| | AGAAGCCCGGCAAGGCCCCCAAGCTGCTGATCTACTGGGCCAGCACCAGGCAGACC |
| | GGCGTGCCCAGCAGGTTCAGCGGCAGCGGCAGCGGCACCGACTTCACCCTGACCAT |
| | CAGCAGCCTGCAGCCCGAGGACTTCGCCACCTACTTCTGCCAGCAGTACAGCAGCTA |
| | CCCCTTCACCTTCGGCCAGGGCACCAAGCTGGAGATCAAGCGAACGGTGGCTGCCC |
| | CCTCCGTGTTCATCTTCCCCCCCAGCGATGAGCAGCTGAAGAGCGGCACAGCCAGCG |
| | TGGTGTGCCTGCTGAACAACTTCTACCCCAGGGAGGCCAAGGTGCAGTGGAAGGTG |
| | GACAACGCCCTGCAGAGCGGCAACAGCCAGGAGTCCGTGACCGAGCAGGACAGCA |
| | AGGACAGCACCTACAGCCTGAGCAGCACACTGACCCTGAGCAAGGCCGACTACGAG |
| | AAGCACAAGGTGTACGCCTGCGAGGTGACCCATCAGGGCCTGAGCAGCCCCGTGAC |
| | CAAGAGCTTCAACAGGGGCGAGTGC (SEQ ID NO: 161) |
| DAB012122 | GACATCCAGATGACCCAGAGCCCCAGCTTCCTGAGCGCCAGCGTGGGCGAGAGGGT |
| | GACCATCACCTGCAAGGCCAGCCAGGACGTGAGCATCGCCGTGGCCTGGTACCAGC |
| | AGAAGCCCGGCAAGGCCCCCAAGCTGCTGATCTACTGGGCCAGCACCAGGCAGACC |
| | GGCGTGCCCAGCAGGTTCAGCGGCAGCGGCAGCGGCACCGACTTCACCCTGACCAT |
| | CAGCAGCCTGCAGCCCGAGGACATCGCCACCTACTTCTGCCAGCAGTACAGCAGCT |
| | ACCCCTTCACCTTCGGCCAGGGCACCAAGCTGGAGATCAAGCGAACGGTGGCTGCC |
| | CCCTCCGTGTTCATCTTCCCCCCCAGCGATGAGCAGCTGAAGAGCGGCACAGCCAGC |
| | GTGGTGTGCCTGCTGAACAACTTCTACCCCAGGGAGGCCAAGGTGCAGTGGAAGGT |
| | GGACAACGCCCTGCAGAGCGGCAACAGCCAGGAGTCCGTGACCGAGCAGGACAGC |
| | AAGGACAGCACCTACAGCCTGAGCAGCACACTGACCCTGAGCAAGGCCGACTACGA |
| | GAAGCACAAGGTGTACGCCTGCGAGGTGACCCATCAGGGCCTGAGCAGCCCCGTGA |
| | CCAAGAGCTTCAACAGGGGCGAGTGC (SEQ ID NO: 162) |
| DAB012123 | GACATCCAGATGACCCAGAGCCCCAGCTTCCTGAGCGCCAGCGTGGGCGACAGGGT |
| | GACCATCACCTGCAAGGCCAGCCAGGACGTGAGCATCGCCGTGGCCTGGTACCAGC |
| | AGAAGCCCGGCAAGGCCCCCAAGCTGCTGATCTACTGGGCCAGCACCAGGCAGACC |
| | GGCGTGCCCAGCAGGTTCAGCGGCAGCGGCAGCGGCACCGACTTCACCCTGACCAT |
| | CAGCAGCCTGCAGCCCGAGGACAGCGCCACCTACTTCTGCCAGCAGTACAGCAGCT |
| | ACCCCTTCACCTTCGGCCAGGGCACCAAGGTGGACATCAAGCGAACGGTGGCTGCC |
| | CCCTCCGTGTTCATCTTCCCCCCCAGCGATGAGCAGCTGAAGAGCGGCACAGCCAGC |
| | GTGGTGTGCCTGCTGAACAACTTCTACCCCAGGGAGGCCAAGGTGCAGTGGAAGGT |
| | GGACAACGCCCTGCAGAGCGGCAACAGCCAGGAGTCCGTGACCGAGCAGGACAGC |
| | AAGGACAGCACCTACAGCCTGAGCAGCACACTGACCCTGAGCAAGGCCGACTACGA |
| | GAAGCACAAGGTGTACGCCTGCGAGGTGACCCATCAGGGCCTGAGCAGCCCCGTGA |
| | CCAAGAGCTTCAACAGGGGCGAGTGC (SEQ ID NO: 163) |
| DAB012124 | GAGATCCAGATGACCCAGAGCCAGAGCACCCTGAGCGTGAGCCTGGGCGACAGGGT |
| | GACCATCACCTGCAAGGCCAGCCAGAACGTGGTGACCGCCGTGGCCTGGTACCAGC |
| | AGAAGCCCGGCCAGGCCCCCAAGCTGCTGATCTACAGCGCCAGCCACAGGTTCACC |
| | GGCGTGCCCAGCAGGTTCAGCGGCAGCGGCAGCGGCACCGACTTCACCCTGATCAT |
| | CAGCTGCATGCAGAGCGAGGACTTCGCCGTGTACTACTGCCAGCAGTACAGCAGCT |
| | ACCCCTACACCTTCGGCCAGGGCACCAAGCTGGAGATCAAGCGAACGGTGGCTGCC |
| | CCCTCCGTGTTCATCTTCCCCCCCAGCGATGAGCAGCTGAAGAGCGGCACAGCCAGC |
| | GTGGTGTGCCTGCTGAACAACTTCTACCCCAGGGAGGCCAAGGTGCAGTGGAAGGT |
| | GGACAACGCCCTGCAGAGCGGCAACAGCCAGGAGTCCGTGACCGAGCAGGACAGC |
| | AAGGACAGCACCTACAGCCTGAGCAGCACACTGACCCTGAGCAAGGCCGACTACGA |
| | GAAGCACAAGGTGTACGCCTGCGAGGTGACCCATCAGGGCCTGAGCAGCCCCGTGA |
| | CCAAGAGCTTCAACAGGGGCGAGTGC (SEQ ID NO: 164) |
| DAB012125 | GACATCCAGATGACCCAGAGCCCCAGCTTCCTGAGCGCCAGCGTGGGCGACAGGGT |
| | GACCATCACCTGCAAGGCCAGCCAGAACGTGGTGACCGCCGTGGCCTGGTACCAGC |
| | AGAAGCCCGGCAAGGCCCCCAAGCTGCTGATCTACAGCGCCAGCCACAGGTTCACC |
| | GGCGTGCCCAGCAGGTTCAGCGGCAGCGGCAGCGGCACCGACTTCACCCTGACCAT |
| | CAGCAACCTGCAGAGCGAGGACCTGGCCACCTACTTCTGCCAGCAGTACAGCAGCT |
| | ACCCCTACACCTTCGGCCAGGGCACCAAGCTGGAGATCAAGCGAACGGTGGCTGCC |
| | CCCTCCGTGTTCATCTTCCCCCCCAGCGATGAGCAGCTGAAGAGCGGCACAGCCAGC |
| | GTGGTGTGCCTGCTGAACAACTTCTACCCCAGGGAGGCCAAGGTGCAGTGGAAGGT |
| | GGACAACGCCCTGCAGAGCGGCAACAGCCAGGAGTCCGTGACCGAGCAGGACAGC |
| | AAGGACAGCACCTACAGCCTGAGCAGCACACTGACCCTGAGCAAGGCCGACTACGA |
| | GAAGCACAAGGTGTACGCCTGCGAGGTGACCCATCAGGGCCTGAGCAGCCCCGTGA |
| | CCAAGAGCTTCAACAGGGGCGAGTGC (SEQ ID NO: 165) |
| DAB012126 | GACATCCAGATGACCCAGAGCCCCAGCTTCCTGAGCGCCAGCGTGGGCGACAGGGT |
| | GACCATCACCTGCAAGGCCAGCCAGAACGTGGTGACCGCCGTGGCCTGGTACCAGC |
| | AGAAGCCCGGCAAGGCCCCCAAGCTGCTGATCTACAGCGCCAGCCACAGGTTCACC |
| | GGCGTGCCCAGCAGGTTCAGCGGCAGCGGCAGCGGCACCGACTTCACCCTGACCAT |
| | CAGCAACCTGCAGCCCGAGGACAGCGCCACCTACTTCTGCCAGCAGTACAGCAGCT |
| | ACCCCTACACCTTCGGCCAGGGCACCAAGCTGGAGATCAAGCGAACGGTGGCTGCC |
| | CCCTCCGTGTTCATCTTCCCCCCCAGCGATGAGCAGCTGAAGAGCGGCACAGCCAGC |
| | GTGGTGTGCCTGCTGAACAACTTCTACCCCAGGGAGGCCAAGGTGCAGTGGAAGGT |
| | GGACAACGCCCTGCAGAGCGGCAACAGCCAGGAGTCCGTGACCGAGCAGGACAGC |
| | AAGGACAGCACCTACAGCCTGAGCAGCACACTGACCCTGAGCAAGGCCGACTACGA |
| | GAAGCACAAGGTGTACGCCTGCGAGGTGACCCATCAGGGCCTGAGCAGCCCCGTGA |
| | CCAAGAGCTTCAACAGGGGCGAGTGC (SEQ ID NO: 166) |
| DAB012127 | GACATCCAGATGACCCAGAGCCCCAGCTTCCTGAGCGCCAGCGTGGGCGACAGGGT |
| | GACCATCACCTGCAAGGCCAGCCAGAACGTGGTGACCGCCGTGGCCTGGTACCAGC |
| | AGAAGCCCGGCAAGGCCCCCAAGCTGCTGATCTACAGCGCCAGCCACAGGTTCACC |
| | GGCGTGCCCAGCAGGTTCAGCGGCAGCGGCAGCGGCACCGACTTCACCCTGACCAT |
| | CAGCAACCTGCAGCCCGACGACGCCGCCACCTACTTCTGCCAGCAGTACAGCAGCT |
| | ACCCCTACACCTTCGGCCAGGGCACCAAGCTGGAGATCAAGCGAACGGTGGCTGCC |
| | CCCTCCGTGTTCATCTTCCCCCCCAGCGATGAGCAGCTGAAGAGCGGCACAGCCAGC |
| | GTGGTGTGCCTGCTGAACAACTTCTACCCCAGGGAGGCCAAGGTGCAGTGGAAGGT |
| | GGACAACGCCCTGCAGAGCGGCAACAGCCAGGAGTCCGTGACCGAGCAGGACAGC |
| | AAGGACAGCACCTACAGCCTGAGCAGCACACTGACCCTGAGCAAGGCCGACTACGA |
| | GAAGCACAAGGTGTACGCCTGCGAGGTGACCCATCAGGGCCTGAGCAGCCCCGTGA |
| | CCAAGAGCTTCAACAGGGGCGAGTGC (SEQ ID NO: 167) |
| DAB012128 | GCCATCCAGATGACCCAGAGCCACAAGAGCTTCAGCGTGAGCACCGGCCAGAGGGT |
| | GACCATCACCTGCAAGGCCAGCCAGGACGTGAGCACCACCGTGGCCTGGTACCAGC |
| | AGAAGCCCGGCCAGGCCCCCAAGCTGCTGATCTACTGGGCCAGCACCAGGCAGACC |
| | GGCGTGCCCAGCAGGTTCACCGGCAGCGGCAGCGGCACCGACTTCACCCTGACCAT |
| | CAGCTGCGTGCAGAGCGAGGACTTCGCCGTGTACTACTGCCAGCAGTACAGCAGCT |
| | ACCCCTTCACCTTCGGCCAGGGCACCAAGCTGGAGATCAAGCGAACGGTGGCTGCC |
| | CCCTCCGTGTTCATCTTCCCCCCCAGCGATGAGCAGCTGAAGAGCGGCACAGCCAGC |
| | GTGGTGTGCCTGCTGAACAACTTCTACCCCAGGGAGGCCAAGGTGCAGTGGAAGGT |
| | GGACAACGCCCTGCAGAGCGGCAACAGCCAGGAGTCCGTGACCGAGCAGGACAGC |
| | AAGGACAGCACCTACAGCCTGAGCAGCACACTGACCCTGAGCAAGGCCGACTACGA |
| | GAAGCACAAGGTGTACGCCTGCGAGGTGACCCATCAGGGCCTGAGCAGCCCCGTGA |
| | CCAAGAGCTTCAACAGGGGCGAGTGC (SEQ ID NO: 168) |
| DAB012129 | GACATCCAGATGACCCAGAGCCCCAGCTTCCTGAGCGCCAGCGTGGGCGACAGGGT |
| | GACCATCACCTGCAAGGCCAGCCAGGACGTGAGCACCACCGTGGCCTGGTACCAGC |
| | AGAAGCCCGGCAAGGCCCCCAAGCTGCTGATCTACTGGGCCAGCACCAGGCAGACC |
| | GGCGTGCCCAGCAGGTTCAGCGGCAGCGGCAGCGGCACCGACTTCACCCTGACCAT |
| | CAGCAACCTGCAGCCCGAGGACTTCGCCACCTACTTCTGCCAGCAGTACAGCAGCTA |
| | CCCCTTCACCTTCGGCCAGGGCACCAAGCTGGAGATCAAGCGAACGGTGGCTGCCC |
| | CCTCCGTGTTCATCTTCCCCCCCAGCGATGAGCAGCTGAAGAGCGGCACAGCCAGCG |
| | TGGTGTGCCTGCTGAACAACTTCTACCCCAGGGAGGCCAAGGTGCAGTGGAAGGTG |
| | GACAACGCCCTGCAGAGCGGCAACAGCCAGGAGTCCGTGACCGAGCAGGACAGCA |
| | AGGACAGCACCTACAGCCTGAGCAGCACACTGACCCTGAGCAAGGCCGACTACGAG |
| | AAGCACAAGGTGTACGCCTGCGAGGTGACCCATCAGGGCCTGAGCAGCCCCGTGAC |
| | CAAGAGCTTCAACAGGGGCGAGTGC (SEQ ID NO: 169) |
| DAB012130 | GACATCCAGATGACCCAGAGCCCCAGCTTCCTGAGCGCCAGCGTGGGCGAGAGGGT |
| | GACCATCACCTGCAAGGCCAGCCAGGACGTGAGCACCACCGTGGCCTGGTACCAGC |
| | AGAAGCCCGGCAAGGCCCCCAAGCTGCTGATCTACTGGGCCAGCACCAGGCAGACC |
| | GGCGTGCCCAGCAGGTTCAGCGGCAGCGGCAGCGGCACCGACTTCACCCTGACCAT |
| | CAACAGCCTGCAGCCCGAGGACGTGGCCACCTACTTCTGCCAGCAGTACAGCAGCT |
| | ACCCCTTCACCTTCGGCCAGGGCACCAAGCTGGAGATCAAGCGAACGGTGGCTGCC |
| | CCCTCCGTGTTCATCTTCCCCCCCAGCGATGAGCAGCTGAAGAGCGGCACAGCCAGC |
| | GTGGTGTGCCTGCTGAACAACTTCTACCCCAGGGAGGCCAAGGTGCAGTGGAAGGT |
| | GGACAACGCCCTGCAGAGCGGCAACAGCCAGGAGTCCGTGACCGAGCAGGACAGC |
| | AAGGACAGCACCTACAGCCTGAGCAGCACACTGACCCTGAGCAAGGCCGACTACGA |
| | GAAGCACAAGGTGTACGCCTGCGAGGTGACCCATCAGGGCCTGAGCAGCCCCGTGA |
| | CCAAGAGCTTCAACAGGGGCGAGTGC (SEQ ID NO: 170) |
| DAB012131 | GACATCCAGATGACCCAGAGCCCCAGCTTCCTGAGCGCCAGCGTGGGCGAGAGGGT |
| | GACCATCACCTGCAAGGCCAGCCAGGACGTGAGCACCACCGTGGCCTGGTACCAGC |
| | AGAAGCCCGGCAAGGCCCCCAAGCTGCTGATCTACTGGGCCAGCACCAGGCAGACC |
| | GGCGTGCCCAGCAGGTTCAGCGGCAGCGGCAGCGGCACCGACTTCACCCTGACCAT |
| | CAGCAACCTGCAGCCCGAGGACGTGGCCACCTACTTCTGCCAGCAGTACAGCAGCT |
| | ACCCCTTCACCTTCGGCCAGGGCACCAAGCTGGAGATCAAGCGAACGGTGGCTGCC |
| | CCCTCCGTGTTCATCTTCCCCCCCAGCGATGAGCAGCTGAAGAGCGGCACAGCCAGC |
| | GTGGTGTGCCTGCTGAACAACTTCTACCCCAGGGAGGCCAAGGTGCAGTGGAAGGT |
| | GGACAACGCCCTGCAGAGCGGCAACAGCCAGGAGTCCGTGACCGAGCAGGACAGC |
| | AAGGACAGCACCTACAGCCTGAGCAGCACACTGACCCTGAGCAAGGCCGACTACGA |
| | GAAGCACAAGGTGTACGCCTGCGAGGTGACCCATCAGGGCCTGAGCAGCCCCGTGA |
| | CCAAGAGCTTCAACAGGGGCGAGTGC (SEQ ID NO: 171) |
| DAB012132 | GACATCCAGATGACCCAGAGCCCCAGCAGCGTGAGCCTGACCGTGGGCCAGAGGGT |
| | GACCATCACCTGCCTGGCCACCGAGGACATCTTCAGCTACCTGGCCTGGTACCAGCA |
| | GAAGCCCGGCAAGGCCCCCAGGCTGCTGATCTACGGCGCCAACAGGCTGAAGGACG |
| | GCGTGCCCAGCAGGTTCAGCGGCAGCGGCAGCGGCACCCAGTACACCCTGAGGATC |
| | AGCAGCATGCAGCCCGAGGACTTCGGCGTGTACTACTGCCTGCAGGGCGCCAAGTT |
| | CCCCCTGACCTTCGGCCCCGGCACCAAGCTGGAGATCAAGCGAACGGTGGCTGCCC |
| | CCTCCGTGTTCATCTTCCCCCCCAGCGATGAGCAGCTGAAGAGCGGCACAGCCAGCG |
| | TGGTGTGCCTGCTGAACAACTTCTACCCCAGGGAGGCCAAGGTGCAGTGGAAGGTG |
| | GACAACGCCCTGCAGAGCGGCAACAGCCAGGAGTCCGTGACCGAGCAGGACAGCA |
| | AGGACAGCACCTACAGCCTGAGCAGCACACTGACCCTGAGCAAGGCCGACTACGAG |
| | AAGCACAAGGTGTACGCCTGCGAGGTGACCCATCAGGGCCTGAGCAGCCCCGTGAC |
| | CAAGAGCTTCAACAGGGGCGAGTGC (SEQ ID NO: 172) |
| DAB012133 | GACATCCAGATGACCCAGAGCCCCAGCAGCCTGAGCGCCAGCATCGGCGACACCGT |
| | GACCATCACCTGCCTGGCCACCGAGGACATCTTCAGCTACCTGGCCTGGTACCAGCA |
| | GAGGCCCGGCAAGGCCCCCAAGCTGCTGATCTACGGCGCCAACAGGCTGAAGGACG |
| | GCGTGCCCAGCAGGTTCAGCGGCAGCGGCAGCGGCACCGACTACACCCTGACCATC |
| | AGCGGCCTGCAGCCCGAGGACTTCGTGACCTACTACTGCCTGCAGGGCGCCAAGTTC |
| | CCCCTGACCTTCGGCCAGGGCACCAAGCTGGACATGAAGCGAACGGTGGCTGCCCC |
| | CTCCGTGTTCATCTTCCCCCCCAGCGATGAGCAGCTGAAGAGCGGCACAGCCAGCGT |
| | GGTGTGCCTGCTGAACAACTTCTACCCCAGGGAGGCCAAGGTGCAGTGGAAGGTGG |
| | ACAACGCCCTGCAGAGCGGCAACAGCCAGGAGTCCGTGACCGAGCAGGACAGCAA |
| | GGACAGCACCTACAGCCTGAGCAGCACACTGACCCTGAGCAAGGCCGACTACGAGA |
| | AGCACAAGGTGTACGCCTGCGAGGTGACCCATCAGGGCCTGAGCAGCCCCGTGACC |
| | AAGAGCTTCAACAGGGGCGAGTGC (SEQ ID NO: 173) |
| DAB012134 | GACATCCAGATGACCCAGAGCCCCAGCAGCCTGAGCGCCAGCGTGGGCGACACCGT |
| | GACCATCACCTGCCTGGCCACCGAGGACATCTTCAGCTACCTGGCCTGGTACCAGCA |
| | GAAGCCCGGCAAGGCCCCCAAGCTGCTGATCTACGGCGCCAACAGGCTGAAGGACG |
| | GCGTGCCCAGCAGGTTCAGCGGCAGCGGCAGCGGCACCGACTACACCCTGACCATC |
| | AGCGGCCTGCAGCCCGAGGACTTCGGCACCTACTACTGCCTGCAGGGCGCCAAGTT |
| | CCCCCTGACCTTCGGCCAGGGCACCAAGCTGGAGATCAAGCGAACGGTGGCTGCCC |
| | CCTCCGTGTTCATCTTCCCCCCCAGCGATGAGCAGCTGAAGAGCGGCACAGCCAGCG |
| | TGGTGTGCCTGCTGAACAACTTCTACCCCAGGGAGGCCAAGGTGCAGTGGAAGGTG |
| | GACAACGCCCTGCAGAGCGGCAACAGCCAGGAGTCCGTGACCGAGCAGGACAGCA |
| | AGGACAGCACCTACAGCCTGAGCAGCACACTGACCCTGAGCAAGGCCGACTACGAG |
| | AAGCACAAGGTGTACGCCTGCGAGGTGACCCATCAGGGCCTGAGCAGCCCCGTGAC |
| | CAAGAGCTTCAACAGGGGCGAGTGC (SEQ ID NO: 174) |
| DAB012135 | GACATCCAGATGACCCAGAGCCCCAGCAGCCTGAGCGCCAGCGTGGGCGACACCGT |
| | GACCATCACCTGCCTGGCCACCGAGGACATCTTCAGCTACCTGGCCTGGTACCAGCA |
| | GAAGCCCGGCAAGGCCCCCAAGCTGCTGATCTACGGCGCCAACAGGCTGAAGGACG |
| | GCGTGCCCAGCAGGTTCAGCGGCGGCGGCAGCGGCACCGAGTACAGCCTGACCATC |
| | AGCGGCCTGCAGCCCGAGGACTTCGGCACCTACTACTGCCTGCAGGGCGCCAAGTT |
| | CCCCCTGACCTTCGGCCAGGGCACCAAGCTGGACATGAAGCGAACGGTGGCTGCCC |
| | CCTCCGTGTTCATCTTCCCCCCCAGCGATGAGCAGCTGAAGAGCGGCACAGCCAGCG |
| | TGGTGTGCCTGCTGAACAACTTCTACCCCAGGGAGGCCAAGGTGCAGTGGAAGGTG |
| | GACAACGCCCTGCAGAGCGGCAACAGCCAGGAGTCCGTGACCGAGCAGGACAGCA |
| | AGGACAGCACCTACAGCCTGAGCAGCACACTGACCCTGAGCAAGGCCGACTACGAG |
| | AAGCACAAGGTGTACGCCTGCGAGGTGACCCATCAGGGCCTGAGCAGCCCCGTGAC |
| | CAAGAGCTTCAACAGGGGCGAGTGC (SEQ ID NO: 175) |
| DAB012135 | GACATCCAGATGACCCAGAGCCCCAGCAGCCTGAGCGCCAGCGTGGGCGACACCGT |
| (modified) | GACCATCACCTGCCTGGCCACCGAGGACATCTTCAGCTACCTGGCCTGGTACCAGCA |
| | GAAGCCCGGCAAGGCCCCCAAGCTGCTGATCTACGGCGCCAACAGGCTGAAGGACA |
| | CCGTGCCCAGCAGGTTCAGCGGCGGCGGCAGCGGCACCGAGTACAGCCTGACCATC |
| | AGCGGCCTGCAGCCCGAGGACTTCGGCACCTACTACTGCCTGCAGGGCGCCAAGTT |
| | CCCCCTGACCTTCGGCCAGGGCACCAAGCTGGACATGAAGCGAACGGTGGCTGCCC |
| | CCTCCGTGTTCATCTTCCCCCCCAGCGATGAGCAGCTGAAGAGCGGCACAGCCAGCG |
| | TGGTGTGCCTGCTGAACAACTTCTACCCCAGGGAGGCCAAGGTGCAGTGGAAGGTG |
| | GACAACGCCCTGCAGAGCGGCAACAGCCAGGAGTCCGTGACCGAGCAGGACAGCA |
| | AGGACAGCACCTACAGCCTGAGCAGCACACTGACCCTGAGCAAGGCCGACTACGAG |
| | AAGCACAAGGTGTACGCCTGCGAGGTGACCCATCAGGGCCTGAGCAGCCCCGTGAC |
| | CAAGAGCTTCAACAGGGGCGAGTGC (SEQ ID NO: 176) |

TABLE 17
Full Length Heavy Chain Nucleotide Sequences of Humanized Antibodies

| Sequence | |
|---|---|
| Name | Heavy chain sequence |
| DAB012120 | CAGGTGCAGCTGCTGGAGAGCGGCGGCGAGCTGGTGCAGCCCGGCGGCAGCCTGAG |
| | GCTGAGCTGCGCCGCCAGCGGCTTCACCTTCAGCGACTACTACATGTTCTGGATCAG |
| | GCAGACCCCCCAGAAGAGGCTGGAGTGGGTGGCCTACATCAGCAACGGCGGCGGCA |
| | ACACCTACTACCCCGACACCGTGAAGGGCAGGTTCACCATCAGCAGGGACAACGCC |
| | AAGAACCAGCTGTACCTGCAGATGAGGAGCCTGACCCCCGAGGACACCGCCATCTA |
| | CTACTGCGCCAGCCCCGAGGCCAGGTACTACGGCAACTACCCCTTCCCCTACTGGGG |
| | CCAGGGCACCCTGGTGACCGTGAGCAGCGCTAGCACCAAGGGCCCATCGGTCTTCC |
| | CCCTGGCACCCTCCTCCAAGAGCACCTCTGGGGGCACAGCGGCCCTGGGCTGCCTGG |
| | TCAAGGACTACTTCCCCGAACCGGTGACGGTGTCGTGGAACTCAGGCGCCCTGACC |
| | AGCGGCGTGCACACCTTCCCGGCTGTCCTACAGTCCTCAGGACTCTACTCCCTCAGC |
| | AGCGTGGTGACCGTGCCCTCCAGCAGCTTGGGCACCCAGACCTACATCTGCAACGTG |
| | AATCACAAGCCCAGCAACACCAAGGTGGACAAGAAAGTTGAGCCCAAATCTTGTGA |
| | CAAAACTCACACATGCCCACCGTGCCCAGCACCTGAACTCCTGGGGGGACCGTCAG |
| | TCTTCCTCTTCCCCCCAAAACCCAAGGACACCCTCATGATCTCCCGGACCCCTGAGG |
| | TCACATGCGTGGTGGTGGACGTGAGCCACGAAGACCCTGAGGTCAAGTTCAACTGG |
| | TACGTGGACGGCGTGGAGGTGCATAATGCCAAGACAAAGCCGCGGGAGGAGCAGT |
| | ACAACAGCACGTACCGGGTGGTCAGCGTCCTCACCGTCCTGCACCAGGACTGGCTG |
| | AATGGCAAGGAGTACAAGTGCAAGGTCTCCAACAAAGCCCTCCCAGCCCCCATCGA |
| | GAAAACCATCTCCAAAGCCAAAGGGCAGCCCCGAGAACCACAGGTGTACACCCTGC |
| | CCCCATCCCGGGATGAGCTGACCAAGAACCAGGTCAGCCTGACCTGCCTGGTCAAA |
| | GGCTTCTATCCCAGCGACATCGCCGTGGAGTGGGAGAGCAATGGGCAGCCGGAGAA |
| | CAACTACAAGACCACGCCTCCCGTGCTGGACTCCGACGGCTCCTTCTTCCTCTACAG |
| | CAAGCTCACCGTGGACAAGAGCAGGTGGCAGCAGGGGAACGTCTTCTCATGCTCCG |
| | TGATGCATGAGGCTCTGCACAACCACTACACGCAGAAGAGCCTCTCCCTGTCTCCGG |
| | GTAAA (SEQ ID NO: 177) |
| DAB012121 | CAGGTGCAGCTGGTGGAGAGCGGCGGCGGCCTGGTGAAGCCCGGCGGCAGCCTGAG |
| | GCTGAGCTGCGCCGCCAGCGGCTTCACCTTCAGCGACTACTACATGTTCTGGATCAG |
| | GCAGGCCCCCGGCAAGGGCCTGGAGTGGGTGGCCTACATCAGCAACGGCGGCGGCA |
| | ACACCTACTACCCCGACACCGTGAAGGGCAGGTTCACCATCAGCAGGGACAACGCC |
| | AAGAACACCCTGTACCTGCAGATGAACAGCCTGAGGGCCGAGGACACCGCCGTGTA |
| | CTACTGCGCCAGCCCCGAGGCCAGGTACTACGGCAACTACCCCTTCCCCTACTGGGG |
| | CCAGGGCACCCTGGTGACCGTGAGCAGCGCTAGCACCAAGGGCCCATCGGTCTTCC |
| | CCCTGGCACCCTCCTCCAAGAGCACCTCTGGGGGCACAGCGGCCCTGGGCTGCCTGG |
| | TCAAGGACTACTTCCCCGAACCGGTGACGGTGTCGTGGAACTCAGGCGCCCTGACC |
| | AGCGGCGTGCACACCTTCCCGGCTGTCCTACAGTCCTCAGGACTCTACTCCCTCAGC |
| | AGCGTGGTGACCGTGCCCTCCAGCAGCTTGGGCACCCAGACCTACATCTGCAACGTG |
| | AATCACAAGCCCAGCAACACCAAGGTGGACAAGAAAGTTGAGCCCAAATCTTGTGA |
| | CAAAACTCACACATGCCCACCGTGCCCAGCACCTGAACTCCTGGGGGGACCGTCAG |
| | TCTTCCTCTTCCCCCCAAAACCCAAGGACACCCTCATGATCTCCCGGACCCCTGAGG |
| | TCACATGCGTGGTGGTGGACGTGAGCCACGAAGACCCTGAGGTCAAGTTCAACTGG |
| | TACGTGGACGGCGTGGAGGTGCATAATGCCAAGACAAAGCCGCGGGAGGAGCAGT |
| | ACAACAGCACGTACCGGGTGGTCAGCGTCCTCACCGTCCTGCACCAGGACTGGCTG |
| | AATGGCAAGGAGTACAAGTGCAAGGTCTCCAACAAAGCCCTCCCAGCCCCCATCGA |
| | GAAAACCATCTCCAAAGCCAAAGGGCAGCCCCGAGAACCACAGGTGTACACCCTGC |
| | CCCCATCCCGGGATGAGCTGACCAAGAACCAGGTCAGCCTGACCTGCCTGGTCAAA |
| | GGCTTCTATCCCAGCGACATCGCCGTGGAGTGGGAGAGCAATGGGCAGCCGGAGAA |
| | CAACTACAAGACCACGCCTCCCGTGCTGGACTCCGACGGCTCCTTCTTCCTCTACAG |
| | CAAGCTCACCGTGGACAAGAGCAGGTGGCAGCAGGGGAACGTCTTCTCATGCTCCG |
| | TGATGCATGAGGCTCTGCACAACCACTACACGCAGAAGAGCCTCTCCCTGTCTCCGG |
| | GTAAA (SEQ ID NO: 178) |
| DAB012122 | CAGGTGCAGCTGGTGGAGAGCGGCGGCGGCGTGGTGAAGCCCGGCGGCAGCCTGA |
| | GGCTGAGCTGCGCCGCCAGCGGCTTCACCTTCAGCGACTACTACATGTTCTGGATCA |
| | GGCAGGCCCCCGGCAAGGGCCTGGAGTGGGTGGCCTACATCAGCAACGGCGGCGGC |
| | AACACCTACTACCCCGACACCGTGAAGGGCAGGTTCACCATCAGCAGGGACAACGC |
| | CAAGAACACCCTGTACCTGCAGATGAACAGCCTGAGGGCCGAGGACACCGCCGTGT |
| | ACTACTGCGCCAGCCCCGAGGCCAGGTACTACGGCAACTACCCCTTCCCCTACTGGG |
| | GCCAGGGCACCCTGGTGACCGTGAGCAGCGCTAGCACCAAGGGCCCATCGGTCTTC |
| | CCCCTGGCACCCTCCTCCAAGAGCACCTCTGGGGGCACAGCGGCCCTGGGCTGCCTG |
| | GTCAAGGACTACTTCCCCGAACCGGTGACGGTGTCGTGGAACTCAGGCGCCCTGAC |
| | CAGCGGCGTGCACACCTTCCCGGCTGTCCTACAGTCCTCAGGACTCTACTCCCTCAG |
| | CAGCGTGGTGACCGTGCCCTCCAGCAGCTTGGGCACCCAGACCTACATCTGCAACGT |
| | GAATCACAAGCCCAGCAACACCAAGGTGGACAAGAAAGTTGAGCCCAAATCTTGTG |
| | ACAAAACTCACACATGCCCACCGTGCCCAGCACCTGAACTCCTGGGGGGACCGTCA |
| | GTCTTCCTCTTCCCCCCAAAACCCAAGGACACCCTCATGATCTCCCGGACCCCTGAG |
| | GTCACATGCGTGGTGGTGGACGTGAGCCACGAAGACCCTGAGGTCAAGTTCAACTG |
| | GTACGTGGACGGCGTGGAGGTGCATAATGCCAAGACAAAGCCGCGGGAGGAGCAG |
| | TACAACAGCACGTACCGGGTGGTCAGCGTCCTCACCGTCCTGCACCAGGACTGGCTG |
| | AATGGCAAGGAGTACAAGTGCAAGGTCTCCAACAAAGCCCTCCCAGCCCCCATCGA |
| | GAAAACCATCTCCAAAGCCAAAGGGCAGCCCCGAGAACCACAGGTGTACACCCTGC |
| | CCCCATCCCGGGATGAGCTGACCAAGAACCAGGTCAGCCTGACCTGCCTGGTCAAA |
| | GGCTTCTATCCCAGCGACATCGCCGTGGAGTGGGAGAGCAATGGGCAGCCGGAGAA |
| | CAACTACAAGACCACGCCTCCCGTGCTGGACTCCGACGGCTCCTTCTTCCTCTACAG |
| | CAAGCTCACCGTGGACAAGAGCAGGTGGCAGCAGGGGAACGTCTTCTCATGCTCCG |
| | TGATGCATGAGGCTCTGCACAACCACTACACGCAGAAGAGCCTCTCCCTGTCTCCGG |
| | GTAAA (SEQ ID NO: 179) |
| DAB012123 | CAGGTGCAGCTGGTGGAGAGCGGCGGCGGCGTGGTGCAGCCCGGCGGCAGCCTGAG |
| | GCTGAGCTGCGCCGCCAGCGGCTTCACCTTCAGCGACTACTACATGTTCTGGATCAG |
| | GCAGGCCCCCGGCAAGGGCCTGGAGTGGGTGGCCTACATCAGCAACGGCGGCGGCA |
| | ACACCTACTACCCCGACACCGTGAAGGGCAGGTTCACCATCAGCAGGGACAACGCC |
| | AAGAACACCCTGTACCTGCAGATGAACAGCCTGAGGGCCGAGGACACCGCCGTGTA |
| | CTACTGCGCCAGCCCCGAGGCCAGGTACTACGGCAACTACCCCTTCCCCTACTGGGG |
| | CCAGGGCACCCTGGTGACCGTGAGCAGCGCTAGCACCAAGGGCCCATCGGTCTTCC |
| | CCCTGGCACCCTCCTCCAAGAGCACCTCTGGGGGCACAGCGGCCCTGGGCTGCCTGG |
| | TCAAGGACTACTTCCCCGAACCGGTGACGGTGTCGTGGAACTCAGGCGCCCTGACC |
| | AGCGGCGTGCACACCTTCCCGGCTGTCCTACAGTCCTCAGGACTCTACTCCCTCAGC |
| | AGCGTGGTGACCGTGCCCTCCAGCAGCTTGGGCACCCAGACCTACATCTGCAACGTG |
| | AATCACAAGCCCAGCAACACCAAGGTGGACAAGAAAGTTGAGCCCAAATCTTGTGA |
| | CAAAACTCACACATGCCCACCGTGCCCAGCACCTGAACTCCTGGGGGGACCGTCAG |
| | TCTTCCTCTTCCCCCCAAAACCCAAGGACACCCTCATGATCTCCCGGACCCCTGAGG |
| | TCACATGCGTGGTGGTGGACGTGAGCCACGAAGACCCTGAGGTCAAGTTCAACTGG |
| | TACGTGGACGGCGTGGAGGTGCATAATGCCAAGACAAAGCCGCGGGAGGAGCAGT |
| | ACAACAGCACGTACCGGGTGGTCAGCGTCCTCACCGTCCTGCACCAGGACTGGCTG |
| | AATGGCAAGGAGTACAAGTGCAAGGTCTCCAACAAAGCCCTCCCAGCCCCCATCGA |
| | GAAAACCATCTCCAAAGCCAAAGGGCAGCCCCGAGAACCACAGGTGTACACCCTGC |
| | CCCCATCCCGGGATGAGCTGACCAAGAACCAGGTCAGCCTGACCTGCCTGGTCAAA |
| | GGCTTCTATCCCAGCGACATCGCCGTGGAGTGGGAGAGCAATGGGCAGCCGGAGAA |
| | CAACTACAAGACCACGCCTCCCGTGCTGGACTCCGACGGCTCCTTCTTCCTCTACAG |
| | CAAGCTCACCGTGGACAAGAGCAGGTGGCAGCAGGGGAACGTCTTCTCATGCTCCG |
| | TGATGCATGAGGCTCTGCACAACCACTACACGCAGAAGAGCCTCTCCCTGTCTCCGG |
| | GTAAA (SEQ ID NO: 180) |
| DAB012124 | CAGGTGCAGCTGCTGCAGAGCGGCCCCGGCCTGGTGAAGCCCAGCGCCACCCTGAG |
| | CCTGACCTGCACCGTGAGCGGCTTCAGCCTGACCACCTACGGCGTGCACTGGATCAG |
| | GCAGCCCCCCGGCAAGGGCCTGGAGTGGATGGGCGTGATCTGGAGCGGCGGCAGCA |
| | CCGACTACAACGCCGCCTTCATCAGCAGGCTGACCATCAGCAAGGACAACAGCAAG |
| | AACCAGGTGTTCCTGCAGCTGTACAGCCTGAGGGCCGAGGACACCGCCATCTACTA |
| | CTGCGCCAGGACCAGCCACTGGTACTTCGACGTGTGGGGCAGGGGCACCCTGGTGA |
| | CCGTGAGCAGCGCTAGCACCAAGGGCCCATCGGTCTTCCCCCTGGCACCCTCCTCCA |
| | AGAGCACCTCTGGGGGCACAGCGGCCCTGGGCTGCCTGGTCAAGGACTACTTCCCC |
| | GAACCGGTGACGGTGTCGTGGAACTCAGGCGCCCTGACCAGCGGCGTGCACACCTT |
| | CCCGGCTGTCCTACAGTCCTCAGGACTCTACTCCCTCAGCAGCGTGGTGACCGTGCC |
| | CTCCAGCAGCTTGGGCACCCAGACCTACATCTGCAACGTGAATCACAAGCCCAGCA |
| | ACACCAAGGTGGACAAGAAAGTTGAGCCCAAATCTTGTGACAAAACTCACACATGC |
| | CCACCGTGCCCAGCACCTGAACTCCTGGGGGGACCGTCAGTCTTCCTCTTCCCCCCA |
| | AAACCCAAGGACACCCTCATGATCTCCCGGACCCCTGAGGTCACATGCGTGGTGGT |
| | GGACGTGAGCCACGAAGACCCTGAGGTCAAGTTCAACTGGTACGTGGACGGCGTGG |
| | AGGTGCATAATGCCAAGACAAAGCCGCGGGAGGAGCAGTACAACAGCACGTACCG |
| | GGTGGTCAGCGTCCTCACCGTCCTGCACCAGGACTGGCTGAATGGCAAGGAGTACA |
| | AGTGCAAGGTCTCCAACAAAGCCCTCCCAGCCCCCATCGAGAAAACCATCTCCAAA |
| | GCCAAAGGGCAGCCCCGAGAACCACAGGTGTACACCCTGCCCCCATCCCGGGATGA |
| | GCTGACCAAGAACCAGGTCAGCCTGACCTGCCTGGTCAAAGGCTTCTATCCCAGCG |
| | ACATCGCCGTGGAGTGGGAGAGCAATGGGCAGCCGGAGAACAACTACAAGACCAC |
| | GCCTCCCGTGCTGGACTCCGACGGCTCCTTCTTCCTCTACAGCAAGCTCACCGTGGA |
| | CAAGAGCAGGTGGCAGCAGGGGAACGTCTTCTCATGCTCCGTGATGCATGAGGCTC |
| | TGCACAACCACTACACGCAGAAGAGCCTCTCCCTGTCTCCGGGTAAA (SEQ ID NO: |
| | 181) |
| DAB012125 | CAGGTGCAGCTGCAGGAGAGCGGCCCCGGCCTGGTGAAGCCCAGCCAGACCCTGAG |
| | CCTGACCTGCACCGTGAGCGGCTTCAGCCTGACCACCTACGGCGTGCACTGGGTGAG |
| | GCAGAGCCCCGGCAAGGGCCTGGAGTGGCTGGGCGTGATCTGGAGCGGCGGCAGCA |
| | CCGACTACAACGCCGCCTTCATCAGCAGGCTGAGCATCAGCAAGGACAACAGCAAG |
| | AGCCAGGTGTTCCTGGAGATGACCAGCCTGACCGCCGCCGACACCGCCATCTACTAC |
| | TGCGCCAGGACCAGCCACTGGTACTTCGACGTGTGGGGCAGGGGCACCCTGGTGAC |
| | CGTGAGCAGCGCTAGCACCAAGGGCCCATCGGTCTTCCCCCTGGCACCCTCCTCCAA |
| | GAGCACCTCTGGGGGCACAGCGGCCCTGGGCTGCCTGGTCAAGGACTACTTCCCCG |
| | AACCGGTGACGGTGTCGTGGAACTCAGGCGCCCTGACCAGCGGCGTGCACACCTTC |
| | CCGGCTGTCCTACAGTCCTCAGGACTCTACTCCCTCAGCAGCGTGGTGACCGTGCCC |
| | TCCAGCAGCTTGGGCACCCAGACCTACATCTGCAACGTGAATCACAAGCCCAGCAA |
| | CACCAAGGTGGACAAGAAAGTTGAGCCCAAATCTTGTGACAAAACTCACACATGCC |
| | CACCGTGCCCAGCACCTGAACTCCTGGGGGGACCGTCAGTCTTCCTCTTCCCCCCAA |
| | AACCCAAGGACACCCTCATGATCTCCCGGACCCCTGAGGTCACATGCGTGGTGGTG |
| | GACGTGAGCCACGAAGACCCTGAGGTCAAGTTCAACTGGTACGTGGACGGCGTGGA |
| | GGTGCATAATGCCAAGACAAAGCCGCGGGAGGAGCAGTACAACAGCACGTACCGG |
| | GTGGTCAGCGTCCTCACCGTCCTGCACCAGGACTGGCTGAATGGCAAGGAGTACAA |
| | GTGCAAGGTCTCCAACAAAGCCCTCCCAGCCCCCATCGAGAAAACCATCTCCAAAG |
| | CCAAAGGGCAGCCCCGAGAACCACAGGTGTACACCCTGCCCCCATCCCGGGATGAG |
| | CTGACCAAGAACCAGGTCAGCCTGACCTGCCTGGTCAAAGGCTTCTATCCCAGCGAC |
| | ATCGCCGTGGAGTGGGAGAGCAATGGGCAGCCGGAGAACAACTACAAGACCACGC |
| | CTCCCGTGCTGGACTCCGACGGCTCCTTCTTCCTCTACAGCAAGCTCACCGTGGACA |
| | AGAGCAGGTGGCAGCAGGGGAACGTCTTCTCATGCTCCGTGATGCATGAGGCTCTG |
| | CACAACCACTACACGCAGAAGAGCCTCTCCCTGTCTCCGGGTAAA (SEQ ID NO: 182) |
| DAB012126 | CAGGTGCAGCTGCAGGAGAGCGGCCCCGGCCTGGTGAAGCCCAGCCAGACCCTGAG |
| | CCTGACCTGCACCGTGAGCGGCTTCAGCCTGACCACCTACGGCGTGCACTGGGTGAG |
| | GCAGGCCCCCGAGAAGGGCCTGGAGTGGCTGGGCGTGATCTGGAGCGGCGGCAGCA |
| | CCGACTACAACGCCGCCTTCATCAGCAGGCTGAGCATCAGCAAGGACAACAGCAAG |
| | AGCCAGGTGTTCCTGGAGATGACCAGCCTGACCGCCGACGACACCGCCATCTACTA |
| | CTGCGCCAGGACCAGCCACTGGTACTTCGACGTGTGGGGCAGGGGCACCCTGGTGA |
| | CCGTGAGCAGCGCTAGCACCAAGGGCCCATCGGTCTTCCCCCTGGCACCCTCCTCCA |
| | AGAGCACCTCTGGGGGCACAGCGGCCCTGGGCTGCCTGGTCAAGGACTACTTCCCC |
| | GAACCGGTGACGGTGTCGTGGAACTCAGGCGCCCTGACCAGCGGCGTGCACACCTT |
| | CCCGGCTGTCCTACAGTCCTCAGGACTCTACTCCCTCAGCAGCGTGGTGACCGTGCC |
| | CTCCAGCAGCTTGGGCACCCAGACCTACATCTGCAACGTGAATCACAAGCCCAGCA |
| | ACACCAAGGTGGACAAGAAAGTTGAGCCCAAATCTTGTGACAAAACTCACACATGC |
| | CCACCGTGCCCAGCACCTGAACTCCTGGGGGGACCGTCAGTCTTCCTCTTCCCCCCA |
| | AAACCCAAGGACACCCTCATGATCTCCCGGACCCCTGAGGTCACATGCGTGGTGGT |
| | GGACGTGAGCCACGAAGACCCTGAGGTCAAGTTCAACTGGTACGTGGACGGCGTGG |
| | AGGTGCATAATGCCAAGACAAAGCCGCGGGAGGAGCAGTACAACAGCACGTACCG |
| | GGTGGTCAGCGTCCTCACCGTCCTGCACCAGGACTGGCTGAATGGCAAGGAGTACA |
| | AGTGCAAGGTCTCCAACAAAGCCCTCCCAGCCCCCATCGAGAAAACCATCTCCAAA |
| | GCCAAAGGGCAGCCCCGAGAACCACAGGTGTACACCCTGCCCCCATCCCGGGATGA |
| | GCTGACCAAGAACCAGGTCAGCCTGACCTGCCTGGTCAAAGGCTTCTATCCCAGCG |
| | ACATCGCCGTGGAGTGGGAGAGCAATGGGCAGCCGGAGAACAACTACAAGACCAC |
| | GCCTCCCGTGCTGGACTCCGACGGCTCCTTCTTCCTCTACAGCAAGCTCACCGTGGA |
| | CAAGAGCAGGTGGCAGCAGGGGAACGTCTTCTCATGCTCCGTGATGCATGAGGCTC |
| | TGCACAACCACTACACGCAGAAGAGCCTCTCCCTGTCTCCGGGTAAA (SEQ ID NO: |
| | 183) |
| DAB012127 | CAGGTGCAGCTGCAGGAGAGCGGCCCCGGCCTGGTGAAGCCCAGCCAGACCCTGAG |
| | CCTGACCTGCACCGTGAGCGGCTTCAGCCTGACCACCTACGGCGTGCACTGGGTGAG |
| | GCAGACCCCCGAGAAGGGCCTGGAGTGGCTGGGCGTGATCTGGAGCGGCGGCAGCA |
| | CCGACTACAACGCCGCCTTCATCAGCAGGCTGAGCATCAGCAAGGACAACAGCAAG |
| | AGCCAGGTGTTCCTGGAGATGACCAGCCTGACCGCCGACGACACCGCCATCTACTA |
| | CTGCGCCAGGACCAGCCACTGGTACTTCGACGTGTGGGGCAGGGGCACCCTGGTGA |
| | CCGTGAGCAGCGCTAGCACCAAGGGCCCATCGGTCTTCCCCCTGGCACCCTCCTCCA |
| | AGAGCACCTCTGGGGGCACAGCGGCCCTGGGCTGCCTGGTCAAGGACTACTTCCCC |
| | GAACCGGTGACGGTGTCGTGGAACTCAGGCGCCCTGACCAGCGGCGTGCACACCTT |
| | CCCGGCTGTCCTACAGTCCTCAGGACTCTACTCCCTCAGCAGCGTGGTGACCGTGCC |
| | CTCCAGCAGCTTGGGCACCCAGACCTACATCTGCAACGTGAATCACAAGCCCAGCA |
| | ACACCAAGGTGGACAAGAAAGTTGAGCCCAAATCTTGTGACAAAACTCACACATGC |
| | CCACCGTGCCCAGCACCTGAACTCCTGGGGGGACCGTCAGTCTTCCTCTTCCCCCCA |
| | AAACCCAAGGACACCCTCATGATCTCCCGGACCCCTGAGGTCACATGCGTGGTGGT |
| | GGACGTGAGCCACGAAGACCCTGAGGTCAAGTTCAACTGGTACGTGGACGGCGTGG |
| | AGGTGCATAATGCCAAGACAAAGCCGCGGGAGGAGCAGTACAACAGCACGTACCG |
| | GGTGGTCAGCGTCCTCACCGTCCTGCACCAGGACTGGCTGAATGGCAAGGAGTACA |
| | AGTGCAAGGTCTCCAACAAAGCCCTCCCAGCCCCCATCGAGAAAACCATCTCCAAA |
| | GCCAAAGGGCAGCCCCGAGAACCACAGGTGTACACCCTGCCCCCATCCCGGGATGA |
| | GCTGACCAAGAACCAGGTCAGCCTGACCTGCCTGGTCAAAGGCTTCTATCCCAGCG |
| | ACATCGCCGTGGAGTGGGAGAGCAATGGGCAGCCGGAGAACAACTACAAGACCAC |
| | GCCTCCCGTGCTGGACTCCGACGGCTCCTTCTTCCTCTACAGCAAGCTCACCGTGGA |
| | CAAGAGCAGGTGGCAGCAGGGGAACGTCTTCTCATGCTCCGTGATGCATGAGGCTC |
| | TGCACAACCACTACACGCAGAAGAGCCTCTCCCTGTCTCCGGGTAAA (SEQ ID NO: |
| | 184) |
| DAB012128 | CAGGTGCAGCTGCTGGAGAGCGGCGGCGGCCTGGTGCAGCCCGGCGGCAGCCTGAG |
| | GCTGAGCTGCGCCGCCAGCGGCTTCACCTTCAGCGACTACTACATGTACTGGATCAG |
| | GCAGACCCCCCAGAAGAGGCTGGAGTGGGTGGCCTACATCAGCAACGGCGGCGGCA |
| | ACACCTACTACAGCGACACCGTGAAGGGCAGGTTCACCATCAGCAGGGACAACGCC |
| | AAGAACCAGCTGTACCTGCAGATGAGGAGCCTGAGGCCCGAGGACACCGCCATCTA |
| | CTACTGCGCCAGCCCCGAGGCCAGGTACTACGGCAACTTCCCCTTCCCCTACTGGGG |
| | CCAGGGCACCCTGGTGACCGTGAGCAGCGCTAGCACCAAGGGCCCATCGGTCTTCC |
| | CCCTGGCACCCTCCTCCAAGAGCACCTCTGGGGGCACAGCGGCCCTGGGCTGCCTGG |
| | TCAAGGACTACTTCCCCGAACCGGTGACGGTGTCGTGGAACTCAGGCGCCCTGACC |
| | AGCGGCGTGCACACCTTCCCGGCTGTCCTACAGTCCTCAGGACTCTACTCCCTCAGC |
| | AGCGTGGTGACCGTGCCCTCCAGCAGCTTGGGCACCCAGACCTACATCTGCAACGTG |
| | AATCACAAGCCCAGCAACACCAAGGTGGACAAGAAAGTTGAGCCCAAATCTTGTGA |
| | CAAAACTCACACATGCCCACCGTGCCCAGCACCTGAACTCCTGGGGGGACCGTCAG |
| | TCTTCCTCTTCCCCCCAAAACCCAAGGACACCCTCATGATCTCCCGGACCCCTGAGG |
| | TCACATGCGTGGTGGTGGACGTGAGCCACGAAGACCCTGAGGTCAAGTTCAACTGG |
| | TACGTGGACGGCGTGGAGGTGCATAATGCCAAGACAAAGCCGCGGGAGGAGCAGT |
| | ACAACAGCACGTACCGGGTGGTCAGCGTCCTCACCGTCCTGCACCAGGACTGGCTG |
| | AATGGCAAGGAGTACAAGTGCAAGGTCTCCAACAAAGCCCTCCCAGCCCCCATCGA |
| | GAAAACCATCTCCAAAGCCAAAGGGCAGCCCCGAGAACCACAGGTGTACACCCTGC |
| | CCCCATCCCGGGATGAGCTGACCAAGAACCAGGTCAGCCTGACCTGCCTGGTCAAA |
| | GGCTTCTATCCCAGCGACATCGCCGTGGAGTGGGAGAGCAATGGGCAGCCGGAGAA |
| | CAACTACAAGACCACGCCTCCCGTGCTGGACTCCGACGGCTCCTTCTTCCTCTACAG |
| | CAAGCTCACCGTGGACAAGAGCAGGTGGCAGCAGGGGAACGTCTTCTCATGCTCCG |
| | TGATGCATGAGGCTCTGCACAACCACTACACGCAGAAGAGCCTCTCCCTGTCTCCGG |
| | GTAAA (SEQ ID NO: 185) |
| DAB012129 | CAGGTGCAGCTGGTGGAGAGCGGCGGCGGCCTGGTGAAGCCCGGCGGCAGCCTGAG |
| | GCTGAGCTGCGCCGCCAGCGGCTTCACCTTCAGCGACTACTACATGTACTGGATCAG |
| | GCAGGCCCCCGGCAAGGGCCTGGAGTGGGTGGCCTACATCAGCAACGGCGGCGGCA |
| | ACACCTACTACAGCGACACCGTGAAGGGCAGGTTCACCATCAGCAGGGACAACGCC |
| | AAGAACACCCTGTACCTGCAGATGAACAGCCTGAGGGCCGAGGACACCGCCGTGTA |
| | CTACTGCGCCAGCCCCGAGGCCAGGTACTACGGCAACTTCCCCTTCCCCTACTGGGG |
| | CCAGGGCACCCTGGTGACCGTGAGCAGCGCTAGCACCAAGGGCCCATCGGTCTTCC |
| | CCCTGGCACCCTCCTCCAAGAGCACCTCTGGGGGCACAGCGGCCCTGGGCTGCCTGG |
| | TCAAGGACTACTTCCCCGAACCGGTGACGGTGTCGTGGAACTCAGGCGCCCTGACC |
| | AGCGGCGTGCACACCTTCCCGGCTGTCCTACAGTCCTCAGGACTCTACTCCCTCAGC |
| | AGCGTGGTGACCGTGCCCTCCAGCAGCTTGGGCACCCAGACCTACATCTGCAACGTG |
| | AATCACAAGCCCAGCAACACCAAGGTGGACAAGAAAGTTGAGCCCAAATCTTGTGA |
| | CAAAACTCACACATGCCCACCGTGCCCAGCACCTGAACTCCTGGGGGGACCGTCAG |
| | TCTTCCTCTTCCCCCCAAAACCCAAGGACACCCTCATGATCTCCCGGACCCCTGAGG |
| | TCACATGCGTGGTGGTGGACGTGAGCCACGAAGACCCTGAGGTCAAGTTCAACTGG |
| | TACGTGGACGGCGTGGAGGTGCATAATGCCAAGACAAAGCCGCGGGAGGAGCAGT |
| | ACAACAGCACGTACCGGGTGGTCAGCGTCCTCACCGTCCTGCACCAGGACTGGCTG |
| | AATGGCAAGGAGTACAAGTGCAAGGTCTCCAACAAAGCCCTCCCAGCCCCCATCGA |
| | GAAAACCATCTCCAAAGCCAAAGGGCAGCCCCGAGAACCACAGGTGTACACCCTGC |
| | CCCCATCCCGGGATGAGCTGACCAAGAACCAGGTCAGCCTGACCTGCCTGGTCAAA |
| | GGCTTCTATCCCAGCGACATCGCCGTGGAGTGGGAGAGCAATGGGCAGCCGGAGAA |
| | CAACTACAAGACCACGCCTCCCGTGCTGGACTCCGACGGCTCCTTCTTCCTCTACAG |
| | CAAGCTCACCGTGGACAAGAGCAGGTGGCAGCAGGGGAACGTCTTCTCATGCTCCG |
| | TGATGCATGAGGCTCTGCACAACCACTACACGCAGAAGAGCCTCTCCCTGTCTCCGG |
| | GTAAA (SEQ ID NO: 186) |
| DAB012130 | CAGGTGCAGCTGGTGGAGAGCGGCGGCGGCCTGGTGAAGCCCGGCGGCAGCCTGAG |
| | GCTGAGCTGCGCCGCCAGCGGCTTCACCTTCAGCGACTACTACATGTACTGGATCAG |
| | GCAGGCCCCCGGCAAGGGCCTGGAGTGGGTGGCCTACATCAGCAACGGCGGCGGCA |
| | ACACCTACTACAGCGACACCGTGAAGGGCAGGTTCACCATCAGCAGGGACAACGCC |
| | AAGAACACCCTGTACCTGCAGATGAACAGCCTGAGGGCCGAGGACACCGCCGTGTA |
| | CTACTGCGCCAGCCCCGAGGCCAGGTACTACGGCAACTTCCCCTTCCCCTACTGGGG |
| | CCAGGGCACCCTGGTGACCGTGAGCAGCGCTAGCACCAAGGGCCCATCGGTCTTCC |
| | CCCTGGCACCCTCCTCCAAGAGCACCTCTGGGGGCACAGCGGCCCTGGGCTGCCTGG |
| | TCAAGGACTACTTCCCCGAACCGGTGACGGTGTCGTGGAACTCAGGCGCCCTGACC |
| | AGCGGCGTGCACACCTTCCCGGCTGTCCTACAGTCCTCAGGACTCTACTCCCTCAGC |
| | AGCGTGGTGACCGTGCCCTCCAGCAGCTTGGGCACCCAGACCTACATCTGCAACGTG |
| | AATCACAAGCCCAGCAACACCAAGGTGGACAAGAAAGTTGAGCCCAAATCTTGTGA |
| | CAAAACTCACACATGCCCACCGTGCCCAGCACCTGAACTCCTGGGGGGACCGTCAG |
| | TCTTCCTCTTCCCCCCAAAACCCAAGGACACCCTCATGATCTCCCGGACCCCTGAGG |
| | TCACATGCGTGGTGGTGGACGTGAGCCACGAAGACCCTGAGGTCAAGTTCAACTGG |
| | TACGTGGACGGCGTGGAGGTGCATAATGCCAAGACAAAGCCGCGGGAGGAGCAGT |
| | ACAACAGCACGTACCGGGTGGTCAGCGTCCTCACCGTCCTGCACCAGGACTGGCTG |
| | AATGGCAAGGAGTACAAGTGCAAGGTCTCCAACAAAGCCCTCCCAGCCCCCATCGA |
| | GAAAACCATCTCCAAAGCCAAAGGGCAGCCCCGAGAACCACAGGTGTACACCCTGC |
| | CCCCATCCCGGGATGAGCTGACCAAGAACCAGGTCAGCCTGACCTGCCTGGTCAAA |
| | GGCTTCTATCCCAGCGACATCGCCGTGGAGTGGGAGAGCAATGGGCAGCCGGAGAA |
| | CAACTACAAGACCACGCCTCCCGTGCTGGACTCCGACGGCTCCTTCTTCCTCTACAG |
| | CAAGCTCACCGTGGACAAGAGCAGGTGGCAGCAGGGGAACGTCTTCTCATGCTCCG |
| | TGATGCATGAGGCTCTGCACAACCACTACACGCAGAAGAGCCTCTCCCTGTCTCCGG |
| | GTAAA (SEQ ID NO: 187) |
| DAB012131 | CAGGTGCAGCTGGTGGAGAGCGGCGGCGGCCTGGTGAAGCCCGGCGGCAGCCTGAG |
| | GCTGAGCTGCGCCGCCAGCGGCTTCACCTTCAGCGACTACTACATGTACTGGATCAG |
| | GCAGGCCCCCGGCAAGGGCCTGGAGTGGGTGGCCTACATCAGCAACGGCGGCGGCA |
| | ACACCTACTACAGCGACACCGTGAAGGGCAGGTTCACCATCAGCAGGGACAACGCC |
| | AAGAACACCCTGTACCTGCAGATGAACAGCCTGAGGGCCGAGGACACCGCCGTGTA |
| | CTACTGCGCCAGCCCCGAGGCCAGGTACTACGGCAACTTCCCCTTCCCCTACTGGGG |
| | CCAGGGCACCCTGGTGACCGTGAGCAGCGCTAGCACCAAGGGCCCATCGGTCTTCC |
| | CCCTGGCACCCTCCTCCAAGAGCACCTCTGGGGGCACAGCGGCCCTGGGCTGCCTGG |
| | TCAAGGACTACTTCCCCGAACCGGTGACGGTGTCGTGGAACTCAGGCGCCCTGACC |
| | AGCGGCGTGCACACCTTCCCGGCTGTCCTACAGTCCTCAGGACTCTACTCCCTCAGC |
| | AGCGTGGTGACCGTGCCCTCCAGCAGCTTGGGCACCCAGACCTACATCTGCAACGTG |
| | AATCACAAGCCCAGCAACACCAAGGTGGACAAGAAAGTTGAGCCCAAATCTTGTGA |
| | CAAAACTCACACATGCCCACCGTGCCCAGCACCTGAACTCCTGGGGGGACCGTCAG |
| | TCTTCCTCTTCCCCCCAAAACCCAAGGACACCCTCATGATCTCCCGGACCCCTGAGG |
| | TCACATGCGTGGTGGTGGACGTGAGCCACGAAGACCCTGAGGTCAAGTTCAACTGG |
| | TACGTGGACGGCGTGGAGGTGCATAATGCCAAGACAAAGCCGCGGGAGGAGCAGT |
| | ACAACAGCACGTACCGGGTGGTCAGCGTCCTCACCGTCCTGCACCAGGACTGGCTG |
| | AATGGCAAGGAGTACAAGTGCAAGGTCTCCAACAAAGCCCTCCCAGCCCCCATCGA |
| | GAAAACCATCTCCAAAGCCAAAGGGCAGCCCCGAGAACCACAGGTGTACACCCTGC |
| | CCCCATCCCGGGATGAGCTGACCAAGAACCAGGTCAGCCTGACCTGCCTGGTCAAA |
| | GGCTTCTATCCCAGCGACATCGCCGTGGAGTGGGAGAGCAATGGGCAGCCGGAGAA |
| | CAACTACAAGACCACGCCTCCCGTGCTGGACTCCGACGGCTCCTTCTTCCTCTACAG |
| | CAAGCTCACCGTGGACAAGAGCAGGTGGCAGCAGGGGAACGTCTTCTCATGCTCCG |
| | TGATGCATGAGGCTCTGCACAACCACTACACGCAGAAGAGCCTCTCCCTGTCTCCGG |
| | GTAAA (SEQ ID NO: 188) |
| DAB012132 | CAGGTGCAGCTGGTGGAGAGCGGCGGCGGCGTGGTGCAGCCCGGCAGGAGCCTGA |
| | GGCTGAGCTGCGCCGCCAGCGGCTTCAGCTTCAGCAACTACTACATGGCCTGGGTGA |
| | GGCAGGCCCCCGGCAAGGGCCTGGAGTGGGTGGCCAGCATCAGCACCGGCGGCGGC |
| | AACATCTACTACAGGGACAGCGTGAAGGGCAGGTTCACCATCAGCAGGGACAACAG |
| | CAAGAGCACCCTGTACCTGCAGATGAGGAGCCTGAGGAGCGAGGACACCGCCATCT |
| | ACTACTGCGCCAGGCAGACCGCCTACTACGTGATGGACGCCTGGGGCCAGGGCACC |
| | ATGGTGACCGTGAGCAGCGCTAGCACCAAGGGCCCATCGGTCTTCCCCCTGGCACCC |
| | TCCTCCAAGAGCACCTCTGGGGGCACAGCGGCCCTGGGCTGCCTGGTCAAGGACTA |
| | CTTCCCCGAACCGGTGACGGTGTCGTGGAACTCAGGCGCCCTGACCAGCGGCGTGC |
| | ACACCTTCCCGGCTGTCCTACAGTCCTCAGGACTCTACTCCCTCAGCAGCGTGGTGA |
| | CCGTGCCCTCCAGCAGCTTGGGCACCCAGACCTACATCTGCAACGTGAATCACAAGC |
| | CCAGCAACACCAAGGTGGACAAGAAAGTTGAGCCCAAATCTTGTGACAAAACTCAC |
| | ACATGCCCACCGTGCCCAGCACCTGAACTCCTGGGGGGACCGTCAGTCTTCCTCTTC |
| | CCCCCAAAACCCAAGGACACCCTCATGATCTCCCGGACCCCTGAGGTCACATGCGTG |
| | GTGGTGGACGTGAGCCACGAAGACCCTGAGGTCAAGTTCAACTGGTACGTGGACGG |
| | CGTGGAGGTGCATAATGCCAAGACAAAGCCGCGGGAGGAGCAGTACAACAGCACG |
| | TACCGGGTGGTCAGCGTCCTCACCGTCCTGCACCAGGACTGGCTGAATGGCAAGGA |
| | GTACAAGTGCAAGGTCTCCAACAAAGCCCTCCCAGCCCCCATCGAGAAAACCATCT |
| | CCAAAGCCAAAGGGCAGCCCCGAGAACCACAGGTGTACACCCTGCCCCCATCCCGG |
| | GATGAGCTGACCAAGAACCAGGTCAGCCTGACCTGCCTGGTCAAAGGCTTCTATCCC |
| | AGCGACATCGCCGTGGAGTGGGAGAGCAATGGGCAGCCGGAGAACAACTACAAGA |
| | CCACGCCTCCCGTGCTGGACTCCGACGGCTCCTTCTTCCTCTACAGCAAGCTCACCG |
| | TGGACAAGAGCAGGTGGCAGCAGGGGAACGTCTTCTCATGCTCCGTGATGCATGAG |
| | GCTCTGCACAACCACTACACGCAGAAGAGCCTCTCCCTGTCTCCGGGTAAA (SEQ ID |
| | NO: 189) |
| DAB012133 | GAGGTGCAGCTGGTGGAGAGCGGCGGCGGCCTGGTGCAGCCCGGCGGCAGCCTGAG |
| | GCTGAGCTGCGCCGCCAGCGGCTTCAGCTTCAGCAACTACTACATGGCCTGGGTGAG |
| | GCAGGCCCCCGGCAAGGGCCTGGAGTGGGTGGCCAGCATCAGCACCGGCGGCGGCA |
| | ACATCTACTACAGGGACAGCGTGAAGGGCAGGTTCACCATCAGCAGGGACAACGCC |
| | AAGAACAGCCTGTACCTGCAGATGAACAGCCTGAGGGCCGAGGACACCGCCGTGTA |
| | CTACTGCGCCAGGCAGACCGCCTACTACGTGATGGACGCCTGGGGCCAGGGCACCA |
| | CCGTGACCGTGAGCAGCGCTAGCACCAAGGGCCCATCGGTCTTCCCCCTGGCACCCT |
| | CCTCCAAGAGCACCTCTGGGGGCACAGCGGCCCTGGGCTGCCTGGTCAAGGACTAC |
| | TTCCCCGAACCGGTGACGGTGTCGTGGAACTCAGGCGCCCTGACCAGCGGCGTGCA |
| | CACCTTCCCGGCTGTCCTACAGTCCTCAGGACTCTACTCCCTCAGCAGCGTGGTGAC |
| | CGTGCCCTCCAGCAGCTTGGGCACCCAGACCTACATCTGCAACGTGAATCACAAGCC |
| | CAGCAACACCAAGGTGGACAAGAAAGTTGAGCCCAAATCTTGTGACAAAACTCACA |
| | CATGCCCACCGTGCCCAGCACCTGAACTCCTGGGGGGACCGTCAGTCTTCCTCTTCC |
| | CCCCAAAACCCAAGGACACCCTCATGATCTCCCGGACCCCTGAGGTCACATGCGTG |
| | GTGGTGGACGTGAGCCACGAAGACCCTGAGGTCAAGTTCAACTGGTACGTGGACGG |
| | CGTGGAGGTGCATAATGCCAAGACAAAGCCGCGGGAGGAGCAGTACAACAGCACG |
| | TACCGGGTGGTCAGCGTCCTCACCGTCCTGCACCAGGACTGGCTGAATGGCAAGGA |
| | GTACAAGTGCAAGGTCTCCAACAAAGCCCTCCCAGCCCCCATCGAGAAAACCATCT |
| | CCAAAGCCAAAGGGCAGCCCCGAGAACCACAGGTGTACACCCTGCCCCCATCCCGG |
| | GATGAGCTGACCAAGAACCAGGTCAGCCTGACCTGCCTGGTCAAAGGCTTCTATCCC |
| | AGCGACATCGCCGTGGAGTGGGAGAGCAATGGGCAGCCGGAGAACAACTACAAGA |
| | CCACGCCTCCCGTGCTGGACTCCGACGGCTCCTTCTTCCTCTACAGCAAGCTCACCG |
| | TGGACAAGAGCAGGTGGCAGCAGGGGAACGTCTTCTCATGCTCCGTGATGCATGAG |
| | GCTCTGCACAACCACTACACGCAGAAGAGCCTCTCCCTGTCTCCGGGTAAA (SEQ ID |
| | NO: 190) |
| DAB012134 | GAGGTGCAGCTGGTGGAGAGCGGCGGCGGCCTGGTGCAGCCCGGCGGCAGCCTGAG |
| | GCTGAGCTGCGCCGCCAGCGGCTTCAGCTTCAGCAACTACTACATGGCCTGGGTGAG |
| | GCAGGCCCCCGGCAAGGGCCTGGAGTGGGTGGCCAGCATCAGCACCGGCGGCGGCA |
| | ACATCTACTACAGGGACAGCGTGAAGGGCAGGTTCACCATCAGCAGGGACAACGCC |
| | AAGAACAGCCTGTACCTGCAGATGAACAGCCTGAGGGCCGAGGACACCGCCGTGTA |
| | CTACTGCGCCAGGCAGACCGCCTACTACGTGATGGACGCCTGGGGCCAGGGCACCA |
| | CCGTGACCGTGAGCAGCGCTAGCACCAAGGGCCCATCGGTCTTCCCCCTGGCACCCT |
| | CCTCCAAGAGCACCTCTGGGGGCACAGCGGCCCTGGGCTGCCTGGTCAAGGACTAC |
| | TTCCCCGAACCGGTGACGGTGTCGTGGAACTCAGGCGCCCTGACCAGCGGCGTGCA |
| | CACCTTCCCGGCTGTCCTACAGTCCTCAGGACTCTACTCCCTCAGCAGCGTGGTGAC |
| | CGTGCCCTCCAGCAGCTTGGGCACCCAGACCTACATCTGCAACGTGAATCACAAGCC |
| | CAGCAACACCAAGGTGGACAAGAAAGTTGAGCCCAAATCTTGTGACAAAACTCACA |
| | CATGCCCACCGTGCCCAGCACCTGAACTCCTGGGGGGACCGTCAGTCTTCCTCTTCC |
| | CCCCAAAACCCAAGGACACCCTCATGATCTCCCGGACCCCTGAGGTCACATGCGTG |
| | GTGGTGGACGTGAGCCACGAAGACCCTGAGGTCAAGTTCAACTGGTACGTGGACGG |
| | CGTGGAGGTGCATAATGCCAAGACAAAGCCGCGGGAGGAGCAGTACAACAGCACG |
| | TACCGGGTGGTCAGCGTCCTCACCGTCCTGCACCAGGACTGGCTGAATGGCAAGGA |
| | GTACAAGTGCAAGGTCTCCAACAAAGCCCTCCCAGCCCCCATCGAGAAAACCATCT |
| | CCAAAGCCAAAGGGCAGCCCCGAGAACCACAGGTGTACACCCTGCCCCCATCCCGG |
| | GATGAGCTGACCAAGAACCAGGTCAGCCTGACCTGCCTGGTCAAAGGCTTCTATCCC |
| | AGCGACATCGCCGTGGAGTGGGAGAGCAATGGGCAGCCGGAGAACAACTACAAGA |
| | CCACGCCTCCCGTGCTGGACTCCGACGGCTCCTTCTTCCTCTACAGCAAGCTCACCG |
| | TGGACAAGAGCAGGTGGCAGCAGGGGAACGTCTTCTCATGCTCCGTGATGCATGAG |
| | GCTCTGCACAACCACTACACGCAGAAGAGCCTCTCCCTGTCTCCGGGTAAA (SEQ ID |
| | NO: 191) |
| DAB012135 | GAGGTGCAGCTGGTGGAGAGCGGCGGCGGCCTGGTGCAGCCCGGCGGCAGCCTGAG |
| | GCTGAGCTGCGCCGCCAGCGGCTTCAGCTTCAGCAACTACTACATGGCCTGGGTGAG |
| | GCAGGCCCCCGGCAAGGGCCTGGAGTGGGTGGCCAGCATCAGCACCGGCGGCGGCA |
| | ACATCTACTACAGGGACAGCGTGAAGGGCAGGTTCACCATCAGCAGGGACAACGCC |
| | AAGAACAGCCTGTACCTGCAGATGAACAGCCTGAGGGCCGAGGACACCGCCGTGTA |
| | CTACTGCGCCAGGCAGACCGCCTACTACGTGATGGACGCCTGGGGCCAGGGCACCA |
| | CCGTGACCGTGAGCAGCGCTAGCACCAAGGGCCCATCGGTCTTCCCCCTGGCACCCT |
| | CCTCCAAGAGCACCTCTGGGGGCACAGCGGCCCTGGGCTGCCTGGTCAAGGACTAC |
| | TTCCCCGAACCGGTGACGGTGTCGTGGAACTCAGGCGCCCTGACCAGCGGCGTGCA |
| | CACCTTCCCGGCTGTCCTACAGTCCTCAGGACTCTACTCCCTCAGCAGCGTGGTGAC |
| | CGTGCCCTCCAGCAGCTTGGGCACCCAGACCTACATCTGCAACGTGAATCACAAGCC |
| | CAGCAACACCAAGGTGGACAAGAAAGTTGAGCCCAAATCTTGTGACAAAACTCACA |
| | CATGCCCACCGTGCCCAGCACCTGAACTCCTGGGGGGACCGTCAGTCTTCCTCTTCC |
| | CCCCAAAACCCAAGGACACCCTCATGATCTCCCGGACCCCTGAGGTCACATGCGTG |
| | GTGGTGGACGTGAGCCACGAAGACCCTGAGGTCAAGTTCAACTGGTACGTGGACGG |
| | CGTGGAGGTGCATAATGCCAAGACAAAGCCGCGGGAGGAGCAGTACAACAGCACG |
| | TACCGGGTGGTCAGCGTCCTCACCGTCCTGCACCAGGACTGGCTGAATGGCAAGGA |
| | GTACAAGTGCAAGGTCTCCAACAAAGCCCTCCCAGCCCCCATCGAGAAAACCATCT |
| | CCAAAGCCAAAGGGCAGCCCCGAGAACCACAGGTGTACACCCTGCCCCCATCCCGG |
| | GATGAGCTGACCAAGAACCAGGTCAGCCTGACCTGCCTGGTCAAAGGCTTCTATCCC |
| | AGCGACATCGCCGTGGAGTGGGAGAGCAATGGGCAGCCGGAGAACAACTACAAGA |
| | CCACGCCTCCCGTGCTGGACTCCGACGGCTCCTTCTTCCTCTACAGCAAGCTCACCG |
| | TGGACAAGAGCAGGTGGCAGCAGGGGAACGTCTTCTCATGCTCCGTGATGCATGAG |
| | GCTCTGCACAACCACTACACGCAGAAGAGCCTCTCCCTGTCTCCGGGTAAA (SEQ ID |
| | NO: 192) |

### Example 5: Testing Binding of Nectin-4 Humanized Antibodies

An surface plasmon resonance (SPR) assay was used to determine the binding affinity of Nectin-4 antibodies to Nectin-4 protein. Antibodies were captured using an anti-human Fc capture antibody (Cytiva, #BR100839) immobilized onto a Biacore CM5 standard surface sensor chip following manufacturer instructions. Recombinant human His-tagged Nectin-4 protein was titrated over captured antibody as a 3-fold serial dilutions starting from 400 nM. Association was monitored for 180 sec and dissociation was monitored for 300 sec. Binding assays were performed at 25° C. using HBS-EP+ running buffer containing 10 mM HEPES, 150 mM NaCl, 3 mM EDTA, 0.05% Tween® 20. The surface of the CM5 chip was regenerated with 3M MgCl2 for 30 sec at 30 mL/min. The assay was run on a Biacore® T200 instrument (Cytiva, Marlborough, Massachusetts, USA). The data were “double referenced” by subtracting the response from the reference control flow cell and that from a buffer injection and were fit to a 1:1 binding model using Cytiva's Biacore® T200 Evaluation Software. The association rate constant (ka, M-1s-1) and dissociation rate constant (kd, s-1) were determined and used to calculate the equilibrium dissociation rate (KD, M)=kd/ka. The reported affinity (KD) was calculated from the average of 2 replicate interactions. Data is shown in Table 18.

TABLE 18
Binding Data for Nectin-4 Humanized Antibodies

| Analyte | | | | | | |
|---|---|---|---|---|---|---|
| Sequence | Human Nectin-4 | | | Cyno Nectin-4 | | |
| name/Lot # | ka (1/M-s) | kd (1/s) | KD (M) | ka (1/M-s) | kd (1/s) | KD (M) |
| DAB012120_001 | 2.00E+05 | 5.00E−04 | 2.50E−09 | 1.60E+05 | 5.30E−04 | 3.30E−09 |
| DAB012121_001 | 2.10E+05 | 5.70E−04 | 2.70E−09 | 1.70E+05 | 6.10E−04 | 3.70E−09 |
| DAB012122_001 | 1.70E+05 | 5.90E−04 | 3.50E−09 | 1.30E+05 | 6.00E−04 | 4.50E−09 |
| DAB012123_001 | 1.50E+05 | 5.40E−04 | 3.70E−09 | 1.10E+05 | 5.70E−04 | 5.10E−09 |
| DAB012124_001 | 8.40E+04 | 3.30E−04 | 3.90E−09 | 6.50E+04 | 3.20E−04 | 5.00E−09 |
| DAB012125_001 | 8.70E+04 | 3.10E−04 | 3.50E−09 | 6.50E+04 | 3.50E−04 | 5.40E−09 |
| DAB012126_001 | 9.60E+04 | 3.00E−04 | 3.10E−09 | 7.10E+04 | 3.20E−04 | 4.50E−09 |
| DAB012127_001 | 9.20E+04 | 3.20E−04 | 3.50E−09 | 7.10E+04 | 3.50E−04 | 5.00E−09 |
| DAB012128_001 | 1.50E+05 | 9.90E−04 | 6.50E−09 | 1.20E+05 | 9.80E−04 | 8.50E−09 |
| DAB012129_001 | 2.50E+05 | 1.60E−03 | 6.50E−09 | 1.90E+05 | 1.60E−03 | 8.60E−09 |
| DAB012130_001 | 2.90E+05 | 2.50E−03 | 8.70E−09 | 2.20E+05 | 2.40E−03 | 1.10E−08 |
| DAB012131_001 | 2.80E+05 | 2.60E−03 | 9.20E−09 | 2.20E+05 | 2.50E−03 | 1.10E−08 |
| DAB012132_001 | 2.00E+05 | 1.10E−03 | 5.30E−09 | 1.30E+05 | 3.20E−03 | 2.40E−08 |
| DAB012133_001 | 3.80E+05 | 1.00E−03 | 2.70E−09 | 2.70E+05 | 3.10E−03 | 1.10E−08 |
| DAB012134_001 | 3.90E+05 | 9.90E−04 | 2.50E−09 | 2.90E+05 | 2.80E−03 | 9.80E−09 |
| DAB012135_001 | 3.80E+05 | 7.70E−04 | 2.00E−09 | 2.80E+05 | 2.20E−03 | 7.80E−09 |

### Example 6: Synthesis of Nectin-4 Mask Sequences

Phage Biopanning: Biopanning with ml3 phagemid p8 or p3 displayed peptide libraries was either performed with immobilized antibody or antibody fragments of interest coated on 96-well ELISA plates or with biotin-conjugated antibody or antibody fragments of interest immobilized on streptavidin coated paramagnetic beads. Following binding to target and washing steps, specifically bound phage were recovered by elution using acidic buffer. Enrichment of specific binding clones was accomplished by 3-4 rounds of successive biopanning and amplification. After 3 or 4 rounds of biopanning phage pools were infected into TGI cells and plated out on LB-ampicillin/agar plates for clonal isolation and subsequent characterization.

Phage Hit Identification: After plating TG1 infected with phage pool eluates, individual colonies were grown in 96 well plates for several hours and infected with helper phage to produce peptide displayed phagemid following an overnight growth. The next day the plates were centrifuged to separate the soluble phagemid from the E. coli cells. The phagemid containing supernatants were then combined with PBS Tween 20 (0.05%)+BSA (1%) pH neutral blocking buffer and incubated in previously target antibody or antibody fragment coated and blocked wells. After a short incubation, plates were washed and bound phage were detected by anti-ml3 HRP conjugated antibodies using standard TMB-based chromogenic ELISA procedures. Daughter plates or individual wells were subjected to standard DNA sequencing for peptide identification. Phagemid peptide clones were next tested to determine whether they bound within the antigen binding space of the antibody, by target-based competition assay. Target antibody or antibody fragments thereof were immobilized and blocked in a 96-well ELISA plate format. Human Nectin-4 antigen was added to the wells to block the antigen binding site of the plated coated antibody or antibody fragment. After a brief incubation period phagemid supernatants were added to the wells. Following another short incubation period the plates were washed and specifically bound phage were detected by anti-ml3 HRP conjugated antibodies using standard TMB-based chromogenic ELISA procedures. Phagemid clones were determined to bind within the antigenic binding pocket of the target antibody if a decrease in the phage binding signal was observed compared to a well lacking nectin-4 antigen blockade.

Peptide Synthesis: Peptides of interest identified through phage biopanning were chemically synthesized by standard solid phase peptide synthesis techniques know in the art.

Peptide Binding Assays for Mask Sequences (ELISA and Octet): Equilibrium binding experiments (ELISA)—Peptides were evaluated for their ability to bind to target anti-Nectin-4 antibody or antibody fragments thereof in a standard enzyme linked immunosorbent assay (ELISA) format. Specifically, peptides were evaluated for their ability to bind antibody or antibody fragments whose cognate antigen is human Nectin-4. Briefly, biotinylated peptides or biotinylated Nectin-4 antigen were captured on neutravidin coated plates. Target antibody or antibody fragments were diluted in buffer and added to the peptide or antigen captured plates. Bound antibody was detected using a standard horse radish peroxidase conjugate secondary antibody. The concentration of antibody or antibody fragment thereof required to achieve 50% maximal signal (EC50) was calculated using Graphpad Prism software.

Kinetic binding experiments (Octet)—Kinetic binding of peptides to target antibody or antibody fragments thereof were evaluated using biolayer interferometry (BLI). Briefly, biotinylated peptides or biotinylated Nectin-4 antigen were loaded onto a streptavidin coated Octet® SAX biosensor, quenched in biocytin, and baselined in buffer. Target antibody or and antibody fragment thereof was titrated in solution and associated onto the peptide loaded sensor. After a short association period, sensors were transferred into buffer and the dissociation of bound antibody or antibody fragment thereof was measured. Association and dissociation signals were recorded in real time and analyzed using a 1:1 binding model within the instrument software. Analysis using a 1:1 binding model enabled the calculation of the on and off rate constants as well as affinity, KD.

Peptide competitive binding assays—The ability of peptides to bind and inhibit target antibody or antibody fragments thereof was determined in standard competitive ELISA binding experiments. Peptides were evaluated for their ability to inhibit anti-Nectin-4 antibodies or fragments thereof from binding to the Nectin-4 antigen in a standard ELISA format. Briefly, biotinylated nectin-4 antigen was captured on neutravidin coated plates. Anti-Nectin4 antibody or antibody fragments fixed at 2 nanomolar (nM) concentration was pre-incubated with 0-100 micromolar (μM) titrated peptides. After a short pre-incubation period, the mixture of titrated peptide with fixed Nectin-4 antibody or antibody fragment were added to the Nectin-4 antigen coated plates. After a short incubation on the plates, bound antibody or antibody fragment was detected with a standard horse radish peroxidase conjugated secondary antibody. The concentration of peptide required to reduce the maximum signal by 50% (IC50) was calculated in Graphpad Prism software.

Alanine Scanning of Peptides in Mask Sequences: Sequence activity relationships of select peptides were established using standard alanine scan techniques known in the art. Each residue of select peptides was mutated to Alanine and resulting mutant peptides were characterized for their ability to bind the antibody or antibody fragment of interest as well as their ability to inhibit antibody or antibody fragment of interest from binding Nectin-4 protein antigen. Critical residues within the peptide were then identified as those that lost significant binding affinity to the antibody or antibody fragment of interest when mutated to alanine. Peptide residues that maintained binding to target antibody or antibody fragment were identified as a non-critical residue. This information was used to synthesize directed evolution peptide libraries to strengthen peptide affinity for the target antibody or antibody fragment of interest.

Peptide Optimization of Mask Sequences Via Directed Evolution of Phage Library: Directed evolution peptide libraries were synthesized with select mutagenesis of non-critical peptide residues. Critical peptide residues were minimally mutated in the directed evolution libraries. The subsequent peptide libraries were displayed on ml3 phagemid via p3 and subject to biopanning as described above.

Select Nectin-4 Mask sequences that were synthesized using this process are shown in in Table 19.

TABLE 19
Nectin-4 Mask Sequences

| Mask Sequence | Peptide Sequence |
|---|---|
| Nectin-4 Mask | GGWSCAPDEDTWLCPAGG |
| sequence #1 (JXP1957) | (SEQ ID NO: 18) |
| Nectin-4 Mask | GGWFCAPDENTWLCPNAGG |
| sequence #2 (JXP1952) | (SEQ ID NO: 23) |
| Nectin-4 Mask | GGWTCADDEDTWLCPIGG |
| sequence #3 (JXP1956) | (SEQ ID NO: 125) |
| Nectin-4 Mask | GGWACAPDEDTWLCPIGG |
| sequence #4 (JXP1963) | (SEQ ID NO: 126) |

### Example 7: Cell Culture of the Nectin-4 TRACTr Molecule

HD-BIOP3 (a rAAV-modified GS double knockout CHO host cell line licensed from Horizon Discovery) was used as the host cell line for transfection. These cells were maintained in CD CHO medium (Cat #10743029 Thermo Fisher) supplemented with 1× HT (Cat #10743029 Thermo Fisher) and 8 mM L-glutamine (Cat #25030081 Thermo Fisher). Cells were passaged every 2-3 days at a seeding density of 0.3×10\hat{ }6 cells/ml in shake flasks then placed on a shaker set to 140 rpm with a 25 mm orbit. Host cells were maintained in an incubator set to 36.5 C, 5% CO2 and 80% humidity.

The heavy chain (SEQ ID NO: 194) (see Table 20)) and light chain (SEQ ID NO: 193 (see Table 20)) genes were separately cloned into different cargo vectors (spB007) which was purchased from Transposagen Biopharmaceuticals. The spB007 vector contained ITR sites to facilitate PiggyBac integration into the host cell genome. Both the LC and HC were controlled by a CMV promoter and the glutamine synthetase gene was controlled by a SV40 promoter. The entire expression cassette (SV40-GS-CMV-HC or LC) was cloned between the ITR sites in the spB007 vector. Helper plasmid coding for the PiggyBac transposase was purchased from Transposagen Biopharmaceuticals (Cat #spb-DNA-25).

Two days prior to transfection, the host cells were seeded at 0.7×10\hat{ }6 cells/ml in the host cell media described above. On the day of transfections cells were counted and then centrifuged at 1000 rpm for 10 minutes. They were then resuspended in the host cell media containing DMSO with a cell concentration of 6×10\hat{ }6 cells/ml. The cells were then placed back on a shaker set to 140 rpm with a 25 mm orbit for 3 hours. The incubator settings were 36.5 C with 5% CO₂ and 80% humidity. Cultures were then co-transfected with the expression vectors SPB007-HC, spB007-LC and the helper plasmid spB-DNA using PEI at (2.5:1, PEI:DNA ratio). Cells were placed back in the incubator for 2 days to recover. After 48-hours of recovery, the cells were passaged into selection media: CDCHO, 1× HT and 12.5 μM MSX (Cat #GSS-1015-F, Sigma). Transfected cells were split to 0.3×10\hat{ }6 cells/ml every 3-4 days in the selection media until the viability reached above 90% which was in 2 weeks. At this point, a stable pool was established.

After recovery from selection, the stable pool was placed in fed-batch production. In this process, the basal medium used was Dynamis (Cat #A2661501 Thermo Fisher), 1× HT and 1 μM CuSO4—Sigma (C8027). The stable pool was seeded in the above production media at 0.5×10\hat{ }6 cells/ml and placed in a 36.5 C, 5% CO2, 80% humidity incubator on a shaker at 140 rpm and 25 mm orbit. Three days post inoculation the stable pools were temp-shifted to 32° C. and maintained there for the duration of the culture. The production culture was fed on day 3, 7 and 10 with 10% Feed C (Cat #A2503104, Thermo Fisher.) Glucose was monitored and maintained at 6 g/L throughout the fed-batch production. After 12 days in fed-batch production, the cell supernatant was prepared for purification by clarification through a 0.2 μm filter.

TABLE 20
Cloning Sequences for Nectin-4 TRACTr Molecule

| | |
|---|---|
| Sequence encoding | GGAGGATGGTCCTGCGCCCCCGACGAGGACACCTGGCTGTGCCCCGCCG |
| light chain of | GCGGAGGAGGCTCTGGAGGACTGTCTGGCAGGTCCGACGCCGGCTCCCC |
| Nectin-4 TRACTr | TCTGGGCCTGGCTGGCAGCGGCGGCTCTGACATCCAGATGACACAGTCC |
| molecule | CCATCCAGCCTGTCCGCCTCCGTGGGCGACACCGTGACAATCACCTGTCT |
| | GGCCACCGAGGACATCTTCTCCTACCTGGCTTGGTATCAGCAGAAGCCAG |
| | GCAAGGCCCCCAAGCTGCTGATCTACGGCGCTAATAGGCTGAAGGACAC |
| | AGTGCCATCCCGGTTCAGCGGAGGAGGCTCCGGCACAGAGTATTCTCTG |
| | ACCATCTCCGGCCTGCAGCCTGAGGATTTTGGCACCTACTATTGCCTGCA |
| | GGGCGCCAAGTTCCCACTGACATTTGGCCAGGGCACCAAGCTGGACATG |
| | AAGAGAACAGTGGCCGCTCCCTCCGTGTTCATCTTTCCCCCTAGCGATGA |
| | GCAGCTGAAGAGCGGCACCGCTTCTGTGGTGTGCCTGCTGAACAATTTCT |
| | ACCCTCGCGAGGCCAAGGTGCAGTGGAAGGTGGACAACGCTCTGCAGTC |
| | TGGCAATTCCCAGGAGAGCGTGACAGAGCAGGACTCTAAGGATTCCACC |
| | TATAGCCTGTCTTCCACACTGACCCTGTCTAAGGCCGATTACGAGAAGCA |
| | CAAGGTGTATGCTTGCGAGGTGACACATCAGGGCCTGTCCTCCCCCGTGA |
| | CCAAGTCCTTTAACAGAGGCGAGTGT (SEQ ID NO: 193) |
| Sequence encoding | GAGGTGCAGCTGGTGGAGTCCGGAGGAGGACTGGTGCAGCCTGGCGGCT |
| heavy chain of | CCCTGAGACTGAGCTGTGCCGCTTCTGGCTCCACATTCTACACCGCCGTG |
| Nectin-4 TRACTr | ATGGGATGGGTGAGGCAGGCTCCAGGCAAGGGCCTGGAGTGGGTGGCTG |
| molecule | CTATCAGGTGGACAGCCCTGACCACATCTTATGCTGACTCCGTGAAGGGC |
| | AGATTCACCATCTCCCGCGATGGCGCCAAGACCACACTGTACCTGCAGAT |
| | GAACAGCCTGAGACCTGAGGACACAGCCGTGTACTATTGCGCTGCTCGC |
| | GGCACCCTGGGACTGTTTACCACAGCTGACTCCTACGATTATTGGGGCCA |
| | GGGCACACTGGTGACCGTGTCCAGCGGCGGAGGAGGCAGCGGAGGAGG |
| | CTCTGGCGGCGTGTACTGCGGCCCAGAGTTCGACGAGTCCGTGGGCTGTA |
| | TGGGCGGCGGAGGCAGCGGAGGAGGACTGTCCGGCAGAAGCGATGCTG |
| | GCTCCCCACTGGGCCTGGCTGGCTCCGGAGGAGGCAGCGAAGTCCAGCT |
| | GGTGGAGAGCGGCGGCGGCCTGGTCCAGCCTGGCGGCTCTCTGAAGCTG |
| | TCCTGTGCCGCCTCCGGCTTCACCTTTAACAAGTATGCCATGAATTGGGT |
| | GCGCCAGGCTCCCGGCAAGGGCCTGGAGTGGGTAGCCAGGATCAGGTCC |
| | AAGTACAACAATTATGCCACCTACTACGCCGACTCCGTGAAGGATAGGT |
| | TCACAATCTCTCGGGACGATTCCAAGAACACCGCCTACCTGCAGATGAA |
| | CAATCTGAAGACAGAGGACACCGCCGTGTACTATTGCGTGAGGCACGGC |
| | AACTTTGGCAATTCTTACATCTCCTATTGGGCTTACTGGGGTCAGGGCAC |
| | ACTGGTCACCGTGTCTTCCGGAGGAGGAGGCTCCGGCGGCGGAGGCAGC |
| | GGCGGCGGCGGCTCTCAGACAGTGGTGACCCAGGAGCCAAGCCTGACCG |
| | TGTCTCCCGGCGGCACCGTGACACTGACCTGTGGCAGCTCTACAGGAGCT |
| | GTGACCAGCGGAAACTATCCAAATTGGGTGCAGCAGAAGCCTGGCCAGG |
| | CTCCTAGAGGCCTGATCGGAGGCACAAAGTTCCTGGCCCCAGGCACCCC |
| | AGCTCGCTTTAGCGGCTCTCTGCTGGGAGGCAAGGCCGCTCTGACCCTGA |
| | GCGGAGTGCAGCCAGAGGATGAGGCCGAGTACTATTGCGTGCTGTGGTA |
| | CTCTAACAGATGGGTGTTTGGCGGCGGCACAAAGCTGACCGTGCTGGGA |
| | GGAGGAGGCAGCGAAGTGCAGCTGGTCGAGTCTGGCGGCGGCTTAGTCC |
| | AACCTGGCGGCTCCCTGAGGCTGTCTTGCGCCGCTTCTGGCTTCTCCTTTA |
| | GCAACTACTATATGGCTTGGGTGCGGCAGGCTCCTGGCAAGGGCCTGGA |
| | GTGGGTCGCCTCTATCTCCACAGGCGGCGGCAATATCTACTATCGGGACT |
| | CTGTGAAGGGCAGGTTCACCATCTCCAGGGACAACGCTAAGAATAGCCT |
| | GTATCTGCAGATGAACTCCCTGAGGGCCGAAGATACTGCCGTGTACTACT |
| | GCGCCCGGCAGACCGCTTACTATGTGATGGATGCCTGGGGCCAGGGCAC |
| | CACAGTGACAGTGTCCAGCGCCTCCACCAAGGGCCCTAGCGTGTTCCCTC |
| | TGGCTCCATCTTCCAAGAGCACATCTGGAGGCACCGCCGCTCTGGGATGT |
| | CTGGTGAAGGACTACTTCCCCGAGCCTGTGACCGTGAGCTGGAACTCTGG |
| | CGCCCTGACATCTGGCGTGCACACCTTTCCCGCTGTGCTGCAGTCCTCCG |
| | GCCTGTATTCCCTGTCCAGCGTGGTGACAGTGCCTTCTTCCAGCCTGGGC |
| | ACACAGACCTACATCTGCAACGTGAATCATAAGCCTAGCAATACCAAGG |
| | TGGATAAGAAGGTGGAGCCAAAGTCTTGT (SEQ ID NO: 194) |

### Example 8: Purification of Nectin-4 TRACTr Molecule

The Nectin-4 TRACTr molecule was expressed in stable CHO-GS cells and purified. The Nectin-4 TRACTr molecule was purified from culture supernatant (HCCF) by affinity chromatography using a Protein A coupled resin (Ampshere A3, JSR Bioscience). Then, it was washed with ten column volume of 1× phosphate buffered saline, pH 7.4. The protein A was eluted with 50 mM sodium acetate buffer, pH 4.0 for 4 to 5 column volume until the absorbance is below 1. The elute was performed SEC to determine sample purity. Next, the eluted was prepare for the CEX polishing step by dialyzing into 50 mM MES buffer, pH 6.0 overnight at 4 degree. In the last polishing step, the protein was purified by cation exchanger chromatography (Capto SP Impres) to remove aggregates and other impurities and the purity was assessed by UPSEC and RP. The protein was then dialyzed into formulation buffer consisting of 10 mM Histidine, 8% Sucrose, pH 6.0, and the final purity of the protein was checked by SEC, CE-SDS, SDSPAGE, SECMALS, Mass spec, and RP chromatography methods.

### Example 9: T-Cell Dependent Cellular Cytotoxicity (TDCC)

TDCC assays were used to measure in vitro potency of the Nectin-4 TRACTr molecule (SEQ ID NO: 1 and SEQ ID NO: 2) in co-cultures of effector cells (primary human pan T cells or peripheral blood mononuclear cells (PBMCs) or non-human primate (NHP) PBMCs) with target cells (luciferase- and Nectin-4-expressing human cancer cell lines, e.g., Sk-Br-3, Panc08.13, MDA-MB-468).

Controls: Controls that were used include a T-cell engager within the antibodies in the Nectin-4 TRACTr molecule (TCE), a non-targeting TCE isotype (RSV TCE), and a Nectin-4 TRACTr molecule in which the protease cleavage sites have been removed (Uncleavable Nectin-4 TRACTr).

The TDCC assays were performed with primary human T cells, primary human PBMC, or primary NHP PBMCs as effectors cells co-cultured with luciferase- and Nectin-4-expressing human cancer cell lines as target cells.

Briefly, effector cells were thawed with warm RPMI-10% FBS, washed twice, and resuspended in culture medium. Target cells were washed with culture medium and resuspended in culture medium. Effectors cells were cocultured with target cells at fixed a effector: target (E:T) cell ratio of 3:1 (human pan T) or 10:1 (human peripheral blood mononuclear cells (PBMC) or non-human primate (NHP) PBMC) in a final volume of 200 μL per well. The cell cocultures were then treated with a serial dilution of Nectin-4 TRACTr (starting at 170 nM, 5-fold dilution, 10-point) or with a serial dilution of TCE (starting at 6 nM, 5-fold dilution, 10-point) and incubated for 96 h at 37° C. Wells containing only effector cells and target cells were used as controls for target cell viability. In some experiments, cell cocultures were also treated with negative control molecules: RSV TCE (non-targeting TCE) and uncleavable Nectin-4 TRACTr molecule.

At the end of incubation period, luciferase reagent was added into each well to quantify viable target cells. The culture plates were incubated for 10 minutes under shaking conditions at room temperature. Luminescence was measured using a Spectromax microplate reader, and specific cytotoxicity of treated wells was calculated relative to the control wells.

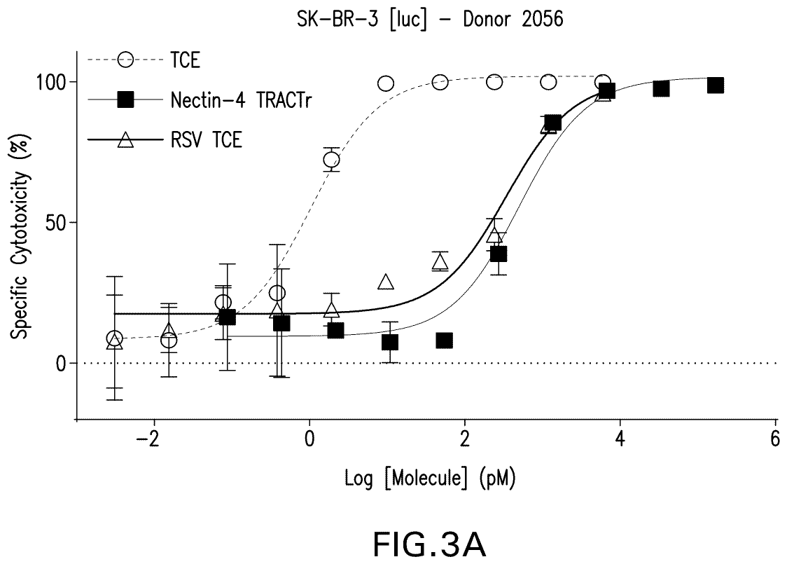

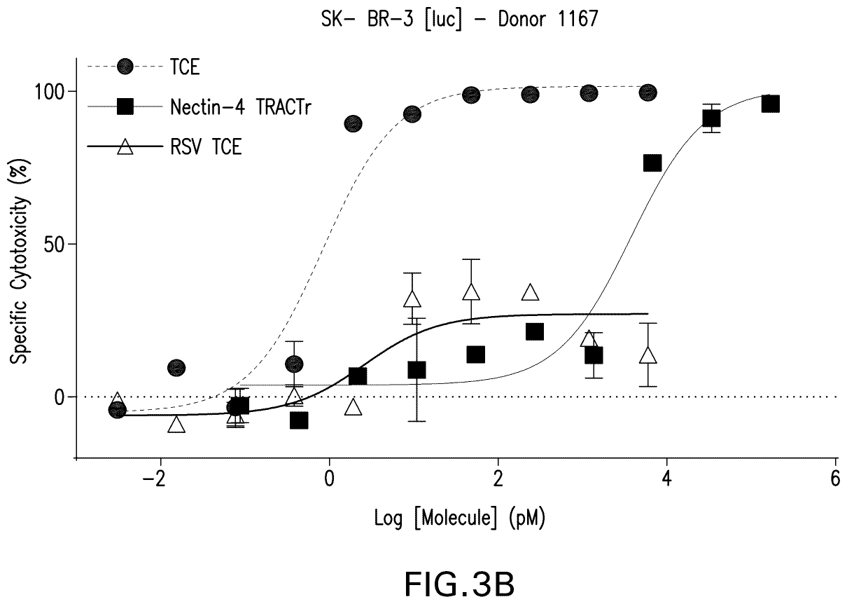

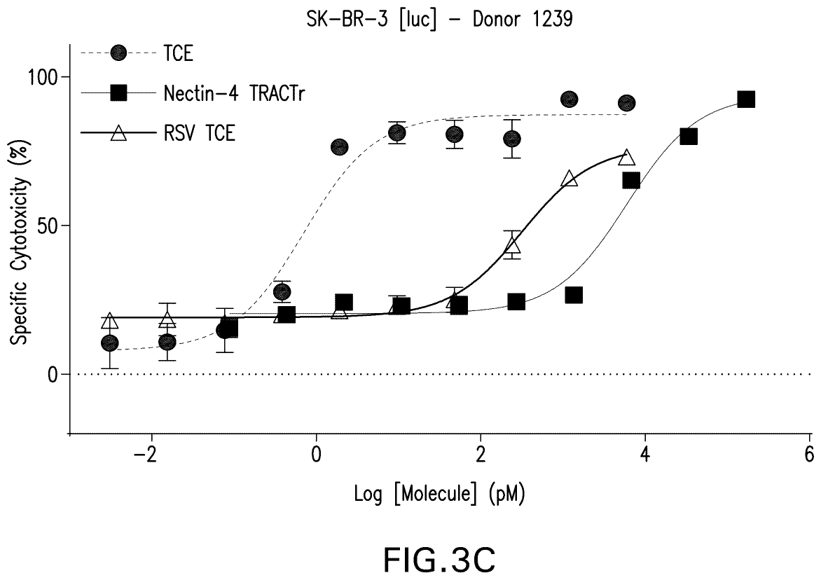

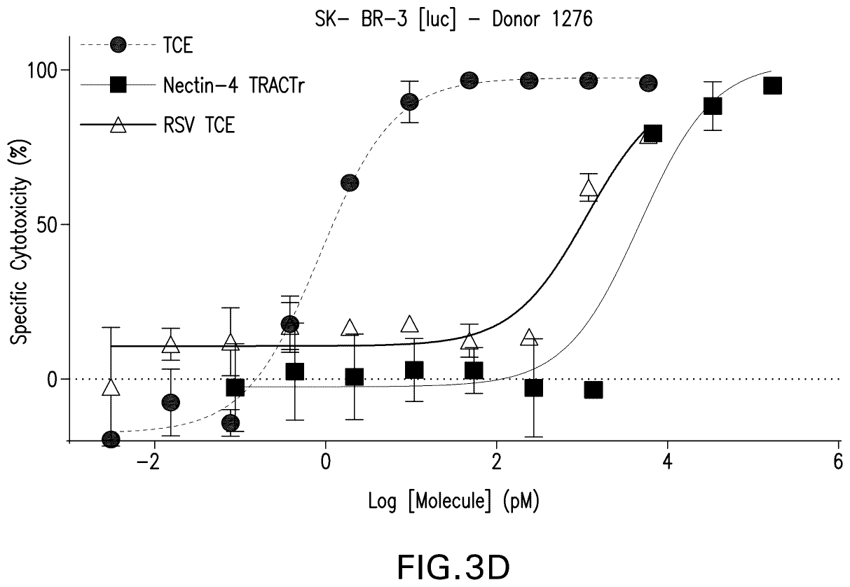

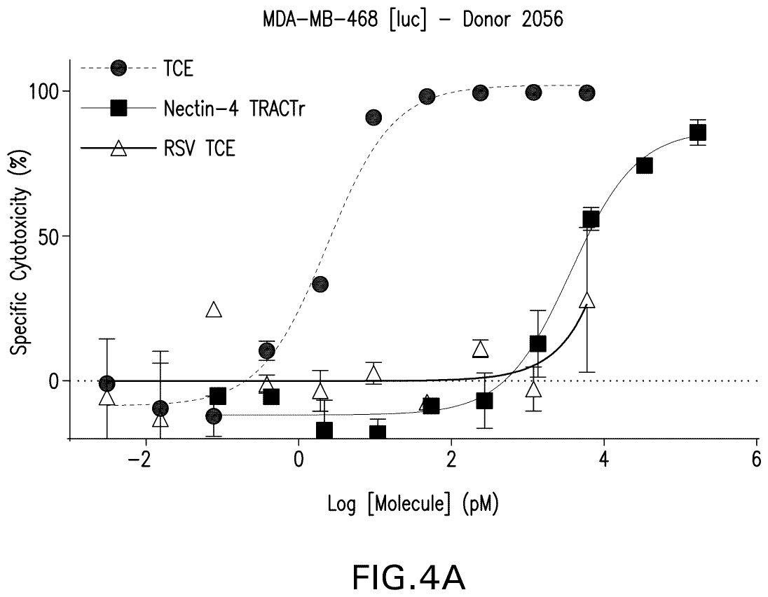

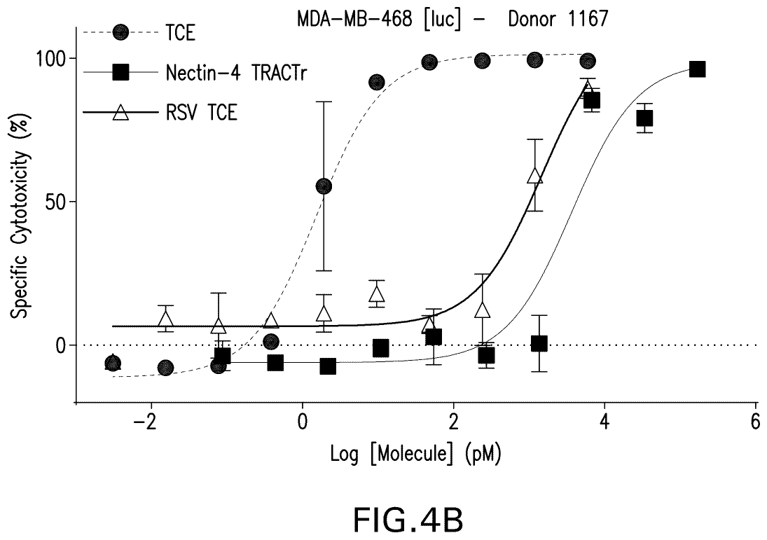

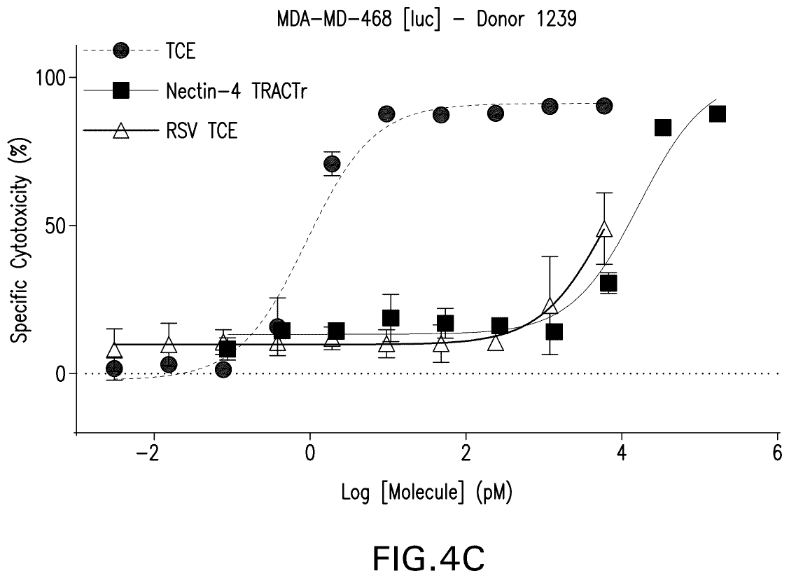

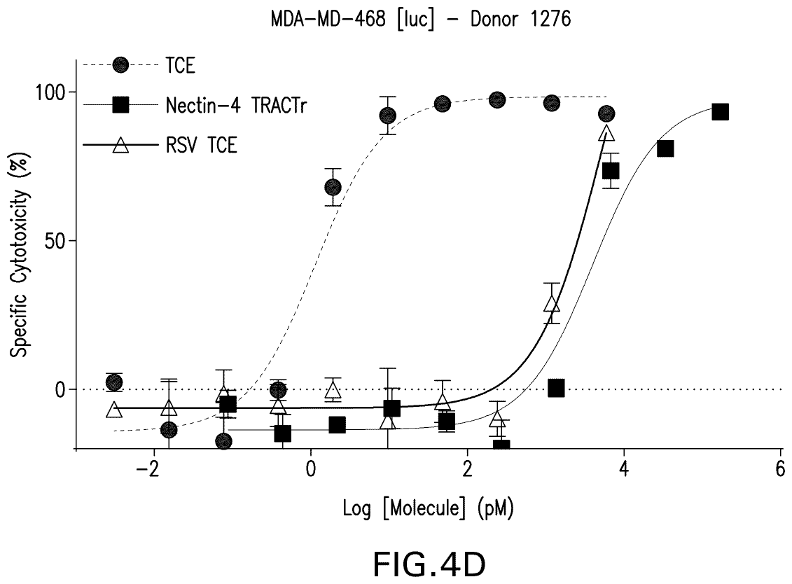

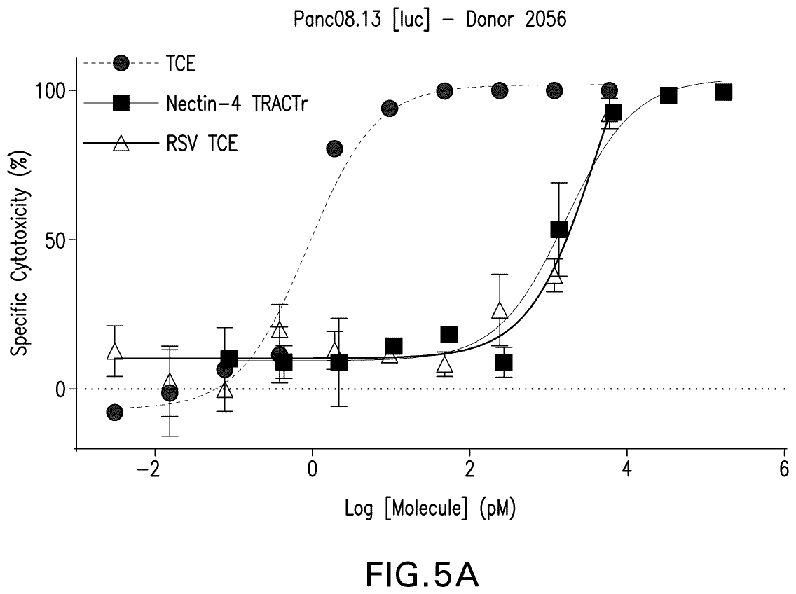

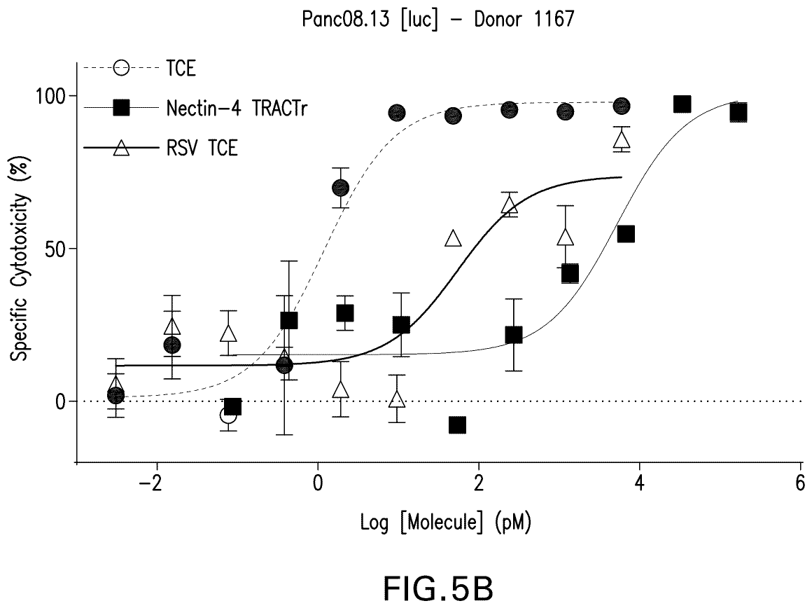

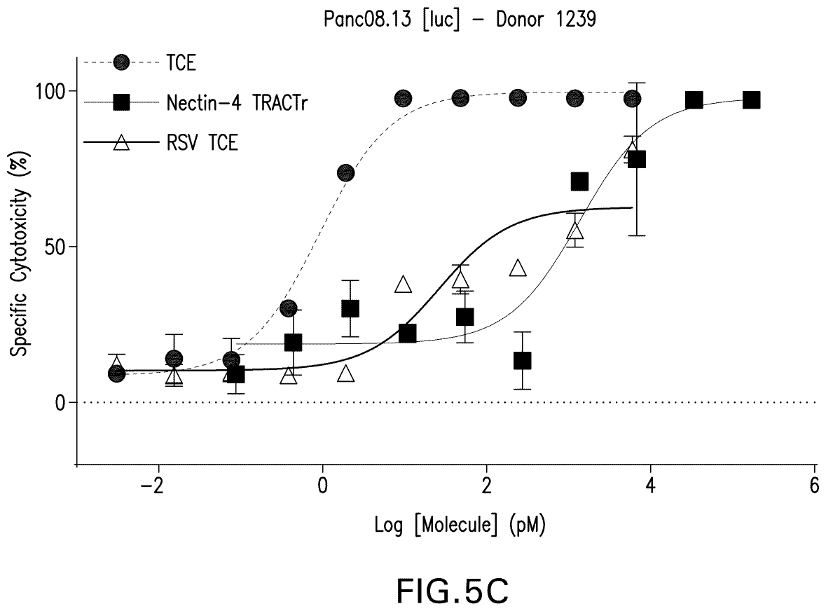

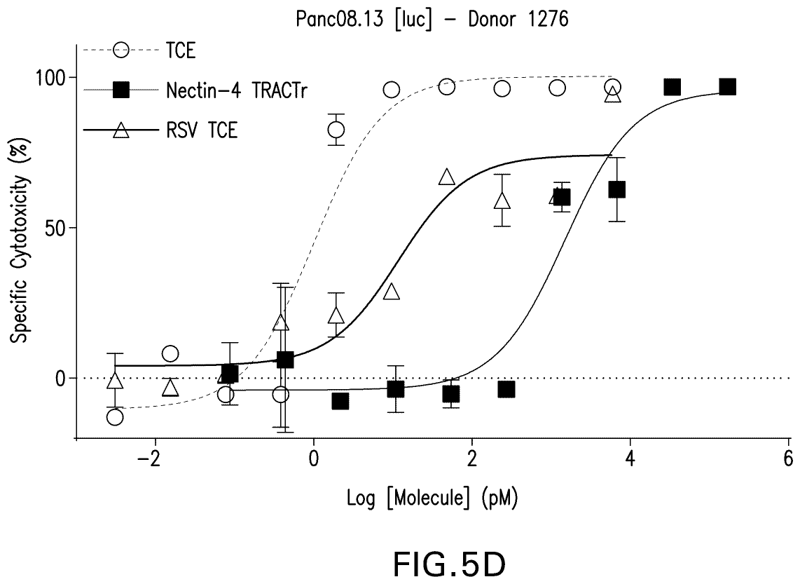

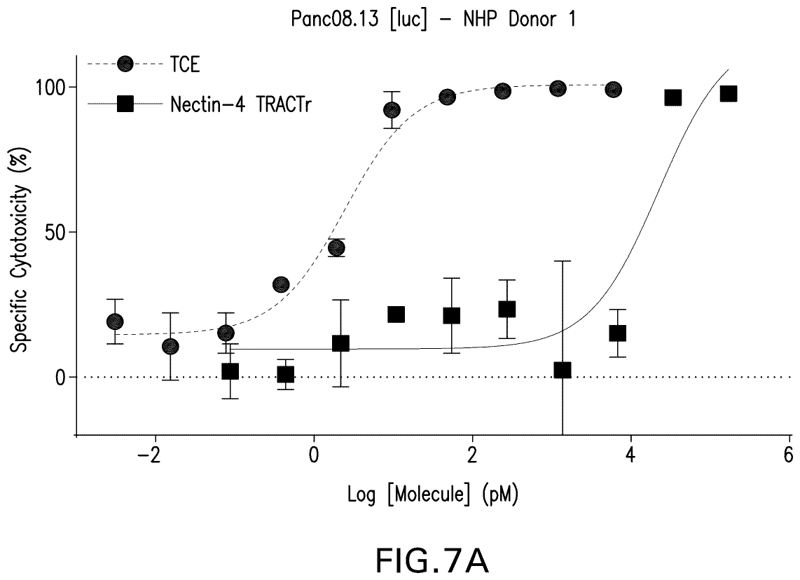

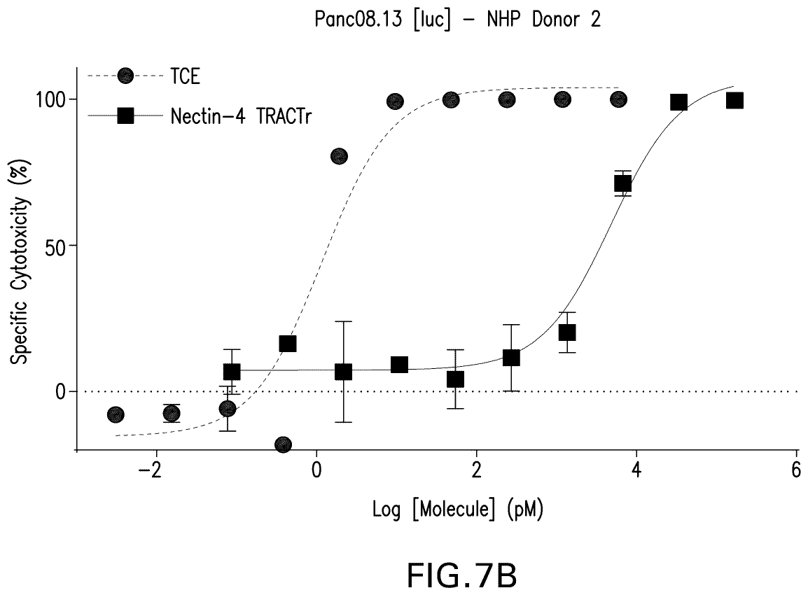

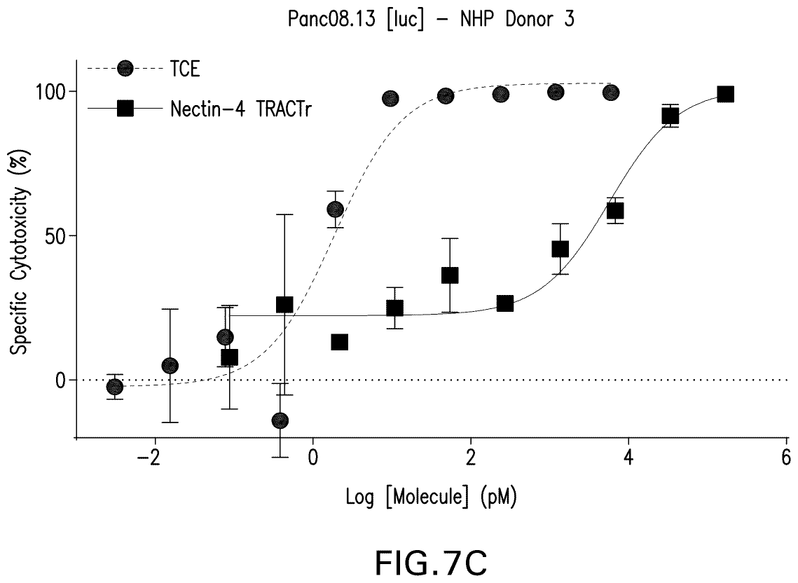

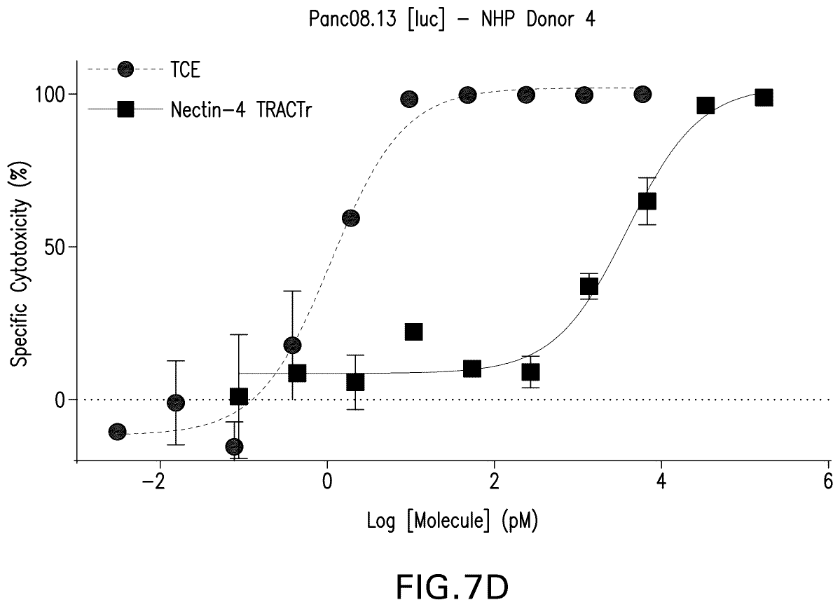

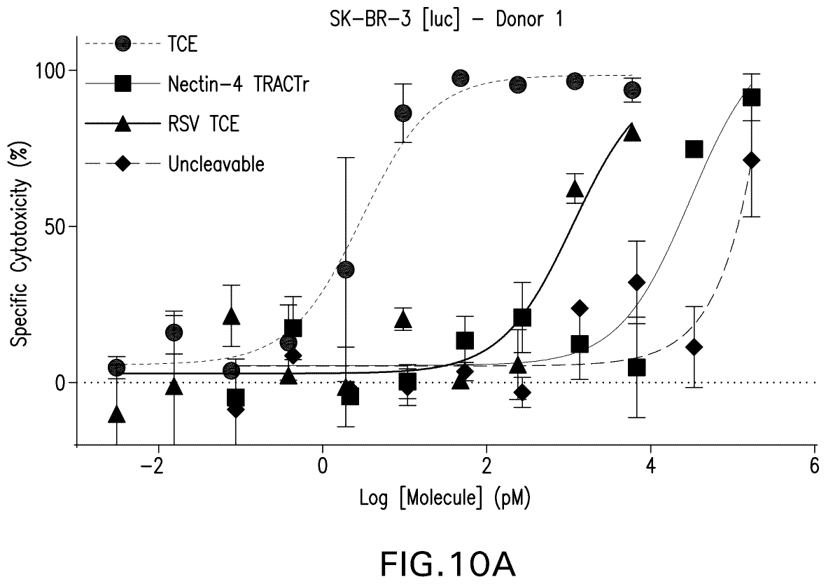

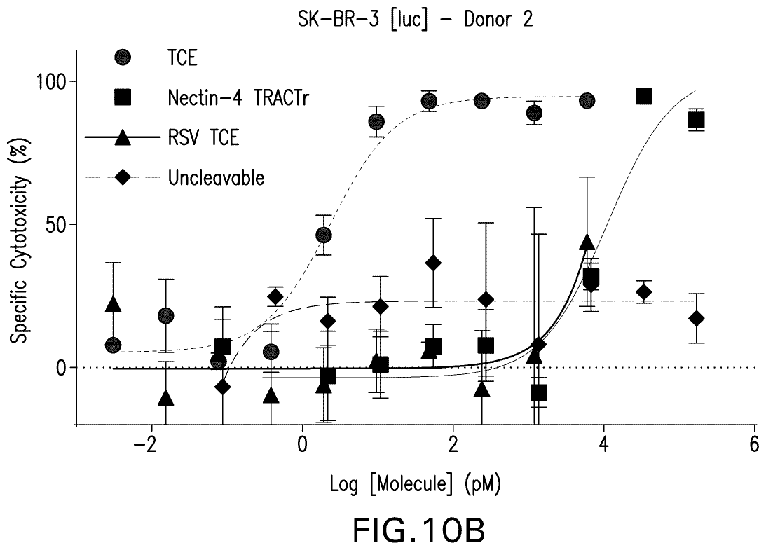

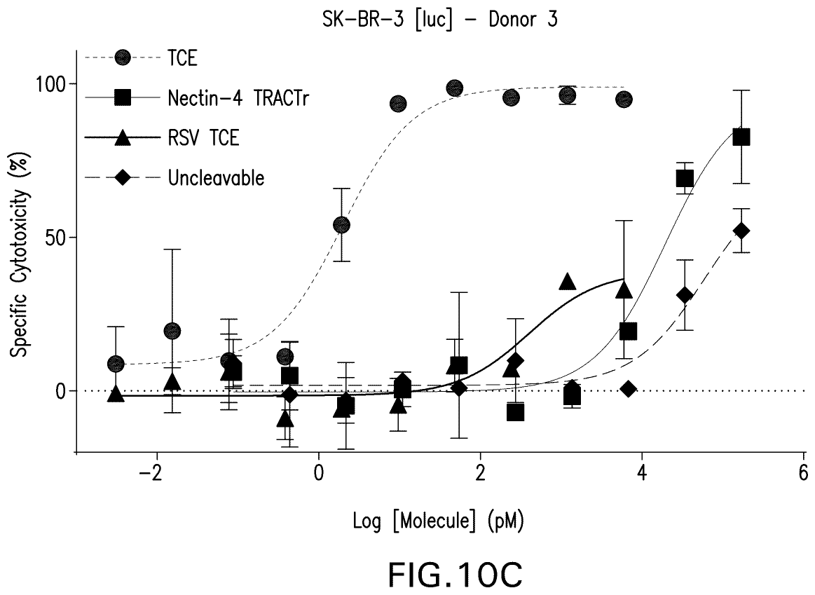

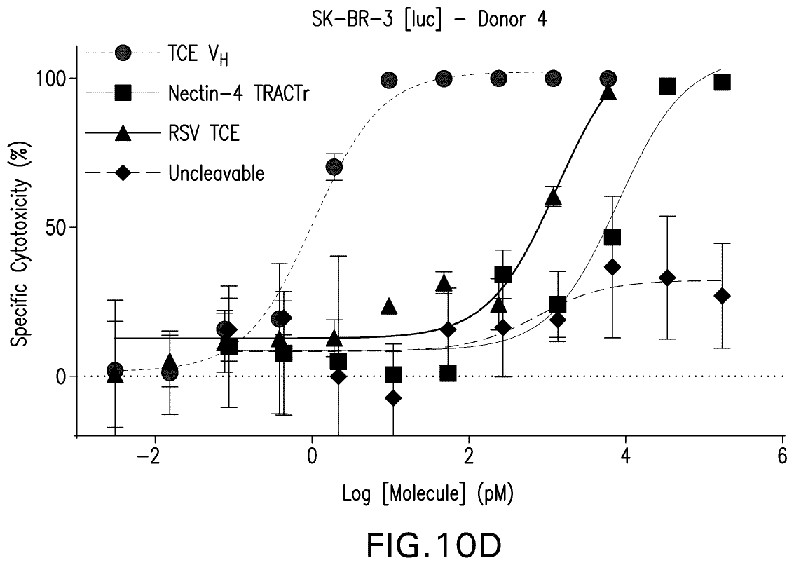

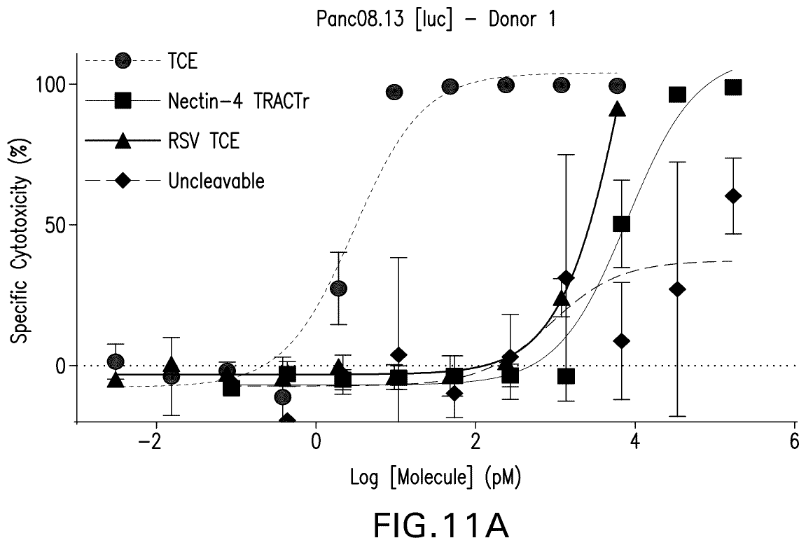

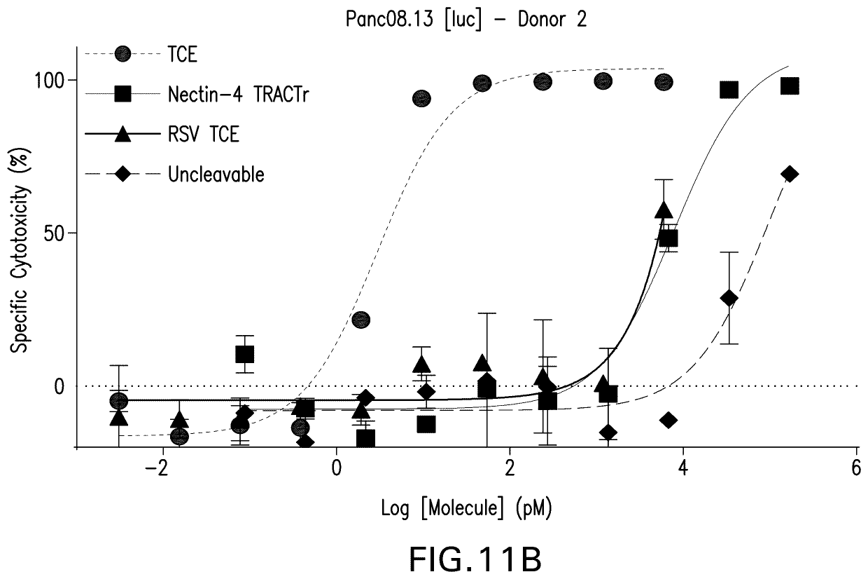

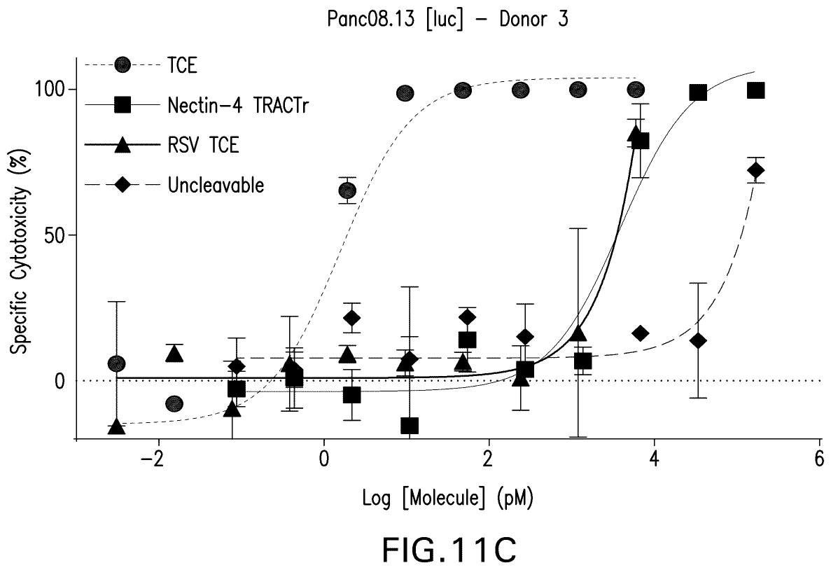

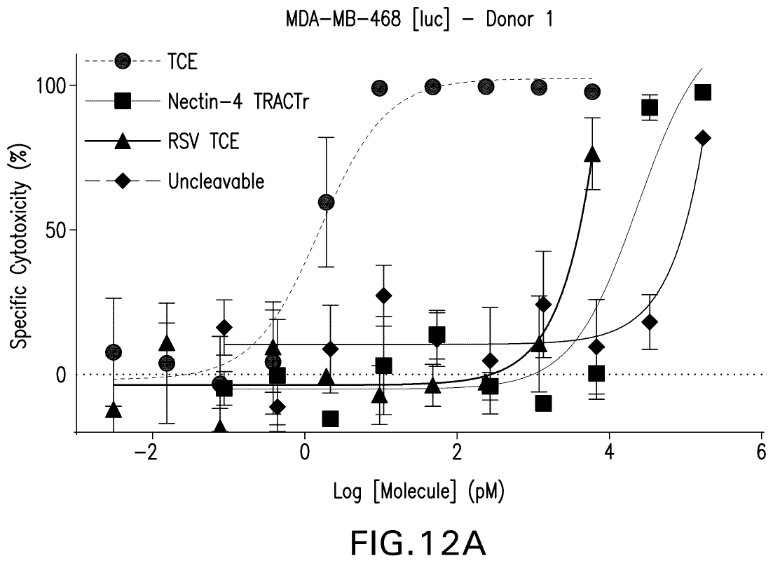

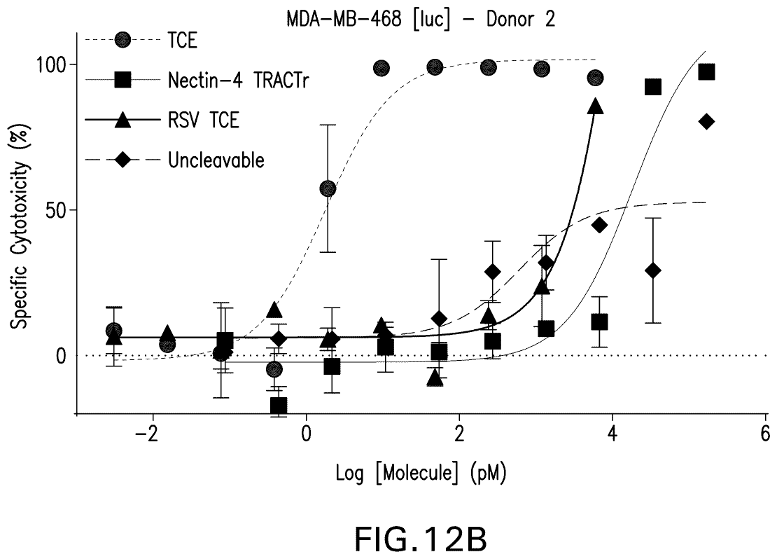

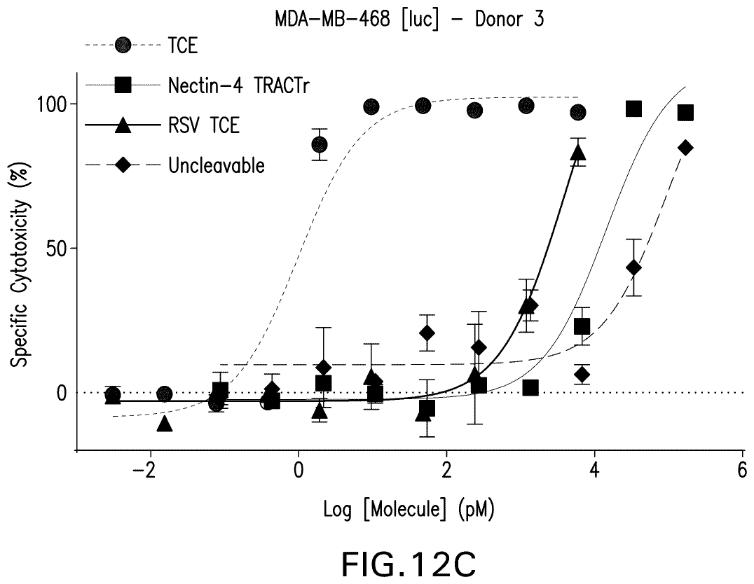

Cytotoxicity was calculated in vitro donor cells from humans and non-human primates. The calculated in vitro cytotoxicity for TDCC with human pan T cells are shown in FIGS. 3A-3D, 4A-4D, and 5A-5D. The calculated in vitro cytotoxicity with NHP PBMCs is shown in FIGS. 7A-7D. The calculated in vitro cytotoxicity with human PBMC is in FIGS. 10A-10D, 11A-11C, and 12A-12C. Nectin-4 TRACTr potency demonstrated as EC50 values are shown in Table 21.

TABLE 21
EC₅₀ Values From TDCC Assays With Four Human
Cancer Cell Lines and Human Effector Cells

| | ^{b}EC₅₀ values (Mean, pM) | | | | | |
|---|---|---|---|---|---|---|
| | ^{c}SK-BR-3 [Luc] | | ^{d} MDA-MB-468 [Luc] | | ^{e} Panc08.13 [Luc] | |
| ^{a} | Nectin-4 | | Nectin-4 | | Nectin-4 | |
| Effector cells | TRACTr | TCE | TRACTr | TCE | TRACTr | TCE |
| Human T cells^{a} | 8.13 | 0.77 | 11.67 | 1.35 | 9.20 × 10³ | 1.06 |
| Human PBMCs^{b} | 17.64 × 10³ | 1.7 | 18.78 × 10³ | 1.4 | 23.88 × 10³ | 2.2 |
| NHP PBMCs^{c} | NA | NA | NA | NA | 9.20 × 10³ | 1.65 |

EC₅₀ = half- maximal efficient concentration; h = hour(s); Luc = luciferase; NA = not applicable; NC = not calculated; NHP = non-human primate; PBMC = peripheral blood mononuclear cell; TCE = T-cell engager; TDCC = T cell-dependent cellular cytotoxicity.
^{a}TDCC assay was performed with human T cells (effector cells) cocultured with human (target cells) at a E:T ratio of 3:1 with a serial dilution of Nectin-4 TRACTr starting at 170 nM, 5-fold dilution, 10 point) or in presence of a serial dilution of TCE (starting at 6 nM, 5-fold dilution, 10-point) and incubated for 96 h at 37° C. Data are shown as the mean from 5 healthy donors.
^{b}TDCC assay was performed with human PBMCs (effector cells) cocultured with human tumor cell lines (target cells) at a E:T ratio of 10:1 with a serial dilution of Nectin-4 TRACTr (starting at 170 nM, 5-fold dilution, 10 point) or with a serial dilution of TCE (starting at 6 nM, 5-fold dilution, 10-point) and incubated for 96 h at 37° C. Data are shown as the mean from 6 healthy donors for SK-BR-3 [Luc], 3 healthy donors for MDA-MB-468 [Luc] and 10 healthy donors for Panc08.13 [Luc] cell lines.
^{c}TDCC assay was performed with rhesus monkey PBMCs (effector cells) cocultured with human tumor cell lines (target cells) at a E:T ratio of 10:1 with a serial dilution of Nectin-4 TRACTr (starting at 170 nM, 5-fold dilution, 10 point) or with a serial dilution of TCE (starting at 6 nM, 5-fold dilution, 10-point) and incubated for 96 h at 37° C. Data are shown as the mean from 4 rhesus monkeys.

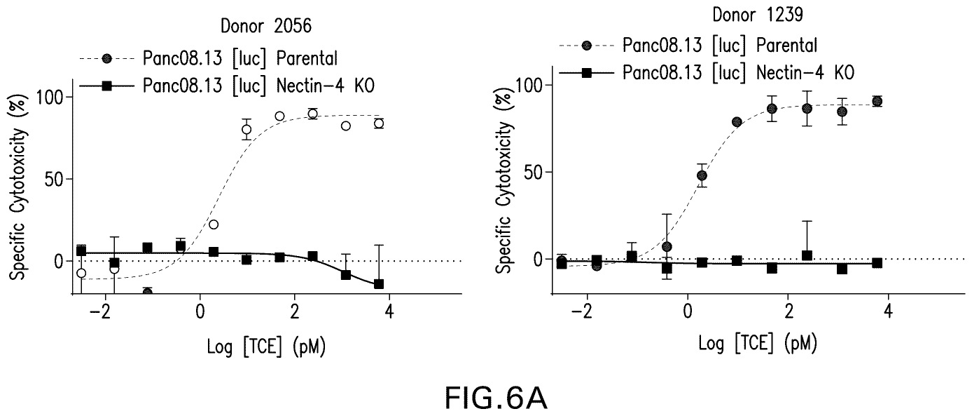

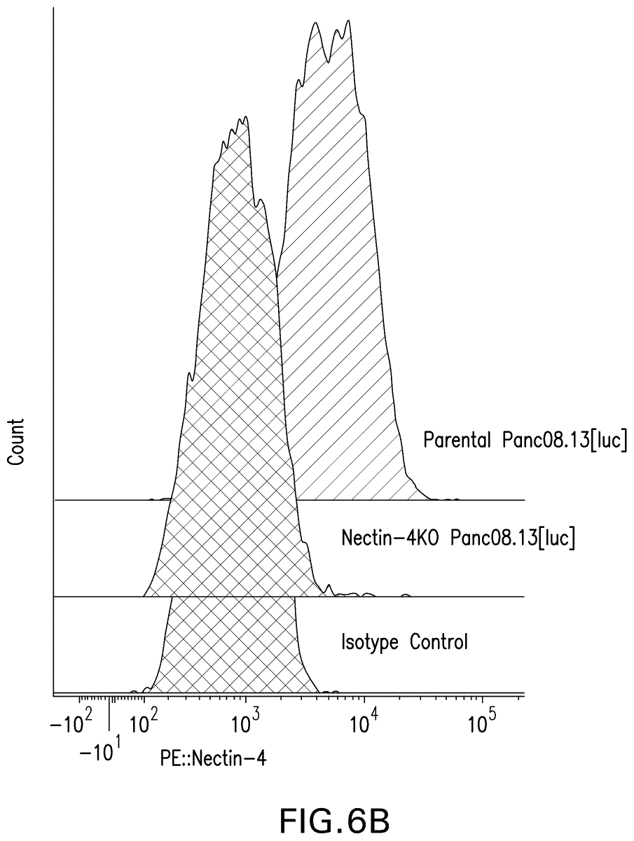

A TDCC assay was performed with human T cells and Panc08.13 [Luc] parental or Panc08.13 [Luc] Nectin-4 knock-out (KO) cell lines to evaluate the impact of Nectin-4 in TCE functional activity. Nectin-4 KO cells were not killed in TDCC, demonstrating the requirement for Nectin-4 expression. Data is shown in FIGS. 6A-6B.

### Example 10: Cytokine Concentration Analyzed by Immunoassay

Immunoassay of supernatants collected from above-described TDCC assays were collected to demonstrate Nectin-4 TRACTr-induced IFNγ and TNFα secretion.

After 48 h of human pan T cell coculture with target cells and Nectin-4 TRACTr, TCE, RSV TCE, or Uncleavable Nectin-4 TRACTr, cell culture supernatants were collected and IFNγ and TNFα concentration were measured using an electro chemiluminescent (ECL)-based immunoassay platform following manufacturer's protocols.

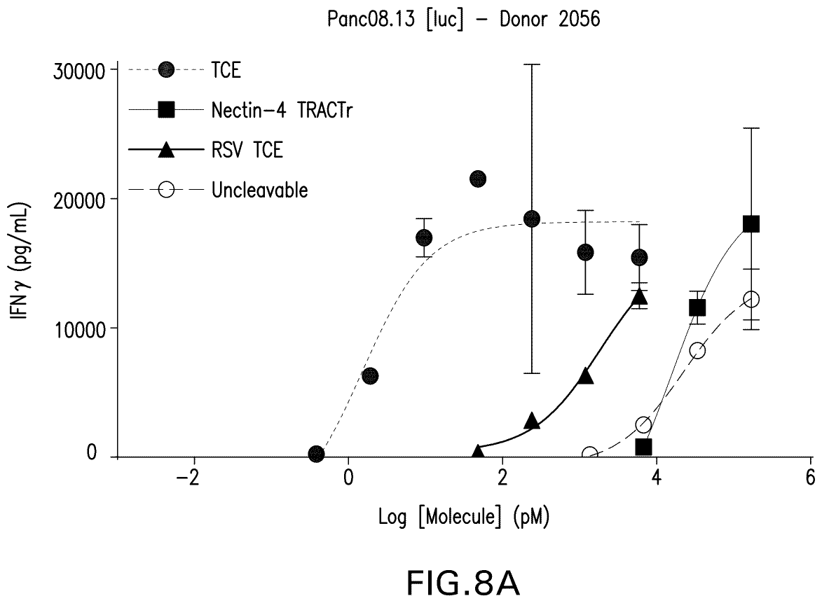

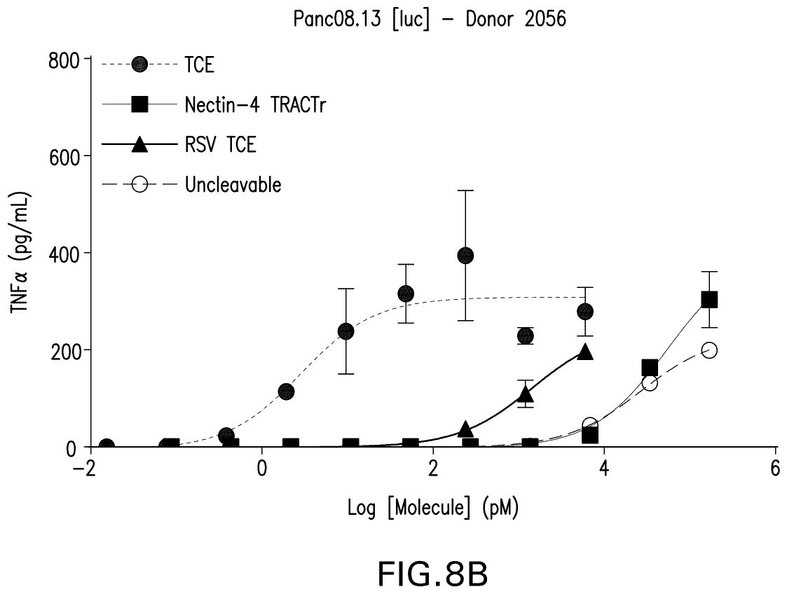

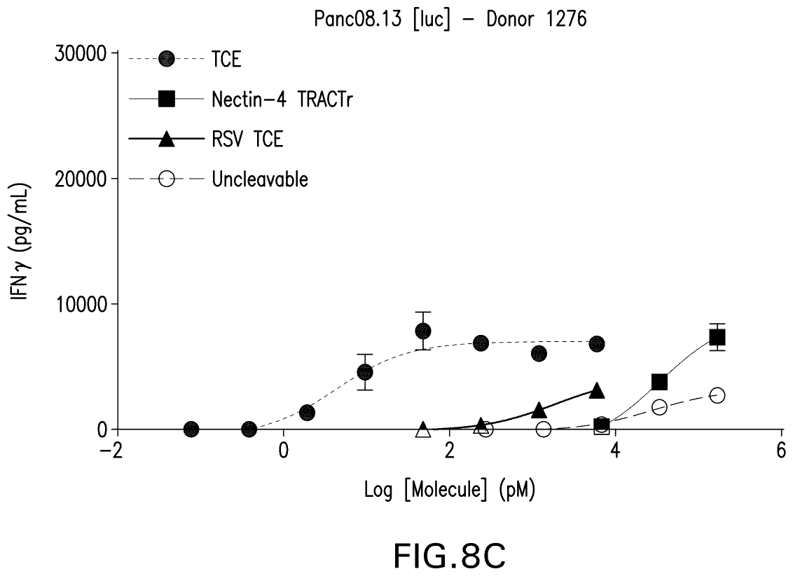

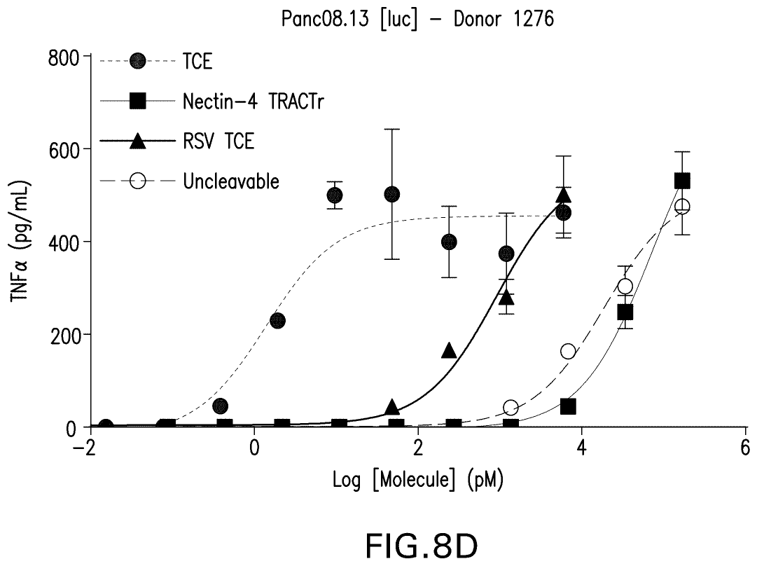

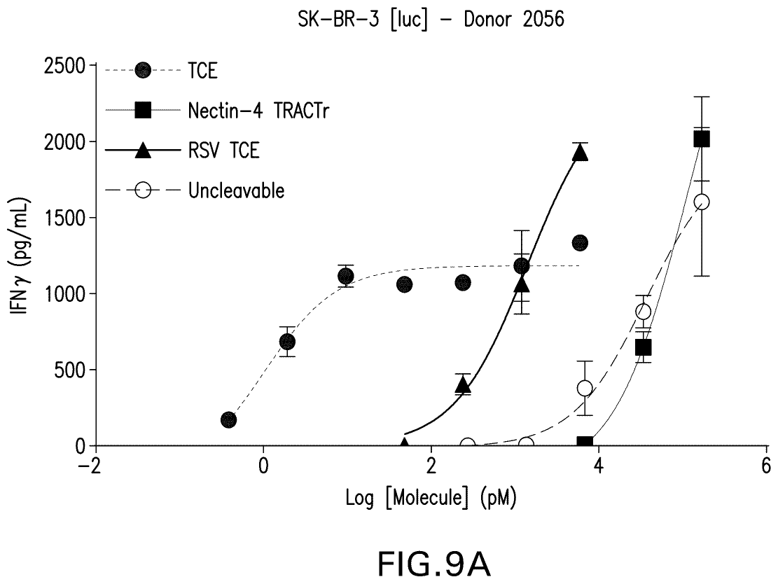

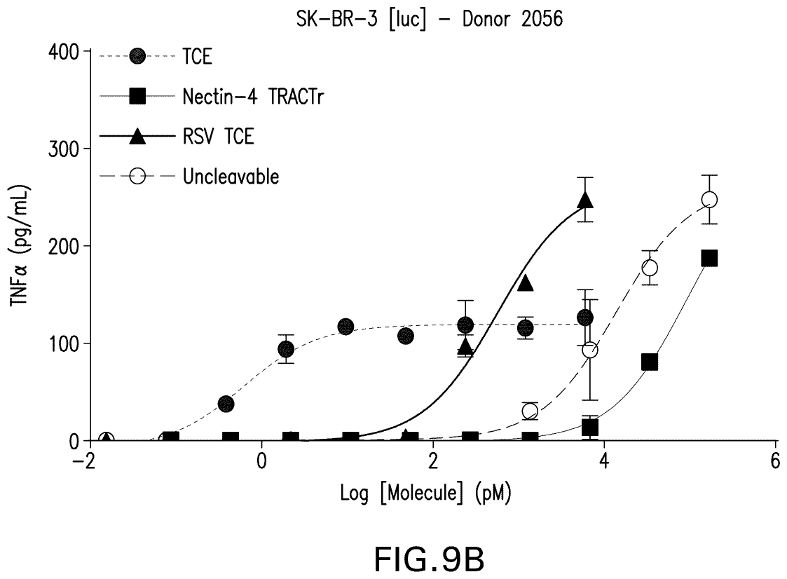

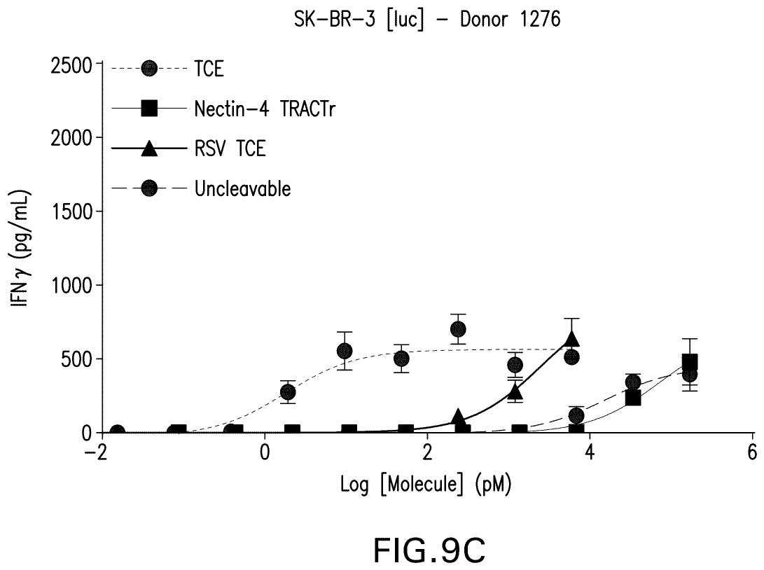

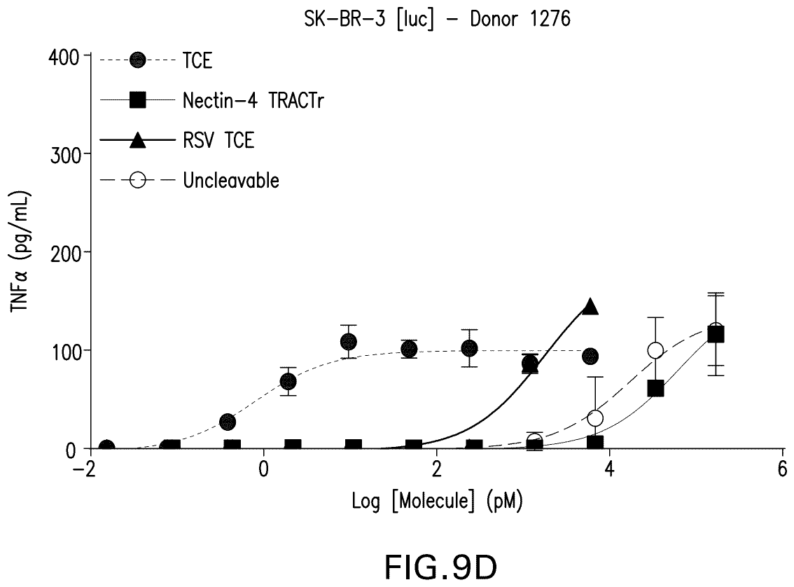

Briefly, multi-array plates pre-coated with anti-cytokine capture antibodies were washed 3 times with wash buffer (provided by manufacturer). Standards, control dilutions and supernatant samples (50 μL) were added to the plates and incubated for 2 hours under shaking conditions at room temperature. After washes with wash buffer, detection antibody solution was added to the wells and incubated for 2 hours at room temperature under shaking conditions. Wells were washed 3 times with wash buffer, then 150 μL of 2× Read Buffer T was added into each well, and ECL was measured on a SECTOR® S600 instrument (Meso Scale Discovery). IFNγ and TNFα concentrations in the samples were calculated using ECL values of samples and a standard curve. Nectin-4 TRACTr-induced IFNγ and TNFα secretion is shown in FIGS. 8A-8D and 9A-9D. EC50 values are shown in Table 22.

TABLE 22
Cytokine Concentration Analyzed From TDCC Assay
Supernatants Treated With Nectin-4 TRACTr

| | ^{g} Nectin-4 TRACTr Treatment, | | ^{h} TCE Treatment, | |
|---|---|---|---|---|
| ^{f} | EC₅₀ (Mean, ng/mL) | | EC₅₀ (Mean, ng/mL) | |
| ⁱ Cell Line | ^{j} IFNγ | ^{k} TNFα | ^{l} IFNγ | ^{m} TNFα |
| SK-BR-3 | 8875 | 7178 | 0.10 | 0.05 |
| [Luc] | | | | |
| Panc08.13 | 2328 | 6011 | 0.21 | 0.16 |
| [Luc] | | | | |

IFNγ = interferon gamma; Luc = luciferase; TCE = t-cell engager; TDCC = T-cell-dependent cellular cytotoxicity; TNFα = tumor necrosis factor alpha.
TDCC assay was performed with human T cells isolated from 2 healthy donors (effector cells) cocultured with human tumor cell lines (target cells) at a E:T ratio of 3:1 with a serial dilution of Nectin-4 TRACTr(starting at 170 nM, 5-fold dilution, 10 point) or with a serial dilution of TCE (starting at 6 nM, 5-fold dilution, 10 point) and incubated for 48 h at 37° C. At the end of incubation, cytokine level was measured by a Meso Scale Discovery immunoassay. Data are shown as the mean from 2 human healthy donors.

A mix of human T cells and human tumor cell lines at a E:T ratio of 3:1 was incubated for 48 h in presence of a serial dilution of Nectin-4 TRACTr, or TCE. At the end of the incubation, supernatants were collected and IFNγ and TNFα concentration level were measured using a Meso Scale Discovery immunoassay.

The disclosed subject matter is not to be limited in scope by the specific embodiments and examples described herein. Indeed, various modifications of the disclosure in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

The following sequences are disclosed herein.

TABLE 23
Sequences
Sequences

| SEQ ID | | |
|---|---|---|
| NO: | Description | Sequence |
| 1 | Nectin-4 TRACTr | MGWSCIILFLVATATGVHSEVQLVESGGGLVQPGGSLRLSCAASGS |
| | heavy chain | TFYTAVMGWVRQAPGKGLEWVAAIRWTALTTSYADSVKGRFTISRD |
| | | GAKTTLYLQMNSLRPEDTAVYYCAARGTLGLFTTADSYDYWGQGT |
| | | LVTVSSGGGGSGGGSGGVYCGPEFDESVGCMGGGGSGGGLSGRSD |
| | | AGSPLGLAGSGGGSEVOLVESGGGLVQPGGSLKLSCAASGFTFNKY |
| | | AMNWVRQAPGKGLEWVARIRSKYNNYATYYADSVKDRFTISRDDS |
| | | KNTAYLQMNNLKTEDTAVYYCVRHGNFGNSYISYWAYWGQGTLVT |
| | | VSSGGGGSGGGGSGGGGSQTVVTQEPSLTVSPGGTVTLTCGSSTGA |
| | | VTSGNYPNWVQQKPGQAPRGLIGGTKFLAPGTPARFSGSLLGGKAA |
| | | LTLSGVQPEDEAEYYCVLWYSNRWVFGGGTKLTVLGGGGSEVQLV |
| | | ESGGGLVQPGGSLRLSCAASGFSFSNYYMAWVRQAPGKGLEWVASI |
| | | STGGGNIYYRDSVKGRFTISRDNAKNSLYLQMNSLRAEDTAVYYCA |
| | | RQTAYYVMDAWGQGTTVTVSSASTKGPSVFPLAPSSKSTSGGTAAL |
| | | GCLVKDYFPEPVTVSWNSGALTSGVHTFPAVLQSSGLYSLSSVVTV |
| | | PSSSLGTQTYICNVNHKPSNTKVDKKVEPKSC |
| 2 | Nectin-4 TRACTr | MGWSCIILFLVATATGVHSGGWSCAPDEDTWLCPAGGGGSGGLSGRS |
| | light chain | DAGSPLGLAGSGGSDIQMTQSPSSLSASVGDTVTITCLATEDIFSYL |
| | | AWYQQKPGKAPKLLIYGANRLKDTVPSRFSGGGSGTEYSLTISGLQP |
| | | EDFGTYYCLQGAKFPLTFGQGTKLDMKRTVAAPSVFIFPPSDEQLKS |
| | | GTASVVCLLNNFYPREAKVQWKVDNALQSGNSQESVTEQDSKDST |
| | | YSLSSTLTLSKADYEKHKVYACEVTHQGLSSPVTKSFNRGEC |
| 3 | Leader Sequence | MGWSCIILFLVATATGVHS |
| | (Nectin-4 TRACTr) | |
| 4 | Anti-Albumin | TAVMG |
| | CDRH1 | |
| | (Nectin-4 TRACTr) | |
| 5 | Anti-Albumin | AAIRWTALTTSYADSVKG |
| | CDRH2 | |
| | (Nectin-4 TRACTr) | |
| 6 | Anti-Albumin | RGTLGLFTTADSYDY |
| | CDRH3 | |
| | (Nectin-4 TRACTr) | |
| 7 | CD3 Mask | GGVYCGPEFDESVGCMGG |
| | (Nectin-4 TRACTr) | |
| 8 | Protease cleavage | LSGRSDAGSPLGLAG |
| | site (MK-8294 | |
| | heavy chain) | |
| | (Nectin-4 TRACTr) | |
| 9 | CD3 CDRH1 | KYAM |
| | (Nectin-4 TRACTr) | |
| 10 | CD3 CDRH2 | RIRSKYNNYATYYADSVKD |
| | (Nectin-4 TRACTr) | |
| 11 | CD3 CDRH3 | HGNFGNSYISYWAY |
| | (Nectin-4 TRACTr) | |
| 12 | CD3 CDRL1 | GSSTGAVT |
| 13 | CD3 CDRL2 | GTKFLAP |
| | (Nectin-4 TRACTr) | |
| 14 | CD3 CDRL3 | VLWYSNRWV |
| | (Nectin-4 TRACTr) | |
| 15 | Nectin-4 CDRH1 | NYYMA |
| | (Nectin-4 TRACTr) | |
| 16 | Nectin-4 CDRH2 | SISTGGGNIYYRDSVKG |
| | (Nectin-4 TRACTr) | |
| 17 | Nectin-4 CDRH3 | QTAYYVMDA |
| | (Nectin-4 TRACTr) | |
| 18 | Nectin-4 Mask #1 | GGWSCAPDEDTWLCPAGG |
| | (Nectin-4 TRACTr) | |
| 19 | Protease cleavage | LSGRSDAGSPLGLAG |
| | site (MK-8294 light | |
| | chain) (Nectin-4 | |
| | TRACTr) | |
| 20 | Nectin-4 CDRL1 | LATEDIFSYLA |
| | (Nectin-4 TRACTr) | |
| 21 | Nectin-4 CDRL2 | GANRLKD |
| | (Nectin-4 TRACTr) | |
| 22 | Nectin-4 CDRL3 | LOGAKFPLT |
| | (Nectin-4 TRACTr) | |
| 23 | Nectin-4 Mask #2 | GGWFCAPDENTWLCPNAGG |
| 24 | Nectin-4 light chain | EIQMTQSHKTFSVSTGQRVTITCKASQDVSIAVAWYQQKPGKAPKLL |
| | variable sequence | IYWASTRQTGVPSRFSGSGSGTDFTLTISCVQSEDFAVYYCQQYSSY |
| | (1) | PFTFGQGTKLEIK |
| | (sequence name: | |
| | DAB012120) | |
| | (humanized) | |
| 25 | Nectin-4 light chain | DIQMTQSPSFLSASVGDRVTITCKASQDVSIAVAWYQQKPGKAPKLL |
| | variable sequence | IYWASTRQTGVPSRFSGSGSGTDFTLTISSLQPEDFATYFCQQYSSY |
| | (2) | PFTFGQGTKLEIK |
| | (sequence name: | |
| | DAB012121) | |
| | (humanized) | |
| 26 | Nectin-4 light chain | DIQMTQSPSFLSASVGERVTITCKASQDVSIAVAWYQQKPGKAPKLL |
| | variable sequence | IYWASTRQTGVPSRFSGSGSGTDFTLTISSLQPEDIATYFCQQYSSY |
| | (3) | PFTFGQGTKLEIK |
| | (sequence name: | |
| | DAB012122) | |
| | (humanized) | |
| 27 | Nectin-4 light chain | DIQMTQSPSFLSASVGDRVTITCKASQDVSIAVAWYQQKPGKAPKLL |
| | variable sequence | IYWASTRQTGVPSRFSGSGSGTDFTLTISSLQPEDSATYFCQQYSSY |
| | (4) | PFTFGQGTKVDIK |
| | (sequence name: | |
| | DAB012123) | |
| | (humanized) | |
| 28 | Nectin-4 CDRL1 | KASQDVSIAVA |
| | (1) (rat) | |
| 29 | Nectin-4 CDRL2 | WASTRQT |
| | (1) (rat) | |
| 30 | Nectin-4 CDRL3 | QQYSSYPFT |
| | (1) (rat) | |
| 31 | Nectin-4 light chain | EIQMTQSQSTLSVSLGDRVTITCKASQNVVTAVAWYQQKPGQAPKLL |
| | variable sequence | IYSASHRFTGVPSRFSGSGSGTDFTLIISCMQSEDFAVYYCQQYSSY |
| | (5) (sequence name: | PYTFGQGTKLEIK |
| | DAB012124) | |
| | (humanized) | |
| 32 | Nectin-4 light chain | DIQMTQSPSFLSASVGDRVTITCKASQNVVTAVAWYQQKPGKAPKLL |
| | variable sequence | IYSASHRFTGVPSRFSGSGSGTDFTLTISNLQSEDLATYFCQQYSSYP |
| | (6) (sequence name: | YTFGQGTKLEIK |
| | DAB012125) | |
| | (humanized) | |
| 33 | Nectin-4 light chain | DIQMTQSPSFLSASVGDRVTITCKASQNVVTAVAWYQQKPGKAPKL |
| | variable sequence | LIYSASHRFTGVPSRFSGSGSGTDFTLTISNLQPEDSATYFCQQYSSYP |
| | (7) (sequence name: | YTFGQGTKLEIK |
| | DAB012126) | |
| | (humanized) | |
| 34 | Nectin-4 light chain | DIQMTQSPSFLSASVGDRVTITCKASQNVVTAVAWYQQKPGKAPKL |
| | variable sequence | LIYSASHRFTGVPSRFSGSGSGTDFTLTISNLQPDDAATYFCQQYSSYP |
| | (8) (sequence name: | YTFGQGTKLEIK |
| | DAB012127) | |
| | (humanized) | |
| 35 | Nectin-4 CDRL1 | KASQNVVTAVA |
| | (2) (rat) | |
| 36 | Nectin-4 CDRL2 | SASHRFT |
| | (2) (rat) | |
| 37 | Nectin-4 CDRL3 | QQYSSYPYT |
| | (2) (rat) | |
| 38 | Nectin-4 light chain | AIQMTQSHKSFSVSTGQRVTITCKASQDVSTTVAWYQQKPGQAPKL |
| | variable sequence | LIYWASTRQTGVPSRFTGSGSGTDFTLTISCVQSEDFAVYYCQQYSSY |
| | (9) (sequence name: | PFTFGQGTKLEIK |
| | DAB012128) | |
| | (humanized) | |
| 39 | Nectin-4 light chain | DIQMTQSPSFLSASVGDRVTITCKASQDVSTTVAWYQQKPGKAPKLL |
| | variable sequence | IYWASTRQTGVPSRFSGSGSGTDFTLTISNLQPEDFATYFCQQYSSYP |
| | (10) (sequence | FTFGQGTKLEIK |
| | name: DAB012129) | |
| | (humanized) | |
| 40 | Nectin-4 light chain | DIQMTQSPSFLSASVGERVTITCKASQDVSTTVAWYQQKPGKAPKLL |
| | variable sequence | IYWASTRQTGVPSRFSGSGSGTDFTLTINSLQPEDVATYFCQQYSSYP |
| | (11) (sequence | FTFGQGTKLEIK |
| | name: DAB012130) | |
| | (humanized) | |
| 41 | Nectin-4 light chain | DIQMTQSPSFLSASVGERVTITCKASQDVSTTVAWYQQKPGKAPKLL |
| | variable sequence | IYWASTRQTGVPSRFSGSGSGTDFTLTISNLQPEDVATYFCQQYSSYP |
| | (12) (sequence | FTFGQGTKLEIK |
| | name: DAB012131) | |
| | (humanized) | |
| 42 | Nectin-4 CDRL1 | KASQDVSTTVA |
| | (3) (rat) | |
| 43 | Nectin-4 light chain | DIQMTQSPSSVSLTVGQRVTITCLATEDIFSYLAWYQQKPGKAPRLLI |
| | variable sequence | YGANRLKDGVPSRFSGSGSGTQYTLRISSMQPEDFGVYYCLQGAKFP |
| | (13) (sequence | LTFGPGTKLEIK |
| | name: DAB012132) | |
| | (humanized) | |
| 44 | Nectin-4 light chain | DIQMTQSPSSLSASIGDTVTITCLATEDIFSYLAWYQQRPGKAPKLLIY |
| | variable sequence | GANRLKDGVPSRFSGSGSGTDYTLTISGLQPEDFVTYYCLQGAKFPL |
| | (14) (sequence | TFGQGTKLDMK |
| | name: DAB012133) | |
| | (humanized) | |
| 45 | Nectin-4 light chain | DIQMTQSPSSLSASVGDTVTITCLATEDIFSYLAWYQQKPGKAPKLLI |
| | variable sequence | YGANRLKDGVPSRFSGSGSGTDYTLTISGLQPEDFGTYYCLQGAKFP |
| | (15) (sequence | LTFGQGTKLEIK |
| | name: DAB012134) | |
| | (humanized) | |
| 46 | Nectin-4 light chain | DIQMTQSPSSLSASVGDTVTITCLATEDIFSYLAWYQQKPGKAPKLLI |
| | variable sequence | YGANRLKDGVPSRFSGGGSGTEYSLTISGLQPEDFGTYYCLQGAKFP |
| | (16) (sequence | LTFGQGTKLDMK |
| | name: DAB012135) | |
| | (humanized) | |
| 47 | Nectin-4 light chain | DIQMTQSPSSLSASVGDTVTITCLATEDIFSYLAWYQQKPGKAPKLLI |
| | variable sequence | YGANRLKDTVPSRFSGGGSGTEYSLTISGLQPEDFGTYYCLQGAKFP |
| | (17) (sequence | LTFGQGTKLDMKR |
| | name: | |
| | DAB0121315) | |
| | (modified) | |
| | (humanized) | |
| 48 | Nectin-4 heavy | QVQLLESGGELVQPGGSLRLSCAASGFTFSDYYMFWIRQTPQKRLE |
| | chain variable | WVAYISNGGGNTYYPDTVKGRFTISRDNAKNQLYLQMRSLTPEDTA |
| | sequence (1) | IYYCASPEARYYGNYPFPYWGQGTLVTVSS |
| | (sequence name: | |
| | DAB012120) | |
| | (humanized) | |
| 49 | Nectin-4 heavy | QVQLVESGGGLVKPGGSLRLSCAASGFTFSDYYMFWIRQAPGKGLE |
| | chain variable | WVAYISNGGGNTYYPDTVKGRFTISRDNAKNTLYLQMNSLRAEDTA |
| | sequence (2) | VYYCASPEARYYGNYPFPYWGQGTLVTVSS |
| | (sequence name: | |
| | DAB012121) | |
| | (humanized) | |
| 50 | Nectin-4 heavy | QVQLVESGGGVVKPGGSLRLSCAASGFTFSDYYMFWIRQAPGKGLE |
| | chain variable | WVAYISNGGGNTYYPDTVKGRFTISRDNAKNTLYLQMNSLRAEDTA |
| | sequence (3) | VYYCASPEARYYGNYPFPYWGQGTLVTVSS |
| | (sequence name: | |
| | DAB012122) | |
| | (humanized) | |
| 51 | Nectin-4 heavy | QVQLVESGGGVVQPGGSLRLSCAASGFTFSDYYMFWIRQAPGKGLE |
| | chain variable | WVAYISNGGGNTYYPDTVKGRFTISRDNAKNTLYLQMNSLRAEDTA |
| | sequence (4) | VYYCASPEARYYGNYPFPYWGQGTLVTVSS |
| | (sequence name: | |
| | DAB012123) | |
| | (humanized) | |
| 52 | Nectin-4 CDRH1 | DYYMF |
| | (1) (rat) | |
| 53 | Nectin-4 CDRH2 | YISNGGGNTYYPDTVKG |
| | (1) (rat) | |
| | Nectin-4 CDRH (2) | |
| | 14E4. H3 (mouse) | |
| 54 | Nectin-4 CDRH3 | PEARYYGNYPFPY |
| | (1) | |
| 55 | Nectin-4 heavy | QVQLLQSGPGLVKPSATLSLTCTVSGFSLTTYGVHWIRQPPGKGLEW |
| | chain variable | MGVIWSGGSTDYNAAFISRLTISKDNSKNQVFLQLYSLRAEDTAIYY |
| | sequence (5) | CARTSHWYFDVWGRGTLVTVSS |
| | (sequence name: | |
| | DAB012124) | |
| | (humanized) | |
| 56 | Nectin-4 heavy | QVQLQESGPGLVKPSQTLSLTCTVSGFSLTTYGVHWVRQSPGKGLE |
| | chain variable | WLGVIWSGGSTDYNAAFISRLSISKDNSKSQVFLEMTSLTAADTAIY |
| | sequence (6) | YCARTSHWYFDVWGRGTLVTVSS |
| | (sequence name: | |
| | DAB012125) | |
| | (humanized) | |
| 57 | Nectin-4 heavy | QVQLQESGPGLVKPSQTLSLTCTVSGFSLTTYGVHWVRQAPEKGLE |
| | chain variable | WLGVIWSGGSTDYNAAFISRLSISKDNSKSQVFLEMTSLTADDTAIY |
| | sequence (7) | YCARTSHWYFDVWGRGTLVTVSS |
| | (sequence name: | |
| | DAB012126) | |
| | (humanized) | |
| 58 | Nectin-4 heavy | QVQLQESGPGLVKPSQTLSLTCTVSGFSLTTYGVHWVRQTPEKGLE |
| | chain variable | WLGVIWSGGSTDYNAAFISRLSISKDNSKSQVFLEMTSLTADDTAIY |
| | sequence (8) | YCARTSHWYFDVWGRGTLVTVSS |
| | (sequence name: | |
| | DAB012127) | |
| | (humanized) | |
| 59 | Nectin-4 CDRH1 | TYGVH |
| | (2) | |
| 60 | Nectin-4 CDRH2 | VIWSGGSTDYNAAFIS |
| | (2) (rat) | |
| | Nectin-4 CDRH2 | |
| | 14F6. B1 (mouse) | |
| 61 | Nectin-4 CDRH3 | TSHWYFDV |
| | (2) (rat) | |
| 62 | Nectin-4 heavy | QVQLLESGGGLVQPGGSLRLSCAASGFTFSDYYMYWIRQTPQKRLE |
| | chain variable | WVAYISNGGGNTYYSDTVKGRFTISRDNAKNQLYLQMRSLRPEDTA |
| | sequence (9) | IYYCASPEARYYGNFPFPYWGQGTLVTVSS |
| | (sequence name: | |
| | DAB012128) | |
| | (humanized) | |
| 63 | Nectin-4 heavy | QVQLVESGGGLVKPGGSLRLSCAASGFTFSDYYMYWIRQAPGKGLE |
| | chain variable | WVAYISNGGGNTYYSDTVKGRFTISRDNAKNTLYLQMNSLRAEDTA |
| | sequence (10) | VYYCASPEARYYGNFPFPYWGQGTLVTVSS |
| | (sequence name: | |
| | DAB012129) | |
| | (humanized) | |
| 64 | Nectin-4 heavy | QVQLVESGGGLVKPGGSLRLSCAASGFTFSDYYMYWIRQAPGKGLE |
| | chain variable | WVAYISNGGGNTYYSDTVKGRFTISRDNAKNTLYLQMNSLRAEDTA |
| | sequence (11) | VYYCASPEARYYGNFPFPYWGQGTLVTVSS |
| | (sequence name: | |
| | DAB012130) | |
| | (humanized) | |
| 65 | Nectin-4 heavy | QVQLVESGGGLVKPGGSLRLSCAASGFTFSDYYMYWIRQAPGKGLE |
| | chain variable | WVAYISNGGGNTYYSDTVKGRFTISRDNAKNTLYLQMNSLRAEDTA |
| | sequence (12) | VYYCASPEARYYGNFPFPYWGQGTLVTVSS |
| | (sequence name: | |
| | DAB012131) | |
| | (humanized) | |
| 66 | Nectin-4 CDRH1 | DYYMY |
| | (3) | |
| 67 | Nectin-4 CDRH2 | YISNGGGNTYYSDTVKG |
| | (3) | |
| | Nectin-4 CDRH2 | |
| | 16H8. B1 (mouse) | |
| 68 | Nectin-4 CDRH3 | PEARYYGNFPFPY |
| | (3) | |
| 69 | Nectin-4 heavy | QVQLVESGGGVVQPGRSLRLSCAASGFSFSNYYMAWVRQAPGKGL |
| | chain variable | EWVASISTGGGNIYYRDSVKGRFTISRDNSKSTLYLQMRSLRSEDTAI |
| | sequence (13) | YYCARQTAYYVMDAWGQGTMVTVSS |
| | (sequence name: | |
| | DAB012132) | |
| | (humanized) | |
| 70 | Nectin-4 heavy | EVOLVESGGGLVQPGGSLRLSCAASGFSFSNYYMAWVRQAPGKGLE |
| | chain variable | WVASISTGGGNIYYRDSVKGRFTISRDNAKNSLYLQMNSLRAEDTA |
| | sequence (14) | VYYCARQTAYYVMDAWGQGTTVTVSS |
| | (sequence name: | |
| | DAB012133) | |
| | (humanized) | |
| 71 | Nectin-4 heavy | EVOLVESGGGLVQPGGSLRLSCAASGFSFSNYYMAWVRQAPGKGLE |
| | chain variable | WVASISTGGGNIYYRDSVKGRFTISRDNAKNSLYLQMNSLRAEDTA |
| | sequence (15) | VYYCARQTAYYVMDAWGQGTTVTVSS |
| | (sequence name: | |
| | DAB012134) | |
| | (humanized) | |
| 72 | Nectin-4 heavy | EVOLVESGGGLVQPGGSLRLSCAASGFSFSNYYMAWVRQAPGKGLE |
| | chain variable | WVASISTGGGNIYYRDSVKGRFTISRDNAKNSLYLQMNSLRAEDTA |
| | sequence (16) | VYYCARQTAYYVMDAWGQGTTVTVSS |
| | (sequence name: | |
| | DAB012135) | |
| | (humanized) | |
| 73 | Rat clone | DIQMTQSPASLSVYLGETVSIECLATEDIFSYLAWYQQKPGKSPQLLI |
| | 3H9.1G1 | YGANRLKDGVPSRFSGSGTGTQYSLRISGMQPEDEGDYYCLQGAKF |
| | Light chain variable | PLTFGSGTKLEIK |
| | sequence | |
| 74 | Rat clone | DIQMTQSPASLSASLGETVSIECLASEDIYSYLAWFQQKSGKSPQLLI |
| | 55D10.1C1 | YAANRLQDGVPSRFSGSGSGTQYSLRISGMQPEDEGDYFCLQGSQFP |
| | Light chain variable | WTFGGGTKLELK |
| | sequence | |
| 75 | Rat clone | DIQMTQSPASLSASLGETVSIECLASEDIHNKLAWYQQKPGKSPQLLI |
| | 15H3.1A1 | YYGSNLQDGVPSRFSGSGSGTQYYLKINSLESEDVATYFCLQDSKNP |
| | Light chain variable | WTFGGGTKLEMK |
| | sequence | |
| 76 | Rat clone | DIVLTQSPALDVSLGQRATISCSASQSVSISRYNLIHWYQQKPGQQPK |
| | 54H11.1A4 | LLIWRTSILTSGIPARFSGRGSGTDFTLTINPVQADDIATYYCQQSRES |
| | Light chain variable | PFTFGAGTRLELK |
| | sequence | |
| 77 | Rat clone | NIVLTQSPATLSVTPGESVSLSCRASQSISTGIHWYQQKSNESPRLLIK |
| | 52E1.1B2 | FASQSISGIPSRFSGSGSGTDFTLSINRVESEDFSIYYCQQRDSSLFTFG |
| | Light chain variable | AGTKLELK |
| | sequence | |
| 78 | Rat clone | TYELIQPPSTSVTLGNTVSLTCVGDDLPRRYAYWYQQKPDQSIVRVI |
| | 54E9.1F1 | YEDSKRPSGISDRFSGSSSGTTATLTIRDAQAEDEADYYCHSTYSDDK |
| | Light chain variable | VRVFGGGTKLTVL |
| | sequence | |
| 79 | Rat clone | EVOLVESGGGLVQPGRSMRLSCAASGFSFSNYYMAWVRQAPRKGL |
| | 3H9.1G1 | DWVASISTGGGNIYYRDSVKGRFTISRDNAKSTLYLQMDSLRSEDTA |
| | Heavy chain | TYYCARQTAYYVMDAWGQGASVTVSS |
| | variable sequence | |
| 80 | Rat clone | EVOLVESGGGLVQPGRSLKLSCAASGFTFSNYDMAWVRQVPTKGLE |
| | 55D10.1C1 | WVASISPRGGRIYYRDSVKGRFTVSRDNAKSCLYLQMESLRSDDTAT |
| | Heavy chain | YYCARIHHGYWYFDFWGPGTMVTVSS |
| | variable sequence | |
| 81 | Rat clone | EVKLLESGGGLVQPGGSMRLSCAASGFTFTDFYMIWIRQPAGKAPE |
| | 15H3.1A1 | WLGFIRNKANGYTTDYNPSVKGRFTISRDNTQNMLYLQMITLRAED |
| | Heavy chain | TATYYCARPLYYGYTPRYWGQGVMVTVSS |
| | variable sequence | |
| 82 | Rat clone | EVOLVESGGGLVQPGRPLKLSCAASGFSFSHYDMAWVRQAPTKGLE |
| | 54H11.1A4 | WVAAISPSGGSTYYRDSVKGRFTVSRDKAKNSLYLQMDSLRSEDTA |
| | Heavy chain | TYYCARQGPSYGYYFDYWGQGVMVTVSS |
| | variable sequence | |
| 83 | Rat clone | EVOLVESGGGLVQPGRSLKVSCAASGFTFSNYDMAWVRQAPTKGL |
| | 52E1.1B2 | EWVASINPGGISTYYRDSVKGRFTVSRDNEKSTLYLQMDSLRSEDTA |
| | Heavy chain | TYYCARRQPYFDYWGQGVMVTVSS |
| | variable sequence | |
| 84 | Rat clone | QVTLKESGTGILQPSQALSLTCSISGFSLNTTGICVSWIRQPLGQGLEW |
| | 54E9.1F1 | LADICWDDGKGYNPSLKNRLSISKDTSNNQAFLKITRVDTTDTATYY |
| | Heavy chain | CARNYGGNPFDYWGQGVMVTVSS |
| | variable sequence | |
| 85 | Rat clone | LASEDIYSYLA |
| | 55D10.1C1 | |
| | CDRLI sequence | |
| 86 | Rat clone | AANRLQD |
| | 55D10.1C1 | |
| | CDRL2 sequence | |
| 87 | Rat clone | LQGSQFPWT |
| | 55D10.1C1 | |
| | CDRL3 sequence | |
| 88 | Rat clone | LASEDIHNKLA |
| | 15H3.1A1 | |
| | CDRL1 sequence | |
| 89 | Rat clone | YGSNLQD |
| | 15H3.1A1 | |
| | CDRL2 sequence | |
| 90 | Rat clone | LQDSKNPWT |
| | 15H3.1A1 | |
| | CDRL3 sequence | |
| 91 | Rat clone | SASQSVSISRYNLIH |
| | 54H11.1A4 | |
| | CDRLI sequence | |
| 92 | Rat clone | RTSILTS |
| | 54H11.1A4 | |
| | CDRL2 sequence | |
| 93 | Rat clone | QQSRESPFT |
| | 54H11.1A4 | |
| | CDRL3 sequence | |
| 94 | Rat clone | RASQSISTGIH |
| | 52E1.1B2 | |
| | CDRLI sequence | |
| 95 | Rat clone | FASQSIS |
| | 52E1.1B2 | |
| | CDRL2 sequence | |
| 96 | Rat clone | QQRDSSLFT |
| | 52E1.1B2 | |
| | CDRL3 sequence | |
| 97 | Rat clone | VGDDLPRRYAY |
| | 54E9.1F1 | |
| | CDRLI sequence | |
| 98 | Rat clone | EDSKRPS |
| | 54E9.1F1 | |
| | CDRL2 sequence | |
| 99 | Rat clone | HSTYSDDKVRV |
| | 54E9.1F1 | |
| | CDRL3 sequence | |
| 100 | Rat clone | NYDMA |
| | 55D10.1C1 | |
| | CDRH1 sequence | |
| | Rat clone | |
| | 52E1.1B2 | |
| | CDRH1 sequence | |
| 101 | Rat clone | SISPRGGRIYYRDSVKG |
| | 55D10.1C1 | |
| | CDRH2 sequence | |
| 102 | Rat clone | IHHGYWYFDF |
| | 55D10.1C1 | |
| | CDRH3 sequence | |
| 103 | Rat clone | DFYMI |
| | 15H3.1A1 | |
| | CDRH1 sequence | |
| 104 | Rat clone | FIRNKANGYTTDYNPSVKG |
| | 15H3.1A1 | |
| | CDRH2 sequence | |
| 105 | Rat clone | PLYYGYTPRY |
| | 15H3.1A1 | |
| | CDRH3 sequence | |
| 106 | Rat clone | HYDMA |
| | 54H11.1A4 | |
| | CDRH1 sequence | |
| 107 | Rat clone | AISPSGGSTYYRDSVKG |
| | 54H11.1A4 | |
| | CDRH2 sequence | |
| 108 | Rat clone | QGPSYGYYFDY |
| | 54H11.1A4 | |
| | CDRH3 sequence | |
| 109 | Rat clone | SINPGGISTYYRDSVKG |
| | 52E1.1B2 | |
| | CDRH2 sequence | |
| 110 | Rat clone | RQPYFDY |
| | 52E1.1B2 | |
| | CDRH3 sequence | |
| 111 | Rat clone | VGDDLPRRYAY |
| | 54E9.1F1 | |
| | CDRH1 sequence | |
| 112 | Rat clone | EDSKRPS |
| | 54E9.1F1 | |
| | CDRH2 sequence | |
| 113 | Rat clone | HSTYSDDKVRV |
| | 54E9.1F1 | |
| | CDRH3 sequence | |
| 114 | Mouse clone 14E4. | EVQLQESGGDLVQPGGSLKLSCAASGFTFSDYYMFWIRQTPQKRLE |
| | H3 | WVAYISNGGGNTYYPDTVKGRFTISRDNAKNTLYLQMSRLKSEDTA |
| | Heavy chain | MYYCASPEARYYGNYPFPYWGQGTLVTVSA |
| | variable sequence | |
| 115 | Mouse clone 14E4. | DIVMTQSHKFMSTSVGGRVTITCKASQDVSIAVAWYQQKPGQSPKL |
| | H3 | LIYWASTRQTGVPDRFAGSGSGTDFTLTISNVQSEDLADYFCQQYSS |
| | Light chain variable | YPFTFGSGTKLEIK |
| | sequence | |
| 116 | Mouse clone 14F6. | QVQLKQSGPGLVQPSQSLSITCTVSGFSLTTYGVHWVRQSPGKGLE |
| | B1 Heavy chain | WLGVIWSGGSTDYNAAFISRLSISKDNSKSQVFFEMYSLQADDTAIY |
| | variable sequence | YCARTSHWYFDVWGTGTTVTVSS |
| 117 | Mouse clone 14F6. | DIVMTQSQKFMSTTVGDRVSITCKASQNVVTAVAWYQQKPGQSPKL |
| | B1 Light chain | LIYSASHRFTGVPDRFTGSGSGTDFTLIISNMQSEDLADYFCQQYSSY |
| | variable sequence | PYTFGGGTKLEIK |
| 118 | Mouse clone 16H8. | EVQLQESGGGLVQPGGSLKLSCAASGFTFSDYYMYWIRQTPQKRLE |
| | B1 | WVAYISNGGGNTYYSDTVKGRFTISRDNAKNTLYLQMSRLKSEDTA |
| | Heavy chain | MYYCASPEARYYGNFPFPYWGQGTLVTVSA |
| | variable sequence | |
| 119 | Mouse clone 16H8. | DIVMTQSHKFMSTSVGGRVTITCKASQDVSTTVAWYQQKPGQSPKL |
| | B1 | LIYWASTRQTGVPDRFTGSGSGTDFTLTISNVQSEDLADYFCQQYSS |
| | Light chain variable | YPFTFGSGTKLEIK |
| | sequence | |
| 120 | CDRH1 | GFTFSDYY |
| | 14E4. H3 (mouse) | |
| | CDRH1 | |
| | Clone 16H8. B1 | |
| | (mouse) | |
| 121 | CDRH3 | ASPEARYYGNYPFPY |
| | Clone 14E4. H3 | |
| | (mouse) | |
| 122 | CDRH1 | GFSLTTYG |
| | Clone 14F6. B1 | |
| | (mouse) | |
| 123 | CDRH3 | ARTSHWYFDV |
| | Clone 14F6. B1 | |
| | (mouse) | |
| 124 | CDRH3 | ASPEARYYGNFPFPY |
| | Clone 16H8. B1 | |
| | (mouse) | |
| 125 | Nectin-4 Mask #3 | GGWTCADDEDTWLCPIGG |
| 126 | Nectin-4 Mask #4 | GGWACAPDEDTWLCPIGG |
| 127 | Full-length light | EIQMTQSHKTFSVSTGQRVTITCKASQDVSIAVAWYQQKPGKAPKLL |
| | chain sequence | IYWASTRQTGVPSRFSGSGSGTDFTLTISCVQSEDFAVYYCQQYSSYP |
| | Sequence Name: | FTFGQGTKLEIKRTVAAPSVFIFPPSDEQLKSGTASVVCLLNNFYPRE |
| | DAB012120 | AKVQWKVDNALQSGNSQESVTEQDSKDSTYSLSSTLTLSKADYEKH |
| | | KVYACEVTHQGLSSPVTKSFNRGEC |
| 128 | Full-length light | DIQMTQSPSFLSASVGDRVTITCKASQDVSIAVAWYQQKPGKAPKLL |
| | chain sequence | IYWASTRQTGVPSRFSGSGSGTDFTLTISSLQPEDFATYFCQQYSSYPF |
| | | TFGQGTKLEIKRTVAAPSVFIFPPSDEQLKSGTASVVCLLNNFYPREA |
| | Sequence Name: | KVQWKVDNALQSGNSQESVTEQDSKDSTYSLSSTLTLSKADYEKHK |
| | DAB012121 | VYACEVTHQGLSSPVTKSFNRGEC |
| 129 | Full-length light | DIQMTQSPSFLSASVGERVTITCKASQDVSIAVAWYQQKPGKAPKLL |
| | chain sequence | IYWASTRQTGVPSRFSGSGSGTDFTLTISSLQPEDIATYFCQQYSSYPF |
| | | TFGQGTKLEIKRTVAAPSVFIFPPSDEQLKSGTASVVCLLNNFYPREA |
| | Sequence Name: | KVQWKVDNALQSGNSQESVTEQDSKDSTYSLSSTLTLSKADYEKHK |
| | DAB012122 | VYACEVTHQGLSSPVTKSFNRGEC |
| 130 | Full-length light | DIQMTQSPSFLSASVGDRVTITCKASQDVSIAVAWYQQKPGKAPKLL |
| | chain sequence | IYWASTRQTGVPSRFSGSGSGTDFTLTISSLOPEDSATYFCQQYSSYPF |
| | | TFGQGTKVDIKRTVAAPSVFIFPPSDEQLKSGTASVVCLLNNFYPREA |
| | Sequence Name: | KVQWKVDNALQSGNSQESVTEQDSKDSTYSLSSTLTLSKADYEKHK |
| | DAB012123 | VYACEVTHQGLSSPVTKSFNRGEC |
| 131 | Full-length light | EIQMTQSQSTLSVSLGDRVTITCKASQNVVTAVAWYQQKPGQAPKL |
| | chain sequence | LIYSASHRFTGVPSRFSGSGSGTDFTLIISCMQSEDFAVYYCQQYSSYP |
| | | YTFGQGTKLEIKRTVAAPSVFIFPPSDEQLKSGTASVVCLLNNFYPRE |
| | Sequence Name: | AKVQWKVDNALQSGNSQESVTEQDSKDSTYSLSSTLTLSKADYEKH |
| | DAB012124 | KVYACEVTHQGLSSPVTKSFNRGEC |
| 132 | Full-length light | DIQMTQSPSFLSASVGDRVTITCKASQNVVTAVAWYQQKPGKAPKL |
| | chain sequence | LIYSASHRFTGVPSRFSGSGSGTDFTLTISNLQSEDLATYFCQQYSSYP |
| | | YTFGQGTKLEIKRTVAAPSVFIFPPSDEQLKSGTASVVCLLNNFYPRE |
| | Sequence Name: | AKVQWKVDNALQSGNSQESVTEQDSKDSTYSLSSTLTLSKADYEKH |
| | DAB012125 | KVYACEVTHQGLSSPVTKSFNRGEC |
| 133 | Full-length light | DIQMTQSPSFLSASVGDRVTITCKASQNVVTAVAWYQQKPGKAPKL |
| | chain sequence | LIYSASHRFTGVPSRFSGSGSGTDFTLTISNLQPEDSATYFCQQYSSYP |
| | | YTFGQGTKLEIKRTVAAPSVFIFPPSDEQLKSGTASVVCLLNNFYPRE |
| | Sequence Name: | AKVQWKVDNALQSGNSQESVTEQDSKDSTYSLSSTLTLSKADYEKH |
| | DAB012126 | KVYACEVTHQGLSSPVTKSFNRGEC |
| 134 | Full-length light | DIQMTQSPSFLSASVGDRVTITCKASQNVVTAVAWYQQKPGKAPKL |
| | chain sequence | LIYSASHRFTGVPSRFSGSGSGTDFTLTISNLQPDDAATYFCQQYSSYP |
| | Sequence Name: | YTFGQGTKLEIKRTVAAPSVFIFPPSDEQLKSGTASVVCLLNNFYPRE |
| | DAB012127 | AKVQWKVDNALQSGNSQESVTEQDSKDSTYSLSSTLTLSKADYEKH |
| | | KVYACEVTHQGLSSPVTKSFNRGEC |
| 135 | Full-length light | AIQMTQSHKSFSVSTGQRVTITCKASQDVSTTVAWYQQKPGQAPKL |
| | chain sequence | LIYWASTRQTGVPSRFTGSGSGTDFTLTISCVQSEDFAVYYCQQYSSY |
| | | PFTFGQGTKLEIKRTVAAPSVFIFPPSDEQLKSGTASVVCLLNNFYPRE |
| | Sequence Name: | AKVQWKVDNALQSGNSQESVTEQDSKDSTYSLSSTLTLSKADYEKH |
| | DAB012128 | KVYACEVTHQGLSSPVTKSFNRGEC |
| 136 | Full-length light | DIQMTQSPSFLSASVGDRVTITCKASQDVSTTVAWYQQKPGKAPKLL |
| | chain sequence | IYWASTRQTGVPSRFSGSGSGTDFTLTISNLQPEDFATYFCQQYSSYP |
| | | FTFGQGTKLEIKRTVAAPSVFIFPPSDEQLKSGTASVVCLLNNFYPRE |
| | Sequence Name: | AKVQWKVDNALQSGNSQESVTEQDSKDSTYSLSSTLTLSKADYEKH |
| | DAB012129 | KVYACEVTHQGLSSPVTKSFNRGEC |
| 137 | Full-length light | DIQMTQSPSFLSASVGERVTITCKASQDVSTTVAWYQQKPGKAPKLL |
| | chain sequence | IYWASTRQTGVPSRFSGSGSGTDFTLTINSLQPEDVATYFCQQYSSYP |
| | | FTFGQGTKLEIKRTVAAPSVFIFPPSDEQLKSGTASVVCLLNNFYPRE |
| | Sequence Name: | AKVQWKVDNALQSGNSQESVTEQDSKDSTYSLSSTLTLSKADYEKH |
| | DAB012130 | KVYACEVTHQGLSSPVTKSFNRGEC |
| 138 | Full-length light | DIQMTQSPSFLSASVGERVTITCKASQDVSTTVAWYQQKPGKAPKLL |
| | chain sequence | IYWASTRQTGVPSRFSGSGSGTDFTLTISNLQPEDVATYFCQQYSSYP |
| | | FTFGQGTKLEIKRTVAAPSVFIFPPSDEQLKSGTASVVCLLNNFYPRE |
| | Sequence Name: | AKVQWKVDNALQSGNSQESVTEQDSKDSTYSLSSTLTLSKADYEKH |
| | DAB012131 | KVYACEVTHQGLSSPVTKSFNRGEC |
| 139 | Full-length light | DIQMTQSPSSVSLTVGQRVTITCLATEDIFSYLAWYQQKPGKAPRLLI |
| | chain sequence | YGANRLKDGVPSRFSGSGSGTQYTLRISSMQPEDFGVYYCLQGAKFP |
| | | LTFGPGTKLEIKRTVAAPSVFIFPPSDEQLKSGTASVVCLLNNFYPRE |
| | Sequence Name: | AKVQWKVDNALQSGNSQESVTEQDSKDSTYSLSSTLTLSKADYEKH |
| | DAB012132 | KVYACEVTHQGLSSPVTKSFNRGEC |
| 140 | Full-length light | DIQMTQSPSSLSASIGDTVTITCLATEDIFSYLAWYQQRPGKAPKLLIY |
| | chain sequence | GANRLKDGVPSRFSGSGSGTDYTLTISGLQPEDFVTYYCLQGAKFPL |
| | | TFGQGTKLDMKRTVAAPSVFIFPPSDEQLKSGTASVVCLLNNFYPRE |
| | Sequence Name: | AKVQWKVDNALQSGNSQESVTEQDSKDSTYSLSSTLTLSKADYEKH |
| | DAB012133 | KVYACEVTHQGLSSPVTKSFNRGEC |
| 141 | Full-length light | DIQMTQSPSSLSASVGDTVTITCLATEDIFSYLAWYQQKPGKAPKLLI |
| | chain sequence | YGANRLKDGVPSRFSGSGSGTDYTLTISGLQPEDFGTYYCLQGAKFP |
| | | LTFGQGTKLEIKRTVAAPSVFIFPPSDEQLKSGTASVVCLLNNFYPRE |
| | Sequence Name: | AKVQWKVDNALQSGNSQESVTEQDSKDSTYSLSSTLTLSKADYEKH |
| | DAB012134 | KVYACEVTHQGLSSPVTKSFNRGEC |
| 142 | Full-length light | DIQMTQSPSSLSASVGDTVTITCLATEDIFSYLAWYQQKPGKAPKLLI |
| | chain sequence | YGANRLKDGVPSRFSGGGSGTEYSLTISGLQPEDFGTYYCLQGAKFP |
| | | LTFGQGTKLDMKRTVAAPSVFIFPPSDEQLKSGTASVVCLLNNFYPR |
| | Sequence Name: | EAKVQWKVDNALQSGNSQESVTEQDSKDSTYSLSSTLTLSKADYEK |
| | DAB012135 | HKVYACEVTHQGLSSPVTKSFNRGEC |
| 143 | Full-length light | DIQMTQSPSSLSASVGDTVTITCLATEDIFSYLAWYQQKPGKAPKLLI |
| | chain sequence | YGANRLKDTVPSRFSGGGSGTEYSLTISGLQPEDFGTYYCLQGAKFP |
| | Sequence Name: | LTFGQGTKLDMKRTVAAPSVFIFPPSDEQLKSGTASVVCLLNNFYPR |
| | DAB012135 | EAKVQWKVDNALQSGNSQESVTEQDSKDSTYSLSSTLTLSKADYEK |
| | (modified) | HKVYACEVTHQGLSSPVTKSFNRGEC |
| 144 | Full-length heavy | QVQLLESGGELVQPGGSLRLSCAASGFTFSDYYMFWIRQTPQKRLE |
| | chain sequence | WVAYISNGGGNTYYPDTVKGRFTISRDNAKNQLYLQMRSLTPEDTA |
| | Sequence Name: | IYYCASPEARYYGNYPFPYWGQGTLVTVSSASTKGPSVFPLAPSSKS |
| | DAB012120 | TSGGTAALGCLVKDYFPEPVTVSWNSGALTSGVHTFPAVLQSSGLYS |
| | | LSSVVTVPSSSLGTQTYICNVNHKPSNTKVDKKVEPKSCDKTHTCPP |
| | | CPAPELLGGPSVFLFPPKPKDTLMISRTPEVTCVVVDVSHEDPEVKFN |
| | | WYVDGVEVHNAKTKPREEQYNSTYRVVSVLTVLHQDWLNGKEYK |
| | | CKVSNKALPAPIEKTISKAKGQPREPQVYTLPPSRDELTKNQVSLTCL |
| | | VKGFYPSDIAVEWESNGQPENNYKTTPPVLDSDGSFFLYSKLTVDKS |
| | | RWQQGNVFSCSVMHEALHNHYTQKSLSLSPGK |
| 145 | Full-length heavy | QVQLVESGGGLVKPGGSLRLSCAASGFTFSDYYMFWIRQAPGKGLE |
| | chain sequence | WVAYISNGGGNTYYPDTVKGRFTISRDNAKNTLYLQMNSLRAEDTA |
| | Sequence Name: | VYYCASPEARYYGNYPFPYWGQGTLVTVSSASTKGPSVFPLAPSSKS |
| | DAB012120 | TSGGTAALGCLVKDYFPEPVTVSWNSGALTSGVHTFPAVLQSSGLYS |
| | | LSSVVTVPSSSLGTQTYICNVNHKPSNTKVDKKVEPKSCDKTHTCPP |
| | | CPAPELLGGPSVFLFPPKPKDTLMISRTPEVTCVVVDVSHEDPEVKFN |
| | | WYVDGVEVHNAKTKPREEQYNSTYRVVSVLTVLHQDWLNGKEYK |
| | | CKVSNKALPAPIEKTISKAKGQPREPQVYTLPPSRDELTKNQVSLTCL |
| | | VKGFYPSDIAVEWESNGQPENNYKTTPPVLDSDGSFFLYSKLTVDKS |
| | | RWQQGNVFSCSVMHEALHNHYTQKSLSLSPGK |
| 146 | Full-length heavy | QVQLVESGGGVVKPGGSLRLSCAASGFTFSDYYMFWIRQAPGKGLE |
| | chain sequence | WVAYISNGGGNTYYPDTVKGRFTISRDNAKNTLYLQMNSLRAEDTA |
| | Sequence Name: | VYYCASPEARYYGNYPFPYWGQGTLVTVSSASTKGPSVFPLAPSSKS |
| | DAB012122 | TSGGTAALGCLVKDYFPEPVTVSWNSGALTSGVHTFPAVLQSSGLYS |
| | | LSSVVTVPSSSLGTQTYICNVNHKPSNTKVDKKVEPKSCDKTHTCPP |
| | | CPAPELLGGPSVFLFPPKPKDTLMISRTPEVTCVVVDVSHEDPEVKFN |
| | | WYVDGVEVHNAKTKPREEQYNSTYRVVSVLTVLHQDWLNGKEYK |
| | | CKVSNKALPAPIEKTISKAKGQPREPQVYTLPPSRDELTKNQVSLTCL |
| | | VKGFYPSDIAVEWESNGQPENNYKTTPPVLDSDGSFFLYSKLTVDKS |
| | | RWQQGNVFSCSVMHEALHNHYTQKSLSLSPGK |
| 147 | Full-length heavy | QVQLVESGGGVVQPGGSLRLSCAASGFTFSDYYMFWIRQAPGKGLE |
| | chain sequence | WVAYISNGGGNTYYPDTVKGRFTISRDNAKNTLYLQMNSLRAEDTA |
| | Sequence Name: | VYYCASPEARYYGNYPFPYWGQGTLVTVSSASTKGPSVFPLAPSSKS |
| | DAB012123 | TSGGTAALGCLVKDYFPEPVTVSWNSGALTSGVHTFPAVLQSSGLYS |
| | | LSSVVTVPSSSLGTQTYICNVNHKPSNTKVDKKVEPKSCDKTHTCPP |
| | | CPAPELLGGPSVFLFPPKPKDTLMISRTPEVTCVVVDVSHEDPEVKFN |
| | | WYVDGVEVHNAKTKPREEQYNSTYRVVSVLTVLHQDWLNGKEYK |
| | | CKVSNKALPAPIEKTISKAKGQPREPQVYTLPPSRDELTKNQVSLTCL |
| | | VKGFYPSDIAVEWESNGQPENNYKTTPPVLDSDGSFFLYSKLTVDKS |
| | | RWQQGNVFSCSVMHEALHNHYTQKSLSLSPGK |
| 148 | Full-length heavy | QVQLLQSGPGLVKPSATLSLTCTVSGFSLTTYGVHWIRQPPGKGLEW |
| | chain sequence | MGVIWSGGSTDYNAAFISRLTISKDNSKNQVFLQLYSLRAEDTAIYY |
| | Sequence Name: | CARTSHWYFDVWGRGTLVTVSSASTKGPSVFPLAPSSKSTSGGTAAL |
| | DAB012124 | GCLVKDYFPEPVTVSWNSGALTSGVHTFPAVLQSSGLYSLSSVVTVP |
| | | SSSLGTQTYICNVNHKPSNTKVDKKVEPKSCDKTHTCPPCPAPELLG |
| | | GPSVFLFPPKPKDTLMISRTPEVTCVVVDVSHEDPEVKFNWYVDGVE |
| | | VHNAKTKPREEQYNSTYRVVSVLTVLHQDWLNGKEYKCKVSNKAL |
| | | PAPIEKTISKAKGQPREPQVYTLPPSRDELTKNQVSLTCLVKGFYPSDI |
| | | AVEWESNGQPENNYKTTPPVLDSDGSFFLYSKLTVDKSRWQQGNVF |
| | | SCSVMHEALHNHYTQKSLSLSPGK |
| 149 | Full-length heavy | QVQLQESGPGLVKPSQTLSLTCTVSGFSLTTYGVHWVRQSPGKGLE |
| | chain sequence | WLGVIWSGGSTDYNAAFISRLSISKDNSKSQVFLEMTSLTAADTAIY |
| | Sequence Name: | YCARTSHWYFDVWGRGTLVTVSSASTKGPSVFPLAPSSKSTSGGTA |
| | DAB012125 | ALGCLVKDYFPEPVTVSWNSGALTSGVHTFPAVLQSSGLYSLSSVVT |
| | | VPSSSLGTQTYICNVNHKPSNTKVDKKVEPKSCDKTHTCPPCPAPEL |
| | | LGGPSVFLFPPKPKDTLMISRTPEVTCVVVDVSHEDPEVKFNWYVDG |
| | | VEVHNAKTKPREEQYNSTYRVVSVLTVLHQDWLNGKEYKCKVSNK |
| | | ALPAPIEKTISKAKGQPREPQVYTLPPSRDELTKNQVSLTCLVKGFYP |
| | | SDIAVEWESNGQPENNYKTTPPVLDSDGSFFLYSKLTVDKSRWQQG |
| | | NVFSCSVMHEALHNHYTQKSLSLSPGK |
| 150 | Full-length heavy | QVQLQESGPGLVKPSQTLSLTCTVSGFSLTTYGVHWVRQAPEKGLE |
| | chain sequence | WLGVIWSGGSTDYNAAFISRLSISKDNSKSQVFLEMTSLTADDTAIY |
| | Sequence Name: | YCARTSHWYFDVWGRGTLVTVSSASTKGPSVFPLAPSSKSTSGGTA |
| | DAB012126 | ALGCLVKDYFPEPVTVSWNSGALTSGVHTFPAVLQSSGLYSLSSVVT |
| | | VPSSSLGTQTYICNVNHKPSNTKVDKKVEPKSCDKTHTCPPCPAPEL |
| | | LGGPSVFLFPPKPKDTLMISRTPEVTCVVVDVSHEDPEVKFNWYVDG |
| | | VEVHNAKTKPREEQYNSTYRVVSVLTVLHQDWLNGKEYKCKVSNK |
| | | ALPAPIEKTISKAKGQPREPQVYTLPPSRDELTKNQVSLTCLVKGFYP |
| | | SDIAVEWESNGQPENNYKTTPPVLDSDGSFFLYSKLTVDKSRWQQG |
| | | NVFSCSVMHEALHNHYTQKSLSLSPGK |
| 151 | Full-length heavy | QVQLQESGPGLVKPSQTLSLTCTVSGFSLTTYGVHWVRQTPEKGLE |
| | chain sequence | WLGVIWSGGSTDYNAAFISRLSISKDNSKSQVFLEMTSLTADDTAIY |
| | Sequence Name: | YCARTSHWYFDVWGRGTLVTVSSASTKGPSVFPLAPSSKSTSGGTA |
| | DAB012127 | ALGCLVKDYFPEPVTVSWNSGALTSGVHTFPAVLQSSGLYSLSSVVT |
| | | VPSSSLGTQTYICNVNHKPSNTKVDKKVEPKSCDKTHTCPPCPAPEL |
| | | LGGPSVFLFPPKPKDTLMISRTPEVTCVVVDVSHEDPEVKFNWYVDG |
| | | VEVHNAKTKPREEQYNSTYRVVSVLTVLHQDWLNGKEYKCKVSNK |
| | | ALPAPIEKTISKAKGQPREPQVYTLPPSRDELTKNQVSLTCLVKGFYP |
| | | SDIAVEWESNGQPENNYKTTPPVLDSDGSFFLYSKLTVDKSRWQQG |
| | | NVFSCSVMHEALHNHYTQKSLSLSPGK |
| 152 | Full-length heavy | QVQLLESGGGLVQPGGSLRLSCAASGFTFSDYYMYWIRQTPQKRLE |
| | chain sequence | WVAYISNGGGNTYYSDTVKGRFTISRDNAKNQLYLQMRSLRPEDTA |
| | Sequence Name: | IYYCASPEARYYGNFPFPYWGQGTLVTVSSASTKGPSVFPLAPSSKST |
| | DAB012128 | SGGTAALGCLVKDYFPEPVTVSWNSGALTSGVHTFPAVLQSSGLYSL |
| | | SSVVTVPSSSLGTQTYICNVNHKPSNTKVDKKVEPKSCDKTHTCPPC |
| | | PAPELLGGPSVFLFPPKPKDTLMISRTPEVTCVVVDVSHEDPEVKFN |
| | | WYVDGVEVHNAKTKPREEQYNSTYRVVSVLTVLHQDWLNGKEYK |
| | | CKVSNKALPAPIEKTISKAKGQPREPQVYTLPPSRDELTKNQVSLTCL |
| | | VKGFYPSDIAVEWESNGQPENNYKTTPPVLDSDGSFFLYSKLTVDKS |
| | | RWQQGNVFSCSVMHEALHNHYTQKSLSLSPGK |
| 153 | Full-length heavy | QVQLVESGGGLVKPGGSLRLSCAASGFTFSDYYMYWIRQAPGKGLE |
| | chain sequence | WVAYISNGGGNTYYSDTVKGRFTISRDNAKNTLYLQMNSLRAEDTA |
| | Sequence Name: | VYYCASPEARYYGNFPFPYWGQGTLVTVSSASTKGPSVFPLAPSSKS |
| | DAB012129 | TSGGTAALGCLVKDYFPEPVTVSWNSGALTSGVHTFPAVLQSSGLYS |
| | | LSSVVTVPSSSLGTQTYICNVNHKPSNTKVDKKVEPKSCDKTHTCPP |
| | | CPAPELLGGPSVFLFPPKPKDTLMISRTPEVTCVVVDVSHEDPEVKFN |
| | | WYVDGVEVHNAKTKPREEQYNSTYRVVSVLTVLHQDWLNGKEYK |
| | | CKVSNKALPAPIEKTISKAKGQPREPQVYTLPPSRDELTKNQVSLTCL |
| | | VKGFYPSDIAVEWESNGQPENNYKTTPPVLDSDGSFFLYSKLTVDKS |
| | | RWQQGNVFSCSVMHEALHNHYTQKSLSLSPGK |
| 154 | Full-length heavy | QVQLVESGGGLVKPGGSLRLSCAASGFTFSDYYMYWIRQAPGKGLE |
| | chain sequence | WVAYISNGGGNTYYSDTVKGRFTISRDNAKNTLYLQMNSLRAEDTA |
| | Sequence Name: | VYYCASPEARYYGNFPFPYWGQGTLVTVSSASTKGPSVFPLAPSSKS |
| | DAB012130 | TSGGTAALGCLVKDYFPEPVTVSWNSGALTSGVHTFPAVLQSSGLYS |
| | | LSSVVTVPSSSLGTQTYICNVNHKPSNTKVDKKVEPKSCDKTHTCPP |
| | | CPAPELLGGPSVFLFPPKPKDTLMISRTPEVTCVVVDVSHEDPEVKFN |
| | | WYVDGVEVHNAKTKPREEQYNSTYRVVSVLTVLHQDWLNGKEYK |
| | | CKVSNKALPAPIEKTISKAKGQPREPQVYTLPPSRDELTKNQVSLTCL |
| | | VKGFYPSDIAVEWESNGQPENNYKTTPPVLDSDGSFFLYSKLTVDKS |
| | | RWQQGNVFSCSVMHEALHNHYTQKSLSLSPGK |
| 155 | Full-length heavy | QVQLVESGGGLVKPGGSLRLSCAASGFTFSDYYMYWIRQAPGKGLE |
| | chain sequence | WVAYISNGGGNTYYSDTVKGRFTISRDNAKNTLYLQMNSLRAEDTA |
| | Sequence Name: | VYYCASPEARYYGNFPFPYWGQGTLVTVSSASTKGPSVFPLAPSSKS |
| | DAB012131 | TSGGTAALGCLVKDYFPEPVTVSWNSGALTSGVHTFPAVLQSSGLYS |
| | | LSSVVTVPSSSLGTQTYICNVNHKPSNTKVDKKVEPKSCDKTHTCPP |
| | | CPAPELLGGPSVFLFPPKPKDTLMISRTPEVTCVVVDVSHEDPEVKFN |
| | | WYVDGVEVHNAKTKPREEQYNSTYRVVSVLTVLHQDWLNGKEYK |
| | | CKVSNKALPAPIEKTISKAKGQPREPQVYTLPPSRDELTKNQVSLTCL |
| | | VKGFYPSDIAVEWESNGQPENNYKTTPPVLDSDGSFFLYSKLTVDKS |
| | | RWQQGNVFSCSVMHEALHNHYTQKSLSLSPGK |
| 156 | Full-length heavy | QVQLVESGGGVVQPGRSLRLSCAASGFSFSNYYMAWVRQAPGKGL |
| | chain sequence | EWVASISTGGGNIYYRDSVKGRFTISRDNSKSTLYLQMRSLRSEDTAI |
| | Sequence Name: | YYCARQTAYYVMDAWGQGTMVTVSSASTKGPSVFPLAPSSKSTSG |
| | DAB012132 | GTAALGCLVKDYFPEPVTVSWNSGALTSGVHTFPAVLQSSGLYSLSS |
| | | VVTVPSSSLGTQTYICNVNHKPSNTKVDKKVEPKSCDKTHTCPPCPA |
| | | PELLGGPSVFLFPPKPKDTLMISRTPEVTCVVVDVSHEDPEVKFNWY |
| | | VDGVEVHNAKTKPREEQYNSTYRVVSVLTVLHQDWLNGKEYKCK |
| | | VSNKALPAPIEKTISKAKGQPREPQVYTLPPSRDELTKNQVSLTCLVK |
| | | GFYPSDIAVEWESNGQPENNYKTTPPVLDSDGSFFLYSKLTVDKSRW |
| | | QQGNVFSCSVMHEALHNHYTQKSLSLSPGK |
| 157 | Full-length heavy | EVOLVESGGGLVQPGGSLRLSCAASGFSFSNYYMAWVRQAPGKGLE |
| | chain sequence | WVASISTGGGNIYYRDSVKGRFTISRDNAKNSLYLQMNSLRAEDTA |
| | Sequence Name: | VYYCARQTAYYVMDAWGQGTTVTVSSASTKGPSVFPLAPSSKSTSG |
| | DAB012133 | GTAALGCLVKDYFPEPVTVSWNSGALTSGVHTFPAVLQSSGLYSLSS |
| | | VVTVPSSSLGTQTYICNVNHKPSNTKVDKKVEPKSCDKTHTCPPCPA |
| | | PELLGGPSVFLFPPKPKDTLMISRTPEVTCVVVDVSHEDPEVKFNWY |
| | | VDGVEVHNAKTKPREEQYNSTYRVVSVLTVLHQDWLNGKEYKCK |
| | | VSNKALPAPIEKTISKAKGQPREPQVYTLPPSRDELTKNQVSLTCLVK |
| | | GFYPSDIAVEWESNGQPENNYKTTPPVLDSDGSFFLYSKLTVDKSRW |
| | | QQGNVFSCSVMHEALHNHYTQKSLSLSPGK |
| 158 | Full-length heavy | EVOLVESGGGLVQPGGSLRLSCAASGFSFSNYYMAWVRQAPGKGLE |
| | chain sequence | WVASISTGGGNIYYRDSVKGRFTISRDNAKNSLYLQMNSLRAEDTA |
| | Sequence Name: | VYYCARQTAYYVMDAWGQGTTVTVSSASTKGPSVFPLAPSSKSTSG |
| | DAB012134 | GTAALGCLVKDYFPEPVTVSWNSGALTSGVHTFPAVLQSSGLYSLSS |
| | | VVTVPSSSLGTQTYICNVNHKPSNTKVDKKVEPKSCDKTHTCPPCPA |
| | | PELLGGPSVFLFPPKPKDTLMISRTPEVTCVVVDVSHEDPEVKFNWY |
| | | VDGVEVHNAKTKPREEQYNSTYRVVSVLTVLHQDWLNGKEYKCK |
| | | VSNKALPAPIEKTISKAKGQPREPQVYTLPPSRDELTKNQVSLTCLVK |
| | | GFYPSDIAVEWESNGQPENNYKTTPPVLDSDGSFFLYSKLTVDKSRW |
| | | QQGNVFSCSVMHEALHNHYTQKSLSLSPGK |
| 159 | Full-length heavy | EVOLVESGGGLVQPGGSLRLSCAASGFSFSNYYMAWVRQAPGKGLE |
| | chain sequence | WVASISTGGGNIYYRDSVKGRFTISRDNAKNSLYLQMNSLRAEDTA |
| | Sequence Name: | VYYCARQTAYYVMDAWGQGTTVTVSSASTKGPSVFPLAPSSKSTSG |
| | DAB012135 | GTAALGCLVKDYFPEPVTVSWNSGALTSGVHTFPAVLQSSGLYSLSS |
| | | VVTVPSSSLGTQTYICNVNHKPSNTKVDKKVEPKSCDKTHTCPPCPA |
| | | PELLGGPSVFLFPPKPKDTLMISRTPEVTCVVVDVSHEDPEVKFNWY |
| | | VDGVEVHNAKTKPREEQYNSTYRVVSVLTVLHQDWLNGKEYKCK |
| | | VSNKALPAPIEKTISKAKGQPREPQVYTLPPSRDELTKNQVSLTCLVK |
| | | GFYPSDIAVEWESNGQPENNYKTTPPVLDSDGSFFLYSKLTVDKSRW |
| | | QQGNVFSCSVMHEALHNHYTQKSLSLSPGK |
| 160 | Full-length light | GAGATCCAGATGACCCAGAGCCACAAGACCTTCAGCGTGAGCAC |
| | chain nucleotide | CGGCCAGAGGGTGACCATCACCTGCAAGGCCAGCCAGGACGTGA |
| | sequence | GCATCGCCGTGGCCTGGTACCAGCAGAAGCCCGGCAAGGCCCCC |
| | Sequence Name: | AAGCTGCTGATCTACTGGGCCAGCACCAGGCAGACCGGCGTGCC |
| | DAB012120 | CAGCAGGTTCAGCGGCAGCGGCAGCGGCACCGACTTCACCCTGA |
| | | CCATCAGCTGCGTGCAGAGCGAGGACTTCGCCGTGTACTACTGCC |
| | | AGCAGTACAGCAGCTACCCCTTCACCTTCGGCCAGGGCACCAAGC |
| | | TGGAGATCAAGCGAACGGTGGCTGCCCCCTCCGTGTTCATCTTCC |
| | | CCCCCAGCGATGAGCAGCTGAAGAGCGGCACAGCCAGCGTGGTG |
| | | TGCCTGCTGAACAACTTCTACCCCAGGGAGGCCAAGGTGCAGTGG |
| | | AAGGTGGACAACGCCCTGCAGAGCGGCAACAGCCAGGAGTCCGT |
| | | GACCGAGCAGGACAGCAAGGACAGCACCTACAGCCTGAGCAGCA |
| | | CACTGACCCTGAGCAAGGCCGACTACGAGAAGCACAAGGTGTAC |
| | | GCCTGCGAGGTGACCCATCAGGGCCTGAGCAGCCCCGTGACCAA |
| | | GAGCTTCAACAGGGGCGAGTGC |
| 161 | Full-length light | GACATCCAGATGACCCAGAGCCCCAGCTTCCTGAGCGCCAGCGTG |
| | chain nucleotide | GGCGACAGGGTGACCATCACCTGCAAGGCCAGCCAGGACGTGAG |
| | sequence | CATCGCCGTGGCCTGGTACCAGCAGAAGCCCGGCAAGGCCCCCA |
| | Sequence Name: | AGCTGCTGATCTACTGGGCCAGCACCAGGCAGACCGGCGTGCCC |
| | DAB012121 | AGCAGGTTCAGCGGCAGCGGCAGCGGCACCGACTTCACCCTGAC |
| | | CATCAGCAGCCTGCAGCCCGAGGACTTCGCCACCTACTTCTGCCA |
| | | GCAGTACAGCAGCTACCCCTTCACCTTCGGCCAGGGCACCAAGCT |
| | | GGAGATCAAGCGAACGGTGGCTGCCCCCTCCGTGTTCATCTTCCC |
| | | CCCCAGCGATGAGCAGCTGAAGAGCGGCACAGCCAGCGTGGTGT |
| | | GCCTGCTGAACAACTTCTACCCCAGGGAGGCCAAGGTGCAGTGG |
| | | AAGGTGGACAACGCCCTGCAGAGCGGCAACAGCCAGGAGTCCGT |
| | | GACCGAGCAGGACAGCAAGGACAGCACCTACAGCCTGAGCAGCA |
| | | CACTGACCCTGAGCAAGGCCGACTACGAGAAGCACAAGGTGTAC |
| | | GCCTGCGAGGTGACCCATCAGGGCCTGAGCAGCCCCGTGACCAA |
| | | GAGCTTCAACAGGGGCGAGTGC |
| 162 | Full-length light | GACATCCAGATGACCCAGAGCCCCAGCTTCCTGAGCGCCAGCGTG |
| | chain nucleotide | GGCGAGAGGGTGACCATCACCTGCAAGGCCAGCCAGGACGTGAG |
| | sequence | CATCGCCGTGGCCTGGTACCAGCAGAAGCCCGGCAAGGCCCCCA |
| | Sequence Name: | AGCTGCTGATCTACTGGGCCAGCACCAGGCAGACCGGCGTGCCC |
| | DAB012122 | AGCAGGTTCAGCGGCAGCGGCAGCGGCACCGACTTCACCCTGAC |
| | | CATCAGCAGCCTGCAGCCCGAGGACATCGCCACCTACTTCTGCCA |
| | | GCAGTACAGCAGCTACCCCTTCACCTTCGGCCAGGGCACCAAGCT |
| | | GGAGATCAAGCGAACGGTGGCTGCCCCCTCCGTGTTCATCTTCCC |
| | | CCCCAGCGATGAGCAGCTGAAGAGCGGCACAGCCAGCGTGGTGT |
| | | GCCTGCTGAACAACTTCTACCCCAGGGAGGCCAAGGTGCAGTGG |
| | | AAGGTGGACAACGCCCTGCAGAGCGGCAACAGCCAGGAGTCCGT |
| | | GACCGAGCAGGACAGCAAGGACAGCACCTACAGCCTGAGCAGCA |
| | | CACTGACCCTGAGCAAGGCCGACTACGAGAAGCACAAGGTGTAC |
| | | GCCTGCGAGGTGACCCATCAGGGCCTGAGCAGCCCCGTGACCAA |
| | | GAGCTTCAACAGGGGCGAGTGC |
| 163 | Full-length light | GACATCCAGATGACCCAGAGCCCCAGCTTCCTGAGCGCCAGCGTG |
| | chain nucleotide | GGCGACAGGGTGACCATCACCTGCAAGGCCAGCCAGGACGTGAG |
| | sequence | CATCGCCGTGGCCTGGTACCAGCAGAAGCCCGGCAAGGCCCCCA |
| | Sequence Name: | AGCTGCTGATCTACTGGGCCAGCACCAGGCAGACCGGCGTGCCC |
| | DAB012123 | AGCAGGTTCAGCGGCAGCGGCAGCGGCACCGACTTCACCCTGAC |
| | | CATCAGCAGCCTGCAGCCCGAGGACAGCGCCACCTACTTCTGCCA |
| | | GCAGTACAGCAGCTACCCCTTCACCTTCGGCCAGGGCACCAAGGT |
| | | GGACATCAAGCGAACGGTGGCTGCCCCCTCCGTGTTCATCTTCCC |
| | | CCCCAGCGATGAGCAGCTGAAGAGCGGCACAGCCAGCGTGGTGT |
| | | GCCTGCTGAACAACTTCTACCCCAGGGAGGCCAAGGTGCAGTGG |
| | | AAGGTGGACAACGCCCTGCAGAGCGGCAACAGCCAGGAGTCCGT |
| | | GACCGAGCAGGACAGCAAGGACAGCACCTACAGCCTGAGCAGCA |
| | | CACTGACCCTGAGCAAGGCCGACTACGAGAAGCACAAGGTGTAC |
| | | GCCTGCGAGGTGACCCATCAGGGCCTGAGCAGCCCCGTGACCAA |
| | | GAGCTTCAACAGGGGCGAGTGC |
| 164 | Full-length light | GAGATCCAGATGACCCAGAGCCAGAGCACCCTGAGCGTGAGCCT |
| | chain nucleotide | GGGCGACAGGGTGACCATCACCTGCAAGGCCAGCCAGAACGTGG |
| | sequence | TGACCGCCGTGGCCTGGTACCAGCAGAAGCCCGGCCAGGCCCCC |
| | Sequence Name: | AAGCTGCTGATCTACAGCGCCAGCCACAGGTTCACCGGCGTGCCC |
| | DAB012124 | AGCAGGTTCAGCGGCAGCGGCAGCGGCACCGACTTCACCCTGAT |
| | | CATCAGCTGCATGCAGAGCGAGGACTTCGCCGTGTACTACTGCCA |
| | | GCAGTACAGCAGCTACCCCTACACCTTCGGCCAGGGCACCAAGCT |
| | | GGAGATCAAGCGAACGGTGGCTGCCCCCTCCGTGTTCATCTTCCC |
| | | CCCCAGCGATGAGCAGCTGAAGAGCGGCACAGCCAGCGTGGTGT |
| | | GCCTGCTGAACAACTTCTACCCCAGGGAGGCCAAGGTGCAGTGG |
| | | AAGGTGGACAACGCCCTGCAGAGCGGCAACAGCCAGGAGTCCGT |
| | | GACCGAGCAGGACAGCAAGGACAGCACCTACAGCCTGAGCAGCA |
| | | CACTGACCCTGAGCAAGGCCGACTACGAGAAGCACAAGGTGTAC |
| | | GCCTGCGAGGTGACCCATCAGGGCCTGAGCAGCCCCGTGACCAA |
| | | GAGCTTCAACAGGGGCGAGTGC |
| 165 | Full-length light | GACATCCAGATGACCCAGAGCCCCAGCTTCCTGAGCGCCAGCGTG |
| | chain nucleotide | GGCGACAGGGTGACCATCACCTGCAAGGCCAGCCAGAACGTGGT |
| | sequence | GACCGCCGTGGCCTGGTACCAGCAGAAGCCCGGCAAGGCCCCCA |
| | Sequence Name: | AGCTGCTGATCTACAGCGCCAGCCACAGGTTCACCGGCGTGCCCA |
| | DAB012125 | GCAGGTTCAGCGGCAGCGGCAGCGGCACCGACTTCACCCTGACC |
| | | ATCAGCAACCTGCAGAGCGAGGACCTGGCCACCTACTTCTGCCAG |
| | | CAGTACAGCAGCTACCCCTACACCTTCGGCCAGGGCACCAAGCTG |
| | | GAGATCAAGCGAACGGTGGCTGCCCCCTCCGTGTTCATCTTCCCC |
| | | CCCAGCGATGAGCAGCTGAAGAGCGGCACAGCCAGCGTGGTGTG |
| | | CCTGCTGAACAACTTCTACCCCAGGGAGGCCAAGGTGCAGTGGA |
| | | AGGTGGACAACGCCCTGCAGAGCGGCAACAGCCAGGAGTCCGTG |
| | | ACCGAGCAGGACAGCAAGGACAGCACCTACAGCCTGAGCAGCAC |
| | | ACTGACCCTGAGCAAGGCCGACTACGAGAAGCACAAGGTGTACG |
| | | CCTGCGAGGTGACCCATCAGGGCCTGAGCAGCCCCGTGACCAAG |
| | | AGCTTCAACAGGGGCGAGTGC |
| 166 | Full-length light | GACATCCAGATGACCCAGAGCCCCAGCTTCCTGAGCGCCAGCGTG |
| | chain nucleotide | GGCGACAGGGTGACCATCACCTGCAAGGCCAGCCAGAACGTGGT |
| | sequence | GACCGCCGTGGCCTGGTACCAGCAGAAGCCCGGCAAGGCCCCCA |
| | Sequence Name: | AGCTGCTGATCTACAGCGCCAGCCACAGGTTCACCGGCGTGCCCA |
| | DAB012126 | GCAGGTTCAGCGGCAGCGGCAGCGGCACCGACTTCACCCTGACC |
| | | ATCAGCAACCTGCAGCCCGAGGACAGCGCCACCTACTTCTGCCAG |
| | | CAGTACAGCAGCTACCCCTACACCTTCGGCCAGGGCACCAAGCTG |
| | | GAGATCAAGCGAACGGTGGCTGCCCCCTCCGTGTTCATCTTCCCC |
| | | CCCAGCGATGAGCAGCTGAAGAGCGGCACAGCCAGCGTGGTGTG |
| | | CCTGCTGAACAACTTCTACCCCAGGGAGGCCAAGGTGCAGTGGA |
| | | AGGTGGACAACGCCCTGCAGAGCGGCAACAGCCAGGAGTCCGTG |
| | | ACCGAGCAGGACAGCAAGGACAGCACCTACAGCCTGAGCAGCAC |
| | | ACTGACCCTGAGCAAGGCCGACTACGAGAAGCACAAGGTGTACG |
| | | CCTGCGAGGTGACCCATCAGGGCCTGAGCAGCCCCGTGACCAAG |
| | | AGCTTCAACAGGGGCGAGTGC |
| 167 | Full-length light | GACATCCAGATGACCCAGAGCCCCAGCTTCCTGAGCGCCAGCGTG |
| | chain nucleotide | GGCGACAGGGTGACCATCACCTGCAAGGCCAGCCAGAACGTGGT |
| | sequence | GACCGCCGTGGCCTGGTACCAGCAGAAGCCCGGCAAGGCCCCCA |
| | Sequence Name: | AGCTGCTGATCTACAGCGCCAGCCACAGGTTCACCGGCGTGCCCA |
| | DAB012127 | GCAGGTTCAGCGGCAGCGGCAGCGGCACCGACTTCACCCTGACC |
| | | ATCAGCAACCTGCAGCCCGACGACGCCGCCACCTACTTCTGCCAG |
| | | CAGTACAGCAGCTACCCCTACACCTTCGGCCAGGGCACCAAGCTG |
| | | GAGATCAAGCGAACGGTGGCTGCCCCCTCCGTGTTCATCTTCCCC |
| | | CCCAGCGATGAGCAGCTGAAGAGCGGCACAGCCAGCGTGGTGTG |
| | | CCTGCTGAACAACTTCTACCCCAGGGAGGCCAAGGTGCAGTGGA |
| | | AGGTGGACAACGCCCTGCAGAGCGGCAACAGCCAGGAGTCCGTG |
| | | ACCGAGCAGGACAGCAAGGACAGCACCTACAGCCTGAGCAGCAC |
| | | ACTGACCCTGAGCAAGGCCGACTACGAGAAGCACAAGGTGTACG |
| | | CCTGCGAGGTGACCCATCAGGGCCTGAGCAGCCCCGTGACCAAG |
| | | AGCTTCAACAGGGGCGAGTGC |
| 168 | Full-length light | GCCATCCAGATGACCCAGAGCCACAAGAGCTTCAGCGTGAGCAC |
| | chain nucleotide | CGGCCAGAGGGTGACCATCACCTGCAAGGCCAGCCAGGACGTGA |
| | sequence | GCACCACCGTGGCCTGGTACCAGCAGAAGCCCGGCCAGGCCCCC |
| | Sequence Name: | AAGCTGCTGATCTACTGGGCCAGCACCAGGCAGACCGGCGTGCC |
| | DAB012128 | CAGCAGGTTCACCGGCAGCGGCAGCGGCACCGACTTCACCCTGA |
| | | CCATCAGCTGCGTGCAGAGCGAGGACTTCGCCGTGTACTACTGCC |
| | | AGCAGTACAGCAGCTACCCCTTCACCTTCGGCCAGGGCACCAAGC |
| | | TGGAGATCAAGCGAACGGTGGCTGCCCCCTCCGTGTTCATCTTCC |
| | | CCCCCAGCGATGAGCAGCTGAAGAGCGGCACAGCCAGCGTGGTG |
| | | TGCCTGCTGAACAACTTCTACCCCAGGGAGGCCAAGGTGCAGTGG |
| | | AAGGTGGACAACGCCCTGCAGAGCGGCAACAGCCAGGAGTCCGT |
| | | GACCGAGCAGGACAGCAAGGACAGCACCTACAGCCTGAGCAGCA |
| | | CACTGACCCTGAGCAAGGCCGACTACGAGAAGCACAAGGTGTAC |
| | | GCCTGCGAGGTGACCCATCAGGGCCTGAGCAGCCCCGTGACCAA |
| | | GAGCTTCAACAGGGGCGAGTGC |
| 169 | Full-length light | GACATCCAGATGACCCAGAGCCCCAGCTTCCTGAGCGCCAGCGTG |
| | chain nucleotide | GGCGACAGGGTGACCATCACCTGCAAGGCCAGCCAGGACGTGAG |
| | sequence | CACCACCGTGGCCTGGTACCAGCAGAAGCCCGGCAAGGCCCCCA |
| | Sequence Name: | AGCTGCTGATCTACTGGGCCAGCACCAGGCAGACCGGCGTGCCC |
| | DAB012129 | AGCAGGTTCAGCGGCAGCGGCAGCGGCACCGACTTCACCCTGAC |
| | | CATCAGCAACCTGCAGCCCGAGGACTTCGCCACCTACTTCTGCCA |
| | | GCAGTACAGCAGCTACCCCTTCACCTTCGGCCAGGGCACCAAGCT |
| | | GGAGATCAAGCGAACGGTGGCTGCCCCCTCCGTGTTCATCTTCCC |
| | | CCCCAGCGATGAGCAGCTGAAGAGCGGCACAGCCAGCGTGGTGT |
| | | GCCTGCTGAACAACTTCTACCCCAGGGAGGCCAAGGTGCAGTGG |
| | | AAGGTGGACAACGCCCTGCAGAGCGGCAACAGCCAGGAGTCCGT |
| | | GACCGAGCAGGACAGCAAGGACAGCACCTACAGCCTGAGCAGCA |
| | | CACTGACCCTGAGCAAGGCCGACTACGAGAAGCACAAGGTGTAC |
| | | GCCTGCGAGGTGACCCATCAGGGCCTGAGCAGCCCCGTGACCAA |
| | | GAGCTTCAACAGGGGCGAGTGC |
| 170 | Full-length light | GACATCCAGATGACCCAGAGCCCCAGCTTCCTGAGCGCCAGCGTG |
| | chain nucleotide | GGCGAGAGGGTGACCATCACCTGCAAGGCCAGCCAGGACGTGAG |
| | sequence | CACCACCGTGGCCTGGTACCAGCAGAAGCCCGGCAAGGCCCCCA |
| | Sequence Name: | AGCTGCTGATCTACTGGGCCAGCACCAGGCAGACCGGCGTGCCC |
| | DAB012130 | AGCAGGTTCAGCGGCAGCGGCAGCGGCACCGACTTCACCCTGAC |
| | | CATCAACAGCCTGCAGCCCGAGGACGTGGCCACCTACTTCTGCCA |
| | | GCAGTACAGCAGCTACCCCTTCACCTTCGGCCAGGGCACCAAGCT |
| | | GGAGATCAAGCGAACGGTGGCTGCCCCCTCCGTGTTCATCTTCCC |
| | | CCCCAGCGATGAGCAGCTGAAGAGCGGCACAGCCAGCGTGGTGT |
| | | GCCTGCTGAACAACTTCTACCCCAGGGAGGCCAAGGTGCAGTGG |
| | | AAGGTGGACAACGCCCTGCAGAGCGGCAACAGCCAGGAGTCCGT |
| | | GACCGAGCAGGACAGCAAGGACAGCACCTACAGCCTGAGCAGCA |
| | | CACTGACCCTGAGCAAGGCCGACTACGAGAAGCACAAGGTGTAC |
| | | GCCTGCGAGGTGACCCATCAGGGCCTGAGCAGCCCCGTGACCAA |
| | | GAGCTTCAACAGGGGCGAGTGC |
| 171 | Full-length light | GACATCCAGATGACCCAGAGCCCCAGCTTCCTGAGCGCCAGCGTG |
| | chain nucleotide | GGCGAGAGGGTGACCATCACCTGCAAGGCCAGCCAGGACGTGAG |
| | sequence | CACCACCGTGGCCTGGTACCAGCAGAAGCCCGGCAAGGCCCCCA |
| | Sequence Name: | AGCTGCTGATCTACTGGGCCAGCACCAGGCAGACCGGCGTGCCC |
| | DAB012131 | AGCAGGTTCAGCGGCAGCGGCAGCGGCACCGACTTCACCCTGAC |
| | | CATCAGCAACCTGCAGCCCGAGGACGTGGCCACCTACTTCTGCCA |
| | | GCAGTACAGCAGCTACCCCTTCACCTTCGGCCAGGGCACCAAGCT |
| | | GGAGATCAAGCGAACGGTGGCTGCCCCCTCCGTGTTCATCTTCCC |
| | | CCCCAGCGATGAGCAGCTGAAGAGCGGCACAGCCAGCGTGGTGT |
| | | GCCTGCTGAACAACTTCTACCCCAGGGAGGCCAAGGTGCAGTGG |
| | | AAGGTGGACAACGCCCTGCAGAGCGGCAACAGCCAGGAGTCCGT |
| | | GACCGAGCAGGACAGCAAGGACAGCACCTACAGCCTGAGCAGCA |
| | | CACTGACCCTGAGCAAGGCCGACTACGAGAAGCACAAGGTGTAC |
| | | GCCTGCGAGGTGACCCATCAGGGCCTGAGCAGCCCCGTGACCAA |
| | | GAGCTTCAACAGGGGCGAGTGC |
| 172 | Full-length light | GACATCCAGATGACCCAGAGCCCCAGCAGCGTGAGCCTGACCGT |
| | chain nucleotide | GGGCCAGAGGGTGACCATCACCTGCCTGGCCACCGAGGACATCTT |
| | sequence | CAGCTACCTGGCCTGGTACCAGCAGAAGCCCGGCAAGGCCCCCA |
| | Sequence Name: | GGCTGCTGATCTACGGCGCCAACAGGCTGAAGGACGGCGTGCCC |
| | DAB012132 | AGCAGGTTCAGCGGCAGCGGCAGCGGCACCCAGTACACCCTGAG |
| | | GATCAGCAGCATGCAGCCCGAGGACTTCGGCGTGTACTACTGCCT |
| | | GCAGGGCGCCAAGTTCCCCCTGACCTTCGGCCCCGGCACCAAGCT |
| | | GGAGATCAAGCGAACGGTGGCTGCCCCCTCCGTGTTCATCTTCCC |
| | | CCCCAGCGATGAGCAGCTGAAGAGCGGCACAGCCAGCGTGGTGT |
| | | GCCTGCTGAACAACTTCTACCCCAGGGAGGCCAAGGTGCAGTGG |
| | | AAGGTGGACAACGCCCTGCAGAGCGGCAACAGCCAGGAGTCCGT |
| | | GACCGAGCAGGACAGCAAGGACAGCACCTACAGCCTGAGCAGCA |
| | | CACTGACCCTGAGCAAGGCCGACTACGAGAAGCACAAGGTGTAC |
| | | GCCTGCGAGGTGACCCATCAGGGCCTGAGCAGCCCCGTGACCAA |
| | | GAGCTTCAACAGGGGCGAGTGC |
| 173 | Full-length light | GACATCCAGATGACCCAGAGCCCCAGCAGCCTGAGCGCCAGCAT |
| | chain nucleotide | CGGCGACACCGTGACCATCACCTGCCTGGCCACCGAGGACATCTT |
| | sequence | CAGCTACCTGGCCTGGTACCAGCAGAGGCCCGGCAAGGCCCCCA |
| | Sequence Name: | AGCTGCTGATCTACGGCGCCAACAGGCTGAAGGACGGCGTGCCC |
| | DAB012133 | AGCAGGTTCAGCGGCAGCGGCAGCGGCACCGACTACACCCTGAC |
| | | CATCAGCGGCCTGCAGCCCGAGGACTTCGTGACCTACTACTGCCT |
| | | GCAGGGCGCCAAGTTCCCCCTGACCTTCGGCCAGGGCACCAAGCT |
| | | GGACATGAAGCGAACGGTGGCTGCCCCCTCCGTGTTCATCTTCCC |
| | | CCCCAGCGATGAGCAGCTGAAGAGCGGCACAGCCAGCGTGGTGT |
| | | GCCTGCTGAACAACTTCTACCCCAGGGAGGCCAAGGTGCAGTGG |
| | | AAGGTGGACAACGCCCTGCAGAGCGGCAACAGCCAGGAGTCCGT |
| | | GACCGAGCAGGACAGCAAGGACAGCACCTACAGCCTGAGCAGCA |
| | | CACTGACCCTGAGCAAGGCCGACTACGAGAAGCACAAGGTGTAC |
| | | GCCTGCGAGGTGACCCATCAGGGCCTGAGCAGCCCCGTGACCAA |
| | | GAGCTTCAACAGGGGCGAGTGC |
| 174 | Full-length light | GACATCCAGATGACCCAGAGCCCCAGCAGCCTGAGCGCCAGCGT |
| | chain nucleotide | GGGCGACACCGTGACCATCACCTGCCTGGCCACCGAGGACATCTT |
| | sequence | CAGCTACCTGGCCTGGTACCAGCAGAAGCCCGGCAAGGCCCCCA |
| | Sequence Name: | AGCTGCTGATCTACGGCGCCAACAGGCTGAAGGACGGCGTGCCC |
| | DAB012134 | AGCAGGTTCAGCGGCAGCGGCAGCGGCACCGACTACACCCTGAC |
| | | CATCAGCGGCCTGCAGCCCGAGGACTTCGGCACCTACTACTGCCT |
| | | GCAGGGCGCCAAGTTCCCCCTGACCTTCGGCCAGGGCACCAAGCT |
| | | GGAGATCAAGCGAACGGTGGCTGCCCCCTCCGTGTTCATCTTCCC |
| | | CCCCAGCGATGAGCAGCTGAAGAGCGGCACAGCCAGCGTGGTGT |
| | | GCCTGCTGAACAACTTCTACCCCAGGGAGGCCAAGGTGCAGTGG |
| | | AAGGTGGACAACGCCCTGCAGAGCGGCAACAGCCAGGAGTCCGT |
| | | GACCGAGCAGGACAGCAAGGACAGCACCTACAGCCTGAGCAGCA |
| | | CACTGACCCTGAGCAAGGCCGACTACGAGAAGCACAAGGTGTAC |
| | | GCCTGCGAGGTGACCCATCAGGGCCTGAGCAGCCCCGTGACCAA |
| | | GAGCTTCAACAGGGGCGAGTGC |
| 175 | Full-length light | GACATCCAGATGACCCAGAGCCCCAGCAGCCTGAGCGCCAGCGT |
| | chain nucleotide | GGGCGACACCGTGACCATCACCTGCCTGGCCACCGAGGACATCTT |
| | sequence | CAGCTACCTGGCCTGGTACCAGCAGAAGCCCGGCAAGGCCCCCA |
| | Sequence Name: | AGCTGCTGATCTACGGCGCCAACAGGCTGAAGGACGGCGTGCCC |
| | DAB012135 | AGCAGGTTCAGCGGCGGCGGCAGCGGCACCGAGTACAGCCTGAC |
| | | CATCAGCGGCCTGCAGCCCGAGGACTTCGGCACCTACTACTGCCT |
| | | GCAGGGCGCCAAGTTCCCCCTGACCTTCGGCCAGGGCACCAAGCT |
| | | GGACATGAAGCGAACGGTGGCTGCCCCCTCCGTGTTCATCTTCCC |
| | | CCCCAGCGATGAGCAGCTGAAGAGCGGCACAGCCAGCGTGGTGT |
| | | GCCTGCTGAACAACTTCTACCCCAGGGAGGCCAAGGTGCAGTGG |
| | | AAGGTGGACAACGCCCTGCAGAGCGGCAACAGCCAGGAGTCCGT |
| | | GACCGAGCAGGACAGCAAGGACAGCACCTACAGCCTGAGCAGCA |
| | | CACTGACCCTGAGCAAGGCCGACTACGAGAAGCACAAGGTGTAC |
| | | GCCTGCGAGGTGACCCATCAGGGCCTGAGCAGCCCCGTGACCAA |
| | | GAGCTTCAACAGGGGCGAGTGC |
| 176 | Full-length light | GACATCCAGATGACCCAGAGCCCCAGCAGCCTGAGCGCCAGCGT |
| | chain nucleotide | GGGCGACACCGTGACCATCACCTGCCTGGCCACCGAGGACATCTT |
| | sequence | CAGCTACCTGGCCTGGTACCAGCAGAAGCCCGGCAAGGCCCCCA |
| | Sequence Name: | AGCTGCTGATCTACGGCGCCAACAGGCTGAAGGACACCGTGCCC |
| | DAB012135 | AGCAGGTTCAGCGGCGGCGGCAGCGGCACCGAGTACAGCCTGAC |
| | (modified) | CATCAGCGGCCTGCAGCCCGAGGACTTCGGCACCTACTACTGCCT |
| | | GCAGGGCGCCAAGTTCCCCCTGACCTTCGGCCAGGGCACCAAGCT |
| | | GGACATGAAGCGAACGGTGGCTGCCCCCTCCGTGTTCATCTTCCC |
| | | CCCCAGCGATGAGCAGCTGAAGAGCGGCACAGCCAGCGTGGTGT |
| | | GCCTGCTGAACAACTTCTACCCCAGGGAGGCCAAGGTGCAGTGG |
| | | AAGGTGGACAACGCCCTGCAGAGCGGCAACAGCCAGGAGTCCGT |
| | | GACCGAGCAGGACAGCAAGGACAGCACCTACAGCCTGAGCAGCA |
| | | CACTGACCCTGAGCAAGGCCGACTACGAGAAGCACAAGGTGTAC |
| | | GCCTGCGAGGTGACCCATCAGGGCCTGAGCAGCCCCGTGACCAA |
| | | GAGCTTCAACAGGGGCGAGTGC |
| 177 | Full-length heavy | CAGGTGCAGCTGCTGGAGAGCGGCGGCGAGCTGGTGCAGCCCGG |
| | chain nucleotide | CGGCAGCCTGAGGCTGAGCTGCGCCGCCAGCGGCTTCACCTTCAG |
| | sequence | CGACTACTACATGTTCTGGATCAGGCAGACCCCCCAGAAGAGGCT |
| | | GGAGTGGGTGGCCTACATCAGCAACGGCGGCGGCAACACCTACT |
| | Sequence Name: | ACCCCGACACCGTGAAGGGCAGGTTCACCATCAGCAGGGACAAC |
| | DAB012120 | GCCAAGAACCAGCTGTACCTGCAGATGAGGAGCCTGACCCCCGA |
| | | GGACACCGCCATCTACTACTGCGCCAGCCCCGAGGCCAGGTACTA |
| | | CGGCAACTACCCCTTCCCCTACTGGGGCCAGGGCACCCTGGTGAC |
| | | CGTGAGCAGCGCTAGCACCAAGGGCCCATCGGTCTTCCCCCTGGC |
| | | ACCCTCCTCCAAGAGCACCTCTGGGGGCACAGCGGCCCTGGGCTG |
| | | CCTGGTCAAGGACTACTTCCCCGAACCGGTGACGGTGTCGTGGAA |
| | | CTCAGGCGCCCTGACCAGCGGCGTGCACACCTTCCCGGCTGTCCT |
| | | ACAGTCCTCAGGACTCTACTCCCTCAGCAGCGTGGTGACCGTGCC |
| | | CTCCAGCAGCTTGGGCACCCAGACCTACATCTGCAACGTGAATCA |
| | | CAAGCCCAGCAACACCAAGGTGGACAAGAAAGTTGAGCCCAAAT |
| | | CTTGTGACAAAACTCACACATGCCCACCGTGCCCAGCACCTGAAC |
| | | TCCTGGGGGGACCGTCAGTCTTCCTCTTCCCCCCAAAACCCAAGG |
| | | ACACCCTCATGATCTCCCGGACCCCTGAGGTCACATGCGTGGTGG |
| | | TGGACGTGAGCCACGAAGACCCTGAGGTCAAGTTCAACTGGTAC |
| | | GTGGACGGCGTGGAGGTGCATAATGCCAAGACAAAGCCGCGGGA |
| | | GGAGCAGTACAACAGCACGTACCGGGTGGTCAGCGTCCTCACCG |
| | | TCCTGCACCAGGACTGGCTGAATGGCAAGGAGTACAAGTGCAAG |
| | | GTCTCCAACAAAGCCCTCCCAGCCCCCATCGAGAAAACCATCTCC |
| | | AAAGCCAAAGGGCAGCCCCGAGAACCACAGGTGTACACCCTGCC |
| | | CCCATCCCGGGATGAGCTGACCAAGAACCAGGTCAGCCTGACCT |
| | | GCCTGGTCAAAGGCTTCTATCCCAGCGACATCGCCGTGGAGTGGG |
| | | AGAGCAATGGGCAGCCGGAGAACAACTACAAGACCACGCCTCCC |
| | | GTGCTGGACTCCGACGGCTCCTTCTTCCTCTACAGCAAGCTCACC |
| | | GTGGACAAGAGCAGGTGGCAGCAGGGGAACGTCTTCTCATGCTC |
| | | CGTGATGCATGAGGCTCTGCACAACCACTACACGCAGAAGAGCC |
| | | TCTCCCTGTCTCCGGGTAAA |
| 178 | Full-length heavy | CAGGTGCAGCTGGTGGAGAGCGGCGGCGGCCTGGTGAAGCCCGG |
| | chain nucleotide | CGGCAGCCTGAGGCTGAGCTGCGCCGCCAGCGGCTTCACCTTCAG |
| | sequence | CGACTACTACATGTTCTGGATCAGGCAGGCCCCCGGCAAGGGCCT |
| | Sequence Name: | GGAGTGGGTGGCCTACATCAGCAACGGCGGCGGCAACACCTACT |
| | DAB012121 | ACCCCGACACCGTGAAGGGCAGGTTCACCATCAGCAGGGACAAC |
| | | GCCAAGAACACCCTGTACCTGCAGATGAACAGCCTGAGGGCCGA |
| | | GGACACCGCCGTGTACTACTGCGCCAGCCCCGAGGCCAGGTACTA |
| | | CGGCAACTACCCCTTCCCCTACTGGGGCCAGGGCACCCTGGTGAC |
| | | CGTGAGCAGCGCTAGCACCAAGGGCCCATCGGTCTTCCCCCTGGC |
| | | ACCCTCCTCCAAGAGCACCTCTGGGGGCACAGCGGCCCTGGGCTG |
| | | CCTGGTCAAGGACTACTTCCCCGAACCGGTGACGGTGTCGTGGAA |
| | | CTCAGGCGCCCTGACCAGCGGCGTGCACACCTTCCCGGCTGTCCT |
| | | ACAGTCCTCAGGACTCTACTCCCTCAGCAGCGTGGTGACCGTGCC |
| | | CTCCAGCAGCTTGGGCACCCAGACCTACATCTGCAACGTGAATCA |
| | | CAAGCCCAGCAACACCAAGGTGGACAAGAAAGTTGAGCCCAAAT |
| | | CTTGTGACAAAACTCACACATGCCCACCGTGCCCAGCACCTGAAC |
| | | TCCTGGGGGGACCGTCAGTCTTCCTCTTCCCCCCAAAACCCAAGG |
| | | ACACCCTCATGATCTCCCGGACCCCTGAGGTCACATGCGTGGTGG |
| | | TGGACGTGAGCCACGAAGACCCTGAGGTCAAGTTCAACTGGTAC |
| | | GTGGACGGCGTGGAGGTGCATAATGCCAAGACAAAGCCGCGGGA |
| | | GGAGCAGTACAACAGCACGTACCGGGTGGTCAGCGTCCTCACCG |
| | | TCCTGCACCAGGACTGGCTGAATGGCAAGGAGTACAAGTGCAAG |
| | | GTCTCCAACAAAGCCCTCCCAGCCCCCATCGAGAAAACCATCTCC |
| | | AAAGCCAAAGGGCAGCCCCGAGAACCACAGGTGTACACCCTGCC |
| | | CCCATCCCGGGATGAGCTGACCAAGAACCAGGTCAGCCTGACCT |
| | | GCCTGGTCAAAGGCTTCTATCCCAGCGACATCGCCGTGGAGTGGG |
| | | AGAGCAATGGGCAGCCGGAGAACAACTACAAGACCACGCCTCCC |
| | | GTGCTGGACTCCGACGGCTCCTTCTTCCTCTACAGCAAGCTCACC |
| | | GTGGACAAGAGCAGGTGGCAGCAGGGGAACGTCTTCTCATGCTC |
| | | CGTGATGCATGAGGCTCTGCACAACCACTACACGCAGAAGAGCC |
| | | TCTCCCTGTCTCCGGGTAAA |
| 179 | Full-length heavy | CAGGTGCAGCTGGTGGAGAGCGGCGGCGGCGTGGTGAAGCCCGG |
| | chain nucleotide | CGGCAGCCTGAGGCTGAGCTGCGCCGCCAGCGGCTTCACCTTCAG |
| | sequence | CGACTACTACATGTTCTGGATCAGGCAGGCCCCCGGCAAGGGCCT |
| | Sequence Name: | GGAGTGGGTGGCCTACATCAGCAACGGCGGCGGCAACACCTACT |
| | DAB012122 | ACCCCGACACCGTGAAGGGCAGGTTCACCATCAGCAGGGACAAC |
| | | GCCAAGAACACCCTGTACCTGCAGATGAACAGCCTGAGGGCCGA |
| | | GGACACCGCCGTGTACTACTGCGCCAGCCCCGAGGCCAGGTACTA |
| | | CGGCAACTACCCCTTCCCCTACTGGGGCCAGGGCACCCTGGTGAC |
| | | CGTGAGCAGCGCTAGCACCAAGGGCCCATCGGTCTTCCCCCTGGC |
| | | ACCCTCCTCCAAGAGCACCTCTGGGGGCACAGCGGCCCTGGGCTG |
| | | CCTGGTCAAGGACTACTTCCCCGAACCGGTGACGGTGTCGTGGAA |
| | | CTCAGGCGCCCTGACCAGCGGCGTGCACACCTTCCCGGCTGTCCT |
| | | ACAGTCCTCAGGACTCTACTCCCTCAGCAGCGTGGTGACCGTGCC |
| | | CTCCAGCAGCTTGGGCACCCAGACCTACATCTGCAACGTGAATCA |
| | | CAAGCCCAGCAACACCAAGGTGGACAAGAAAGTTGAGCCCAAAT |
| | | CTTGTGACAAAACTCACACATGCCCACCGTGCCCAGCACCTGAAC |
| | | TCCTGGGGGGACCGTCAGTCTTCCTCTTCCCCCCAAAACCCAAGG |
| | | ACACCCTCATGATCTCCCGGACCCCTGAGGTCACATGCGTGGTGG |
| | | TGGACGTGAGCCACGAAGACCCTGAGGTCAAGTTCAACTGGTAC |
| | | GTGGACGGCGTGGAGGTGCATAATGCCAAGACAAAGCCGCGGGA |
| | | GGAGCAGTACAACAGCACGTACCGGGTGGTCAGCGTCCTCACCG |
| | | TCCTGCACCAGGACTGGCTGAATGGCAAGGAGTACAAGTGCAAG |
| | | GTCTCCAACAAAGCCCTCCCAGCCCCCATCGAGAAAACCATCTCC |
| | | AAAGCCAAAGGGCAGCCCCGAGAACCACAGGTGTACACCCTGCC |
| | | CCCATCCCGGGATGAGCTGACCAAGAACCAGGTCAGCCTGACCT |
| | | GCCTGGTCAAAGGCTTCTATCCCAGCGACATCGCCGTGGAGTGGG |
| | | AGAGCAATGGGCAGCCGGAGAACAACTACAAGACCACGCCTCCC |
| | | GTGCTGGACTCCGACGGCTCCTTCTTCCTCTACAGCAAGCTCACC |
| | | GTGGACAAGAGCAGGTGGCAGCAGGGGAACGTCTTCTCATGCTC |
| | | CGTGATGCATGAGGCTCTGCACAACCACTACACGCAGAAGAGCC |
| | | TCTCCCTGTCTCCGGGTAAA |
| 180 | Full-length heavy | CAGGTGCAGCTGGTGGAGAGCGGCGGCGGCGTGGTGCAGCCCGG |
| | chain nucleotide | CGGCAGCCTGAGGCTGAGCTGCGCCGCCAGCGGCTTCACCTTCAG |
| | sequence | CGACTACTACATGTTCTGGATCAGGCAGGCCCCCGGCAAGGGCCT |
| | Sequence Name: | GGAGTGGGTGGCCTACATCAGCAACGGCGGCGGCAACACCTACT |
| | DAB012123 | ACCCCGACACCGTGAAGGGCAGGTTCACCATCAGCAGGGACAAC |
| | | GCCAAGAACACCCTGTACCTGCAGATGAACAGCCTGAGGGCCGA |
| | | GGACACCGCCGTGTACTACTGCGCCAGCCCCGAGGCCAGGTACTA |
| | | CGGCAACTACCCCTTCCCCTACTGGGGCCAGGGCACCCTGGTGAC |
| | | CGTGAGCAGCGCTAGCACCAAGGGCCCATCGGTCTTCCCCCTGGC |
| | | ACCCTCCTCCAAGAGCACCTCTGGGGGCACAGCGGCCCTGGGCTG |
| | | CCTGGTCAAGGACTACTTCCCCGAACCGGTGACGGTGTCGTGGAA |
| | | CTCAGGCGCCCTGACCAGCGGCGTGCACACCTTCCCGGCTGTCCT |
| | | ACAGTCCTCAGGACTCTACTCCCTCAGCAGCGTGGTGACCGTGCC |
| | | CTCCAGCAGCTTGGGCACCCAGACCTACATCTGCAACGTGAATCA |
| | | CAAGCCCAGCAACACCAAGGTGGACAAGAAAGTTGAGCCCAAAT |
| | | CTTGTGACAAAACTCACACATGCCCACCGTGCCCAGCACCTGAAC |
| | | TCCTGGGGGGACCGTCAGTCTTCCTCTTCCCCCCAAAACCCAAGG |
| | | ACACCCTCATGATCTCCCGGACCCCTGAGGTCACATGCGTGGTGG |
| | | TGGACGTGAGCCACGAAGACCCTGAGGTCAAGTTCAACTGGTAC |
| | | GTGGACGGCGTGGAGGTGCATAATGCCAAGACAAAGCCGCGGGA |
| | | GGAGCAGTACAACAGCACGTACCGGGTGGTCAGCGTCCTCACCG |
| | | TCCTGCACCAGGACTGGCTGAATGGCAAGGAGTACAAGTGCAAG |
| | | GTCTCCAACAAAGCCCTCCCAGCCCCCATCGAGAAAACCATCTCC |
| | | AAAGCCAAAGGGCAGCCCCGAGAACCACAGGTGTACACCCTGCC |
| | | CCCATCCCGGGATGAGCTGACCAAGAACCAGGTCAGCCTGACCT |
| | | GCCTGGTCAAAGGCTTCTATCCCAGCGACATCGCCGTGGAGTGGG |
| | | AGAGCAATGGGCAGCCGGAGAACAACTACAAGACCACGCCTCCC |
| | | GTGCTGGACTCCGACGGCTCCTTCTTCCTCTACAGCAAGCTCACC |
| | | GTGGACAAGAGCAGGTGGCAGCAGGGGAACGTCTTCTCATGCTC |
| | | CGTGATGCATGAGGCTCTGCACAACCACTACACGCAGAAGAGCC |
| | | TCTCCCTGTCTCCGGGTAAA |
| 181 | Full-length heavy | CAGGTGCAGCTGCTGCAGAGCGGCCCCGGCCTGGTGAAGCCCAG |
| | chain nucleotide | CGCCACCCTGAGCCTGACCTGCACCGTGAGCGGCTTCAGCCTGAC |
| | sequence | CACCTACGGCGTGCACTGGATCAGGCAGCCCCCCGGCAAGGGCC |
| | Sequence Name: | TGGAGTGGATGGGCGTGATCTGGAGCGGCGGCAGCACCGACTAC |
| | DAB012124 | AACGCCGCCTTCATCAGCAGGCTGACCATCAGCAAGGACAACAG |
| | | CAAGAACCAGGTGTTCCTGCAGCTGTACAGCCTGAGGGCCGAGG |
| | | ACACCGCCATCTACTACTGCGCCAGGACCAGCCACTGGTACTTCG |
| | | ACGTGTGGGGCAGGGGCACCCTGGTGACCGTGAGCAGCGCTAGC |
| | | ACCAAGGGCCCATCGGTCTTCCCCCTGGCACCCTCCTCCAAGAGC |
| | | ACCTCTGGGGGCACAGCGGCCCTGGGCTGCCTGGTCAAGGACTAC |
| | | TTCCCCGAACCGGTGACGGTGTCGTGGAACTCAGGCGCCCTGACC |
| | | AGCGGCGTGCACACCTTCCCGGCTGTCCTACAGTCCTCAGGACTC |
| | | TACTCCCTCAGCAGCGTGGTGACCGTGCCCTCCAGCAGCTTGGGC |
| | | ACCCAGACCTACATCTGCAACGTGAATCACAAGCCCAGCAACAC |
| | | CAAGGTGGACAAGAAAGTTGAGCCCAAATCTTGTGACAAAACTC |
| | | ACACATGCCCACCGTGCCCAGCACCTGAACTCCTGGGGGGACCGT |
| | | CAGTCTTCCTCTTCCCCCCAAAACCCAAGGACACCCTCATGATCT |
| | | CCCGGACCCCTGAGGTCACATGCGTGGTGGTGGACGTGAGCCAC |
| | | GAAGACCCTGAGGTCAAGTTCAACTGGTACGTGGACGGCGTGGA |
| | | GGTGCATAATGCCAAGACAAAGCCGCGGGAGGAGCAGTACAACA |
| | | GCACGTACCGGGTGGTCAGCGTCCTCACCGTCCTGCACCAGGACT |
| | | GGCTGAATGGCAAGGAGTACAAGTGCAAGGTCTCCAACAAAGCC |
| | | CTCCCAGCCCCCATCGAGAAAACCATCTCCAAAGCCAAAGGGCA |
| | | GCCCCGAGAACCACAGGTGTACACCCTGCCCCCATCCCGGGATGA |
| | | GCTGACCAAGAACCAGGTCAGCCTGACCTGCCTGGTCAAAGGCTT |
| | | CTATCCCAGCGACATCGCCGTGGAGTGGGAGAGCAATGGGCAGC |
| | | CGGAGAACAACTACAAGACCACGCCTCCCGTGCTGGACTCCGAC |
| | | GGCTCCTTCTTCCTCTACAGCAAGCTCACCGTGGACAAGAGCAGG |
| | | TGGCAGCAGGGGAACGTCTTCTCATGCTCCGTGATGCATGAGGCT |
| | | CTGCACAACCACTACACGCAGAAGAGCCTCTCCCTGTCTCCGGGT |
| | | AAA |
| 182 | Full-length heavy | CAGGTGCAGCTGCAGGAGAGCGGCCCCGGCCTGGTGAAGCCCAG |
| | chain nucleotide | CCAGACCCTGAGCCTGACCTGCACCGTGAGCGGCTTCAGCCTGAC |
| | sequence | CACCTACGGCGTGCACTGGGTGAGGCAGAGCCCCGGCAAGGGCC |
| | Sequence Name: | TGGAGTGGCTGGGCGTGATCTGGAGCGGCGGCAGCACCGACTAC |
| | DAB012125 | AACGCCGCCTTCATCAGCAGGCTGAGCATCAGCAAGGACAACAG |
| | | CAAGAGCCAGGTGTTCCTGGAGATGACCAGCCTGACCGCCGCCG |
| | | ACACCGCCATCTACTACTGCGCCAGGACCAGCCACTGGTACTTCG |
| | | ACGTGTGGGGCAGGGGCACCCTGGTGACCGTGAGCAGCGCTAGC |
| | | ACCAAGGGCCCATCGGTCTTCCCCCTGGCACCCTCCTCCAAGAGC |
| | | ACCTCTGGGGGCACAGCGGCCCTGGGCTGCCTGGTCAAGGACTAC |
| | | TTCCCCGAACCGGTGACGGTGTCGTGGAACTCAGGCGCCCTGACC |
| | | AGCGGCGTGCACACCTTCCCGGCTGTCCTACAGTCCTCAGGACTC |
| | | TACTCCCTCAGCAGCGTGGTGACCGTGCCCTCCAGCAGCTTGGGC |
| | | ACCCAGACCTACATCTGCAACGTGAATCACAAGCCCAGCAACAC |
| | | CAAGGTGGACAAGAAAGTTGAGCCCAAATCTTGTGACAAAACTC |
| | | ACACATGCCCACCGTGCCCAGCACCTGAACTCCTGGGGGGACCGT |
| | | CAGTCTTCCTCTTCCCCCCAAAACCCAAGGACACCCTCATGATCT |
| | | CCCGGACCCCTGAGGTCACATGCGTGGTGGTGGACGTGAGCCAC |
| | | GAAGACCCTGAGGTCAAGTTCAACTGGTACGTGGACGGCGTGGA |
| | | GGTGCATAATGCCAAGACAAAGCCGCGGGAGGAGCAGTACAACA |
| | | GCACGTACCGGGTGGTCAGCGTCCTCACCGTCCTGCACCAGGACT |
| | | GGCTGAATGGCAAGGAGTACAAGTGCAAGGTCTCCAACAAAGCC |
| | | CTCCCAGCCCCCATCGAGAAAACCATCTCCAAAGCCAAAGGGCA |
| | | GCCCCGAGAACCACAGGTGTACACCCTGCCCCCATCCCGGGATGA |
| | | GCTGACCAAGAACCAGGTCAGCCTGACCTGCCTGGTCAAAGGCTT |
| | | CTATCCCAGCGACATCGCCGTGGAGTGGGAGAGCAATGGGCAGC |
| | | CGGAGAACAACTACAAGACCACGCCTCCCGTGCTGGACTCCGAC |
| | | GGCTCCTTCTTCCTCTACAGCAAGCTCACCGTGGACAAGAGCAGG |
| | | TGGCAGCAGGGGAACGTCTTCTCATGCTCCGTGATGCATGAGGCT |
| | | CTGCACAACCACTACACGCAGAAGAGCCTCTCCCTGTCTCCGGGT |
| | | AAA |
| 183 | Full-length heavy | CAGGTGCAGCTGCAGGAGAGCGGCCCCGGCCTGGTGAAGCCCAG |
| | chain nucleotide | CCAGACCCTGAGCCTGACCTGCACCGTGAGCGGCTTCAGCCTGAC |
| | sequence | CACCTACGGCGTGCACTGGGTGAGGCAGGCCCCCGAGAAGGGCC |
| | Sequence Name: | TGGAGTGGCTGGGCGTGATCTGGAGCGGCGGCAGCACCGACTAC |
| | DAB012126 | AACGCCGCCTTCATCAGCAGGCTGAGCATCAGCAAGGACAACAG |
| | | CAAGAGCCAGGTGTTCCTGGAGATGACCAGCCTGACCGCCGACG |
| | | ACACCGCCATCTACTACTGCGCCAGGACCAGCCACTGGTACTTCG |
| | | ACGTGTGGGGCAGGGGCACCCTGGTGACCGTGAGCAGCGCTAGC |
| | | ACCAAGGGCCCATCGGTCTTCCCCCTGGCACCCTCCTCCAAGAGC |
| | | ACCTCTGGGGGCACAGCGGCCCTGGGCTGCCTGGTCAAGGACTAC |
| | | TTCCCCGAACCGGTGACGGTGTCGTGGAACTCAGGCGCCCTGACC |
| | | AGCGGCGTGCACACCTTCCCGGCTGTCCTACAGTCCTCAGGACTC |
| | | TACTCCCTCAGCAGCGTGGTGACCGTGCCCTCCAGCAGCTTGGGC |
| | | ACCCAGACCTACATCTGCAACGTGAATCACAAGCCCAGCAACAC |
| | | CAAGGTGGACAAGAAAGTTGAGCCCAAATCTTGTGACAAAACTC |
| | | ACACATGCCCACCGTGCCCAGCACCTGAACTCCTGGGGGGACCGT |
| | | CAGTCTTCCTCTTCCCCCCAAAACCCAAGGACACCCTCATGATCT |
| | | CCCGGACCCCTGAGGTCACATGCGTGGTGGTGGACGTGAGCCAC |
| | | GAAGACCCTGAGGTCAAGTTCAACTGGTACGTGGACGGCGTGGA |
| | | GGTGCATAATGCCAAGACAAAGCCGCGGGAGGAGCAGTACAACA |
| | | GCACGTACCGGGTGGTCAGCGTCCTCACCGTCCTGCACCAGGACT |
| | | GGCTGAATGGCAAGGAGTACAAGTGCAAGGTCTCCAACAAAGCC |
| | | CTCCCAGCCCCCATCGAGAAAACCATCTCCAAAGCCAAAGGGCA |
| | | GCCCCGAGAACCACAGGTGTACACCCTGCCCCCATCCCGGGATGA |
| | | GCTGACCAAGAACCAGGTCAGCCTGACCTGCCTGGTCAAAGGCTT |
| | | CTATCCCAGCGACATCGCCGTGGAGTGGGAGAGCAATGGGCAGC |
| | | CGGAGAACAACTACAAGACCACGCCTCCCGTGCTGGACTCCGAC |
| | | GGCTCCTTCTTCCTCTACAGCAAGCTCACCGTGGACAAGAGCAGG |
| | | TGGCAGCAGGGGAACGTCTTCTCATGCTCCGTGATGCATGAGGCT |
| | | CTGCACAACCACTACACGCAGAAGAGCCTCTCCCTGTCTCCGGGT |
| | | AAA |
| 184 | Full-length heavy | CAGGTGCAGCTGCAGGAGAGCGGCCCCGGCCTGGTGAAGCCCAG |
| | chain nucleotide | CCAGACCCTGAGCCTGACCTGCACCGTGAGCGGCTTCAGCCTGAC |
| | sequence | CACCTACGGCGTGCACTGGGTGAGGCAGACCCCCGAGAAGGGCC |
| | Sequence Name: | TGGAGTGGCTGGGCGTGATCTGGAGCGGCGGCAGCACCGACTAC |
| | DAB012127 | AACGCCGCCTTCATCAGCAGGCTGAGCATCAGCAAGGACAACAG |
| | | CAAGAGCCAGGTGTTCCTGGAGATGACCAGCCTGACCGCCGACG |
| | | ACACCGCCATCTACTACTGCGCCAGGACCAGCCACTGGTACTTCG |
| | | ACGTGTGGGGCAGGGGCACCCTGGTGACCGTGAGCAGCGCTAGC |
| | | ACCAAGGGCCCATCGGTCTTCCCCCTGGCACCCTCCTCCAAGAGC |
| | | ACCTCTGGGGGCACAGCGGCCCTGGGCTGCCTGGTCAAGGACTAC |
| | | TTCCCCGAACCGGTGACGGTGTCGTGGAACTCAGGCGCCCTGACC |
| | | AGCGGCGTGCACACCTTCCCGGCTGTCCTACAGTCCTCAGGACTC |
| | | TACTCCCTCAGCAGCGTGGTGACCGTGCCCTCCAGCAGCTTGGGC |
| | | ACCCAGACCTACATCTGCAACGTGAATCACAAGCCCAGCAACAC |
| | | CAAGGTGGACAAGAAAGTTGAGCCCAAATCTTGTGACAAAACTC |
| | | ACACATGCCCACCGTGCCCAGCACCTGAACTCCTGGGGGGACCGT |
| | | CAGTCTTCCTCTTCCCCCCAAAACCCAAGGACACCCTCATGATCT |
| | | CCCGGACCCCTGAGGTCACATGCGTGGTGGTGGACGTGAGCCAC |
| | | GAAGACCCTGAGGTCAAGTTCAACTGGTACGTGGACGGCGTGGA |
| | | GGTGCATAATGCCAAGACAAAGCCGCGGGAGGAGCAGTACAACA |
| | | GCACGTACCGGGTGGTCAGCGTCCTCACCGTCCTGCACCAGGACT |
| | | GGCTGAATGGCAAGGAGTACAAGTGCAAGGTCTCCAACAAAGCC |
| | | CTCCCAGCCCCCATCGAGAAAACCATCTCCAAAGCCAAAGGGCA |
| | | GCCCCGAGAACCACAGGTGTACACCCTGCCCCCATCCCGGGATGA |
| | | GCTGACCAAGAACCAGGTCAGCCTGACCTGCCTGGTCAAAGGCTT |
| | | CTATCCCAGCGACATCGCCGTGGAGTGGGAGAGCAATGGGCAGC |
| | | CGGAGAACAACTACAAGACCACGCCTCCCGTGCTGGACTCCGAC |
| | | GGCTCCTTCTTCCTCTACAGCAAGCTCACCGTGGACAAGAGCAGG |
| | | TGGCAGCAGGGGAACGTCTTCTCATGCTCCGTGATGCATGAGGCT |
| | | CTGCACAACCACTACACGCAGAAGAGCCTCTCCCTGTCTCCGGGT |
| | | AAA |
| 185 | Full-length heavy | CAGGTGCAGCTGCTGGAGAGCGGCGGCGGCCTGGTGCAGCCCGG |
| | chain nucleotide | CGGCAGCCTGAGGCTGAGCTGCGCCGCCAGCGGCTTCACCTTCAG |
| | sequence | CGACTACTACATGTACTGGATCAGGCAGACCCCCCAGAAGAGGC |
| | Sequence Name: | TGGAGTGGGTGGCCTACATCAGCAACGGCGGCGGCAACACCTAC |
| | DAB012128 | TACAGCGACACCGTGAAGGGCAGGTTCACCATCAGCAGGGACAA |
| | | CGCCAAGAACCAGCTGTACCTGCAGATGAGGAGCCTGAGGCCCG |
| | | AGGACACCGCCATCTACTACTGCGCCAGCCCCGAGGCCAGGTACT |
| | | ACGGCAACTTCCCCTTCCCCTACTGGGGCCAGGGCACCCTGGTGA |
| | | CCGTGAGCAGCGCTAGCACCAAGGGCCCATCGGTCTTCCCCCTGG |
| | | CACCCTCCTCCAAGAGCACCTCTGGGGGCACAGCGGCCCTGGGCT |
| | | GCCTGGTCAAGGACTACTTCCCCGAACCGGTGACGGTGTCGTGGA |
| | | ACTCAGGCGCCCTGACCAGCGGCGTGCACACCTTCCCGGCTGTCC |
| | | TACAGTCCTCAGGACTCTACTCCCTCAGCAGCGTGGTGACCGTGC |
| | | CCTCCAGCAGCTTGGGCACCCAGACCTACATCTGCAACGTGAATC |
| | | ACAAGCCCAGCAACACCAAGGTGGACAAGAAAGTTGAGCCCAAA |
| | | TCTTGTGACAAAACTCACACATGCCCACCGTGCCCAGCACCTGAA |
| | | CTCCTGGGGGGACCGTCAGTCTTCCTCTTCCCCCCAAAACCCAAG |
| | | GACACCCTCATGATCTCCCGGACCCCTGAGGTCACATGCGTGGTG |
| | | GTGGACGTGAGCCACGAAGACCCTGAGGTCAAGTTCAACTGGTA |
| | | CGTGGACGGCGTGGAGGTGCATAATGCCAAGACAAAGCCGCGGG |
| | | AGGAGCAGTACAACAGCACGTACCGGGTGGTCAGCGTCCTCACC |
| | | GTCCTGCACCAGGACTGGCTGAATGGCAAGGAGTACAAGTGCAA |
| | | GGTCTCCAACAAAGCCCTCCCAGCCCCCATCGAGAAAACCATCTC |
| | | CAAAGCCAAAGGGCAGCCCCGAGAACCACAGGTGTACACCCTGC |
| | | CCCCATCCCGGGATGAGCTGACCAAGAACCAGGTCAGCCTGACCT |
| | | GCCTGGTCAAAGGCTTCTATCCCAGCGACATCGCCGTGGAGTGGG |
| | | AGAGCAATGGGCAGCCGGAGAACAACTACAAGACCACGCCTCCC |
| | | GTGCTGGACTCCGACGGCTCCTTCTTCCTCTACAGCAAGCTCACC |
| | | GTGGACAAGAGCAGGTGGCAGCAGGGGAACGTCTTCTCATGCTC |
| | | CGTGATGCATGAGGCTCTGCACAACCACTACACGCAGAAGAGCC |
| | | TCTCCCTGTCTCCGGGTAAA |
| 186 | Full-length heavy | CAGGTGCAGCTGGTGGAGAGCGGCGGCGGCCTGGTGAAGCCCGG |
| | chain nucleotide | CGGCAGCCTGAGGCTGAGCTGCGCCGCCAGCGGCTTCACCTTCAG |
| | sequence | CGACTACTACATGTACTGGATCAGGCAGGCCCCCGGCAAGGGCCT |
| | Sequence Name: | GGAGTGGGTGGCCTACATCAGCAACGGCGGCGGCAACACCTACT |
| | DAB012129 | ACAGCGACACCGTGAAGGGCAGGTTCACCATCAGCAGGGACAAC |
| | | GCCAAGAACACCCTGTACCTGCAGATGAACAGCCTGAGGGCCGA |
| | | GGACACCGCCGTGTACTACTGCGCCAGCCCCGAGGCCAGGTACTA |
| | | CGGCAACTTCCCCTTCCCCTACTGGGGCCAGGGCACCCTGGTGAC |
| | | CGTGAGCAGCGCTAGCACCAAGGGCCCATCGGTCTTCCCCCTGGC |
| | | ACCCTCCTCCAAGAGCACCTCTGGGGGCACAGCGGCCCTGGGCTG |
| | | CCTGGTCAAGGACTACTTCCCCGAACCGGTGACGGTGTCGTGGAA |
| | | CTCAGGCGCCCTGACCAGCGGCGTGCACACCTTCCCGGCTGTCCT |
| | | ACAGTCCTCAGGACTCTACTCCCTCAGCAGCGTGGTGACCGTGCC |
| | | CTCCAGCAGCTTGGGCACCCAGACCTACATCTGCAACGTGAATCA |
| | | CAAGCCCAGCAACACCAAGGTGGACAAGAAAGTTGAGCCCAAAT |
| | | CTTGTGACAAAACTCACACATGCCCACCGTGCCCAGCACCTGAAC |
| | | TCCTGGGGGGACCGTCAGTCTTCCTCTTCCCCCCAAAACCCAAGG |
| | | ACACCCTCATGATCTCCCGGACCCCTGAGGTCACATGCGTGGTGG |
| | | TGGACGTGAGCCACGAAGACCCTGAGGTCAAGTTCAACTGGTAC |
| | | GTGGACGGCGTGGAGGTGCATAATGCCAAGACAAAGCCGCGGGA |
| | | GGAGCAGTACAACAGCACGTACCGGGTGGTCAGCGTCCTCACCG |
| | | TCCTGCACCAGGACTGGCTGAATGGCAAGGAGTACAAGTGCAAG |
| | | GTCTCCAACAAAGCCCTCCCAGCCCCCATCGAGAAAACCATCTCC |
| | | AAAGCCAAAGGGCAGCCCCGAGAACCACAGGTGTACACCCTGCC |
| | | CCCATCCCGGGATGAGCTGACCAAGAACCAGGTCAGCCTGACCT |
| | | GCCTGGTCAAAGGCTTCTATCCCAGCGACATCGCCGTGGAGTGGG |
| | | AGAGCAATGGGCAGCCGGAGAACAACTACAAGACCACGCCTCCC |
| | | GTGCTGGACTCCGACGGCTCCTTCTTCCTCTACAGCAAGCTCACC |
| | | GTGGACAAGAGCAGGTGGCAGCAGGGGAACGTCTTCTCATGCTC |
| | | CGTGATGCATGAGGCTCTGCACAACCACTACACGCAGAAGAGCC |
| | | TCTCCCTGTCTCCGGGTAAA |
| 187 | Full-length heavy | CAGGTGCAGCTGGTGGAGAGCGGCGGCGGCCTGGTGAAGCCCGG |
| | chain nucleotide | CGGCAGCCTGAGGCTGAGCTGCGCCGCCAGCGGCTTCACCTTCAG |
| | sequence | CGACTACTACATGTACTGGATCAGGCAGGCCCCCGGCAAGGGCCT |
| | Sequence Name: | GGAGTGGGTGGCCTACATCAGCAACGGCGGCGGCAACACCTACT |
| | DAB012130 | ACAGCGACACCGTGAAGGGCAGGTTCACCATCAGCAGGGACAAC |
| | | GCCAAGAACACCCTGTACCTGCAGATGAACAGCCTGAGGGCCGA |
| | | GGACACCGCCGTGTACTACTGCGCCAGCCCCGAGGCCAGGTACTA |
| | | CGGCAACTTCCCCTTCCCCTACTGGGGCCAGGGCACCCTGGTGAC |
| | | CGTGAGCAGCGCTAGCACCAAGGGCCCATCGGTCTTCCCCCTGGC |
| | | ACCCTCCTCCAAGAGCACCTCTGGGGGCACAGCGGCCCTGGGCTG |
| | | CCTGGTCAAGGACTACTTCCCCGAACCGGTGACGGTGTCGTGGAA |
| | | CTCAGGCGCCCTGACCAGCGGCGTGCACACCTTCCCGGCTGTCCT |
| | | ACAGTCCTCAGGACTCTACTCCCTCAGCAGCGTGGTGACCGTGCC |
| | | CTCCAGCAGCTTGGGCACCCAGACCTACATCTGCAACGTGAATCA |
| | | CAAGCCCAGCAACACCAAGGTGGACAAGAAAGTTGAGCCCAAAT |
| | | CTTGTGACAAAACTCACACATGCCCACCGTGCCCAGCACCTGAAC |
| | | TCCTGGGGGGACCGTCAGTCTTCCTCTTCCCCCCAAAACCCAAGG |
| | | ACACCCTCATGATCTCCCGGACCCCTGAGGTCACATGCGTGGTGG |
| | | TGGACGTGAGCCACGAAGACCCTGAGGTCAAGTTCAACTGGTAC |
| | | GTGGACGGCGTGGAGGTGCATAATGCCAAGACAAAGCCGCGGGA |
| | | GGAGCAGTACAACAGCACGTACCGGGTGGTCAGCGTCCTCACCG |
| | | TCCTGCACCAGGACTGGCTGAATGGCAAGGAGTACAAGTGCAAG |
| | | GTCTCCAACAAAGCCCTCCCAGCCCCCATCGAGAAAACCATCTCC |
| | | AAAGCCAAAGGGCAGCCCCGAGAACCACAGGTGTACACCCTGCC |
| | | CCCATCCCGGGATGAGCTGACCAAGAACCAGGTCAGCCTGACCT |
| | | GCCTGGTCAAAGGCTTCTATCCCAGCGACATCGCCGTGGAGTGGG |
| | | AGAGCAATGGGCAGCCGGAGAACAACTACAAGACCACGCCTCCC |
| | | GTGCTGGACTCCGACGGCTCCTTCTTCCTCTACAGCAAGCTCACC |
| | | GTGGACAAGAGCAGGTGGCAGCAGGGGAACGTCTTCTCATGCTC |
| | | CGTGATGCATGAGGCTCTGCACAACCACTACACGCAGAAGAGCC |
| | | TCTCCCTGTCTCCGGGTAAA |
| 188 | Full-length heavy | CAGGTGCAGCTGGTGGAGAGCGGCGGCGGCCTGGTGAAGCCCGG |
| | chain nucleotide | CGGCAGCCTGAGGCTGAGCTGCGCCGCCAGCGGCTTCACCTTCAG |
| | sequence | CGACTACTACATGTACTGGATCAGGCAGGCCCCCGGCAAGGGCCT |
| | Sequence Name: | GGAGTGGGTGGCCTACATCAGCAACGGCGGCGGCAACACCTACT |
| | DAB012131 | ACAGCGACACCGTGAAGGGCAGGTTCACCATCAGCAGGGACAAC |
| | | GCCAAGAACACCCTGTACCTGCAGATGAACAGCCTGAGGGCCGA |
| | | GGACACCGCCGTGTACTACTGCGCCAGCCCCGAGGCCAGGTACTA |
| | | CGGCAACTTCCCCTTCCCCTACTGGGGCCAGGGCACCCTGGTGAC |
| | | CGTGAGCAGCGCTAGCACCAAGGGCCCATCGGTCTTCCCCCTGGC |
| | | ACCCTCCTCCAAGAGCACCTCTGGGGGCACAGCGGCCCTGGGCTG |
| | | CCTGGTCAAGGACTACTTCCCCGAACCGGTGACGGTGTCGTGGAA |
| | | CTCAGGCGCCCTGACCAGCGGCGTGCACACCTTCCCGGCTGTCCT |
| | | ACAGTCCTCAGGACTCTACTCCCTCAGCAGCGTGGTGACCGTGCC |
| | | CTCCAGCAGCTTGGGCACCCAGACCTACATCTGCAACGTGAATCA |
| | | CAAGCCCAGCAACACCAAGGTGGACAAGAAAGTTGAGCCCAAAT |
| | | CTTGTGACAAAACTCACACATGCCCACCGTGCCCAGCACCTGAAC |
| | | TCCTGGGGGGACCGTCAGTCTTCCTCTTCCCCCCAAAACCCAAGG |
| | | ACACCCTCATGATCTCCCGGACCCCTGAGGTCACATGCGTGGTGG |
| | | TGGACGTGAGCCACGAAGACCCTGAGGTCAAGTTCAACTGGTAC |
| | | GTGGACGGCGTGGAGGTGCATAATGCCAAGACAAAGCCGCGGGA |
| | | GGAGCAGTACAACAGCACGTACCGGGTGGTCAGCGTCCTCACCG |
| | | TCCTGCACCAGGACTGGCTGAATGGCAAGGAGTACAAGTGCAAG |
| | | GTCTCCAACAAAGCCCTCCCAGCCCCCATCGAGAAAACCATCTCC |
| | | AAAGCCAAAGGGCAGCCCCGAGAACCACAGGTGTACACCCTGCC |
| | | CCCATCCCGGGATGAGCTGACCAAGAACCAGGTCAGCCTGACCT |
| | | GCCTGGTCAAAGGCTTCTATCCCAGCGACATCGCCGTGGAGTGGG |
| | | AGAGCAATGGGCAGCCGGAGAACAACTACAAGACCACGCCTCCC |
| | | GTGCTGGACTCCGACGGCTCCTTCTTCCTCTACAGCAAGCTCACC |
| | | GTGGACAAGAGCAGGTGGCAGCAGGGGAACGTCTTCTCATGCTC |
| | | CGTGATGCATGAGGCTCTGCACAACCACTACACGCAGAAGAGCC |
| | | TCTCCCTGTCTCCGGGTAAA |
| 189 | Full-length heavy | CAGGTGCAGCTGGTGGAGAGCGGCGGCGGCGTGGTGCAGCCCGG |
| | chain nucleotide | CAGGAGCCTGAGGCTGAGCTGCGCCGCCAGCGGCTTCAGCTTCAG |
| | sequence | CAACTACTACATGGCCTGGGTGAGGCAGGCCCCCGGCAAGGGCC |
| | Sequence Name: | TGGAGTGGGTGGCCAGCATCAGCACCGGCGGCGGCAACATCTAC |
| | DAB012132 | TACAGGGACAGCGTGAAGGGCAGGTTCACCATCAGCAGGGACAA |
| | | CAGCAAGAGCACCCTGTACCTGCAGATGAGGAGCCTGAGGAGCG |
| | | AGGACACCGCCATCTACTACTGCGCCAGGCAGACCGCCTACTACG |
| | | TGATGGACGCCTGGGGCCAGGGCACCATGGTGACCGTGAGCAGC |
| | | GCTAGCACCAAGGGCCCATCGGTCTTCCCCCTGGCACCCTCCTCC |
| | | AAGAGCACCTCTGGGGGCACAGCGGCCCTGGGCTGCCTGGTCAA |
| | | GGACTACTTCCCCGAACCGGTGACGGTGTCGTGGAACTCAGGCGC |
| | | CCTGACCAGCGGCGTGCACACCTTCCCGGCTGTCCTACAGTCCTC |
| | | AGGACTCTACTCCCTCAGCAGCGTGGTGACCGTGCCCTCCAGCAG |
| | | CTTGGGCACCCAGACCTACATCTGCAACGTGAATCACAAGCCCAG |
| | | CAACACCAAGGTGGACAAGAAAGTTGAGCCCAAATCTTGTGACA |
| | | AAACTCACACATGCCCACCGTGCCCAGCACCTGAACTCCTGGGGG |
| | | GACCGTCAGTCTTCCTCTTCCCCCCAAAACCCAAGGACACCCTCA |
| | | TGATCTCCCGGACCCCTGAGGTCACATGCGTGGTGGTGGACGTGA |
| | | GCCACGAAGACCCTGAGGTCAAGTTCAACTGGTACGTGGACGGC |
| | | GTGGAGGTGCATAATGCCAAGACAAAGCCGCGGGAGGAGCAGTA |
| | | CAACAGCACGTACCGGGTGGTCAGCGTCCTCACCGTCCTGCACCA |
| | | GGACTGGCTGAATGGCAAGGAGTACAAGTGCAAGGTCTCCAACA |
| | | AAGCCCTCCCAGCCCCCATCGAGAAAACCATCTCCAAAGCCAAA |
| | | GGGCAGCCCCGAGAACCACAGGTGTACACCCTGCCCCCATCCCG |
| | | GGATGAGCTGACCAAGAACCAGGTCAGCCTGACCTGCCTGGTCA |
| | | AAGGCTTCTATCCCAGCGACATCGCCGTGGAGTGGGAGAGCAAT |
| | | GGGCAGCCGGAGAACAACTACAAGACCACGCCTCCCGTGCTGGA |
| | | CTCCGACGGCTCCTTCTTCCTCTACAGCAAGCTCACCGTGGACAA |
| | | GAGCAGGTGGCAGCAGGGGAACGTCTTCTCATGCTCCGTGATGCA |
| | | TGAGGCTCTGCACAACCACTACACGCAGAAGAGCCTCTCCCTGTC |
| | | TCCGGGTAAA |
| 190 | Full-length heavy | GAGGTGCAGCTGGTGGAGAGCGGCGGCGGCCTGGTGCAGCCCGG |
| | chain nucleotide | CGGCAGCCTGAGGCTGAGCTGCGCCGCCAGCGGCTTCAGCTTCAG |
| | sequence | CAACTACTACATGGCCTGGGTGAGGCAGGCCCCCGGCAAGGGCC |
| | Sequence Name: | TGGAGTGGGTGGCCAGCATCAGCACCGGCGGCGGCAACATCTAC |
| | DAB012133 | TACAGGGACAGCGTGAAGGGCAGGTTCACCATCAGCAGGGACAA |
| | | CGCCAAGAACAGCCTGTACCTGCAGATGAACAGCCTGAGGGCCG |
| | | AGGACACCGCCGTGTACTACTGCGCCAGGCAGACCGCCTACTACG |
| | | TGATGGACGCCTGGGGCCAGGGCACCACCGTGACCGTGAGCAGC |
| | | GCTAGCACCAAGGGCCCATCGGTCTTCCCCCTGGCACCCTCCTCC |
| | | AAGAGCACCTCTGGGGGCACAGCGGCCCTGGGCTGCCTGGTCAA |
| | | GGACTACTTCCCCGAACCGGTGACGGTGTCGTGGAACTCAGGCGC |
| | | CCTGACCAGCGGCGTGCACACCTTCCCGGCTGTCCTACAGTCCTC |
| | | AGGACTCTACTCCCTCAGCAGCGTGGTGACCGTGCCCTCCAGCAG |
| | | CTTGGGCACCCAGACCTACATCTGCAACGTGAATCACAAGCCCAG |
| | | CAACACCAAGGTGGACAAGAAAGTTGAGCCCAAATCTTGTGACA |
| | | AAACTCACACATGCCCACCGTGCCCAGCACCTGAACTCCTGGGGG |
| | | GACCGTCAGTCTTCCTCTTCCCCCCAAAACCCAAGGACACCCTCA |
| | | TGATCTCCCGGACCCCTGAGGTCACATGCGTGGTGGTGGACGTGA |
| | | GCCACGAAGACCCTGAGGTCAAGTTCAACTGGTACGTGGACGGC |
| | | GTGGAGGTGCATAATGCCAAGACAAAGCCGCGGGAGGAGCAGTA |
| | | CAACAGCACGTACCGGGTGGTCAGCGTCCTCACCGTCCTGCACCA |
| | | GGACTGGCTGAATGGCAAGGAGTACAAGTGCAAGGTCTCCAACA |
| | | AAGCCCTCCCAGCCCCCATCGAGAAAACCATCTCCAAAGCCAAA |
| | | GGGCAGCCCCGAGAACCACAGGTGTACACCCTGCCCCCATCCCG |
| | | GGATGAGCTGACCAAGAACCAGGTCAGCCTGACCTGCCTGGTCA |
| | | AAGGCTTCTATCCCAGCGACATCGCCGTGGAGTGGGAGAGCAAT |
| | | GGGCAGCCGGAGAACAACTACAAGACCACGCCTCCCGTGCTGGA |
| | | CTCCGACGGCTCCTTCTTCCTCTACAGCAAGCTCACCGTGGACAA |
| | | GAGCAGGTGGCAGCAGGGGAACGTCTTCTCATGCTCCGTGATGCA |
| | | TGAGGCTCTGCACAACCACTACACGCAGAAGAGCCTCTCCCTGTC |
| | | TCCGGGTAAA |
| 191 | Full-length heavy | GAGGTGCAGCTGGTGGAGAGCGGCGGCGGCCTGGTGCAGCCCGG |
| | chain nucleotide | CGGCAGCCTGAGGCTGAGCTGCGCCGCCAGCGGCTTCAGCTTCAG |
| | sequence | CAACTACTACATGGCCTGGGTGAGGCAGGCCCCCGGCAAGGGCC |
| | Sequence Name: | TGGAGTGGGTGGCCAGCATCAGCACCGGCGGCGGCAACATCTAC |
| | DAB012134 | TACAGGGACAGCGTGAAGGGCAGGTTCACCATCAGCAGGGACAA |
| | | CGCCAAGAACAGCCTGTACCTGCAGATGAACAGCCTGAGGGCCG |
| | | AGGACACCGCCGTGTACTACTGCGCCAGGCAGACCGCCTACTACG |
| | | TGATGGACGCCTGGGGCCAGGGCACCACCGTGACCGTGAGCAGC |
| | | GCTAGCACCAAGGGCCCATCGGTCTTCCCCCTGGCACCCTCCTCC |
| | | AAGAGCACCTCTGGGGGCACAGCGGCCCTGGGCTGCCTGGTCAA |
| | | GGACTACTTCCCCGAACCGGTGACGGTGTCGTGGAACTCAGGCGC |
| | | CCTGACCAGCGGCGTGCACACCTTCCCGGCTGTCCTACAGTCCTC |
| | | AGGACTCTACTCCCTCAGCAGCGTGGTGACCGTGCCCTCCAGCAG |
| | | CTTGGGCACCCAGACCTACATCTGCAACGTGAATCACAAGCCCAG |
| | | CAACACCAAGGTGGACAAGAAAGTTGAGCCCAAATCTTGTGACA |
| | | AAACTCACACATGCCCACCGTGCCCAGCACCTGAACTCCTGGGGG |
| | | GACCGTCAGTCTTCCTCTTCCCCCCAAAACCCAAGGACACCCTCA |
| | | TGATCTCCCGGACCCCTGAGGTCACATGCGTGGTGGTGGACGTGA |
| | | GCCACGAAGACCCTGAGGTCAAGTTCAACTGGTACGTGGACGGC |
| | | GTGGAGGTGCATAATGCCAAGACAAAGCCGCGGGAGGAGCAGTA |
| | | CAACAGCACGTACCGGGTGGTCAGCGTCCTCACCGTCCTGCACCA |
| | | GGACTGGCTGAATGGCAAGGAGTACAAGTGCAAGGTCTCCAACA |
| | | AAGCCCTCCCAGCCCCCATCGAGAAAACCATCTCCAAAGCCAAA |
| | | GGGCAGCCCCGAGAACCACAGGTGTACACCCTGCCCCCATCCCG |
| | | GGATGAGCTGACCAAGAACCAGGTCAGCCTGACCTGCCTGGTCA |
| | | AAGGCTTCTATCCCAGCGACATCGCCGTGGAGTGGGAGAGCAAT |
| | | GGGCAGCCGGAGAACAACTACAAGACCACGCCTCCCGTGCTGGA |
| | | CTCCGACGGCTCCTTCTTCCTCTACAGCAAGCTCACCGTGGACAA |
| | | GAGCAGGTGGCAGCAGGGGAACGTCTTCTCATGCTCCGTGATGCA |
| | | TGAGGCTCTGCACAACCACTACACGCAGAAGAGCCTCTCCCTGTC |
| | | TCCGGGTAAA |
| 192 | Full-length heavy | GAGGTGCAGCTGGTGGAGAGCGGCGGCGGCCTGGTGCAGCCCGG |
| | chain nucleotide | CGGCAGCCTGAGGCTGAGCTGCGCCGCCAGCGGCTTCAGCTTCAG |
| | sequence | CAACTACTACATGGCCTGGGTGAGGCAGGCCCCCGGCAAGGGCC |
| | Sequence Name: | TGGAGTGGGTGGCCAGCATCAGCACCGGCGGCGGCAACATCTAC |
| | DAB012135 | TACAGGGACAGCGTGAAGGGCAGGTTCACCATCAGCAGGGACAA |
| | | CGCCAAGAACAGCCTGTACCTGCAGATGAACAGCCTGAGGGCCG |
| | | AGGACACCGCCGTGTACTACTGCGCCAGGCAGACCGCCTACTACG |
| | | TGATGGACGCCTGGGGCCAGGGCACCACCGTGACCGTGAGCAGC |
| | | GCTAGCACCAAGGGCCCATCGGTCTTCCCCCTGGCACCCTCCTCC |
| | | AAGAGCACCTCTGGGGGCACAGCGGCCCTGGGCTGCCTGGTCAA |
| | | GGACTACTTCCCCGAACCGGTGACGGTGTCGTGGAACTCAGGCGC |
| | | CCTGACCAGCGGCGTGCACACCTTCCCGGCTGTCCTACAGTCCTC |
| | | AGGACTCTACTCCCTCAGCAGCGTGGTGACCGTGCCCTCCAGCAG |
| | | CTTGGGCACCCAGACCTACATCTGCAACGTGAATCACAAGCCCAG |
| | | CAACACCAAGGTGGACAAGAAAGTTGAGCCCAAATCTTGTGACA |
| | | AAACTCACACATGCCCACCGTGCCCAGCACCTGAACTCCTGGGGG |
| | | GACCGTCAGTCTTCCTCTTCCCCCCAAAACCCAAGGACACCCTCA |
| | | TGATCTCCCGGACCCCTGAGGTCACATGCGTGGTGGTGGACGTGA |
| | | GCCACGAAGACCCTGAGGTCAAGTTCAACTGGTACGTGGACGGC |
| | | GTGGAGGTGCATAATGCCAAGACAAAGCCGCGGGAGGAGCAGTA |
| | | CAACAGCACGTACCGGGTGGTCAGCGTCCTCACCGTCCTGCACCA |
| | | GGACTGGCTGAATGGCAAGGAGTACAAGTGCAAGGTCTCCAACA |
| | | AAGCCCTCCCAGCCCCCATCGAGAAAACCATCTCCAAAGCCAAA |
| | | GGGCAGCCCCGAGAACCACAGGTGTACACCCTGCCCCCATCCCG |
| | | GGATGAGCTGACCAAGAACCAGGTCAGCCTGACCTGCCTGGTCA |
| | | AAGGCTTCTATCCCAGCGACATCGCCGTGGAGTGGGAGAGCAAT |
| | | GGGCAGCCGGAGAACAACTACAAGACCACGCCTCCCGTGCTGGA |
| | | CTCCGACGGCTCCTTCTTCCTCTACAGCAAGCTCACCGTGGACAA |
| | | GAGCAGGTGGCAGCAGGGGAACGTCTTCTCATGCTCCGTGATGCA |
| | | TGAGGCTCTGCACAACCACTACACGCAGAAGAGCCTCTCCCTGTC |
| | | TCCGGGTAAA |
| 193 | Sequence encoding | GGAGGATGGTCCTGCGCCCCCGACGAGGACACCTGGCTGTGCCCC |
| | the light | GCCGGCGGAGGAGGCTCTGGAGGACTGTCTGGCAGGTCCGACGC |
| | chain of the | CGGCTCCCCTCTGGGCCTGGCTGGCAGCGGCGGCTCTGACATCCA |
| | Nectin-4 TRACTr | GATGACACAGTCCCCATCCAGCCTGTCCGCCTCCGTGGGCGACAC |
| | molecule | CGTGACAATCACCTGTCTGGCCACCGAGGACATCTTCTCCTACCT |
| | | GGCTTGGTATCAGCAGAAGCCAGGCAAGGCCCCCAAGCTGCTGA |
| | | TCTACGGCGCTAATAGGCTGAAGGACACAGTGCCATCCCGGTTCA |
| | | GCGGAGGAGGCTCCGGCACAGAGTATTCTCTGACCATCTCCGGCC |
| | | TGCAGCCTGAGGATTTTGGCACCTACTATTGCCTGCAGGGCGCCA |
| | | AGTTCCCACTGACATTTGGCCAGGGCACCAAGCTGGACATGAAG |
| | | AGAACAGTGGCCGCTCCCTCCGTGTTCATCTTTCCCCCTAGCGAT |
| | | GAGCAGCTGAAGAGCGGCACCGCTTCTGTGGTGTGCCTGCTGAAC |
| | | AATTTCTACCCTCGCGAGGCCAAGGTGCAGTGGAAGGTGGACAA |
| | | CGCTCTGCAGTCTGGCAATTCCCAGGAGAGCGTGACAGAGCAGG |
| | | ACTCTAAGGATTCCACCTATAGCCTGTCTTCCACACTGACCCTGTC |
| | | TAAGGCCGATTACGAGAAGCACAAGGTGTATGCTTGCGAGGTGA |
| | | CACATCAGGGCCTGTCCTCCCCCGTGACCAAGTCCTTTAACAGAG |
| | | GCGAGTGT |
| 194 | Sequence encoding | GAGGTGCAGCTGGTGGAGTCCGGAGGAGGACTGGTGCAGCCTGG |
| | the heavy chain of | CGGCTCCCTGAGACTGAGCTGTGCCGCTTCTGGCTCCACATTCTA |
| | the Nectin-4 | CACCGCCGTGATGGGATGGGTGAGGCAGGCTCCAGGCAAGGGCC |
| | TRACTr molecule | TGGAGTGGGTGGCTGCTATCAGGTGGACAGCCCTGACCACATCTT |
| | | ATGCTGACTCCGTGAAGGGCAGATTCACCATCTCCCGCGATGGCG |
| | | CCAAGACCACACTGTACCTGCAGATGAACAGCCTGAGACCTGAG |
| | | GACACAGCCGTGTACTATTGCGCTGCTCGCGGCACCCTGGGACTG |
| | | TTTACCACAGCTGACTCCTACGATTATTGGGGCCAGGGCACACTG |
| | | GTGACCGTGTCCAGCGGCGGAGGAGGCAGCGGAGGAGGCTCTGG |
| | | CGGCGTGTACTGCGGCCCAGAGTTCGACGAGTCCGTGGGCTGTAT |
| | | GGGCGGCGGAGGCAGCGGAGGAGGACTGTCCGGCAGAAGCGAT |
| | | GCTGGCTCCCCACTGGGCCTGGCTGGCTCCGGAGGAGGCAGCGA |
| | | AGTCCAGCTGGTGGAGAGCGGCGGCGGCCTGGTCCAGCCTGGCG |
| | | GCTCTCTGAAGCTGTCCTGTGCCGCCTCCGGCTTCACCTTTAACAA |
| | | GTATGCCATGAATTGGGTGCGCCAGGCTCCCGGCAAGGGCCTGG |
| | | AGTGGGTAGCCAGGATCAGGTCCAAGTACAACAATTATGCCACCT |
| | | ACTACGCCGACTCCGTGAAGGATAGGTTCACAATCTCTCGGGACG |
| | | ATTCCAAGAACACCGCCTACCTGCAGATGAACAATCTGAAGACA |
| | | GAGGACACCGCCGTGTACTATTGCGTGAGGCACGGCAACTTTGGC |
| | | AATTCTTACATCTCCTATTGGGCTTACTGGGGTCAGGGCACACTG |
| | | GTCACCGTGTCTTCCGGAGGAGGAGGCTCCGGCGGCGGAGGCAG |
| | | CGGCGGCGGCGGCTCTCAGACAGTGGTGACCCAGGAGCCAAGCC |
| | | TGACCGTGTCTCCCGGCGGCACCGTGACACTGACCTGTGGCAGCT |
| | | CTACAGGAGCTGTGACCAGCGGAAACTATCCAAATTGGGTGCAG |
| | | CAGAAGCCTGGCCAGGCTCCTAGAGGCCTGATCGGAGGCACAAA |
| | | GTTCCTGGCCCCAGGCACCCCAGCTCGCTTTAGCGGCTCTCTGCT |
| | | GGGAGGCAAGGCCGCTCTGACCCTGAGCGGAGTGCAGCCAGAGG |
| | | ATGAGGCCGAGTACTATTGCGTGCTGTGGTACTCTAACAGATGGG |
| | | TGTTTGGCGGCGGCACAAAGCTGACCGTGCTGGGAGGAGGAGGC |
| | | AGCGAAGTGCAGCTGGTCGAGTCTGGCGGCGGCTTAGTCCAACCT |
| | | GGCGGCTCCCTGAGGCTGTCTTGCGCCGCTTCTGGCTTCTCCTTTA |
| | | GCAACTACTATATGGCTTGGGTGCGGCAGGCTCCTGGCAAGGGCC |
| | | TGGAGTGGGTCGCCTCTATCTCCACAGGCGGCGGCAATATCTACT |
| | | ATCGGGACTCTGTGAAGGGCAGGTTCACCATCTCCAGGGACAAC |
| | | GCTAAGAATAGCCTGTATCTGCAGATGAACTCCCTGAGGGCCGAA |
| | | GATACTGCCGTGTACTACTGCGCCCGGCAGACCGCTTACTATGTG |
| | | ATGGATGCCTGGGGCCAGGGCACCACAGTGACAGTGTCCAGCGC |
| | | CTCCACCAAGGGCCCTAGCGTGTTCCCTCTGGCTCCATCTTCCAA |
| | | GAGCACATCTGGAGGCACCGCCGCTCTGGGATGTCTGGTGAAGG |
| | | ACTACTTCCCCGAGCCTGTGACCGTGAGCTGGAACTCTGGCGCCC |
| | | TGACATCTGGCGTGCACACCTTTCCCGCTGTGCTGCAGTCCTCCG |
| | | GCCTGTATTCCCTGTCCAGCGTGGTGACAGTGCCTTCTTCCAGCCT |
| | | GGGCACACAGACCTACATCTGCAACGTGAATCATAAGCCTAGCA |
| | | ATACCAAGGTGGATAAGAAGGTGGAGCCAAAGTCTTGT |

What is claimed is:

1. An antibody or antigen binding fragment thereof that binds to Nectin-4, comprising:
(a) a heavy chain variable region comprising: a CDRH1 comprising the amino acid sequence of SEQ ID NO: 15; a CDRH2 comprising the amino acid sequence of SEQ ID NO: 16; and a CDRH3 comprising the amino acid sequence of SEQ ID NO: 17; and
a light chain variable comprising: a CDR1-L1 comprising the amino acid sequence of SEQ ID NO: 20; a CDR1-L2 comprising the amino acid sequence of SEQ ID NO: 21; and a CDR1-L3 comprising the amino acid sequence of SEQ ID NO: 22;
(b) a heavy chain variable region comprising: a CDRH1 comprising the amino acid sequence of SEQ ID NO: 52; a CDR1-H2 comprising the amino acid sequence of SEQ ID NO: 53; and a CDRH3 comprising the amino acid sequence of SEQ ID NO: 54; and
a light chain variable comprising: a CDRL1 comprising the amino acid sequence of SEQ ID NO: 28; a CDRL2 comprising the amino acid sequence of SEQ ID NO: 29; and a CDRL3 comprising the amino acid sequence of SEQ ID NO: 30;
(c) a heavy chain variable region comprising: a CDRH1 comprising the amino acid sequence of SEQ ID NO: 59; a CDR1-H2 comprising the amino acid sequence of SEQ ID NO: 60; and a CDRH3 comprising the amino acid sequence of SEQ ID NO: 61; and
a light chain variable comprising: a CDRL1 comprising the amino acid sequence of SEQ ID NO: 35; a CDRL2 comprising the amino acid sequence of SEQ ID NO: 36; and a CDRL3 comprising the amino acid sequence of SEQ ID NO: 37; or
(d) a heavy chain variable region comprising: a CDRH1 comprising the amino acid sequence of SEQ ID NO: 66; a CDR1-H2 comprising the amino acid sequence of SEQ ID NO: 67; and a CDRH3 comprising the amino acid sequence of SEQ ID NO: 68; and
a light chain variable comprising: a CDRL1 comprising the amino acid sequence of SEQ ID NO: 42; a CDRL2 comprising the amino acid sequence of SEQ ID NO: 29; and a CDRL3 comprising the amino acid sequence of SEQ ID NO: 30.

2. The antibody or antigen binding fragment of claim 1, comprising:
a heavy chain variable region comprising: a CDRH1 comprising the amino acid sequence of SEQ ID NO: 15; a CDRH2 comprising the amino acid sequence of SEQ ID NO: 16; and a CDRH3 comprising the amino acid sequence of SEQ ID NO: 17 and a light chain variable comprising: a CDR1-L1 comprising the amino acid sequence of SEQ ID NO: 20; a CDR1-L2 comprising the amino acid sequence of SEQ ID NO: 21; and a CDR1-L3 comprising the amino acid sequence of SEQ ID NO: 22.

3. The antibody or antigen binding fragment of claim 1, comprising:
a heavy chain variable region comprising: a CDRH1 comprising the amino acid sequence of SEQ ID NO: 52; a CDR1-H2 comprising the amino acid sequence of SEQ ID NO: 53; and a CDRH3 comprising the amino acid sequence of SEQ ID NO: 54 and a light chain variable comprising: a CDRL1 comprising the amino acid sequence of SEQ ID NO: 28; a CDRL2 comprising the amino acid sequence of SEQ ID NO: 29; and a CDRL3 comprising the amino acid sequence of SEQ ID NO: 30.

4. The antibody or antigen binding fragment of claim 1, comprising:
a heavy chain variable region comprising: a CDRH1 comprising the amino acid sequence of SEQ ID NO: 59; a CDR1-H2 comprising the amino acid sequence of SEQ ID NO: 60; and a CDRH3 comprising the amino acid sequence of SEQ ID NO: 61 and a light chain variable comprising: a CDRL1 comprising the amino acid sequence of SEQ ID NO: 35; a CDRL2 comprising the amino acid sequence of SEQ ID NO: 36; and a CDRL3 comprising the amino acid sequence of SEQ ID NO: 37.

5. The antibody or antigen binding fragment of claim 1, comprising:
a heavy chain variable region comprising: a CDRH1 comprising the amino acid sequence of SEQ ID NO: 66; a CDR1-H2 comprising the amino acid sequence of SEQ ID NO: 67; and a CDRH3 comprising the amino acid sequence of SEQ ID NO: 68 and a light chain variable comprising: a CDRL1 comprising the amino acid sequence of SEQ ID NO: 42; a CDRL2 comprising the amino acid sequence of SEQ ID NO: 29; and a CDRL3 comprising the amino acid sequence of SEQ ID NO: 30.

6. The antibody or antigen binding fragment of claim 2, wherein the heavy chain variable region comprises an amino acid sequence set forth SEQ ID NO: 72 and wherein the light chain variable region comprises an amino acid sequence set forth in SEQ ID NO: 47.

7. The antibody or antigen-binding fragment of claim 1, comprising:
(a) a light chain variable region comprising an amino acid sequence having at least 95% sequence identity to a V_{L} domain comprising the amino acid sequence of any one of SEQ ID NOs: 43, 44, 45, 46, and 47, and a heavy chain variable region comprising an amino acid sequence having at least 95% sequence identity to a V_{H} domain comprising the amino acid sequence of any one of SEQ ID NOs: 69, 70, 71, and 72;
(b) a light chain variable region comprising an amino acid sequence having at least 95% sequence identity to a V_{L} domain comprising the amino acid sequence of any one of SEQ ID NOs: 24, 25, 26, and 27, and a heavy chain variable region comprising an amino acid sequence having at least 95% amino acid sequence identity to a V_{H} domain comprising the amino acid sequence of any one of SEQ ID NOs: 48, 49, 50, and 51;
(c) a light chain variable region comprising an amino acid sequence having at least 95% amino acid sequence identity to a V_{L} domain comprising the amino acid sequence of any one of SEQ ID NOs: 31, 32, 33, and 34, and a heavy chain variable region comprising an amino acid sequence having at least 95% amino acid sequence identity to a V_{H} domain comprising the amino acid sequence of any one of SEQ ID NOs: 55, 56, 57, and 58; or
(d) a light chain variable region comprising an amino acid sequence having at least 95% amino acid sequence identity to a V_{L} domain comprising the amino acid sequence of any one of SEQ ID NOs: 38, 39, 40, and 41, and a heavy chain variable region comprising an amino acid sequence having at least 95% amino acid sequence identity to a V_{H} domain comprising the amino acid sequence of any one of SEQ ID NOs: 62, 63, 64, and 65.

8. The antibody or antigen-binding fragment of claim 7, comprising a light chain variable region comprising an amino acid sequence having at least 95% sequence identity to a V_{L} domain comprising the amino acid sequence of any one of SEQ ID NOs: 43, 44, 45, 46, and 47, and a heavy chain variable region comprising an amino acid sequence having at least 95% sequence identity to a V_{H} domain comprising the amino acid sequence of any one of SEQ ID NOs: 69, 70, 71, and 72.

9. An antibody or antigen binding fragment thereof that binds to Nectin-4, comprising:
(a.) a V_{L} domain comprising the amino acid sequence of any one of SEQ ID NOs: 43, 44, 45, 46, and 47 and a V_{H} domain comprising the amino acid sequence of any one of SEQ ID NOs: 69, 70, 71, and 72;
(b.) a V_{L} domain comprising the amino acid sequence of any one of SEQ ID NOs: 24, 25, 26, and 27 and a V_{H} domain comprising the amino acid sequence of any one of SEQ ID NOs: 48, 49, 50, and 51;
(c.) a V_{L} domain comprising the amino acid sequence of any one of SEQ ID NOs: 31, 32, 33, and 34 and a V_{H} domain comprising the amino acid sequence of any one of SEQ ID NOs: 55, 56, 57, and 58; or
(d.) a V_{L} domain comprising the amino acid sequence of any one of SEQ ID NOs: 38, 39, 40, and 41 and a V_{H} domain comprising the amino acid sequence of any one of SEQ ID NOs: 62, 63, 64, and 65.

10. The antibody or antigen binding fragment of claim 9, comprising a V_{L} domain comprising the amino acid sequence of any one of SEQ ID NOs: 43, 44, 45, 46, and 47 and a V_{H} domain comprising the amino acid sequence of any one of SEQ ID NOs: 69, 70, 71, and 72.

11. An antibody that binds to Nectin-4, comprising:
(a.) a light chain comprising an amino acid sequence having at least 95% sequence identity to the amino acid sequence of any one of SEQ ID NOs: 139, 140, 141, 142, and 143, and a heavy chain comprising an amino acid sequence having at least 95% sequence identity to the amino acid sequence of any one of SEQ ID NOs: 156, 157, 158, and 159;
(b.) a light chain comprising an amino acid sequence having at least 95% sequence identity to the amino acid sequence of any one of SEQ ID NOs: 127, 128, 129, and 130 and a heavy chain comprising an amino acid sequence having at least 95% sequence identity to any one of SEQ ID NOs: 144, 145, 146, and 147;
(c.) a light chain comprising an amino acid sequence having at least 95% sequence identity to the amino acid sequence of any one of SEQ ID NOs: 131, 132, 133, and 134 and a heavy chain comprising an amino acid sequence having at least 95% sequence identity to the amino acid sequence of any one of SEQ ID NOs: 148, 149, 150, and 151; or
(d.) a light chain comprising an amino acid sequence having at least 95% sequence identity to the amino acid sequence of any one of SEQ ID NOs: 135, 136, 137, and 138 and a heavy chain comprising an amino acid sequence having at least 95% sequence identity to the amino acid sequence of any one of SEQ ID NOs: 152, 153, 154, and 155.

12. The antibody that binds to Nectin-4 of claim 11, comprising a light chain comprising an amino acid sequence having at least 95% sequence identity to the amino acid sequence of any one of SEQ ID NOs: 139, 140, 141, 142, and 143, and a heavy chain comprising an amino acid sequence having at least 95% sequence identity to the amino acid sequence of any one of SEQ ID NOs: 156, 157, 158, and 159.

13. The antibody of claim 11, comprising:
(a.) a light chain comprising the amino acid sequence of any one of SEQ ID NOs: 139, 140, 141, 142, and 143, and a heavy chain comprising the amino acid sequence of any one of SEQ ID NOs: 156, 157, 158, and 159;
(b.) a light chain comprising the amino acid sequence of any one of SEQ ID NOs: 127, 128, 129, and 130 and a heavy chain comprising the amino acid sequence of any one of SEQ ID NOs: 144, 145, 146, and 147;
(c.) a light chain comprising the amino acid sequence of any one of SEQ ID NOs: 131, 132, 133, and 134 and a heavy chain comprising the amino acid sequence of any one of SEQ ID NOs: 148, 149, 150, and 151; or
(d.) a light chain comprising the amino acid sequence of any one of SEQ ID NOs: 135, 136, 137, and 138 and a heavy chain comprising the amino acid sequence of any one of SEQ ID NOs: 152, 153, 154, and 155.

14. The antibody of claim 13, comprising a light chain comprising the amino acid sequence of any one of SEQ ID NOs: 139, 140, 141, 142, and 143, and a heavy chain comprising the amino acid sequence of any one of SEQ ID NOs: 156, 157, 158, and 159.

15. An antibody that binds to Nectin-4, consisting of:
(a.) two light chains having at least 95% sequence identity to the amino acid sequence of any one of SEQ ID NOs: 139, 140, 141, 142, and 143, and two heavy chains having at least 95% sequence identity to the amino acid sequence of any one of SEQ ID NOs: 156, 157, 158, and 159;
(b.) two light chains having at least 95% sequence identity to the amino acid sequence of any one of SEQ ID NOs: 127, 128, 129, and 130 and two heavy chains having at least 95% sequence identity to the amino acid sequence of any one of SEQ ID NOs: 144, 145, 146, and 147;
(c.) two light chains having at least 95% sequence identity to the amino acid sequence of any one of SEQ ID NOs: 131, 132, 133, and 134 and two heavy chains having at least 95% sequence identity to the amino acid sequence of any one of SEQ ID NOs: 148, 149, 150, and 151; or
(d.) two light chains having at least 95% sequence identity to the amino acid sequence of any one of SEQ ID NOs: 135, 136, 137, and 138 and two heavy chains having at least 95% sequence identity to the amino acid sequence of any one of SEQ ID NOs: 152, 153, 154, and 155.

16. The antibody of claim 15, consisting of two light chains having at least 95% sequence identity to the amino acid sequence of any one of SEQ ID NOs: 139, 140, 141, 142, and 143, and two heavy chains having at least 95% sequence identity to the amino acid sequence of any one of SEQ ID NOs: 156, 157, 158, and 159.

17. The antibody of claim 15, consisting of:
(a.) two light chains comprising the amino acid sequence of any one of SEQ ID NOs: 139, 140, 141, 142, and 143, and two heavy chains comprising the amino acid sequence of any one of SEQ ID NOs: 156, 157, 158, and 159;
(b.) two light chains comprising the amino acid sequence of any one of SEQ ID NOs: 127, 128, 129, and 130 and two heavy chains comprising the amino acid sequence of any one of SEQ ID NOs: 144, 145, 146, and 147;
(c.) two light chains comprising the amino acid sequence of any one of SEQ ID NOs: 131, 132, 133, and 134 and two heavy chains comprising the amino acid sequence of any one of SEQ ID NOs: 148, 149, 150, and 151; or
(d.) two light chains comprising the amino acid sequence of any one of SEQ ID NOs: 135, 136, 137, and 138 and two heavy chains comprising the amino acid sequence of any one of SEQ ID NOs: 152, 153, 154, and 155.

18. The antibody of claim 17, consisting of two light chains comprising the amino acid sequence of any one of SEQ ID NOs: 139, 140, 141, 142, and 143, and two heavy chains comprising the amino acid sequence of any one of SEQ ID NOs: 156, 157, 158, and 159.

19. An antibody consisting of:
(a.) two light chains consisting of the amino acid sequence of any one of SEQ ID NOs: 139, 140, 141, 142, and 143 and two heavy chains consisting of the amino acid sequence of any one of SEQ ID NOs: 156, 157, 158, and 159;
(b.) two light chains consisting of the amino acid sequence of any one of SEQ ID NOs: 127, 128, 129, and 130 and two heavy chains consisting of any one of SEQ ID NOs: 144, 145, 146, and 147;
(c.) two light chains consisting of the amino acid sequence of any one of SEQ ID NOs: 131, 132, 133, and 134 and two heavy chains consisting of the amino acid sequence of any one of SEQ ID NOs: 148, 149, 150, and 151; or
(d.) two light chains consisting of the amino acid sequence of any one of SEQ ID NOs: 135, 136, 137, and 138 and two heavy chains consisting of the amino acid sequence of any one of SEQ ID NOs: 152, 153, 154, and 155.

20. The antibody of claim 19 consisting of two light chains consisting of the amino acid sequence of any one of SEQ ID NOs: 139, 140, 141, 142, and 143 and two heavy chains consisting of the amino acid sequence of any one of SEQ ID NOs: 156, 157, 158, and 159.

21. A polypeptide comprising the V_{L} domains or the V_{H} domains of any one of the antibodies or antigen binding fragments of claim 1.

22. An isolated nucleic acid encoding a V_{L} domain, a V_{H} domain, or both a V_{L} domain and a V_{H} domain, of any one of the antibodies or antigen binding fragments of claim 1.

23. An expression vector comprising the isolated nucleic acid of claim 22.

24. A host cell comprising the isolated nucleic acid of claim 22.

25. A composition comprising the antibody or antigen binding fragment of claim 1 and a pharmaceutically acceptable carrier.

26. A method of treating cancer in a subject in need thereof, comprising administering to the subject an effective amount of the antibody or antigen binding fragment of claim 1.

27. The method of claim 26, wherein the cancer is breast cancer, bladder cancer, lung cancer, urothelial cancer, colorectal cancer, cervical cancer, pancreatic cancer, head and neck squamous cell carcinoma (HNSCC), or ovarian cancer.

28. The method of claim 27, wherein the cancer is bladder cancer, HNSCC, cervical cancer, breast cancer, or pancreatic cancer.

29. The method of claim 28, wherein the cancer is bladder cancer.

30. The method of claim 28, wherein the cancer is HNSCC.

31. The method of claim 28, wherein the cancer is cervical cancer.

32. The method of claim 28, wherein the cancer is breast cancer.

33. The method of claim 28, wherein the cancer is pancreatic cancer.

34. An antibody or antigen binding fragment of claim 1 for use in treating cancer.

35. The antibody or antigen binding fragment of claim 34, wherein the cancer comprises breast cancer, bladder cancer, lung cancer, urothelial cancer, colorectal cancer, cervical cancer, pancreatic cancer, head and neck squamous cell carcinoma (HNSCC), or ovarian cancer.

36. The antibody or antigen binding fragment of claim 35, wherein the cancer is bladder cancer, HNSCC, cervical cancer, breast cancer, or pancreatic cancer.

37. The antibody or antigen binding fragment of claim 35, wherein the cancer is bladder cancer.

38. The antibody or antigen binding fragment of claim 35, wherein the cancer is HNSCC.

39. The antibody or antigen binding fragment of claim 35, wherein the cancer is cervical cancer.

40. The antibody or antigen binding fragment of claim 35, wherein the cancer is breast cancer.

41. The antibody or antigen binding fragment of claim 35, wherein the cancer is pancreatic cancer.

42. A method of producing an antibody or antigen binding fragment of claim 1, comprising:
(a.) culturing a host cell in culture medium, wherein the host cell comprises one or more vectors comprising a polynucleotide encoding a V_{L} domain and V_{H} domain of claim 1, under conditions that allow expression of the V_{L} domain and V_{H} domain, and
(b.) optionally, recovering the antibody or antigen binding fragment from the host cell or the culture medium.


SEQUENCE LISTING (194 sequences)

SEQ ID NO: 1 - protein, 671 aa, synthetic construct
MGWSCIILFLVATATGVHSEVQLVESGGGLVQPGGSLRLSCAASGSTFYTAVMGWVRQAPGKGLEWVAAIRWTALTTSYADSVKGRFTISRDGAKTTLYLQMNSLRPEDTAVYYCAARGTLGLFTTADSYDYWGQGTLVTVSSGGGGSGGGSGGVYCGPEFDESVGCMGGGGSGGGLSGRSDAGSPLGLAGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSEVQLVESGGGLVQPGGSLRLSCAASGFSFSNYYMAWVRQAPGKGLEWVASISTGGGNIYYRDSVKGRFTISRDNAKNSLYLQMNSLRAEDTAVYYCARQTAYYVMDAWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 2 - protein, 275 aa, synthetic construct
MGWSCIILFLVATATGVHSGGWSCAPDEDTWLCPAGGGGSGGLSGRSDAGSPLGLAGSGGSDIQMTQSPSSLSASVGDTVTITCLATEDIFSYLAWYQQKPGKAPKLLIYGANRLKDTVPSRFSGGGSGTEYSLTISGLQPEDFGTYYCLQGAKFPLTFGQGTKLDMKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 19 aa, synthetic construct
MGWSCIILFLVATATGVHS

SEQ ID NO: 4 - protein, 5 aa, synthetic construct
TAVMG

SEQ ID NO: 5 - protein, 18 aa, synthetic construct
AAIRWTALTTSYADSVKG

SEQ ID NO: 6 - protein, 15 aa, synthetic construct
RGTLGLFTTADSYDY

SEQ ID NO: 7 - protein, 18 aa, synthetic construct
GGVYCGPEFDESVGCMGG

SEQ ID NO: 8 - protein, 15 aa, synthetic construct
LSGRSDAGSPLGLAG

SEQ ID NO: 9 - protein, 4 aa, synthetic construct
KYAM

SEQ ID NO: 10 - protein, 19 aa, synthetic construct
RIRSKYNNYATYYADSVKD

SEQ ID NO: 11 - protein, 14 aa, synthetic construct
HGNFGNSYISYWAY

SEQ ID NO: 12 - protein, 8 aa, synthetic construct
GSSTGAVT

SEQ ID NO: 13 - protein, 7 aa, synthetic construct
GTKFLAP

SEQ ID NO: 14 - protein, 9 aa, synthetic construct
VLWYSNRWV

SEQ ID NO: 15 - protein, 5 aa, synthetic construct
NYYMA

SEQ ID NO: 16 - protein, 17 aa, synthetic construct
SISTGGGNIYYRDSVKG

SEQ ID NO: 17 - protein, 9 aa, synthetic construct
QTAYYVMDA

SEQ ID NO: 18 - protein, 18 aa, synthetic construct
GGWSCAPDEDTWLCPAGG

SEQ ID NO: 19 - protein, 15 aa, synthetic construct
LSGRSDAGSPLGLAG

SEQ ID NO: 20 - protein, 11 aa, synthetic construct
LATEDIFSYLA

SEQ ID NO: 21 - protein, 7 aa, synthetic construct
GANRLKD

SEQ ID NO: 22 - protein, 9 aa, synthetic construct
LQGAKFPLT

SEQ ID NO: 23 - protein, 19 aa, synthetic construct
GGWFCAPDENTWLCPNAGG

SEQ ID NO: 24 - protein, 107 aa, synthetic construct
EIQMTQSHKTFSVSTGQRVTITCKASQDVSIAVAWYQQKPGKAPKLLIYWASTRQTGVPSRFSGSGSGTDFTLTISCVQSEDFAVYYCQQYSSYPFTFGQGTKLEIK

SEQ ID NO: 25 - protein, 107 aa, synthetic construct
DIQMTQSPSFLSASVGDRVTITCKASQDVSIAVAWYQQKPGKAPKLLIYWASTRQTGVPSRFSGSGSGTDFTLTISSLQPEDFATYFCQQYSSYPFTFGQGTKLEIK

SEQ ID NO: 26 - protein, 107 aa, synthetic construct
DIQMTQSPSFLSASVGERVTITCKASQDVSIAVAWYQQKPGKAPKLLIYWASTRQTGVPSRFSGSGSGTDFTLTISSLQPEDIATYFCQQYSSYPFTFGQGTKLEIK

SEQ ID NO: 27 - protein, 107 aa, synthetic construct
DIQMTQSPSFLSASVGDRVTITCKASQDVSIAVAWYQQKPGKAPKLLIYWASTRQTGVPSRFSGSGSGTDFTLTISSLQPEDSATYFCQQYSSYPFTFGQGTKVDIK

SEQ ID NO: 28 - protein, 11 aa, synthetic construct
KASQDVSIAVA

SEQ ID NO: 29 - protein, 7 aa, synthetic construct
WASTRQT

SEQ ID NO: 30 - protein, 9 aa, synthetic construct
QQYSSYPFT

SEQ ID NO: 31 - protein, 107 aa, synthetic construct
EIQMTQSQSTLSVSLGDRVTITCKASQNVVTAVAWYQQKPGQAPKLLIYSASHRFTGVPSRFSGSGSGTDFTLIISCMQSEDFAVYYCQQYSSYPYTFGQGTKLEIK

SEQ ID NO: 32 - protein, 107 aa, synthetic construct
DIQMTQSPSFLSASVGDRVTITCKASQNVVTAVAWYQQKPGKAPKLLIYSASHRFTGVPSRFSGSGSGTDFTLTISNLQSEDLATYFCQQYSSYPYTFGQGTKLEIK

SEQ ID NO: 33 - protein, 107 aa, synthetic construct
DIQMTQSPSFLSASVGDRVTITCKASQNVVTAVAWYQQKPGKAPKLLIYSASHRFTGVPSRFSGSGSGTDFTLTISNLQPEDSATYFCQQYSSYPYTFGQGTKLEIK

SEQ ID NO: 34 - protein, 107 aa, synthetic construct
DIQMTQSPSFLSASVGDRVTITCKASQNVVTAVAWYQQKPGKAPKLLIYSASHRFTGVPSRFSGSGSGTDFTLTISNLQPDDAATYFCQQYSSYPYTFGQGTKLEIK

SEQ ID NO: 35 - protein, 11 aa, synthetic construct
KASQNVVTAVA

SEQ ID NO: 36 - protein, 7 aa, synthetic construct
SASHRFT

SEQ ID NO: 37 - protein, 9 aa, synthetic construct
QQYSSYPYT

SEQ ID NO: 38 - protein, 107 aa, synthetic construct
AIQMTQSHKSFSVSTGQRVTITCKASQDVSTTVAWYQQKPGQAPKLLIYWASTRQTGVPSRFTGSGSGTDFTLTISCVQSEDFAVYYCQQYSSYPFTFGQGTKLEIK

SEQ ID NO: 39 - protein, 107 aa, synthetic construct
DIQMTQSPSFLSASVGDRVTITCKASQDVSTTVAWYQQKPGKAPKLLIYWASTRQTGVPSRFSGSGSGTDFTLTISNLQPEDFATYFCQQYSSYPFTFGQGTKLEIK

SEQ ID NO: 40 - protein, 107 aa, synthetic construct
DIQMTQSPSFLSASVGERVTITCKASQDVSTTVAWYQQKPGKAPKLLIYWASTRQTGVPSRFSGSGSGTDFTLTINSLQPEDVATYFCQQYSSYPFTFGQGTKLEIK

SEQ ID NO: 41 - protein, 107 aa, synthetic construct
DIQMTQSPSFLSASVGERVTITCKASQDVSTTVAWYQQKPGKAPKLLIYWASTRQTGVPSRFSGSGSGTDFTLTISNLQPEDVATYFCQQYSSYPFTFGQGTKLEIK

SEQ ID NO: 42 - protein, 11 aa, synthetic construct
KASQDVSTTVA

SEQ ID NO: 43 - protein, 107 aa, synthetic construct
DIQMTQSPSSVSLTVGQRVTITCLATEDIFSYLAWYQQKPGKAPRLLIYGANRLKDGVPSRFSGSGSGTQYTLRISSMQPEDFGVYYCLQGAKFPLTFGPGTKLEIK

SEQ ID NO: 44 - protein, 107 aa, synthetic construct
DIQMTQSPSSLSASIGDTVTITCLATEDIFSYLAWYQQRPGKAPKLLIYGANRLKDGVPSRFSGSGSGTDYTLTISGLQPEDFVTYYCLQGAKFPLTFGQGTKLDMK

SEQ ID NO: 45 - protein, 107 aa, synthetic construct
DIQMTQSPSSLSASVGDTVTITCLATEDIFSYLAWYQQKPGKAPKLLIYGANRLKDGVPSRFSGSGSGTDYTLTISGLQPEDFGTYYCLQGAKFPLTFGQGTKLEIK

SEQ ID NO: 46 - protein, 107 aa, synthetic construct
DIQMTQSPSSLSASVGDTVTITCLATEDIFSYLAWYQQKPGKAPKLLIYGANRLKDGVPSRFSGGGSGTEYSLTISGLQPEDFGTYYCLQGAKFPLTFGQGTKLDMK

SEQ ID NO: 47 - protein, 108 aa, synthetic construct
DIQMTQSPSSLSASVGDTVTITCLATEDIFSYLAWYQQKPGKAPKLLIYGANRLKDTVPSRFSGGGSGTEYSLTISGLQPEDFGTYYCLQGAKFPLTFGQGTKLDMKR

SEQ ID NO: 48 - protein, 122 aa, synthetic construct
QVQLLESGGELVQPGGSLRLSCAASGFTFSDYYMFWIRQTPQKRLEWVAYISNGGGNTYYPDTVKGRFTISRDNAKNQLYLQMRSLTPEDTAIYYCASPEARYYGNYPFPYWGQGTLVTVSS

SEQ ID NO: 49 - protein, 122 aa, synthetic construct
QVQLVESGGGLVKPGGSLRLSCAASGFTFSDYYMFWIRQAPGKGLEWVAYISNGGGNTYYPDTVKGRFTISRDNAKNTLYLQMNSLRAEDTAVYYCASPEARYYGNYPFPYWGQGTLVTVSS

SEQ ID NO: 50 - protein, 122 aa, synthetic construct
QVQLVESGGGVVKPGGSLRLSCAASGFTFSDYYMFWIRQAPGKGLEWVAYISNGGGNTYYPDTVKGRFTISRDNAKNTLYLQMNSLRAEDTAVYYCASPEARYYGNYPFPYWGQGTLVTVSS

SEQ ID NO: 51 - protein, 122 aa, synthetic construct
QVQLVESGGGVVQPGGSLRLSCAASGFTFSDYYMFWIRQAPGKGLEWVAYISNGGGNTYYPDTVKGRFTISRDNAKNTLYLQMNSLRAEDTAVYYCASPEARYYGNYPFPYWGQGTLVTVSS

SEQ ID NO: 52 - protein, 5 aa, synthetic construct
DYYMF

SEQ ID NO: 53 - protein, 17 aa, synthetic construct
YISNGGGNTYYPDTVKG

SEQ ID NO: 54 - protein, 13 aa, synthetic construct
PEARYYGNYPFPY

SEQ ID NO: 55 - protein, 116 aa, synthetic construct
QVQLLQSGPGLVKPSATLSLTCTVSGFSLTTYGVHWIRQPPGKGLEWMGVIWSGGSTDYNAAFISRLTISKDNSKNQVFLQLYSLRAEDTAIYYCARTSHWYFDVWGRGTLVTVSS

SEQ ID NO: 56 - protein, 116 aa, synthetic construct
QVQLQESGPGLVKPSQTLSLTCTVSGFSLTTYGVHWVRQSPGKGLEWLGVIWSGGSTDYNAAFISRLSISKDNSKSQVFLEMTSLTAADTAIYYCARTSHWYFDVWGRGTLVTVSS

SEQ ID NO: 57 - protein, 116 aa, synthetic construct
QVQLQESGPGLVKPSQTLSLTCTVSGFSLTTYGVHWVRQAPEKGLEWLGVIWSGGSTDYNAAFISRLSISKDNSKSQVFLEMTSLTADDTAIYYCARTSHWYFDVWGRGTLVTVSS

SEQ ID NO: 58 - protein, 116 aa, synthetic construct
QVQLQESGPGLVKPSQTLSLTCTVSGFSLTTYGVHWVRQTPEKGLEWLGVIWSGGSTDYNAAFISRLSISKDNSKSQVFLEMTSLTADDTAIYYCARTSHWYFDVWGRGTLVTVSS

SEQ ID NO: 59 - protein, 5 aa, synthetic construct
TYGVH

SEQ ID NO: 60 - protein, 16 aa, synthetic construct
VIWSGGSTDYNAAFIS

SEQ ID NO: 61 - protein, 8 aa, synthetic construct
TSHWYFDV

SEQ ID NO: 62 - protein, 122 aa, synthetic construct
QVQLLESGGGLVQPGGSLRLSCAASGFTFSDYYMYWIRQTPQKRLEWVAYISNGGGNTYYSDTVKGRFTISRDNAKNQLYLQMRSLRPEDTAIYYCASPEARYYGNFPFPYWGQGTLVTVSS

SEQ ID NO: 63 - protein, 122 aa, synthetic construct
QVQLVESGGGLVKPGGSLRLSCAASGFTFSDYYMYWIRQAPGKGLEWVAYISNGGGNTYYSDTVKGRFTISRDNAKNTLYLQMNSLRAEDTAVYYCASPEARYYGNFPFPYWGQGTLVTVSS

SEQ ID NO: 64 - protein, 122 aa, synthetic construct; identical to SEQ ID NO: 63

SEQ ID NO: 65 - protein, 122 aa, synthetic construct; identical to SEQ ID NO: 63

SEQ ID NO: 66 - protein, 5 aa, synthetic construct
DYYMY

SEQ ID NO: 67 - protein, 17 aa, synthetic construct
YISNGGGNTYYSDTVKG

SEQ ID NO: 68 - protein, 13 aa, synthetic construct
PEARYYGNFPFPY

SEQ ID NO: 69 - protein, 118 aa, synthetic construct
QVQLVESGGGVVQPGRSLRLSCAASGFSFSNYYMAWVRQAPGKGLEWVASISTGGGNIYYRDSVKGRFTISRDNSKSTLYLQMRSLRSEDTAIYYCARQTAYYVMDAWGQGTMVTVSS

SEQ ID NO: 70 - protein, 118 aa, synthetic construct
EVQLVESGGGLVQPGGSLRLSCAASGFSFSNYYMAWVRQAPGKGLEWVASISTGGGNIYYRDSVKGRFTISRDNAKNSLYLQMNSLRAEDTAVYYCARQTAYYVMDAWGQGTTVTVSS

SEQ ID NO: 71 - protein, 118 aa, synthetic construct; identical to SEQ ID NO: 70

SEQ ID NO: 72 - protein, 118 aa, synthetic construct; identical to SEQ ID NO: 70

SEQ ID NO: 73 - protein, 107 aa, synthetic construct
DIQMTQSPASLSVYLGETVSIECLATEDIFSYLAWYQQKPGKSPQLLIYGANRLKDGVPSRFSGSGTGTQYSLRISGMQPEDEGDYYCLQGAKFPLTFGSGTKLEIK

SEQ ID NO: 74 - protein, 107 aa, synthetic construct
DIQMTQSPASLSASLGETVSIECLASEDIYSYLAWFQQKSGKSPQLLIYAANRLQDGVPSRFSGSGSGTQYSLRISGMQPEDEGDYFCLQGSQFPWTFGGGTKLELK

SEQ ID NO: 75 - protein, 107 aa, synthetic construct
DIQMTQSPASLSASLGETVSIECLASEDIHNKLAWYQQKPGKSPQLLIYYGSNLQDGVPSRFSGSGSGTQYYLKINSLESEDVATYFCLQDSKNPWTFGGGTKLEMK

SEQ ID NO: 76 - protein, 110 aa, synthetic construct
DIVLTQSPALDVSLGQRATISCSASQSVSISRYNLIHWYQQKPGQQPKLLIWRTSILTSGIPARFSGRGSGTDFTLTINPVQADDIATYYCQQSRESPFTFGAGTRLELK

SEQ ID NO: 77 - protein, 107 aa, synthetic construct
NIVLTQSPATLSVTPGESVSLSCRASQSISTGIHWYQQKSNESPRLLIKFASQSISGIPSRFSGSGSGTDFTLSINRVESEDFSIYYCQQRDSSLFTFGAGTKLELK

SEQ ID NO: 78 - protein, 108 aa, synthetic construct
TYELIQPPSTSVTLGNTVSLTCVGDDLPRRYAYWYQQKPDQSIVRVIYEDSKRPSGISDRFSGSSSGTTATLTIRDAQAEDEADYYCHSTYSDDKVRVFGGGTKLTVL

SEQ ID NO: 79 - protein, 118 aa, synthetic construct
EVQLVESGGGLVQPGRSMRLSCAASGFSFSNYYMAWVRQAPRKGLDWVASISTGGGNIYYRDSVKGRFTISRDNAKSTLYLQMDSLRSEDTATYYCARQTAYYVMDAWGQGASVTVSS

SEQ ID NO: 80 - protein, 119 aa, synthetic construct
EVQLVESGGGLVQPGRSLKLSCAASGFTFSNYDMAWVRQVPTKGLEWVASISPRGGRIYYRDSVKGRFTVSRDNAKSCLYLQMESLRSDDTATYYCARIHHGYWYFDFWGPGTMVTVSS

SEQ ID NO: 81 - protein, 121 aa, synthetic construct
EVKLLESGGGLVQPGGSMRLSCAASGFTFTDFYMIWIRQPAGKAPEWLGFIRNKANGYTTDYNPSVKGRFTISRDNTQNMLYLQMITLRAEDTATYYCARPLYYGYTPRYWGQGVMVTVSS

SEQ ID NO: 82 - protein, 120 aa, synthetic construct
EVQLVESGGGLVQPGRPLKLSCAASGFSFSHYDMAWVRQAPTKGLEWVAAISPSGGSTYYRDSVKGRFTVSRDKAKNSLYLQMDSLRSEDTATYYCARQGPSYGYYFDYWGQGVMVTVSS

SEQ ID NO: 83 - protein, 116 aa, synthetic construct
EVQLVESGGGLVQPGRSLKVSCAASGFTFSNYDMAWVRQAPTKGLEWVASINPGGISTYYRDSVKGRFTVSRDNEKSTLYLQMDSLRSEDTATYYCARRQPYFDYWGQGVMVTVSS

SEQ ID NO: 84 - protein, 119 aa, synthetic construct
QVTLKESGTGILQPSQALSLTCSISGFSLNTTGICVSWIRQPLGQGLEWLADICWDDGKGYNPSLKNRLSISKDTSNNQAFLKITRVDTTDTATYYCARNYGGNPFDYWGQGVMVTVSS

SEQ ID NO: 85 - protein, 11 aa, synthetic construct
LASEDIYSYLA

SEQ ID NO: 86 - protein, 7 aa, synthetic construct
AANRLQD

SEQ ID NO: 87 - protein, 9 aa, synthetic construct
LQGSQFPWT

SEQ ID NO: 88 - protein, 11 aa, synthetic construct
LASEDIHNKLA

SEQ ID NO: 89 - protein, 7 aa, synthetic construct
YGSNLQD

SEQ ID NO: 90 - protein, 9 aa, synthetic construct
LQDSKNPWT

SEQ ID NO: 91 - protein, 15 aa, synthetic construct
SASQSVSISRYNLIH

SEQ ID NO: 92 - protein, 7 aa, synthetic construct
RTSILTS

SEQ ID NO: 93 - protein, 9 aa, synthetic construct
QQSRESPFT

SEQ ID NO: 94 - protein, 11 aa, synthetic construct
RASQSISTGIH

SEQ ID NO: 95 - protein, 7 aa, synthetic construct
FASQSIS

SEQ ID NO: 96 - protein, 9 aa, synthetic construct
QQRDSSLFT

SEQ ID NO: 97 - protein, 11 aa, synthetic construct
VGDDLPRRYAY

SEQ ID NO: 98 - protein, 7 aa, synthetic construct
EDSKRPS

SEQ ID NO: 99 - protein, 11 aa, synthetic construct
HSTYSDDKVRV

SEQ ID NO: 100 - protein, 5 aa, synthetic construct
NYDMA

SEQ ID NO: 101 - protein, 17 aa, synthetic construct
SISPRGGRIYYRDSVKG

SEQ ID NO: 102 - protein, 10 aa, synthetic construct
IHHGYWYFDF

SEQ ID NO: 103 - protein, 5 aa, synthetic construct
DFYMI

SEQ ID NO: 104 - protein, 19 aa, synthetic construct
FIRNKANGYTTDYNPSVKG

SEQ ID NO: 105 - protein, 10 aa, synthetic construct
PLYYGYTPRY

SEQ ID NO: 106 - protein, 5 aa, synthetic construct
HYDMA

SEQ ID NO: 107 - protein, 17 aa, synthetic construct
AISPSGGSTYYRDSVKG

SEQ ID NO: 108 - protein, 11 aa, synthetic construct
QGPSYGYYFDY

SEQ ID NO: 109 - protein, 17 aa, synthetic construct
SINPGGISTYYRDSVKG

SEQ ID NO: 110 - protein, 7 aa, synthetic construct
RQPYFDY

SEQ ID NO: 111 - protein, 11 aa, synthetic construct
VGDDLPRRYAY

SEQ ID NO: 112 - protein, 7 aa, synthetic construct
EDSKRPS

SEQ ID NO: 113 - protein, 11 aa, synthetic construct
HSTYSDDKVRV

SEQ ID NO: 114 - protein, 122 aa, synthetic construct
EVQLQESGGDLVQPGGSLKLSCAASGFTFSDYYMFWIRQTPQKRLEWVAYISNGGGNTYYPDTVKGRFTISRDNAKNTLYLQMSRLKSEDTAMYYCASPEARYYGNYPFPYWGQGTLVTVSA

SEQ ID NO: 115 - protein, 107 aa, synthetic construct
DIVMTQSHKFMSTSVGGRVTITCKASQDVSIAVAWYQQKPGQSPKLLIYWASTRQTGVPDRFAGSGSGTDFTLTISNVQSEDLADYFCQQYSSYPFTFGSGTKLEIK

SEQ ID NO: 116 - protein, 116 aa, synthetic construct
QVQLKQSGPGLVQPSQSLSITCTVSGFSLTTYGVHWVRQSPGKGLEWLGVIWSGGSTDYNAAFISRLSISKDNSKSQVFFEMYSLQADDTAIYYCARTSHWYFDVWGTGTTVTVSS

SEQ ID NO: 117 - protein, 107 aa, synthetic construct
DIVMTQSQKFMSTTVGDRVSITCKASQNVVTAVAWYQQKPGQSPKLLIYSASHRFTGVPDRFTGSGSGTDFTLIISNMQSEDLADYFCQQYSSYPYTFGGGTKLEIK

SEQ ID NO: 118 - protein, 122 aa, synthetic construct
EVQLQESGGGLVQPGGSLKLSCAASGFTFSDYYMYWIRQTPQKRLEWVAYISNGGGNTYYSDTVKGRFTISRDNAKNTLYLQMSRLKSEDTAMYYCASPEARYYGNFPFPYWGQGTLVTVSA

SEQ ID NO: 119 - protein, 107 aa, synthetic construct
DIVMTQSHKFMSTSVGGRVTITCKASQDVSTTVAWYQQKPGQSPKLLIYWASTRQTGVPDRFTGSGSGTDFTLTISNVQSEDLADYFCQQYSSYPFTFGSGTKLEIK

SEQ ID NO: 120 - protein, 8 aa, synthetic construct
GFTFSDYY

SEQ ID NO: 121 - protein, 15 aa, synthetic construct
ASPEARYYGNYPFPY

SEQ ID NO: 122 - protein, 8 aa, synthetic construct
GFSLTTYG

SEQ ID NO: 123 - protein, 10 aa, synthetic construct
ARTSHWYFDV

SEQ ID NO: 124 - protein, 15 aa, synthetic construct
ASPEARYYGNFPFPY

SEQ ID NO: 125 - protein, 18 aa, synthetic construct
GGWTCADDEDTWLCPIGG

SEQ ID NO: 126 - protein, 18 aa, synthetic construct
GGWACAPDEDTWLCPIGG

SEQ ID NO: 127 - protein, 214 aa, synthetic construct
EIQMTQSHKTFSVSTGQRVTITCKASQDVSIAVAWYQQKPGKAPKLLIYWASTRQTGVPSRFSGSGSGTDFTLTISCVQSEDFAVYYCQQYSSYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 128 - protein, 214 aa, synthetic construct
DIQMTQSPSFLSASVGDRVTITCKASQDVSIAVAWYQQKPGKAPKLLIYWASTRQTGVPSRFSGSGSGTDFTLTISSLQPEDFATYFCQQYSSYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 129 - protein, 214 aa, synthetic construct
DIQMTQSPSFLSASVGERVTITCKASQDVSIAVAWYQQKPGKAPKLLIYWASTRQTGVPSRFSGSGSGTDFTLTISSLQPEDIATYFCQQYSSYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 130 - protein, 214 aa, synthetic construct
DIQMTQSPSFLSASVGDRVTITCKASQDVSIAVAWYQQKPGKAPKLLIYWASTRQTGVPSRFSGSGSGTDFTLTISSLQPEDSATYFCQQYSSYPFTFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 131 - protein, 214 aa, synthetic construct
EIQMTQSQSTLSVSLGDRVTITCKASQNVVTAVAWYQQKPGQAPKLLIYSASHRFTGVPSRFSGSGSGTDFTLIISCMQSEDFAVYYCQQYSSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 132 - protein, 214 aa, synthetic construct
DIQMTQSPSFLSASVGDRVTITCKASQNVVTAVAWYQQKPGKAPKLLIYSASHRFTGVPSRFSGSGSGTDFTLTISNLQSEDLATYFCQQYSSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 133 - protein, 214 aa, synthetic construct
DIQMTQSPSFLSASVGDRVTITCKASQNVVTAVAWYQQKPGKAPKLLIYSASHRFTGVPSRFSGSGSGTDFTLTISNLQPEDSATYFCQQYSSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 134 - protein, 214 aa, synthetic construct
DIQMTQSPSFLSASVGDRVTITCKASQNVVTAVAWYQQKPGKAPKLLIYSASHRFTGVPSRFSGSGSGTDFTLTISNLQPDDAATYFCQQYSSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 135 - protein, 214 aa, synthetic construct
AIQMTQSHKSFSVSTGQRVTITCKASQDVSTTVAWYQQKPGQAPKLLIYWASTRQTGVPSRFTGSGSGTDFTLTISCVQSEDFAVYYCQQYSSYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 136 - protein, 214 aa, synthetic construct
DIQMTQSPSFLSASVGDRVTITCKASQDVSTTVAWYQQKPGKAPKLLIYWASTRQTGVPSRFSGSGSGTDFTLTISNLQPEDFATYFCQQYSSYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 137 - protein, 214 aa, synthetic construct
DIQMTQSPSFLSASVGERVTITCKASQDVSTTVAWYQQKPGKAPKLLIYWASTRQTGVPSRFSGSGSGTDFTLTINSLQPEDVATYFCQQYSSYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 138 - protein, 214 aa, synthetic construct
DIQMTQSPSFLSASVGERVTITCKASQDVSTTVAWYQQKPGKAPKLLIYWASTRQTGVPSRFSGSGSGTDFTLTISNLQPEDVATYFCQQYSSYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 139 - protein, 214 aa, synthetic construct
DIQMTQSPSSVSLTVGQRVTITCLATEDIFSYLAWYQQKPGKAPRLLIYGANRLKDGVPSRFSGSGSGTQYTLRISSMQPEDFGVYYCLQGAKFPLTFGPGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 140 - protein, 214 aa, synthetic construct
DIQMTQSPSSLSASIGDTVTITCLATEDIFSYLAWYQQRPGKAPKLLIYGANRLKDGVPSRFSGSGSGTDYTLTISGLQPEDFVTYYCLQGAKFPLTFGQGTKLDMKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 141 - protein, 214 aa, synthetic construct
DIQMTQSPSSLSASVGDTVTITCLATEDIFSYLAWYQQKPGKAPKLLIYGANRLKDGVPSRFSGSGSGTDYTLTISGLQPEDFGTYYCLQGAKFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 142 - protein, 214 aa, synthetic construct
DIQMTQSPSSLSASVGDTVTITCLATEDIFSYLAWYQQKPGKAPKLLIYGANRLKDGVPSRFSGGGSGTEYSLTISGLQPEDFGTYYCLQGAKFPLTFGQGTKLDMKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 143 - protein, 214 aa, synthetic construct
DIQMTQSPSSLSASVGDTVTITCLATEDIFSYLAWYQQKPGKAPKLLIYGANRLKDTVPSRFSGGGSGTEYSLTISGLQPEDFGTYYCLQGAKFPLTFGQGTKLDMKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 144 - protein, 452 aa, synthetic construct
QVQLLESGGELVQPGGSLRLSCAASGFTFSDYYMFWIRQTPQKRLEWVAYISNGGGNTYYPDTVKGRFTISRDNAKNQLYLQMRSLTPEDTAIYYCASPEARYYGNYPFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 145 - protein, 452 aa, synthetic construct
QVQLVESGGGLVKPGGSLRLSCAASGFTFSDYYMFWIRQAPGKGLEWVAYISNGGGNTYYPDTVKGRFTISRDNAKNTLYLQMNSLRAEDTAVYYCASPEARYYGNYPFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 146 - protein, 452 aa, synthetic construct
QVQLVESGGGVVKPGGSLRLSCAASGFTFSDYYMFWIRQAPGKGLEWVAYISNGGGNTYYPDTVKGRFTISRDNAKNTLYLQMNSLRAEDTAVYYCASPEARYYGNYPFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 147 - protein, 452 aa, synthetic construct
QVQLVESGGGVVQPGGSLRLSCAASGFTFSDYYMFWIRQAPGKGLEWVAYISNGGGNTYYPDTVKGRFTISRDNAKNTLYLQMNSLRAEDTAVYYCASPEARYYGNYPFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 148 - protein, 446 aa, synthetic construct
QVQLLQSGPGLVKPSATLSLTCTVSGFSLTTYGVHWIRQPPGKGLEWMGVIWSGGSTDYNAAFISRLTISKDNSKNQVFLQLYSLRAEDTAIYYCARTSHWYFDVWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 149 - protein, 446 aa, synthetic construct
QVQLQESGPGLVKPSQTLSLTCTVSGFSLTTYGVHWVRQSPGKGLEWLGVIWSGGSTDYNAAFISRLSISKDNSKSQVFLEMTSLTAADTAIYYCARTSHWYFDVWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 150 - protein, 446 aa, synthetic construct
QVQLQESGPGLVKPSQTLSLTCTVSGFSLTTYGVHWVRQAPEKGLEWLGVIWSGGSTDYNAAFISRLSISKDNSKSQVFLEMTSLTADDTAIYYCARTSHWYFDVWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 151 - protein, 446 aa, synthetic construct
QVQLQESGPGLVKPSQTLSLTCTVSGFSLTTYGVHWVRQTPEKGLEWLGVIWSGGSTDYNAAFISRLSISKDNSKSQVFLEMTSLTADDTAIYYCARTSHWYFDVWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 152 - protein, 452 aa, synthetic construct
QVQLLESGGGLVQPGGSLRLSCAASGFTFSDYYMYWIRQTPQKRLEWVAYISNGGGNTYYSDTVKGRFTISRDNAKNQLYLQMRSLRPEDTAIYYCASPEARYYGNFPFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 153 - protein, 452 aa, synthetic construct
QVQLVESGGGLVKPGGSLRLSCAASGFTFSDYYMYWIRQAPGKGLEWVAYISNGGGNTYYSDTVKGRFTISRDNAKNTLYLQMNSLRAEDTAVYYCASPEARYYGNFPFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 154 - protein, 452 aa, synthetic construct; identical to SEQ ID NO: 153

SEQ ID NO: 155 - protein, 452 aa, synthetic construct; identical to SEQ ID NO: 153

SEQ ID NO: 156 - protein, 448 aa, synthetic construct
QVQLVESGGGVVQPGRSLRLSCAASGFSFSNYYMAWVRQAPGKGLEWVASISTGGGNIYYRDSVKGRFTISRDNSKSTLYLQMRSLRSEDTAIYYCARQTAYYVMDAWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 448 aa, synthetic construct
EVQLVESGGGLVQPGGSLRLSCAASGFSFSNYYMAWVRQAPGKGLEWVASISTGGGNIYYRDSVKGRFTISRDNAKNSLYLQMNSLRAEDTAVYYCARQTAYYVMDAWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 158 - protein, 448 aa, synthetic construct; identical to SEQ ID NO: 157

SEQ ID NO: 159 - protein, 448 aa, synthetic construct; identical to SEQ ID NO: 157

SEQ ID NO: 160 - DNA, 642 bp, synthetic construct
  source 1..642: mol_type = other DNA
gagatccagatgacccagagccacaagaccttcagcgtgagcaccggccagagggtgaccatcacctgcaaggccagccaggacgtgagcatcgccgtggcctggtaccagcagaagcccggcaaggcccccaagctgctgatctactgggccagcaccaggcagaccggcgtgcccagcaggttcagcggcagcggcagcggcaccgacttcaccctgaccatcagctgcgtgcagagcgaggacttcgccgtgtactactgccagcagtacagcagctaccccttcaccttcggccagggcaccaagctggagatcaagcgaacggtggctgccccctccgtgttcatcttcccccccagcgatgagcagctgaagagcggcacagccagcgtggtgtgcctgctgaacaacttctaccccagggaggccaaggtgcagtggaaggtggacaacgccctgcagagcggcaacagccaggagtccgtgaccgagcaggacagcaaggacagcacctacagcctgagcagcacactgaccctgagcaaggccgactacgagaagcacaaggtgtacgcctgcgaggtgacccatcagggcctgagcagccccgtgaccaagagcttcaacaggggcgagtgc

SEQ ID NO: 161 - DNA, 642 bp, synthetic construct
  source 1..642: mol_type = other DNA
gacatccagatgacccagagccccagcttcctgagcgccagcgtgggcgacagggtgaccatcacctgcaaggccagccaggacgtgagcatcgccgtggcctggtaccagcagaagcccggcaaggcccccaagctgctgatctactgggccagcaccaggcagaccggcgtgcccagcaggttcagcggcagcggcagcggcaccgacttcaccctgaccatcagcagcctgcagcccgaggacttcgccacctacttctgccagcagtacagcagctaccccttcaccttcggccagggcaccaagctggagatcaagcgaacggtggctgccccctccgtgttcatcttcccccccagcgatgagcagctgaagagcggcacagccagcgtggtgtgcctgctgaacaacttctaccccagggaggccaaggtgcagtggaaggtggacaacgccctgcagagcggcaacagccaggagtccgtgaccgagcaggacagcaaggacagcacctacagcctgagcagcacactgaccctgagcaaggccgactacgagaagcacaaggtgtacgcctgcgaggtgacccatcagggcctgagcagccccgtgaccaagagcttcaacaggggcgagtgc

SEQ ID NO: 162 - DNA, 642 bp, synthetic construct
  source 1..642: mol_type = other DNA
gacatccagatgacccagagccccagcttcctgagcgccagcgtgggcgagagggtgaccatcacctgcaaggccagccaggacgtgagcatcgccgtggcctggtaccagcagaagcccggcaaggcccccaagctgctgatctactgggccagcaccaggcagaccggcgtgcccagcaggttcagcggcagcggcagcggcaccgacttcaccctgaccatcagcagcctgcagcccgaggacatcgccacctacttctgccagcagtacagcagctaccccttcaccttcggccagggcaccaagctggagatcaagcgaacggtggctgccccctccgtgttcatcttcccccccagcgatgagcagctgaagagcggcacagccagcgtggtgtgcctgctgaacaacttctaccccagggaggccaaggtgcagtggaaggtggacaacgccctgcagagcggcaacagccaggagtccgtgaccgagcaggacagcaaggacagcacctacagcctgagcagcacactgaccctgagcaaggccgactacgagaagcacaaggtgtacgcctgcgaggtgacccatcagggcctgagcagccccgtgaccaagagcttcaacaggggcgagtgc

SEQ ID NO: 163 - DNA, 642 bp, synthetic construct
  source 1..642: mol_type = other DNA
gacatccagatgacccagagccccagcttcctgagcgccagcgtgggcgacagggtgaccatcacctgcaaggccagccaggacgtgagcatcgccgtggcctggtaccagcagaagcccggcaaggcccccaagctgctgatctactgggccagcaccaggcagaccggcgtgcccagcaggttcagcggcagcggcagcggcaccgacttcaccctgaccatcagcagcctgcagcccgaggacagcgccacctacttctgccagcagtacagcagctaccccttcaccttcggccagggcaccaaggtggacatcaagcgaacggtggctgccccctccgtgttcatcttcccccccagcgatgagcagctgaagagcggcacagccagcgtggtgtgcctgctgaacaacttctaccccagggaggccaaggtgcagtggaaggtggacaacgccctgcagagcggcaacagccaggagtccgtgaccgagcaggacagcaaggacagcacctacagcctgagcagcacactgaccctgagcaaggccgactacgagaagcacaaggtgtacgcctgcgaggtgacccatcagggcctgagcagccccgtgaccaagagcttcaacaggggcgagtgc

SEQ ID NO: 164 - DNA, 642 bp, synthetic construct
  source 1..642: mol_type = other DNA
gagatccagatgacccagagccagagcaccctgagcgtgagcctgggcgacagggtgaccatcacctgcaaggccagccagaacgtggtgaccgccgtggcctggtaccagcagaagcccggccaggcccccaagctgctgatctacagcgccagccacaggttcaccggcgtgcccagcaggttcagcggcagcggcagcggcaccgacttcaccctgatcatcagctgcatgcagagcgaggacttcgccgtgtactactgccagcagtacagcagctacccctacaccttcggccagggcaccaagctggagatcaagcgaacggtggctgccccctccgtgttcatcttcccccccagcgatgagcagctgaagagcggcacagccagcgtggtgtgcctgctgaacaacttctaccccagggaggccaaggtgcagtggaaggtggacaacgccctgcagagcggcaacagccaggagtccgtgaccgagcaggacagcaaggacagcacctacagcctgagcagcacactgaccctgagcaaggccgactacgagaagcacaaggtgtacgcctgcgaggtgacccatcagggcctgagcagccccgtgaccaagagcttcaacaggggcgagtgc

SEQ ID NO: 165 - DNA, 642 bp, synthetic construct
  source 1..642: mol_type = other DNA
gacatccagatgacccagagccccagcttcctgagcgccagcgtgggcgacagggtgaccatcacctgcaaggccagccagaacgtggtgaccgccgtggcctggtaccagcagaagcccggcaaggcccccaagctgctgatctacagcgccagccacaggttcaccggcgtgcccagcaggttcagcggcagcggcagcggcaccgacttcaccctgaccatcagcaacctgcagagcgaggacctggccacctacttctgccagcagtacagcagctacccctacaccttcggccagggcaccaagctggagatcaagcgaacggtggctgccccctccgtgttcatcttcccccccagcgatgagcagctgaagagcggcacagccagcgtggtgtgcctgctgaacaacttctaccccagggaggccaaggtgcagtggaaggtggacaacgccctgcagagcggcaacagccaggagtccgtgaccgagcaggacagcaaggacagcacctacagcctgagcagcacactgaccctgagcaaggccgactacgagaagcacaaggtgtacgcctgcgaggtgacccatcagggcctgagcagccccgtgaccaagagcttcaacaggggcgagtgc

SEQ ID NO: 166 - DNA, 642 bp, synthetic construct
  source 1..642: mol_type = other DNA
gacatccagatgacccagagccccagcttcctgagcgccagcgtgggcgacagggtgaccatcacctgcaaggccagccagaacgtggtgaccgccgtggcctggtaccagcagaagcccggcaaggcccccaagctgctgatctacagcgccagccacaggttcaccggcgtgcccagcaggttcagcggcagcggcagcggcaccgacttcaccctgaccatcagcaacctgcagcccgaggacagcgccacctacttctgccagcagtacagcagctacccctacaccttcggccagggcaccaagctggagatcaagcgaacggtggctgccccctccgtgttcatcttcccccccagcgatgagcagctgaagagcggcacagccagcgtggtgtgcctgctgaacaacttctaccccagggaggccaaggtgcagtggaaggtggacaacgccctgcagagcggcaacagccaggagtccgtgaccgagcaggacagcaaggacagcacctacagcctgagcagcacactgaccctgagcaaggccgactacgagaagcacaaggtgtacgcctgcgaggtgacccatcagggcctgagcagccccgtgaccaagagcttcaacaggggcgagtgc

SEQ ID NO: 167 - DNA, 642 bp, synthetic construct
  source 1..642: mol_type = other DNA
gacatccagatgacccagagccccagcttcctgagcgccagcgtgggcgacagggtgaccatcacctgcaaggccagccagaacgtggtgaccgccgtggcctggtaccagcagaagcccggcaaggcccccaagctgctgatctacagcgccagccacaggttcaccggcgtgcccagcaggttcagcggcagcggcagcggcaccgacttcaccctgaccatcagcaacctgcagcccgacgacgccgccacctacttctgccagcagtacagcagctacccctacaccttcggccagggcaccaagctggagatcaagcgaacggtggctgccccctccgtgttcatcttcccccccagcgatgagcagctgaagagcggcacagccagcgtggtgtgcctgctgaacaacttctaccccagggaggccaaggtgcagtggaaggtggacaacgccctgcagagcggcaacagccaggagtccgtgaccgagcaggacagcaaggacagcacctacagcctgagcagcacactgaccctgagcaaggccgactacgagaagcacaaggtgtacgcctgcgaggtgacccatcagggcctgagcagccccgtgaccaagagcttcaacaggggcgagtgc

SEQ ID NO: 168 - DNA, 642 bp, synthetic construct
  source 1..642: mol_type = other DNA
gccatccagatgacccagagccacaagagcttcagcgtgagcaccggccagagggtgaccatcacctgcaaggccagccaggacgtgagcaccaccgtggcctggtaccagcagaagcccggccaggcccccaagctgctgatctactgggccagcaccaggcagaccggcgtgcccagcaggttcaccggcagcggcagcggcaccgacttcaccctgaccatcagctgcgtgcagagcgaggacttcgccgtgtactactgccagcagtacagcagctaccccttcaccttcggccagggcaccaagctggagatcaagcgaacggtggctgccccctccgtgttcatcttcccccccagcgatgagcagctgaagagcggcacagccagcgtggtgtgcctgctgaacaacttctaccccagggaggccaaggtgcagtggaaggtggacaacgccctgcagagcggcaacagccaggagtccgtgaccgagcaggacagcaaggacagcacctacagcctgagcagcacactgaccctgagcaaggccgactacgagaagcacaaggtgtacgcctgcgaggtgacccatcagggcctgagcagccccgtgaccaagagcttcaacaggggcgagtgc

SEQ ID NO: 169 - DNA, 642 bp, synthetic construct
  source 1..642: mol_type = other DNA
gacatccagatgacccagagccccagcttcctgagcgccagcgtgggcgacagggtgaccatcacctgcaaggccagccaggacgtgagcaccaccgtggcctggtaccagcagaagcccggcaaggcccccaagctgctgatctactgggccagcaccaggcagaccggcgtgcccagcaggttcagcggcagcggcagcggcaccgacttcaccctgaccatcagcaacctgcagcccgaggacttcgccacctacttctgccagcagtacagcagctaccccttcaccttcggccagggcaccaagctggagatcaagcgaacggtggctgccccctccgtgttcatcttcccccccagcgatgagcagctgaagagcggcacagccagcgtggtgtgcctgctgaacaacttctaccccagggaggccaaggtgcagtggaaggtggacaacgccctgcagagcggcaacagccaggagtccgtgaccgagcaggacagcaaggacagcacctacagcctgagcagcacactgaccctgagcaaggccgactacgagaagcacaaggtgtacgcctgcgaggtgacccatcagggcctgagcagccccgtgaccaagagcttcaacaggggcgagtgc

SEQ ID NO: 170 - DNA, 642 bp, synthetic construct
  source 1..642: mol_type = other DNA
gacatccagatgacccagagccccagcttcctgagcgccagcgtgggcgagagggtgaccatcacctgcaaggccagccaggacgtgagcaccaccgtggcctggtaccagcagaagcccggcaaggcccccaagctgctgatctactgggccagcaccaggcagaccggcgtgcccagcaggttcagcggcagcggcagcggcaccgacttcaccctgaccatcaacagcctgcagcccgaggacgtggccacctacttctgccagcagtacagcagctaccccttcaccttcggccagggcaccaagctggagatcaagcgaacggtggctgccccctccgtgttcatcttcccccccagcgatgagcagctgaagagcggcacagccagcgtggtgtgcctgctgaacaacttctaccccagggaggccaaggtgcagtggaaggtggacaacgccctgcagagcggcaacagccaggagtccgtgaccgagcaggacagcaaggacagcacctacagcctgagcagcacactgaccctgagcaaggccgactacgagaagcacaaggtgtacgcctgcgaggtgacccatcagggcctgagcagccccgtgaccaagagcttcaacaggggcgagtgc

SEQ ID NO: 171 - DNA, 642 bp, synthetic construct
  source 1..642: mol_type = other DNA
gacatccagatgacccagagccccagcttcctgagcgccagcgtgggcgagagggtgaccatcacctgcaaggccagccaggacgtgagcaccaccgtggcctggtaccagcagaagcccggcaaggcccccaagctgctgatctactgggccagcaccaggcagaccggcgtgcccagcaggttcagcggcagcggcagcggcaccgacttcaccctgaccatcagcaacctgcagcccgaggacgtggccacctacttctgccagcagtacagcagctaccccttcaccttcggccagggcaccaagctggagatcaagcgaacggtggctgccccctccgtgttcatcttcccccccagcgatgagcagctgaagagcggcacagccagcgtggtgtgcctgctgaacaacttctaccccagggaggccaaggtgcagtggaaggtggacaacgccctgcagagcggcaacagccaggagtccgtgaccgagcaggacagcaaggacagcacctacagcctgagcagcacactgaccctgagcaaggccgactacgagaagcacaaggtgtacgcctgcgaggtgacccatcagggcctgagcagccccgtgaccaagagcttcaacaggggcgagtgc

SEQ ID NO: 172 - DNA, 642 bp, synthetic construct
  source 1..642: mol_type = other DNA
gacatccagatgacccagagccccagcagcgtgagcctgaccgtgggccagagggtgaccatcacctgcctggccaccgaggacatcttcagctacctggcctggtaccagcagaagcccggcaaggcccccaggctgctgatctacggcgccaacaggctgaaggacggcgtgcccagcaggttcagcggcagcggcagcggcacccagtacaccctgaggatcagcagcatgcagcccgaggacttcggcgtgtactactgcctgcagggcgccaagttccccctgaccttcggccccggcaccaagctggagatcaagcgaacggtggctgccccctccgtgttcatcttcccccccagcgatgagcagctgaagagcggcacagccagcgtggtgtgcctgctgaacaacttctaccccagggaggccaaggtgcagtggaaggtggacaacgccctgcagagcggcaacagccaggagtccgtgaccgagcaggacagcaaggacagcacctacagcctgagcagcacactgaccctgagcaaggccgactacgagaagcacaaggtgtacgcctgcgaggtgacccatcagggcctgagcagccccgtgaccaagagcttcaacaggggcgagtgc

SEQ ID NO: 173 - DNA, 642 bp, synthetic construct
  source 1..642: mol_type = other DNA
gacatccagatgacccagagccccagcagcctgagcgccagcatcggcgacaccgtgaccatcacctgcctggccaccgaggacatcttcagctacctggcctggtaccagcagaggcccggcaaggcccccaagctgctgatctacggcgccaacaggctgaaggacggcgtgcccagcaggttcagcggcagcggcagcggcaccgactacaccctgaccatcagcggcctgcagcccgaggacttcgtgacctactactgcctgcagggcgccaagttccccctgaccttcggccagggcaccaagctggacatgaagcgaacggtggctgccccctccgtgttcatcttcccccccagcgatgagcagctgaagagcggcacagccagcgtggtgtgcctgctgaacaacttctaccccagggaggccaaggtgcagtggaaggtggacaacgccctgcagagcggcaacagccaggagtccgtgaccgagcaggacagcaaggacagcacctacagcctgagcagcacactgaccctgagcaaggccgactacgagaagcacaaggtgtacgcctgcgaggtgacccatcagggcctgagcagccccgtgaccaagagcttcaacaggggcgagtgc

SEQ ID NO: 174 - DNA, 642 bp, synthetic construct
  source 1..642: mol_type = other DNA
gacatccagatgacccagagccccagcagcctgagcgccagcgtgggcgacaccgtgaccatcacctgcctggccaccgaggacatcttcagctacctggcctggtaccagcagaagcccggcaaggcccccaagctgctgatctacggcgccaacaggctgaaggacggcgtgcccagcaggttcagcggcagcggcagcggcaccgactacaccctgaccatcagcggcctgcagcccgaggacttcggcacctactactgcctgcagggcgccaagttccccctgaccttcggccagggcaccaagctggagatcaagcgaacggtggctgccccctccgtgttcatcttcccccccagcgatgagcagctgaagagcggcacagccagcgtggtgtgcctgctgaacaacttctaccccagggaggccaaggtgcagtggaaggtggacaacgccctgcagagcggcaacagccaggagtccgtgaccgagcaggacagcaaggacagcacctacagcctgagcagcacactgaccctgagcaaggccgactacgagaagcacaaggtgtacgcctgcgaggtgacccatcagggcctgagcagccccgtgaccaagagcttcaacaggggcgagtgc

SEQ ID NO: 175 - DNA, 642 bp, synthetic construct
  source 1..642: mol_type = other DNA
gacatccagatgacccagagccccagcagcctgagcgccagcgtgggcgacaccgtgaccatcacctgcctggccaccgaggacatcttcagctacctggcctggtaccagcagaagcccggcaaggcccccaagctgctgatctacggcgccaacaggctgaaggacggcgtgcccagcaggttcagcggcggcggcagcggcaccgagtacagcctgaccatcagcggcctgcagcccgaggacttcggcacctactactgcctgcagggcgccaagttccccctgaccttcggccagggcaccaagctggacatgaagcgaacggtggctgccccctccgtgttcatcttcccccccagcgatgagcagctgaagagcggcacagccagcgtggtgtgcctgctgaacaacttctaccccagggaggccaaggtgcagtggaaggtggacaacgccctgcagagcggcaacagccaggagtccgtgaccgagcaggacagcaaggacagcacctacagcctgagcagcacactgaccctgagcaaggccgactacgagaagcacaaggtgtacgcctgcgaggtgacccatcagggcctgagcagccccgtgaccaagagcttcaacaggggcgagtgc

SEQ ID NO: 176 - DNA, 642 bp, synthetic construct
  source 1..642: mol_type = other DNA
gacatccagatgacccagagccccagcagcctgagcgccagcgtgggcgacaccgtgaccatcacctgcctggccaccgaggacatcttcagctacctggcctggtaccagcagaagcccggcaaggcccccaagctgctgatctacggcgccaacaggctgaaggacaccgtgcccagcaggttcagcggcggcggcagcggcaccgagtacagcctgaccatcagcggcctgcagcccgaggacttcggcacctactactgcctgcagggcgccaagttccccctgaccttcggccagggcaccaagctggacatgaagcgaacggtggctgccccctccgtgttcatcttcccccccagcgatgagcagctgaagagcggcacagccagcgtggtgtgcctgctgaacaacttctaccccagggaggccaaggtgcagtggaaggtggacaacgccctgcagagcggcaacagccaggagtccgtgaccgagcaggacagcaaggacagcacctacagcctgagcagcacactgaccctgagcaaggccgactacgagaagcacaaggtgtacgcctgcgaggtgacccatcagggcctgagcagccccgtgaccaagagcttcaacaggggcgagtgc

SEQ ID NO: 177 - DNA, 1356 bp, synthetic construct
  source 1..1356: mol_type = other DNA
caggtgcagctgctggagagcggcggcgagctggtgcagcccggcggcagcctgaggctgagctgcgccgccagcggcttcaccttcagcgactactacatgttctggatcaggcagaccccccagaagaggctggagtgggtggcctacatcagcaacggcggcggcaacacctactaccccgacaccgtgaagggcaggttcaccatcagcagggacaacgccaagaaccagctgtacctgcagatgaggagcctgacccccgaggacaccgccatctactactgcgccagccccgaggccaggtactacggcaactaccccttcccctactggggccagggcaccctggtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 178 - DNA, 1356 bp, synthetic construct
  source 1..1356: mol_type = other DNA
caggtgcagctggtggagagcggcggcggcctggtgaagcccggcggcagcctgaggctgagctgcgccgccagcggcttcaccttcagcgactactacatgttctggatcaggcaggcccccggcaagggcctggagtgggtggcctacatcagcaacggcggcggcaacacctactaccccgacaccgtgaagggcaggttcaccatcagcagggacaacgccaagaacaccctgtacctgcagatgaacagcctgagggccgaggacaccgccgtgtactactgcgccagccccgaggccaggtactacggcaactaccccttcccctactggggccagggcaccctggtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 179 - DNA, 1356 bp, synthetic construct
  source 1..1356: mol_type = other DNA
caggtgcagctggtggagagcggcggcggcgtggtgaagcccggcggcagcctgaggctgagctgcgccgccagcggcttcaccttcagcgactactacatgttctggatcaggcaggcccccggcaagggcctggagtgggtggcctacatcagcaacggcggcggcaacacctactaccccgacaccgtgaagggcaggttcaccatcagcagggacaacgccaagaacaccctgtacctgcagatgaacagcctgagggccgaggacaccgccgtgtactactgcgccagccccgaggccaggtactacggcaactaccccttcccctactggggccagggcaccctggtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 180 - DNA, 1356 bp, synthetic construct
  source 1..1356: mol_type = other DNA
caggtgcagctggtggagagcggcggcggcgtggtgcagcccggcggcagcctgaggctgagctgcgccgccagcggcttcaccttcagcgactactacatgttctggatcaggcaggcccccggcaagggcctggagtgggtggcctacatcagcaacggcggcggcaacacctactaccccgacaccgtgaagggcaggttcaccatcagcagggacaacgccaagaacaccctgtacctgcagatgaacagcctgagggccgaggacaccgccgtgtactactgcgccagccccgaggccaggtactacggcaactaccccttcccctactggggccagggcaccctggtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 181 - DNA, 1338 bp, synthetic construct
  source 1..1338: mol_type = other DNA
caggtgcagctgctgcagagcggccccggcctggtgaagcccagcgccaccctgagcctgacctgcaccgtgagcggcttcagcctgaccacctacggcgtgcactggatcaggcagccccccggcaagggcctggagtggatgggcgtgatctggagcggcggcagcaccgactacaacgccgccttcatcagcaggctgaccatcagcaaggacaacagcaagaaccaggtgttcctgcagctgtacagcctgagggccgaggacaccgccatctactactgcgccaggaccagccactggtacttcgacgtgtggggcaggggcaccctggtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 182 - DNA, 1338 bp, synthetic construct
  source 1..1338: mol_type = other DNA
caggtgcagctgcaggagagcggccccggcctggtgaagcccagccagaccctgagcctgacctgcaccgtgagcggcttcagcctgaccacctacggcgtgcactgggtgaggcagagccccggcaagggcctggagtggctgggcgtgatctggagcggcggcagcaccgactacaacgccgccttcatcagcaggctgagcatcagcaaggacaacagcaagagccaggtgttcctggagatgaccagcctgaccgccgccgacaccgccatctactactgcgccaggaccagccactggtacttcgacgtgtggggcaggggcaccctggtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 183 - DNA, 1338 bp, synthetic construct
  source 1..1338: mol_type = other DNA
caggtgcagctgcaggagagcggccccggcctggtgaagcccagccagaccctgagcctgacctgcaccgtgagcggcttcagcctgaccacctacggcgtgcactgggtgaggcaggcccccgagaagggcctggagtggctgggcgtgatctggagcggcggcagcaccgactacaacgccgccttcatcagcaggctgagcatcagcaaggacaacagcaagagccaggtgttcctggagatgaccagcctgaccgccgacgacaccgccatctactactgcgccaggaccagccactggtacttcgacgtgtggggcaggggcaccctggtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 184 - DNA, 1338 bp, synthetic construct
  source 1..1338: mol_type = other DNA
caggtgcagctgcaggagagcggccccggcctggtgaagcccagccagaccctgagcctgacctgcaccgtgagcggcttcagcctgaccacctacggcgtgcactgggtgaggcagacccccgagaagggcctggagtggctgggcgtgatctggagcggcggcagcaccgactacaacgccgccttcatcagcaggctgagcatcagcaaggacaacagcaagagccaggtgttcctggagatgaccagcctgaccgccgacgacaccgccatctactactgcgccaggaccagccactggtacttcgacgtgtggggcaggggcaccctggtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 185 - DNA, 1356 bp, synthetic construct
  source 1..1356: mol_type = other DNA
caggtgcagctgctggagagcggcggcggcctggtgcagcccggcggcagcctgaggctgagctgcgccgccagcggcttcaccttcagcgactactacatgtactggatcaggcagaccccccagaagaggctggagtgggtggcctacatcagcaacggcggcggcaacacctactacagcgacaccgtgaagggcaggttcaccatcagcagggacaacgccaagaaccagctgtacctgcagatgaggagcctgaggcccgaggacaccgccatctactactgcgccagccccgaggccaggtactacggcaacttccccttcccctactggggccagggcaccctggtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 186 - DNA, 1356 bp, synthetic construct
  source 1..1356: mol_type = other DNA
caggtgcagctggtggagagcggcggcggcctggtgaagcccggcggcagcctgaggctgagctgcgccgccagcggcttcaccttcagcgactactacatgtactggatcaggcaggcccccggcaagggcctggagtgggtggcctacatcagcaacggcggcggcaacacctactacagcgacaccgtgaagggcaggttcaccatcagcagggacaacgccaagaacaccctgtacctgcagatgaacagcctgagggccgaggacaccgccgtgtactactgcgccagccccgaggccaggtactacggcaacttccccttcccctactggggccagggcaccctggtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 187 - DNA, 1356 bp, synthetic construct; identical to SEQ ID NO: 186
  source 1..1356: mol_type = other DNA

SEQ ID NO: 188 - DNA, 1356 bp, synthetic construct; identical to SEQ ID NO: 186
  source 1..1356: mol_type = other DNA

SEQ ID NO: 189 - DNA, 1344 bp, synthetic construct
  source 1..1344: mol_type = other DNA
caggtgcagctggtggagagcggcggcggcgtggtgcagcccggcaggagcctgaggctgagctgcgccgccagcggcttcagcttcagcaactactacatggcctgggtgaggcaggcccccggcaagggcctggagtgggtggccagcatcagcaccggcggcggcaacatctactacagggacagcgtgaagggcaggttcaccatcagcagggacaacagcaagagcaccctgtacctgcagatgaggagcctgaggagcgaggacaccgccatctactactgcgccaggcagaccgcctactacgtgatggacgcctggggccagggcaccatggtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 190 - DNA, 1344 bp, synthetic construct
  source 1..1344: mol_type = other DNA
gaggtgcagctggtggagagcggcggcggcctggtgcagcccggcggcagcctgaggctgagctgcgccgccagcggcttcagcttcagcaactactacatggcctgggtgaggcaggcccccggcaagggcctggagtgggtggccagcatcagcaccggcggcggcaacatctactacagggacagcgtgaagggcaggttcaccatcagcagggacaacgccaagaacagcctgtacctgcagatgaacagcctgagggccgaggacaccgccgtgtactactgcgccaggcagaccgcctactacgtgatggacgcctggggccagggcaccaccgtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 191 - DNA, 1344 bp, synthetic construct; identical to SEQ ID NO: 190
  source 1..1344: mol_type = other DNA

SEQ ID NO: 192 - DNA, 1344 bp, synthetic construct; identical to SEQ ID NO: 190
  source 1..1344: mol_type = other DNA

SEQ ID NO: 193 - DNA, 768 bp, synthetic construct
  source 1..768: mol_type = other DNA
ggaggatggtcctgcgcccccgacgaggacacctggctgtgccccgccggcggaggaggctctggaggactgtctggcaggtccgacgccggctcccctctgggcctggctggcagcggcggctctgacatccagatgacacagtccccatccagcctgtccgcctccgtgggcgacaccgtgacaatcacctgtctggccaccgaggacatcttctcctacctggcttggtatcagcagaagccaggcaaggcccccaagctgctgatctacggcgctaataggctgaaggacacagtgccatcccggttcagcggaggaggctccggcacagagtattctctgaccatctccggcctgcagcctgaggattttggcacctactattgcctgcagggcgccaagttcccactgacatttggccagggcaccaagctggacatgaagagaacagtggccgctccctccgtgttcatctttccccctagcgatgagcagctgaagagcggcaccgcttctgtggtgtgcctgctgaacaatttctaccctcgcgaggccaaggtgcagtggaaggtggacaacgctctgcagtctggcaattcccaggagagcgtgacagagcaggactctaaggattccacctatagcctgtcttccacactgaccctgtctaaggccgattacgagaagcacaaggtgtatgcttgcgaggtgacacatcagggcctgtcctcccccgtgaccaagtcctttaacagaggcgagtgt

SEQ ID NO: 194 - DNA, 1956 bp, synthetic construct
  source 1..1956: mol_type = other DNA
gaggtgcagctggtggagtccggaggaggactggtgcagcctggcggctccctgagactgagctgtgccgcttctggctccacattctacaccgccgtgatgggatgggtgaggcaggctccaggcaagggcctggagtgggtggctgctatcaggtggacagccctgaccacatcttatgctgactccgtgaagggcagattcaccatctcccgcgatggcgccaagaccacactgtacctgcagatgaacagcctgagacctgaggacacagccgtgtactattgcgctgctcgcggcaccctgggactgtttaccacagctgactcctacgattattggggccagggcacactggtgaccgtgtccagcggcggaggaggcagcggaggaggctctggcggcgtgtactgcggcccagagttcgacgagtccgtgggctgtatgggcggcggaggcagcggaggaggactgtccggcagaagcgatgctggctccccactgggcctggctggctccggaggaggcagcgaagtccagctggtggagagcggcggcggcctggtccagcctggcggctctctgaagctgtcctgtgccgcctccggcttcacctttaacaagtatgccatgaattgggtgcgccaggctcccggcaagggcctggagtgggtagccaggatcaggtccaagtacaacaattatgccacctactacgccgactccgtgaaggataggttcacaatctctcgggacgattccaagaacaccgcctacctgcagatgaacaatctgaagacagaggacaccgccgtgtactattgcgtgaggcacggcaactttggcaattcttacatctcctattgggcttactggggtcagggcacactggtcaccgtgtcttccggaggaggaggctccggcggcggaggcagcggcggcggcggctctcagacagtggtgacccaggagccaagcctgaccgtgtctcccggcggcaccgtgacactgacctgtggcagctctacaggagctgtgaccagcggaaactatccaaattgggtgcagcagaagcctggccaggctcctagaggcctgatcggaggcacaaagttcctggccccaggcaccccagctcgctttagcggctctctgctgggaggcaaggccgctctgaccctgagcggagtgcagccagaggatgaggccgagtactattgcgtgctgtggtactctaacagatgggtgtttggcggcggcacaaagctgaccgtgctgggaggaggaggcagcgaagtgcagctggtcgagtctggcggcggcttagtccaacctggcggctccctgaggctgtcttgcgccgcttctggcttctcctttagcaactactatatggcttgggtgcggcaggctcctggcaagggcctggagtgggtcgcctctatctccacaggcggcggcaatatctactatcgggactctgtgaagggcaggttcaccatctccagggacaacgctaagaatagcctgtatctgcagatgaactccctgagggccgaagatactgccgtgtactactgcgcccggcagaccgcttactatgtgatggatgcctggggccagggcaccacagtgacagtgtccagcgcctccaccaagggccctagcgtgttccctctggctccatcttccaagagcacatctggaggcaccgccgctctgggatgtctggtgaaggactacttccccgagcctgtgaccgtgagctggaactctggcgccctgacatctggcgtgcacacctttcccgctgtgctgcagtcctccggcctgtattccctgtccagcgtggtgacagtgccttcttccagcctgggcacacagacctacatctgcaacgtgaatcataagcctagcaataccaaggtggataagaaggtggagccaaagtcttgt